SEQUENCE LISTING (257 sequences)

SEQ ID NO: 1 - DNA, 608 bp, Bacillus subtilis
  source 1..608: mol_type = unassigned DNA
gaattctccattttcttctgctatcaaaataacagactcgtgattttccaaacgagctttcaaaaaagcctctgccccttgcaaatcggatgcctgtctataaaattcccgatattggttaaacagcggcgcaatggcggccgcatctgatgtctttgcttggcgaatgttcatcttatttcttcctccctctcaataattttttcattctatcccttttctgtaaagtttatttttcagaatacttttatcatcatgctttgaaaaaatatcacgataatatccattgttctcacggaagcacacgcaggtcatttgaacgaattttttcgacaggaatttgccgggactcaggagcatttaacctaaaaaagcatgacatttcagcataatgaacatttactcatgtctattttcgttcttttctgtatgaaaatagttatttcgagtctctacggaaatagcgagagatgatatacctaaatagagataaaatcatctcaaaaaaatgggtctactaaaatattattccatctattacaataaattcacagaatagtcttttaagtaagtctactctgaatttttttaaaaggagagggtaaaga

SEQ ID NO: 2 - DNA, 87 bp, Bacillus subtilis
  source 1..87: mol_type = unassigned DNA
gtgagaagcaaaaaattgtggatcagcttgttgtttgcgttaacgttaatctttacgatggcgttcagcaacatgtctgctagcgca

SEQ ID NO: 3 - protein, 29 aa, Bacillus subtilis
MRSKKLWISLLFALTLIFTMAFSNMSASA

SEQ ID NO: 4 - DNA, 261 bp, Bacillus gibsonii
  source 1..261: mol_type = unassigned DNA
gcagaagaaaaagtaaaatacttaataggtttcgaagaagaagcagaacttgaagccttcactgaggaaattgaccaagttggtgtattttctgttgaagaacaaagtgtagctgaggatacgttagatattgatgtagacattattgatgaatatgattatattgatgtgttagctgtagaattagatcctgaggatgtagatgcgttaagtgaagaagcaggtatctcatttattgaagaagacattgaactgtctatt

SEQ ID NO: 5 - DNA, 252 bp, Bacillus lentus
  source 1..252: mol_type = unassigned DNA
gctgaagaagcaaaagaaaaatatttaattggctttaatgagcaggaagctgtcagtgagtttgtagaacaagtagaggcaaatgacgaggtcgccattctctctgaggaagaggaagtcgaaattgaattgcttcatgaatttgaaacgattcctgttttatccgttgagttaagcccagaagatgtggacgcgcttgagctcgatccagcgatttcttatattgaagaggatgcagaagtaacgacaatg

SEQ ID NO: 6 - DNA, 807 bp, Bacillus gibsonii
  source 1..807: mol_type = unassigned DNA
caacaaacagtgccatggggaattactcgtgtgcaagccccagctgttcataaccgtggaattacaggttctggtgtaagagttgctatcctcgattcaggtatttccacacatgaagacttaaatgttcgtggtggcgttagctttgtaccaggggaaccaacgactgctgatttaaatgggcatggcacgcatgtggctgggacggtagctgctttaaacaattcgattggcgttgttggcgtagcaccgtcagcggatctatacgctgttaaagtattaggggcgaatggtagaggttcggtcagcgggattgcccaaggattggaatgggcagcagcaaataacatgcacattgctaatatgagtttaggaagcgatgcaccaagttctacacttgagcgtgctgttaattatgcgacttctagagatgttcttgttattgcggcaactgggaataacggttctggctcagtaggctatccggcccgttatgcgaacgcaatggcagtcggagctactgaccaaaacaacagacgcgccaacttttcacagtatggcacggggattgacattgtcgcaccaggtgtaaacgtgcagagcacatacccaggtaaccgttatgtgagcatgaacggtacatcgatggctactcctcatgttgcaggtgcagcagcccttgttaaacaacgctatccatcttggaatgcgactcaaatccgcaatcatctaaagaatacggcaacgaatttaggaaactcttcacaatttggaagcggacttgtcaatgcagaagcggcaacacgc

SEQ ID NO: 7 - DNA, 42 bp, synthetic construct
  misc_feature 1..42: note = primer
  source 1..42: mol_type = other DNA
gaggatgcagaagtaacgacaatgcaacaaacagtgccatgg

SEQ ID NO: 8 - DNA, 52 bp, synthetic construct
  misc_feature 1..52: note = primer
  source 1..52: mol_type = other DNA
ccaaggccggttttttatgtatctagattagcgtgttgccgcttctgcattg

SEQ ID NO: 9 - DNA, 42 bp, synthetic construct
  misc_feature 1..42: note = primer
  source 1..42: mol_type = other DNA
gaagaagacattgaactgtctattcaacaaacagtgccatgg

SEQ ID NO: 10 - DNA, 52 bp, synthetic construct
  misc_feature 1..52: note = primer
  source 1..52: mol_type = other DNA
caatgcagaagcggcaacacgctaatctagatacataaaaaaccggccttgg

SEQ ID NO: 11 - DNA, 42 bp, synthetic construct
  misc_feature 1..42: note = primer
  source 1..42: mol_type = other DNA
ccatggcactgtttgttgcattgtcgttacttctgcatcctc

SEQ ID NO: 12 - DNA, 42 bp, synthetic construct
  misc_feature 1..42: note = primer
  source 1..42: mol_type = other DNA
ccatggcactgtttgttgaatagacagttcaatgtcttcttc

SEQ ID NO: 13 - DNA, 93 bp, synthetic construct
  misc_feature 1..93: note = terminator
  source 1..93: mol_type = other DNA
ggttaccttgaatgtatataaacattctcaaagggatttctaataaaaaacgctcggttgccgccgggcgttttttatgcatcgatggaattc

SEQ ID NO: 14 - DNA, 21 bp, synthetic construct
  misc_feature 1..21: note = Bacillus gibsonii-clade subtilisin
  source 1..21: mol_type = other DNA
ggatcctgactgcctgagctt

SEQ ID NO: 15 - DNA, 1155 bp, synthetic construct
  misc_feature 1..1155: note = Bacillus gibsonii-clade subtilisin
  source 1..1155: mol_type = other DNA
atgagaagcaaaaaattgtggatcagcttgttgtttgcgttaacgttaatctttacgatggcgttcagcaacatgtctgctagcgcagcagaagaaaaagtaaaatacttaataggtttcgaagaagaagcagaacttgaagccttcactgaggaaattgaccaagttggtgtattttctgttgaagaacaaagtgtagctgaggatacgttagatattgatgtagacattattgatgaatatgattatattgatgtgttagctgtagaattagatcctgaggatgtagatgcgttaagtgaagaagcaggtatctcatttattgaagaagacattgaactgtctattcaacaaacagtgccatggggaattactcgtgtgcaagccccagctgttcataaccgtggaattacaggttctggtgtaagagttgctatcctcgattcaggtatttccacacatgaagacttaaatgttcgtggtggcgttagctttgtaccaggggaaccaacgactgctgatttaaatgggcatggcacgcatgtggctgggacggtagctgctttaaacaattcgattggcgttgttggcgtagcaccgtcagcggatctatacgctgttaaagtattaggggcgaatggtagaggttcggtcagcgggattgcccaaggattggaatgggcagcagcaaataacatgcacattgctaatatgagtttaggaagcgatgcaccaagttctacacttgagcgtgctgttaattatgcgacttctagagatgttcttgttattgcggcaactgggaataacggttctggctcagtaggctatccggcccgttatgcgaacgcaatggcagtcggagctactgaccaaaacaacagacgcgccaacttttcacagtatggcacggggattgacattgtcgcaccaggtgtaaacgtgcagagcacatacccaggtaaccgttatgtgagcatgaacggtacatcgatggctactcctcatgttgcaggtgcagcagcccttgttaaacaacgctatccatcttggaatgcgactcaaatccgcaatcatctaaagaatacggcaacgaatttaggaaactcttcacaatttggaagcggacttgtcaatgcagaagcggcaacacgc

SEQ ID NO: 16 - protein, 385 aa, synthetic construct
  REGION 1..385: note = Bacillus gibsonii-clade subtilisin
MRSKKLWISLLFALTLIFTMAFSNMSASAAEEKVKYLIGFEEEAELEAFTEEIDQVGVFSVEEQSVAEDTLDIDVDIIDEYDYIDVLAVELDPEDVDALSEEAGISFIEEDIELSIQQTVPWGITRVQAPAVHNRGITGSGVRVAILDSGISTHEDLNVRGGVSFVPGEPTTADLNGHGTHVAGTVAALNNSIGVVGVAPSADLYAVKVLGANGRGSVSGIAQGLEWAAANNMHIANMSLGSDAPSSTLERAVNYATSRDVLVIAATGNNGSGSVGYPARYANAMAVGATDQNNRRANFSQYGTGIDIVAPGVNVQSTYPGNRYVSMNGTSMATPHVAGAAALVKQRYPSWNATQIRNHLKNTATNLGNSSQFGSGLVNAEAATR

SEQ ID NO: 17 - protein, 356 aa, synthetic construct
  REGION 1..356: note = Bacillus gibsonii-clade subtilisin
AEEKVKYLIGFEEEAELEAFTEEIDQVGVFSVEEQSVAEDTLDIDVDIIDEYDYIDVLAVELDPEDVDALSEEAGISFIEEDIELSIQQTVPWGITRVQAPAVHNRGITGSGVRVAILDSGISTHEDLNVRGGVSFVPGEPTTADLNGHGTHVAGTVAALNNSIGVVGVAPSADLYAVKVLGANGRGSVSGIAQGLEWAAANNMHIANMSLGSDAPSSTLERAVNYATSRDVLVIAATGNNGSGSVGYPARYANAMAVGATDQNNRRANFSQYGTGIDIVAPGVNVQSTYPGNRYVSMNGTSMATPHVAGAAALVKQRYPSWNATQIRNHLKNTATNLGNSSQFGSGLVNAEAATR

SEQ ID NO: 18 - protein, 269 aa, synthetic construct
  REGION 1..269: note = Bacillus gibsonii-clade subtilisin
QQTVPWGITRVQAPAVHNRGITGSGVRVAILDSGISTHEDLNVRGGVSFVPGEPTTADLNGHGTHVAGTVAALNNSIGVVGVAPSADLYAVKVLGANGRGSVSGIAQGLEWAAANNMHIANMSLGSDAPSSTLERAVNYATSRDVLVIAATGNNGSGSVGYPARYANAMAVGATDQNNRRANFSQYGTGIDIVAPGVNVQSTYPGNRYVSMNGTSMATPHVAGAAALVKQRYPSWNATQIRNHLKNTATNLGNSSQFGSGLVNAEAATR

SEQ ID NO: 19 - protein, 268 aa, Bacillus gibsonii
QTVPWGITRVQAPAVHNRGITGSGVRVAILDSGISAHSDLNIRGGASFVPGEPTTADLNGHGTHVAGTVAALNNSIGVIGVAPNAELYAVKVLGANGSGSVSGIAQGLEWAATNNMHIANMSLGSDFPSSTLERAVNYATSRDVLVIAATGNNGSGSVGYPARYANAMAVGATDQNNRRANFSQYGTGIDIVAPGVNVQSTYPGNRYVSMNGTSMATPHVAGAAALVKQRYPSWNATQIRNHLKNTATNLGNSSQFGSGLVNAEAATR

SEQ ID NO: 20 - protein, 268 aa, synthetic construct
  REGION 1..268: note = Bacillus gibsonii-clade subtilisin
QTVPWGITRVQAPAVHNRGITGSGVRVAILDSGISTHEDLNVRGGVSFVPGEPTTADLNGHGTHVAGTVAALNNSIGVVGVAPSADLYAVKVLGANGRGSVSGIAQGLEWAAANNMHIANMSLGSDAPSSTLERAVNYATSRDVLVIAATGNNGSGSVGYPARYANAMAVGATDQNNRRANFSQYGTGIDIVAPGVNVQSTYPGNRYVSMNGTSMATPHVAGAAALVKQRYPSWNATQIRNHLKNTATNLGNSSQFGSGLVNAEAATR

SEQ ID NO: 21 - DNA, 807 bp, synthetic construct
  misc_feature 1..807: note = Bacillus gibsonii-clade subtilisin
  source 1..807: mol_type = other DNA
caacaaacagtgccatggggaattactcgtgtgcaagccccagctgttcataaccgtggaattacaggttctggtgtaagagttgctatcctcgattcaggtatttccacacatgaagacttaaatattcgtggtggcgttagctttgtaccaggggaaccaacgactgctgatttaaatgggcatggcacgcatgtggctgggacggtagctgctttagacaattcgattggcgttgttggcgtagcaccgtcagcggatctatacgctgttaaagtattaggggcgaatggtagaggttcgattagcgggattgcccaaggattggaatgggcagcacaaaataacatgcacattgctaatatgagtttaggaacagatgcaccaagttctacacttgagcgtgctgttaattatgcgacttctagagatgttcttgttattgcggcaactgggaataacggttctggcacagtaggctatccggcccgttatgcgaacgcaatggcagtcggagctactgaccaaaacaacagacgcgccaacttttcacagtatggcacggggattgacattgtcgcaccaggtgtaaacgtgcagagcacatacccaggtaaccgttatgtgagcatgaacggtacatcgatggctactcctcatgttgcaggtgcagcagcccttgttaaacaacgctatccatcttggaatgcgactcaaatccgcgaccatctaaagaatacggcaacgaatttaggaaactcttcacaatttggaagcggacttgtcaatgcagaagcggcaacacgc

SEQ ID NO: 22 - protein, 269 aa, synthetic construct
  REGION 1..269: note = Bacillus gibsonii-clade subtilisin
QQTVPWGITRVQAPAVHNRGITGSGVRVAILDSGISTHEDLNIRGGVSFVPGEPTTADLNGHGTHVAGTVAALDNSIGVVGVAPSADLYAVKVLGANGRGSISGIAQGLEWAAQNNMHIANMSLGTDAPSSTLERAVNYATSRDVLVIAATGNNGSGTVGYPARYANAMAVGATDQNNRRANFSQYGTGIDIVAPGVNVQSTYPGNRYVSMNGTSMATPHVAGAAALVKQRYPSWNATQIRDHLKNTATNLGNSSQFGSGLVNAEAATR

SEQ ID NO: 23 - DNA, 807 bp, synthetic construct
  misc_feature 1..807: note = Bacillus gibsonii-clade subtilisin
  source 1..807: mol_type = other DNA
caacaaacagtgccatggggaattactcgtgtgcaagccccagctgttcataaccgtggaattacaggttctggtgtaagagttgctatcctcgattcaggtatttccacacatgaagacttaaatgttcgtggtggcgttagctttgtaccaggggaaccaggcactgctgatttaaatgggcatggcacgcatgtggctgggacggtagctgctttagacaattcgattggcgttgttggcgtagcaccgaacgcggaactatacgctgttaaagtattaggggcgaatggtagaggttcgattagcgggattgcccaaggattggaatgggcagcacaaaataacatgcacattgctaatatgagtttaggaacagatgcaccaagttctacacttgagcgtgctgttaattatgcgacttctagagatgttcttgttattgcggcaactgggaataacggttctggctcagtaggctatccggcccgttatgcgaacgcaatggcagtcggagctactgaccaaaacaacagacgcgccaacttttcacagtatggcacggggattgacattgtcgcaccaggtgtaaacgtgcagagcacatacccaggtaaccgttatgtgagcatgaacggtacatcgatggctactcctcatgttgcaggtgcagcagcccttgttaaacaacgctatccatcttggaatgcgactcaaatccgcaatcatctaaagaatacggcaacgaatttaggaaactcttcacaatttggaagcggacttgtcaatgcagaagcggcaacacgc

SEQ ID NO: 24 - protein, 269 aa, synthetic construct
  REGION 1..269: note = Bacillus gibsonii-clade subtilisin
QQTVPWGITRVQAPAVHNRGITGSGVRVAILDSGISTHEDLNVRGGVSFVPGEPGTADLNGHGTHVAGTVAALDNSIGVVGVAPNAELYAVKVLGANGRGSISGIAQGLEWAAQNNMHIANMSLGTDAPSSTLERAVNYATSRDVLVIAATGNNGSGSVGYPARYANAMAVGATDQNNRRANFSQYGTGIDIVAPGVNVQSTYPGNRYVSMNGTSMATPHVAGAAALVKQRYPSWNATQIRNHLKNTATNLGNSSQFGSGLVNAEAATR

SEQ ID NO: 25 - DNA, 807 bp, synthetic construct
  misc_feature 1..807: note = Bacillus gibsonii-clade subtilisin
  source 1..807: mol_type = other DNA
caacaaacagtgccatggggaattactcgtgtgcaagccccagctgttcataaccgtggaattacaggttctggtgtaagagttgctatcctcgattcaggtatttccacacatgaagacttaacaattcgtggtggcgctagctttgtaccaggggaaccaacgactgctgatttaaatgggcatggcacgcatgtggctgggacggtagctgctttagacaattcgattggcgttattggcgtagcaccgaacgcggaactatacgctgttaaagtattaggggcgaatggtagaggttcgattagcgggattgcccaaggattggaatgggcagcaaccaataacatgcacattgctaatatgagtttaggaacagatgcaccaagttctacacttgagcgtgctgttaattatgcgacttctagagatgttcttgttattgcggcaactgggaataacggttctggcacagtaggctatccggcccgttatgcgaacgcaatggcagtcggagctactgaccaaaacaacagacgcgccaacttttcacagtatggcacggggattgacattgtcgcaccaggtgtaaacgtgcagagcacatacccaggtaaccgttatgtgagcatgaacggtacatcgatggctactcctcatgttgcaggtgcagcagcccttgttaaacaacgctatccatcttggaatgcgactcaaatccgcgaccatctaaagaatacggcaacgaatttaggaaactcttcacaatttggaagcggacttgtcaatgcagaagcggcaacacgc

SEQ ID NO: 26 - protein, 269 aa, synthetic construct
  REGION 1..269: note = Bacillus gibsonii-clade subtilisin
QQTVPWGITRVQAPAVHNRGITGSGVRVAILDSGISTHEDLTIRGGASFVPGEPTTADLNGHGTHVAGTVAALDNSIGVIGVAPNAELYAVKVLGANGRGSISGIAQGLEWAATNNMHIANMSLGTDAPSSTLERAVNYATSRDVLVIAATGNNGSGTVGYPARYANAMAVGATDQNNRRANFSQYGTGIDIVAPGVNVQSTYPGNRYVSMNGTSMATPHVAGAAALVKQRYPSWNATQIRDHLKNTATNLGNSSQFGSGLVNAEAATR

SEQ ID NO: 27 - DNA, 807 bp, synthetic construct
  misc_feature 1..807: note = Bacillus gibsonii-clade subtilisin
  source 1..807: mol_type = other DNA
caacaaacagtgccatggggaattactcgtgtgcaagccccagctgttcataaccgtggaattacaggttctggtgtaagagttgctatcctcgattcaggtatttccacacatgaagacttaaatgttcgtggtggcgttagctttgtaccaggggaaccaacgactgctgatttaaatgggcatggcacgcatgtggctgggacggtagctgctttagacaattcgattggcgttgttggcgtagcaccgaacgcggaactatacgctgttaaagtattaggggcgaatggtagaggttcgattagcgggattgcccaaggattggaatgggcagcacaaaataacatgcacattgctaatatgagtttaggaacagatgcaccaagttctacacttgagcgtgctgttaattatgcgacttctagagatgttcttgttattgcggcaactgggaataacggttctggctcagtaggctatccggcccgttatgcgaacgcaatggcagtcggagctactgaccaaaacaacagacgcgccaacttttcacagtatggcacggggattgacattgtcgcaccaggtgtaaacgtgcagagcacatacccaggtaaccgttatgtgagcatgaacggtacatcgatggctactcctcatgttgcaggtgcagcagcccttgttaaacaacgctatccatcttggaatgcgactcaaatccgcgaccatctaaagaatacggcaacgaatttaggaaactcttcacaatttggaagcggacttgtcaatgcagaagcggcaacacgc

SEQ ID NO: 28 - protein, 269 aa, synthetic construct
  REGION 1..269: note = Bacillus gibsonii-clade subtilisin
QQTVPWGITRVQAPAVHNRGITGSGVRVAILDSGISTHEDLNVRGGVSFVPGEPTTADLNGHGTHVAGTVAALDNSIGVVGVAPNAELYAVKVLGANGRGSISGIAQGLEWAAQNNMHIANMSLGTDAPSSTLERAVNYATSRDVLVIAATGNNGSGSVGYPARYANAMAVGATDQNNRRANFSQYGTGIDIVAPGVNVQSTYPGNRYVSMNGTSMATPHVAGAAALVKQRYPSWNATQIRDHLKNTATNLGNSSQFGSGLVNAEAATR

SEQ ID NO: 29 - DNA, 807 bp, synthetic construct
  misc_feature 1..807: note = Bacillus gibsonii-clade subtilisin
  source 1..807: mol_type = other DNA
caacaaacagtgccatggggaattactcgtgtgcaagccccagctgttcataaccgtggaattacaggttctggtgtaaaagttgctatcctcgattcaggtatttccacacatgaagacttaaatattcgtggtggcgttagctttgtaccaggggaaccaggctatgctgatttaaatgggcatggcacgcatgtggctgggacggtagctgctttagacaattcgattggcgttattggcgtagcaccgaacgcggaactatacgctgttaaagtattaggggcgaatggtagaggttcgattagcgggattgcccaaggattggaatgggcagcaaccaataacatgcacattgctaatatgagtttaggaacagatgcaccaagttctacacttgagcgtgctgttaattatgcgacttctagagatgttcttgttattgcggcaactgggaataacggttctggctcagtaggctatccggcccgttatgcgaacgcaatggcagtcggagctactgaccaaaacaacagacgcgccaacttttcacagtatggcacggggattgacattgtcgcaccaggtgtaaacgtgcagagcacatacccaggtaaccgttatgtgagcatgaacggtacatcgatggctactcctcatgttgcaggtgcagcagcccttgttaaacaacgctatccatcttggaatgcgactcaaatccgcgaccatctaaagaatacggcaacgaatttaggaaactcttcacaatttggaagcggacttgtcaatgcagaagcggcaacacgc

SEQ ID NO: 30 - protein, 269 aa, synthetic construct
  REGION 1..269: note = Bacillus gibsonii-clade subtilisin
QQTVPWGITRVQAPAVHNRGITGSGVKVAILDSGISTHEDLNIRGGVSFVPGEPGYADLNGHGTHVAGTVAALDNSIGVIGVAPNAELYAVKVLGANGRGSISGIAQGLEWAATNNMHIANMSLGTDAPSSTLERAVNYATSRDVLVIAATGNNGSGSVGYPARYANAMAVGATDQNNRRANFSQYGTGIDIVAPGVNVQSTYPGNRYVSMNGTSMATPHVAGAAALVKQRYPSWNATQIRDHLKNTATNLGNSSQFGSGLVNAEAATR

SEQ ID NO: 31 - DNA, 807 bp, synthetic construct
  misc_feature 1..807: note = Bacillus gibsonii-clade subtilisin
  source 1..807: mol_type = other DNA
caacaaacagtgccatggggaattactcgtgtgcaagccccagctgttcataaccgtggaattacaggttctggtgtaagagttgctatcctcgactcaggtatttccacacatgaagacttaacaattcgtggtggcgttagctttgtaccaggggaaccaacgtatgctgatttaaatgggcatggcacgcatgtggctgggacggtagctgctttagacaattcgattggcgttgttggcgtagcaccgtcagcggatctatacgctgttaaagtattaggggcgaatggtagaggttcggtcagcgggattgcccaaggattggaatgggcagcacaaaataacatgcacattgctaatatgagtttaggaacagatgcaccaagttctacacttgagcgtgctgttaattatgcgacttctagagatgttcttgttattgcggcaactgggaataacggttctggctcagtaggctatccggcccgttatgcgaacgcaatggcagtcggagctactgaccaaaacaacagacgcgccaacttttcacagtatggcacggggattgacattgtcgcaccaggtgtaaacgtgcagagcacatacccaggtaaccgttatgtgagcatgaacggtacatcgatggctactcctcatgttgcaggtgcagcagcccttgttaaacaacgctatccatcttggaatgcgactcaaatccgcaatcatctaaagaatacggcaacgaatttaggaaactcttcacaatttggaagcggacttgtcaatgcagaagcggcaacacgc

SEQ ID NO: 32 - protein, 269 aa, synthetic construct
  REGION 1..269: note = Bacillus gibsonii-clade subtilisin
QQTVPWGITRVQAPAVHNRGITGSGVRVAILDSGISTHEDLTIRGGVSFVPGEPTYADLNGHGTHVAGTVAALDNSIGVVGVAPSADLYAVKVLGANGRGSVSGIAQGLEWAAQNNMHIANMSLGTDAPSSTLERAVNYATSRDVLVIAATGNNGSGSVGYPARYANAMAVGATDQNNRRANFSQYGTGIDIVAPGVNVQSTYPGNRYVSMNGTSMATPHVAGAAALVKQRYPSWNATQIRNHLKNTATNLGNSSQFGSGLVNAEAATR

SEQ ID NO: 33 - DNA, 807 bp, synthetic construct
  misc_feature 1..807: note = Bacillus gibsonii-clade subtilisin
  source 1..807: mol_type = other DNA
caacaaacagtgccatggggaattactcgtgtgcaagccccagctgttcataaccgtggaattacaggttctggtgtaagagttgctatcctcgattcaggtatttccacacatgaagacttaaatgttcgtggtggcgttagctttgtaccaggggaaccaggctatgctgatttaaatgggcatggcacgcatgtggctgggacggtagctgctttagacaattcgattggcgttgttggcgtagcaccgtcagcggatctatacgctgttaaagtattaggggcgaatggtagaggttcgattagcgggattgcccaaggattggaatgggcagcacaaaataacatgcacattgctaatatgagtttaggaacagatgcaccaagttctacacttgagcgtgctgttaattatgcgacttctagagatgttcttgttattgcggcaactgggaataacggttctggctcagtaggctatccggcccgttatgcgaacgcaatggcagtcggagctactgaccaaaacaacagacgcgccaacttttcacagtatggcacggggattgacattgtcgcaccaggtgtaaacgtgcagagcacatacccaggtaaccgttatgtgagcatgaacggtacatcgatggctactcctcatgttgcaggtgcagcagcccttgttaaacaacgctatccatcttggaatgcgactcaaatccgcgaccatctaaagaatacggcaacgaatttaggaaactcttcacaatttggaagcggacttgtcaatgcagaagcggcaacacgc

SEQ ID NO: 34 - protein, 269 aa, synthetic construct
  REGION 1..269: note = Bacillus gibsonii-clade subtilisin
QQTVPWGITRVQAPAVHNRGITGSGVRVAILDSGISTHEDLNVRGGVSFVPGEPGYADLNGHGTHVAGTVAALDNSIGVVGVAPSADLYAVKVLGANGRGSISGIAQGLEWAAQNNMHIANMSLGTDAPSSTLERAVNYATSRDVLVIAATGNNGSGSVGYPARYANAMAVGATDQNNRRANFSQYGTGIDIVAPGVNVQSTYPGNRYVSMNGTSMATPHVAGAAALVKQRYPSWNATQIRDHLKNTATNLGNSSQFGSGLVNAEAATR

SEQ ID NO: 35 - DNA, 807 bp, synthetic construct
  misc_feature 1..807: note = Bacillus gibsonii-clade subtilisin
  source 1..807: mol_type = other DNA
caacaaacagtgccatggggaattactcgtgtgcaagccccagctgttcataaccgtggaattacaggttctggtgtaagagttgctatcctcgattcaggtatttccacacatgaagacttaaatgttcgtggtggcgttagctttgtaccaggggaaccaggcactgctgatttaaatgggcatggcacgcatgtggctgggacggtagctgctttaaacaattcgattggcgttgttggcgtagcaccgtcagcggatctatacgctgttaaagtattaggggcgaatggtagaggttcgattagcgggattgcccaaggattggaatgggcagcagcaaataacatgcacattgctaatatgagtttaggaacagatgcaccaagttctacacttgagcgtgctgttaattatgcgacttctagagatgttcttgttattgcggcaactgggaataacggttctggcacagtaggctatccggcccgttatgcgaacgcaatggcagtcggagctactgaccaaaacaacagacgcgccaacttttcacagtatggcacggggattgacattgtcgcaccaggtgtaaacgtgcagagcacatacccaggtaaccgttatgtgagcatgaacggtacatcgatggctactcctcatgttgcaggtgcagcagcccttgttaaacaacgctatccatcttggaatgcgactcaaatccgcaatcatctaaagaatacggcaacgaatttaggaaactcttcacaatttggaagcggacttgtcaatgcagaagcggcaacacgc

SEQ ID NO: 36 - protein, 269 aa, synthetic construct
  REGION 1..269: note = Bacillus gibsonii-clade subtilisin
QQTVPWGITRVQAPAVHNRGITGSGVRVAILDSGISTHEDLNVRGGVSFVPGEPGTADLNGHGTHVAGTVAALNNSIGVVGVAPSADLYAVKVLGANGRGSISGIAQGLEWAAANNMHIANMSLGTDAPSSTLERAVNYATSRDVLVIAATGNNGSGTVGYPARYANAMAVGATDQNNRRANFSQYGTGIDIVAPGVNVQSTYPGNRYVSMNGTSMATPHVAGAAALVKQRYPSWNATQIRNHLKNTATNLGNSSQFGSGLVNAEAATR

SEQ ID NO: 37 - DNA, 807 bp, synthetic construct
  misc_feature 1..807: note = Bacillus gibsonii-clade subtilisin
  source 1..807: mol_type = other DNA
caacaaacagtgccatggggaattactcgtgtgcaagccccagctgttcataaccgtggaattacaggttctggtgtaagagttgctatcctcgattcaggtatttccacacatgaagacttaaatgttcgtggtggcgttagctttgtaccaggggaaccaacgactgctgatttaaatgggcatggcacgcatgtggctgggacggtagctgctttaaacaattcgattggcgttgttggcgtagcaccgtcagcggatctatacgctgttaaagtattaggggcgaatggtagaggttcggtcagcgggattgcccaaggattggaatgggcagcagcaaataacatgcacattgctaatatgagtttaggaacagatgcaccaagttctacacttgagcgtgctgttaattatgcgacttctagagatgttcttgttattgcggcaactgggaataacggttctggctcagtaggctatccggctcgttatgcgaacgcaatggcagtcggagctactgaccaaaacaacagacgcgccaacttttcacagtatggcacggggattgacattgtcgcaccaggtgtaaacgtgcagagcacatacccaggtaaccgttatgtgagcatgaacggtacatcgatggctactcctcatgttgcaggtgcagcagcccttgttaaacaacgctatccatcttggaatgcgactcaaatccgcgaccatctaaagaatacggcaacgaatttaggaaactcttcacaatttggaagcggacttgtcaatgcagaagcggcaacacgc

SEQ ID NO: 38 - protein, 269 aa, synthetic construct
  REGION 1..269: note = Bacillus gibsonii-clade subtilisin
QQTVPWGITRVQAPAVHNRGITGSGVRVAILDSGISTHEDLNVRGGVSFVPGEPTTADLNGHGTHVAGTVAALNNSIGVVGVAPSADLYAVKVLGANGRGSVSGIAQGLEWAAANNMHIANMSLGTDAPSSTLERAVNYATSRDVLVIAATGNNGSGSVGYPARYANAMAVGATDQNNRRANFSQYGTGIDIVAPGVNVQSTYPGNRYVSMNGTSMATPHVAGAAALVKQRYPSWNATQIRDHLKNTATNLGNSSQFGSGLVNAEAATR

SEQ ID NO: 39 - DNA, 807 bp, synthetic construct
  misc_feature 1..807: note = Bacillus gibsonii-clade subtilisin
  source 1..807: mol_type = other DNA
caacaaacagtgccatggggaattactcgtgtgcaagccccagctgttcataaccgtggaattacaggttctggtgtaagagttgctatcctcgattcaggtatttccacacatgaagacttaaatgttcgtggtggcgttagctttgtaccaggggaaccaacgactgctgatttaaatgggcatggcacgcatgtggctgggacggtagctgctttaaacaattcgattggcgttgttggcgtagcaccgtcagcggatctatacgctgttaaagtattaggggcgaatggtagaggttcggtcagcgggattgcccaaggattggaatgggcagcacaaaataacatgcacattgctaatatgagtttaggaacagatgcaccaagttctacacttgagcgtgctgttaattatgcgacttctgcagatgttcttgttattgcggcaactgggaataacggttctggctcagtaggctatccggcccgttatgcgaacgcaatggcagtcggagctactgaccaaaacaacagacgcgccaacttttcacagtatggcacggggattgacattgtcgcaccaggtgtaaacgtgcagagcacatacccaggtaaccgttatgtgagcatgaacggtacatcgatggctactcctcatgttgcaggtgcagcagcccttgttaaacaacgctatccatcttggaatgcgactcaaatccgcaatcatctaaagaatacggcaacgaatttaggaaactcttcacaatttggaagcggacttgtcaatgcagaagcggcaacacgc

SEQ ID NO: 40 - protein, 269 aa, synthetic construct
  REGION 1..269: note = Bacillus gibsonii-clade subtilisin
QQTVPWGITRVQAPAVHNRGITGSGVRVAILDSGISTHEDLNVRGGVSFVPGEPTTADLNGHGTHVAGTVAALNNSIGVVGVAPSADLYAVKVLGANGRGSVSGIAQGLEWAAQNNMHIANMSLGTDAPSSTLERAVNYATSADVLVIAATGNNGSGSVGYPARYANAMAVGATDQNNRRANFSQYGTGIDIVAPGVNVQSTYPGNRYVSMNGTSMATPHVAGAAALVKQRYPSWNATQIRNHLKNTATNLGNSSQFGSGLVNAEAATR

SEQ ID NO: 41 - DNA, 807 bp, synthetic construct
  misc_feature 1..807: note = Bacillus gibsonii-clade subtilisin
  source 1..807: mol_type = other DNA
caacaaacagtgccatggggaattactcgtgtgcaagccccagctgttcataaccgtggaattacaggttctggtgtaagagttgctatcctcgattcaggtatttccacacatgaagacttaaatgttcgtggtggcgttagctttgtaccaggggaaccaacgactgctgatttaaatgggcatggcacgcatgtggctgggactgtagctgctttaaacaattcgattggcgttgttggcgtagcaccgtcagcggatctatacgctgttaaagtattaggggcgaatggtagaggttcggtcagcgggattgcccaaggattggaatgggcagcacaaaataacatgcacattgctaatatgagtttaggaagcgatgcaccaagttctacacttgagcgtgctgttaattatgcgacttctagagatgttcttgttattgcggcaactgggaataacggttctggctcagtaggctatccggcccgttatgcgaacgcaatggcagtcggagctactgaccaaaacaacagacgcgccaacttttcacagtatggcacggggattgacattgtcgcaccaggtgtaaacgtgcagagcacatacccaggtaaccgttatgtgagcatgaacggtacatcgatggctactcctcatgttgcaggtgcagcagcccttgttaaacaacgctatccatcttggaatgcgactcaaatccgcgaccatctaaagaatacggcaacgaatttaggaaactcttcacaatttggaagcggacttgtcaatgcagaagcggcaacacgc

SEQ ID NO: 42 - protein, 269 aa, synthetic construct
  REGION 1..269: note = Bacillus gibsonii-clade subtilisin
QQTVPWGITRVQAPAVHNRGITGSGVRVAILDSGISTHEDLNVRGGVSFVPGEPTTADLNGHGTHVAGTVAALNNSIGVVGVAPSADLYAVKVLGANGRGSVSGIAQGLEWAAQNNMHIANMSLGSDAPSSTLERAVNYATSRDVLVIAATGNNGSGSVGYPARYANAMAVGATDQNNRRANFSQYGTGIDIVAPGVNVQSTYPGNRYVSMNGTSMATPHVAGAAALVKQRYPSWNATQIRDHLKNTATNLGNSSQFGSGLVNAEAATR

SEQ ID NO: 43 - DNA, 807 bp, synthetic construct
  misc_feature 1..807: note = Bacillus gibsonii-clade subtilisin
  source 1..807: mol_type = other DNA
caacaaacagtgccatggggaattactcgtgtgcaagccccagctgttcataaccgtggaattacaggttctggtgtaagagttgctatcctcgattcaggtatttccacacatgaagacttaaatgttcgtggtggcgttagctttgtaccaggggaaccaacgtatgctgatttaaatgggcatggcacgcatgtggctgggacggtagctgctttaaacaattcgattggcgttgttggcgtagcaccgtcagcggatctatacgctgttaaagtattaggggcgaatggtagaggttcggtcagcgggattgcccaaggattggaatgggcagcacaaaataacatgcacattgctaatatgagtttaggaacagatgcaccaagttctacacttgagcgtgctgttaattatgcgacttctagagatgttcttgttattgcggcaactgggaataacggttctggcacagtaggctatccggcccgttatgcgaacgcaatggcagtcggagctactgaccaaaacaacagacgcgccaacttttcacagtatggcacggggattgacattgtcgcaccaggtgtaaacgtgcagagcacatacccaggtaaccgttatgtgagcatgaacggtacatcgatggctactcctcatgttgcaggtgcagcagcccttgttaaacaacgctatccatcttggaatgcgactcaaatccgcgaccatctaaagaatacggcaacgaatttaggaaactcttcacaatttggaagcggacttgtcaatgcagaagcggcaacacgc

SEQ ID NO: 44 - protein, 269 aa, synthetic construct
  REGION 1..269: note = Bacillus gibsonii-clade subtilisin
QQTVPWGITRVQAPAVHNRGITGSGVRVAILDSGISTHEDLNVRGGVSFVPGEPTYADLNGHGTHVAGTVAALNNSIGVVGVAPSADLYAVKVLGANGRGSVSGIAQGLEWAAQNNMHIANMSLGTDAPSSTLERAVNYATSRDVLVIAATGNNGSGTVGYPARYANAMAVGATDQNNRRANFSQYGTGIDIVAPGVNVQSTYPGNRYVSMNGTSMATPHVAGAAALVKQRYPSWNATQIRDHLKNTATNLGNSSQFGSGLVNAEAATR

SEQ ID NO: 45 - DNA, 807 bp, synthetic construct
  misc_feature 1..807: note = Bacillus gibsonii-clade subtilisin
  source 1..807: mol_type = other DNA
caacaaacagtgccatggggaattactcgtgtgcaagccccagctgttcataaccgtggaattacaggttctggtgtaagagttgctatcctcgattcaggtatttccacacatgaagacttaacagtttcaggtggcgttagctttgtaccaggggaaccaacgactgctgatttaaatgggcatggcacgcatgtggctgggacggtagctgctttagacaattcgattggcgttgttggcgtagcaccgtcagcggatctatacgctgttaaagtattaggggcgaatggtagaggttcggtcagcgggattgcccaaggattggaatgggcagcagcaaataacatgcacattgctaatatgagtttaggaacagatgcaccaagttctacacttgagcgtgctgttaattatgcgacttctagagatgttcttgttattgcggcaactgggaataacggttctggctcagtaggctatccggcccgttatgcgaacgcaatggcagtcggagctactgaccaaaacaacagacgcgccaacttttcacagtatggcacggggattgacattgtcgcaccaggtgtaaacgtgcagagcacatacccaggtaaccgttatgtgagcatgaacggtacatcgatggctactcctcatgttgcaggtgcagcagcccttgttaaacaacgctatccatcttggaatgcgactcaaatccgcaatcatctaaagaatacggcaacgaatttaggaaactcttcacaatttggaagcggacttgtcaatgcagaagcggcaacacgc

SEQ ID NO: 46 - protein, 269 aa, synthetic construct
  REGION 1..269: note = Bacillus gibsonii-clade subtilisin
QQTVPWGITRVQAPAVHNRGITGSGVRVAILDSGISTHEDLTVSGGVSFVPGEPTTADLNGHGTHVAGTVAALDNSIGVVGVAPSADLYAVKVLGANGRGSVSGIAQGLEWAAANNMHIANMSLGTDAPSSTLERAVNYATSRDVLVIAATGNNGSGSVGYPARYANAMAVGATDQNNRRANFSQYGTGIDIVAPGVNVQSTYPGNRYVSMNGTSMATPHVAGAAALVKQRYPSWNATQIRNHLKNTATNLGNSSQFGSGLVNAEAATR

SEQ ID NO: 47-48 - skipped (2 entries)

SEQ ID NO: 49 - DNA, 807 bp, synthetic construct
  misc_feature 1..807: note = Bacillus gibsonii-clade subtilisin
  source 1..807: mol_type = other DNA
caacaaacagtgccatggggaattactcgtgtgcaagccccagctgttcataaccgtggaattacaggttctggtgtaagagttgctatcctcgattcaggtatttccacacatgaagacttaacagtttcaggtggcgttagctttgtaccaggggaaccaacgactgctgatttaaatgggcatggcacgcatgtggctgggacggtagctgctttaaacaattcgattggcgttgttggcgtagcaccgtcagcggatctatacgctgttaaagtattaggggcgaatggtagaggttcggtcagcgggattgcccaaggattggaatgggcagcagcaaataacatgcacattgctaatatgagtttaggaacagatgcaccaagttctacacttgagcgtgctgttaattatgcgacttctagagatgttcttgttattgcggcaactgggaataacggttctggctcagtaggctatccggcccgttatgcgaacgcaatggcagtcggagctactgaccaaaacaacagacgcgccaacttttcacagtatggcacggggattgacattgtcgcaccaggtgtaaacgtgcagagcacatacccaggtaaccgttatgtgagcatgaacggtacatcgatggctactcctcatgttgcaggtgcagcagcccttgttaaacaacgctatccatcttggaatgcgactcaaatccgcgaccatctaaagaatacggcaacgaatttaggaaactcttcacaatttggaagcggacttgtcaatgcagaagcggcaacacgc

SEQ ID NO: 50 - protein, 269 aa, synthetic construct
  REGION 1..269: note = Bacillus gibsonii-clade subtilisin
QQTVPWGITRVQAPAVHNRGITGSGVRVAILDSGISTHEDLTVSGGVSFVPGEPTTADLNGHGTHVAGTVAALNNSIGVVGVAPSADLYAVKVLGANGRGSVSGIAQGLEWAAANNMHIANMSLGTDAPSSTLERAVNYATSRDVLVIAATGNNGSGSVGYPARYANAMAVGATDQNNRRANFSQYGTGIDIVAPGVNVQSTYPGNRYVSMNGTSMATPHVAGAAALVKQRYPSWNATQIRDHLKNTATNLGNSSQFGSGLVNAEAATR

SEQ ID NO: 51 - DNA, 807 bp, synthetic construct
  misc_feature 1..807: note = Bacillus gibsonii-clade subtilisin
  source 1..807: mol_type = other DNA
caacaaacagtgccatggggaattactcgtgtgcaagccccagctgttcataaccgtggaattacaggttctggtgtaagagttgctatcctcgattcaggtatttccacacatgaagacttaaatgttcgtggtggcgttagctttgtaccaggggaaccaggcactgctgatttaaatgggcatggcacgcatgtggctgggacggtagctgctttaaacaattcgattggcgttgttggcgtagcaccgtcagcggatctatacgctgttaaagtattaggggcgaatggtagaggttcggtcagcgggattgcccaaggattggaatgggcagcacaaaataacatgcacattgctaatatgagtttaggaacagatgcaccaagttctacacttgagcgtgctgttaattatgcgacttctagagatgttcttgttattgcggcaactgggaataacggttctggctcagtaggctatccggcccgttatgcgaacgcaatggcagtcggagctactgaccaaaacaacagacgcgccaacttttcacagtatggcacggggattgacattgtcgcaccaggtgtaaacgtgcagagcacatacccaggtaaccgttatgtgagcatgaacggtacatcgatggctactcctcatgttgcaggtgcagcagcccttgttaaacaacgctatccatcttggaatgcgactcaaatccgcaatcatctaaagaatacggcaacgaatttaggaaactcttcacaatttggaagcggacttgtcaatgcagaagcggcaacacgc

SEQ ID NO: 52 - protein, 269 aa, synthetic construct
  REGION 1..269: note = Bacillus gibsonii-clade subtilisin
QQTVPWGITRVQAPAVHNRGITGSGVRVAILDSGISTHEDLNVRGGVSFVPGEPGTADLNGHGTHVAGTVAALNNSIGVVGVAPSADLYAVKVLGANGRGSVSGIAQGLEWAAQNNMHIANMSLGTDAPSSTLERAVNYATSRDVLVIAATGNNGSGSVGYPARYANAMAVGATDQNNRRANFSQYGTGIDIVAPGVNVQSTYPGNRYVSMNGTSMATPHVAGAAALVKQRYPSWNATQIRNHLKNTATNLGNSSQFGSGLVNAEAATR

SEQ ID NO: 53 - DNA, 807 bp, synthetic construct
  misc_feature 1..807: note = Bacillus gibsonii-clade subtilisin
  source 1..807: mol_type = other DNA
caacaaacagtgccatggggaattactcgtgtgcaagccccagctgttcataaccgtggaattacaggttctggtgtaaaagttgctatcctcgattcaggtatttccacacatgaagacttaaatgttcgtggtggcgttagctttgtaccaggggaaccaacgtatgctgatttaaatgggcatggcacgcatgtggctgggacggtagctgctttaaacaattcgattggcgttgttggcgtagcaccgtcagcggatctatacgctgttaaagtattaggggcgaatggtagaggttcggtcagcgggattgcccaaggattggaatgggcagcacaaaataacatgcacattgctaatatgagtttaggaacagatgcaccaagttctacacttgagcgtgctgttaattatgcgacttctagagatgttcttgttattgcggcaactgggaataacggttctggcacagtaagctatccggcccgttatgcgaacgcaatggcagtcggagctactgaccaaaacaacagacgcgccaacttttcacagtatggcacggggattgacattgtcgcaccaggtgtaaacgtgcagagcacatacccaggtaaccgttatgtgagcatgaacggtacatcgatggctactcctcatgttgcaggtgcagcagcccttgttaaacaacgctatccatcttggaatgcgactcaaatccgcgaccatctaaagaatacggcaacgaatttaggaaactcttcacaatatggaagcggacttgtcaatgcagaagcggcaacacgc

SEQ ID NO: 54 - protein, 269 aa, synthetic construct
  REGION 1..269: note = Bacillus gibsonii-clade subtilisin
QQTVPWGITRVQAPAVHNRGITGSGVKVAILDSGISTHEDLNVRGGVSFVPGEPTYADLNGHGTHVAGTVAALNNSIGVVGVAPSADLYAVKVLGANGRGSVSGIAQGLEWAAQNNMHIANMSLGTDAPSSTLERAVNYATSRDVLVIAATGNNGSGTVSYPARYANAMAVGATDQNNRRANFSQYGTGIDIVAPGVNVQSTYPGNRYVSMNGTSMATPHVAGAAALVKQRYPSWNATQIRDHLKNTATNLGNSSQYGSGLVNAEAATR

SEQ ID NO: 55 - DNA, 807 bp, synthetic construct
  misc_feature 1..807: note = Bacillus gibsonii-clade subtilisin
  source 1..807: mol_type = other DNA
caacaaacagtgccatggggaattactcgtgtgcaagccccagctgttcataaccgtggaattacaggttctggtgtaagagttgctatcctcgattcaggtatttccacacatgaagacttaaatgttcgtggtggcgttagctttgtaccaggggaaccaacgactgctgatttaaatgggcatggcacgcatgtggctgggacggtagctgctttagacaattcgattggcgttgttggcgtagcaccgtcagcggatctatacgctgttaaagtattaggggcgaatggtagaggttcggtcagcgggattgcccaaggattggaatgggcagcagcaaataacatgcacattgctaatatgagtttaggaacagatgcaccaagttctacacttgagcgtgctgttaattatgcgacttctagagatgttcttgttattgcggcaactgggaataacggttctggctcagtaggctatccggcccgttatgcgaacgcaatggcagtcggagctactgaccaaaacaacagacgcgccaacttttcacagtatggcacggggattgacattgtcgcaccaggtgtaaacgtgcagagcacatacccaggtaaccgttatgtgagcatgaacggtacatcgatggctactcctcatgttgcaggtgcagcagcccttgttaaacaacgctatccatcttggaatgcgactcaaatccgcgaccatctaaagaatacggcaacgaatttaggaaactcttcacaatttggaagcggacttgtcaatgcagaagcggcaacacgc

SEQ ID NO: 56 - protein, 269 aa, synthetic construct
  REGION 1..269: note = Bacillus gibsonii-clade subtilisin
QQTVPWGITRVQAPAVHNRGITGSGVRVAILDSGISTHEDLNVRGGVSFVPGEPTTADLNGHGTHVAGTVAALDNSIGVVGVAPSADLYAVKVLGANGRGSVSGIAQGLEWAAANNMHIANMSLGTDAPSSTLERAVNYATSRDVLVIAATGNNGSGSVGYPARYANAMAVGATDQNNRRANFSQYGTGIDIVAPGVNVQSTYPGNRYVSMNGTSMATPHVAGAAALVKQRYPSWNATQIRDHLKNTATNLGNSSQFGSGLVNAEAATR

SEQ ID NO: 57 - DNA, 807 bp, synthetic construct
  misc_feature 1..807: note = Bacillus gibsonii-clade subtilisin
  source 1..807: mol_type = other DNA
caacaaacagtgccatggggaattactcgtgtgcaagccccagctgttcataaccgtggaattacaggttctggtgtaaaagttgctatcctcgattcaggtatttccacacatgaagacttaaatattcgtggtggcgttagctttgtaccaggggaaccaacgtatgctgatttaaatgggcatggcacgcatgtggctgggacggtagctgctttagacaattcgattggcgttgttggcgtagcaccgtcagcggatctatacgctgttaaagtattaggggcgaatggtagaggttcggtcagcgggattgcccaaggattggaatgggcagcacaaaataacatgcacgttgctaatatgagtttaggaacagatgcaccaagttctacacttgagcgtgctgttaattatgcgacttctagagatgttcttgttattgcggcaactgggaataacggttctggctcagtaggctatccggcccgttatgcgaacgcaatggcagtcggagctactgaccaaaacaacagacgcgccaacttttcacagtatggcacggggattgacattgtcgcaccaggtgtaaacgtgcagagcacatacccaggtaaccgttatgtgagcatgaacggtacatcgatggctactcctcatgttgcaggtgcagcagcccttgttaaacaacgctatccatcttggaatgcgactcaaatccgcgaccatctaaagaatacggcaacgaatttaggaaactcttcacaatatggaagcggacttgtcaatgcagaagcggcaacacgc

SEQ ID NO: 58 - protein, 269 aa, synthetic construct
  REGION 1..269: note = Bacillus gibsonii-clade subtilisin
QQTVPWGITRVQAPAVHNRGITGSGVKVAILDSGISTHEDLNIRGGVSFVPGEPTYADLNGHGTHVAGTVAALDNSIGVVGVAPSADLYAVKVLGANGRGSVSGIAQGLEWAAQNNMHVANMSLGTDAPSSTLERAVNYATSRDVLVIAATGNNGSGSVGYPARYANAMAVGATDQNNRRANFSQYGTGIDIVAPGVNVQSTYPGNRYVSMNGTSMATPHVAGAAALVKQRYPSWNATQIRDHLKNTATNLGNSSQYGSGLVNAEAATR

SEQ ID NO: 59 - DNA, 807 bp, synthetic construct
  misc_feature 1..807: note = Bacillus gibsonii-clade subtilisin
  source 1..807: mol_type = other DNA
caacaaacagtgccatggggaattactcgtgtgcaagccccagctgttcataaccgtggaattacaggttctggtgtaaaagttgctatcctcgattcaggtatttccacacatgaagacttaaatgttcgtggtggcgttagctttgtaccaggggaaccaacgactgctgatttaaatgggcatggcacgcatgtggctgggacggtagctgctttaaacaattcgattggcgttgttggcgtagcaccgtcagcggatctatacgctgttaaagtattaggggcgaatggtagaggttcggtcagcgggattgcccaaggattggaatgggcagcagcaaataacatgcacattgctaatatgagtttaggaacagatgcaccaagttctacacttgagcgtgctgttaattatgcgacttctagagatgttcttgttattgcggcaactgggaataacggttctggctcagtaggctatccggcccgttatgcgaacgcaatggcagtcggagctactgaccaaaacaacagacgcgccaacttttcacagtatggcacggggattgacattgtcgcaccaggtgtaaacgtgcagagcacatacccaggtaaccgttatgtgagcatgaacggtacatcgatggctactcctcatgttgcaggtgcagcagcccttgttaaacaacgctatccatcttggaatgcgactcaaatccgcgaccatctaaagaatacggcaacgaatttaggaaactcttcacaatttggaagcggacttgtcaatgcagaagcggcaacacgc

SEQ ID NO: 60 - protein, 269 aa, synthetic construct
  REGION 1..269: note = Bacillus gibsonii-clade subtilisin
QQTVPWGITRVQAPAVHNRGITGSGVKVAILDSGISTHEDLNVRGGVSFVPGEPTTADLNGHGTHVAGTVAALNNSIGVVGVAPSADLYAVKVLGANGRGSVSGIAQGLEWAAANNMHIANMSLGTDAPSSTLERAVNYATSRDVLVIAATGNNGSGSVGYPARYANAMAVGATDQNNRRANFSQYGTGIDIVAPGVNVQSTYPGNRYVSMNGTSMATPHVAGAAALVKQRYPSWNATQIRDHLKNTATNLGNSSQFGSGLVNAEAATR

SEQ ID NO: 61 - DNA, 807 bp, synthetic construct
  misc_feature 1..807: note = Bacillus gibsonii-clade subtilisin
  source 1..807: mol_type = other DNA
caacaaacagtgccatggggaattactcgtgtgcaagccccagctgttcataaccgtggaattacaggttctggtgtaagagttgctatcctcgattcaggtatttccacacatgaagacttaaatgttcgtggtggcgttagctttgtaccaggggaaccaacgtatgctgatttaaatgggcatggcacgcatgtggctgggacggtagctgctttaaacaattcgattggcgttgttggcgtagcaccgtcagcggatctatacgctgttaaagtattaggggcgaatggtagaggttcggtcagcgggattgcccaaggattggaatgggcagcagcaaataacatgcacattgctaatatgagtttaggaacagatgcaccaagttctacacttgagcgtgctgttaattatgcgacttctgcagatgttcttgttattgcggcaactgggaataacggttctggctcagtaggctatccggcccgttatgcgaacgcaatggcagtcggagctactgaccaaaacaacagacgcgccaacttttcacagtatggcacggggattgacattgtcgcaccaggtgtaaacgtgcagagcacatacccaggtaaccgttatgtgagcatgaacggtacatcgatggctactcctcatgttgcaggtgcagcagcccttgttaaacaacgctatccatcttggaatgcgactcaaatccgcaatcatctaaagaatacggcaacgaatttaggaaactcttcacaatttggaagcggacttgtcaatgcagaagcggcaacacgc

SEQ ID NO: 62 - protein, 269 aa, synthetic construct
  REGION 1..269: note = Bacillus gibsonii-clade subtilisin
QQTVPWGITRVQAPAVHNRGITGSGVRVAILDSGISTHEDLNVRGGVSFVPGEPTYADLNGHGTHVAGTVAALNNSIGVVGVAPSADLYAVKVLGANGRGSVSGIAQGLEWAAANNMHIANMSLGTDAPSSTLERAVNYATSADVLVIAATGNNGSGSVGYPARYANAMAVGATDQNNRRANFSQYGTGIDIVAPGVNVQSTYPGNRYVSMNGTSMATPHVAGAAALVKQRYPSWNATQIRNHLKNTATNLGNSSQFGSGLVNAEAATR

SEQ ID NO: 63 - DNA, 807 bp, synthetic construct
  misc_feature 1..807: note = Bacillus gibsonii-clade subtilisin
  source 1..807: mol_type = other DNA
caacaaacaattccatggggaattagccgtgtgcaagccccagctgttcataaccgtggaattacaggttctggtgtaagagttgctatcctcgattcaggtatttccacacatgaagacttaaatgttcgtggtggcgttagctttgtaccaggggaaccaacgactgctgatttaaatgggcatggcacgcatgtggctgggacggtagctgctttaaacaattcgattggcgttgttggcgtagcaccgtcagcggatctatacgctgttaaagtattaggggcgaatggtagaggttcggtcagcgggattgcccaaggattggaatgggcagcagcaaataacatgcacattgctaatatgagtttaggaagcgatgcaccaagttctacacttgagcgtgctgttaattatgcgacttctagagatgttcttgttattgcggcaactgggaataacggttctggctcagtaggctatccggcccgttatgcgaacgcaatggcagtcggagctactgaccaaaacaacagacgcgccaacttttcacagtatggcacggggattgacattgtcgcaccaggtgtaaacgtgcagagcacatacccaggtaaccgttatgtgagcatgaacggtacatcgatggctactcctcatgttgcaggtgttgcagcccttgttaaacaacgctatccatcttggaatgcgactcaaatccgcaatcatctactgaatacggcaacgaatttaggaaactctaaccaatttggaagcggacttgtcaatgcagaagcggcaacacgc

SEQ ID NO: 64 - protein, 269 aa, synthetic construct
  REGION 1..269: note = Bacillus gibsonii-clade subtilisin
QQTIPWGISRVQAPAVHNRGITGSGVRVAILDSGISTHEDLNVRGGVSFVPGEPTTADLNGHGTHVAGTVAALNNSIGVVGVAPSADLYAVKVLGANGRGSVSGIAQGLEWAAANNMHIANMSLGSDAPSSTLERAVNYATSRDVLVIAATGNNGSGSVGYPARYANAMAVGATDQNNRRANFSQYGTGIDIVAPGVNVQSTYPGNRYVSMNGTSMATPHVAGVAALVKQRYPSWNATQIRNHLLNTATNLGNSNQFGSGLVNAEAATR

SEQ ID NO: 65 - DNA, 807 bp, synthetic construct
  misc_feature 1..807: note = Bacillus gibsonii-clade subtilisin
  source 1..807: mol_type = other DNA
caacaaacagtgccatggggaattactcgtgtgcaagccccagctgttcataaccgtggaattacaggttctggtgtaagagttgctatcctcgattcaggtatttccacacatgaagacttaaatgttcgtggtggcgttagctttgtaccaggggaaccaacgtatgctgatttaaatgggcatggcacgcatgtggctgggacggtagctgctttaaacaattcgattggcgttgttggcgtagcaccgtcagcggatctatacgctgttaaagtattaggggcgaatggtagaggttcggtcagcgggattgcccaaggattggaatgggcagcacaaaataacatgcacattgctaatatgagtttaggaacagatgcaccaagttctacacttgagcgtgctgttaattatgcgacttctagagatgttcttgttattgcggcaactgggaataacggttctggctcagtaggctatccggcccgttatgcgaacgcaatggcagtcggagctactgaccaaaacaacagacgcgccaacttttcacagtatggcacggggattgacattgtcgcaccaggtgtaaacgtgcagagcacatacccaggtaaccgttatgtgagcatgaacggtacatcgatggctactcctcatgttgcaggtgcagcagcccttgttaaacaacgctatccatcttggaatgcgactcaaatccgcgaccatctaaagaatacggcaacgaatttaggaaactcttcacaatttggaagcggacttgtcaatgcagaagcggcaacacgc

SEQ ID NO: 66 - protein, 269 aa, synthetic construct
  REGION 1..269: note = Bacillus gibsonii-clade subtilisin
QQTVPWGITRVQAPAVHNRGITGSGVRVAILDSGISTHEDLNVRGGVSFVPGEPTYADLNGHGTHVAGTVAALNNSIGVVGVAPSADLYAVKVLGANGRGSVSGIAQGLEWAAQNNMHIANMSLGTDAPSSTLERAVNYATSRDVLVIAATGNNGSGSVGYPARYANAMAVGATDQNNRRANFSQYGTGIDIVAPGVNVQSTYPGNRYVSMNGTSMATPHVAGAAALVKQRYPSWNATQIRDHLKNTATNLGNSSQFGSGLVNAEAATR

SEQ ID NO: 67 - DNA, 807 bp, synthetic construct
  misc_feature 1..807: note = Bacillus gibsonii-clade subtilisin
  source 1..807: mol_type = other DNA
caacaaacagtgccatggggaattactcgtgtgcaagccccagctgttcataaccgtggaattacaggttctggtgtaagagttgctatcctcgattcaggtatttccacacatgaagacttaaatgttcgtggtggcgttagctttgtaccaggggaaccaacgtatgctgatttaaatgggcatggcacgcatgtggctgggacggtagctgctttaaacaattcgattggcgttgttggcgtagcaccgtcagcggatctatacgctgttaaagtattaggggcgaatggtagaggttcggtcagcgggattgcccaaggattggaatgggcagcacaaaataacatgcacgttgctaatatgagtttaggaacagatgcaccaagttctacacttgagcgtgctgttaattatgcgacttctagagatgttcttgttattgcggcaactgggaataacggttctggctcagtaggctatccggcccgttatgcgaacgcaatggcagtcggagctactgaccaaaacaacagacgcgccaacttttcacagtatggcacggggattgacattgtcgcaccaggtgtaaacgtgcagagcacatacccaggtaaccgttatgtgagcatgaacggtacatcgatggctactcctcatgttgcaggtgcagcagcccttgttaaacaacgctatccatcttggaatgcgactcaaatccgcgaccatctaaagaatacggcaacgaatttaggaaactcttcacaatttggaagcggacttgtcaatgcagaagcggcaacacgc

SEQ ID NO: 68 - protein, 269 aa, synthetic construct
  REGION 1..269: note = Bacillus gibsonii-clade subtilisin
QQTVPWGITRVQAPAVHNRGITGSGVRVAILDSGISTHEDLNVRGGVSFVPGEPTYADLNGHGTHVAGTVAALNNSIGVVGVAPSADLYAVKVLGANGRGSVSGIAQGLEWAAQNNMHVANMSLGTDAPSSTLERAVNYATSRDVLVIAATGNNGSGSVGYPARYANAMAVGATDQNNRRANFSQYGTGIDIVAPGVNVQSTYPGNRYVSMNGTSMATPHVAGAAALVKQRYPSWNATQIRDHLKNTATNLGNSSQFGSGLVNAEAATR

SEQ ID NO: 69 - DNA, 807 bp, synthetic construct
  misc_feature 1..807: note = Bacillus gibsonii-clade subtilisin
  source 1..807: mol_type = other DNA
caacaaacagtgccatggggaattactcgtgtgcaagccccagctgttcataaccgtggaattacaggttctggtgtaagagttgctatcctcgattcaggtatttccacacatgaagacttaaatgttcgtggtggcgttagctttgtaccaggggaaccaacgactgctgatttaaatgggcatggcacgcatgtggctgggacggtagctgctttagacaattcgattggcgttgttggcgtagcaccgtcagcggatctatacgctgttaaagtattaggggcgaatggtagaggttcgattagcgggattgcccaaggattggaatgggcagcagcaaataacatgcacattgctaatatgagtttaggaagcgatgcaccaagttctacacttgagcgtgctgttaattatgcgacttctcaagatgttcttgttattgcggcaactgggaataacggttctggcacagtaggctatccggcccgttatgcgaacgcaatggcagtcggagctactgaccaaaacaacagacgcgccaacttttcacagtatggcacggggattgacattgtcgcaccaggtgtaaacgtgcagagcacatacccaggtaaccgttatgtgagcatgaacggtacatcgatggctactcctcatgttgcaggtgttgcagcccttgttaaacaacgctatccatcttggaatgcgactcaaatccgcaatcatctaaagaatacggcaacgaatttaggaaactctaaccaatttggaagcggacttgtcaatgcagaagcggcaacacgc

SEQ ID NO: 70 - protein, 269 aa, synthetic construct
  REGION 1..269: note = Bacillus gibsonii-clade subtilisin
QQTVPWGITRVQAPAVHNRGITGSGVRVAILDSGISTHEDLNVRGGVSFVPGEPTTADLNGHGTHVAGTVAALDNSIGVVGVAPSADLYAVKVLGANGRGSISGIAQGLEWAAANNMHIANMSLGSDAPSSTLERAVNYATSQDVLVIAATGNNGSGTVGYPARYANAMAVGATDQNNRRANFSQYGTGIDIVAPGVNVQSTYPGNRYVSMNGTSMATPHVAGVAALVKQRYPSWNATQIRNHLKNTATNLGNSNQFGSGLVNAEAATR

SEQ ID NO: 71 - protein, 34 aa, synthetic construct
  REGION 1..34: note = motif
  REGION 1..34: note = MISC_FEATURE - where the initial D is the active site Aspartic acid residue, the penultimate H is the active site Histidine, and X is any amino acid, and where the amino acid positions of the variant are numbered by correspondence with the amino acid sequence of SEQ ID
  SITE 2: note = MISC_FEATURE - Xaa is any amino acid
  REGION 5..6: note = MISC_FEATURE - Xaa is any amino acid
  REGION 11..13: note = MISC_FEATURE - Xaa is any amino acid
  REGION 18..22: note = MISC_FEATURE - Xaa is any amino acid
  SITE 30: note = MISC_FEATURE - Xaa is any amino acid
DXGIXXHSDLXXXGGASXXXXXPTTADLNXHGTH

SEQ ID NO: 72 - protein, 34 aa, synthetic construct
  REGION 1..34: note = motif
  REGION 1..34: note = MISC_FEATURE - where the initial D is the active site Aspartic acid residue, the penultimate H is the active site Histidine, and X is any amino acid, and where the amino acid positions of the variant are numbered by correspondence with the amino acid sequence of SEQ ID
  SITE 2: note = MISC_FEATURE - Xaa is any amino acid
  REGION 5..6: note = MISC_FEATURE - Xaa is any amino acid
  REGION 11..13: note = MISC_FEATURE - Xaa is any amino acid
  REGION 18..23: note = MISC_FEATURE - Xaa is any amino acid
  REGION 29..30: note = MISC_FEATURE - Xaa is any amino acid
DXGIXXHSDLXXXGGASXXXXXXTTADLXXHGTH

SEQ ID NO: 73 - skipped

SEQ ID NO: 74 - protein, 275 aa, Bacillus lentus [sequence omitted]

SEQ ID NO: 75 - protein, 275 aa, Geobacillus stearothermophilus
AQSVPYGVSQIKAPALHSQGFTGSNVKVAVIDSGIDSSHPDLKVAGGASMVPSETNPFQDNNSHGTHVAGTVAALNNSVGVLGVAPSASLYAVKVLGADGSGQYSWIINGIEWAIAYNMDVINMSLGGPSGSAALKAAVDKAVASGIVVVAAAGNEGTSGSSSTVGYPGKYPSVIAVGAVNSSNQRASFSSVGSELDVMAPGVSIQSTLPGNKYGAYNGTSMASPHVAGAAALILSKHPNWTNTQVRSSLENTTTKLGDAFYYGKGLINVQAAAQ

SEQ ID NO: 76 - protein, 275 aa, Bacillus subtilis
AQSVPYGISQIKAPALHSQGYTGSNVKVAVIDSGIDSSHPDLNVRGGASFVPSETNPYQDGSSHGTHVAGTVAALNNTIGVLGVAPSASLYAVKVLDSTGSGQYSWIINGIEWAISNNMDVINMSLGGPTGSTALKTVVDKAVASGIVVVAAAGNEGSSGSTSTVGYPAKYPSTIAVGAVNSSNQRASFSSAGSELDVMAPGVSIQSTLPGGTYGSYNGTSMATPHVAGAAALILSKHPTWSNAQVRDRLESTATNLGSSFYYGKGLINVQAAAQ

SEQ ID NO: 77 - protein, 275 aa, Bacillus lentus
AQSVPYGISQIKAPAVHSQGYTGSNVKVAVIDSGIDSSHPDLKVSGGASFVPSEPNPFQDGNSHGTHVAGTVAALNNSVGVLGVAPSASLYAVKVLSSSGSGDYSWIINGIEWAISNNMDVINMSLGGPQGSTALKAVVDKAVSQGIVVVAAAGNSGSSGSTSTVGYPAKYPSVIAVGAVDSNNQRASFSSAGSELDVMAPGVSIQSTLPGSSYGSYNGTSMASPHVAGAAALVLSKHPNWTNSQVRNSLESTATNLGNSFYYGKGLINVQAAAQ

SEQ ID NO: 78 - protein, 275 aa, Bacillus lentus
AQTVPYGIPQIKAPAVHAQGYKGANVKVAVLDTGIHAAHPDLNVAGGASFVPSEPNATQDFQSHGTHVAGTIAALDNTIGVLGVAPSASLYAVKVLDRNGDGQYSWIISGIEWAVANNMDVINMSLGGPNGSTALKNAVDTANNRGVVVVAAAGNSGSTGSTSTVGYPAKYDSTIAVANVNSSNVRNSSSSAGPELDVSAPGTSILSTVPSRGYTSYTGTSMASPHVAGAAALILSKNPNLSNSQVRQRLENTATPLGNSFYYGKGLINVQAASN

SEQ ID NO: 79 - protein, 275 aa, Bacillus lentus
AQTVPYGIPQIKAPAVHAQGYKGANVKVAVLDTGIHAAHPDLNVAGGASFVPSEPNATQDFQSHGTHVAGTIAALDNTIGVLGVAPSASLYAVKVLDRNGDGQYSWIISGIEWAVANNMDVINMSLGGASGSTALKNAVDTANNRGVVVVAAAGNSGSSGSRSTVGYPAKYESTIAVANVNSNNVRNSSSSAGPELDVSAPGTSILSTVPSSGYTSYTGTSMASPHVAGAAALILSKNPNLTNSQVRQRLENTATPLGDSFYYGKGLINVQAASN

SEQ ID NO: 80 - protein, 274 aa, Bacillus lentus
AQTVPYGIPLIKADKVQAQGFKGANVKVAVLDTGIQASHPDLNVVGGASFVAGEAYNTDGNGHGTHVAGTVAALDNTTGVLGVAPSVSLYAVKVLNSSGSGSYSGIVSGIEWATTNGMDVINMSLGGASGSTAMKQAVDNAYARGVVVVAAAGNSGSSGNTNTIGYPAKYDSVIAVGAVDSNSNRASFSSVGAELEVMAPGAGVYSTYPTNTYATLNGTSMASPHVAGAAALILSKHPNLSASQVRNRLSSTATYLGSSFYYGKGLINVEAAAQ

SEQ ID NO: 81 - protein, 275 aa, unidentified
  REGION 1..275: note = Bacillus sp.
AQTTPWGVTHINAHRAHSSGVTGSGVKVAILDTGIHASHPDLNVRGGASFISGESNPYIDSNGHGTHVAGTVAALNNTVGVLGVAYNAELYAVKVLSASGSGTLSGIAQGVEWSIANKMDVINMSLGGSSGSTALQRAVDNAYRNNIVVVAAAGNSGAQGNRNTIGYPARYSSVIAVGAVDSNNNRASFSSVGSELEVMAPGVSILSTVPGSSYASYNGTSMASPHVAGAAALLKAKYPNWSAAQIRNKLNSTTTYLGSSFYYGNGVINVERALQ

SEQ ID NO: 82 - protein, 275 aa, unidentified
  REGION 1..275: note = bacillus sp.
AQTVPWGIPHIKADKAHAAGVTGSGVKVAILDTGIDANHADLNVKGGASFVSGEPNALQDGNGHGTHVAGTVAALNNTTGVLGVAYNADLYAVKVLSASGSGTLSGIAQGIEWSISNGMNVINMSLGGSSGSTALQQACNNAYNRGIVVIAAAGNSGSSGNRNTMGYPARYSSVIAVGAVSSNNTRASFSSVGSELEVMAPGVNILSTTPGNNYASFNGTSMAAPHVAGAAALIKAKYPSMTNVQIRERLKNTATNLGDPFFYGKGVINVESALQ

SEQ ID NO: 83 - protein, 269 aa, unidentified
  REGION 1..269: note = bacillus sp.
SQTVPWGISFINTQQAHNRGIFGNGARVAVLDTGIASHPDLRIAGGASFISSEPSYHDNNGHGTHVAGTIAALNNSIGVLGVAPSADLYAVKVLDRNGSGSLASVAQGIEWAINNNMHIINMSLGSTSGSSTLELAVNRANNAGILLVGAAGNTGRQGVNYPARYSGVMAVAAVDQNGQRASFSTYGPEIEISAPGVNVNSTYTGNRYVSLSGTSMATPHVAGVAALVKSRYPSYTNNQIRQRINQTATYLGSPSLYGNGLVHAGRATQ

SEQ ID NO: 84 - protein, 269 aa, Bacillus lentus
AQSVPWGISRVQAPAAHNRGLTGSGVKVAVLDTGISTHPDLNIRGGASFVPGEPSTQDGNGHGTHVAGTIAALNNSIGVLGVAPSAELYAVKVLGASGSGSVSSIAQGLEWAGNNGMHVANLSLGSPSPSATLEQAVNSATSRGVLVVAASGNSGAGSISYPARYANAMAVGATDQNNNRASFSQYGAGLDIVAPGVNVQSTYPGSTYASLNGTSMATPHVAGAAALVKQKNPSWSNVQIRNHLKNTATSLGSTNLYGSGLVNAEAATR

SEQ ID NO: 85 - protein, 269 aa, Bacillus gibsonii
QQTVPWGITRVQAPAVHNRGITGSGVRVAILDSGISAHSDLNIRGGASFVPGEPTTADLNGHGTHVAGTVAALNNSIGVIGVAPNAELYAVKVLGANGSGSVSGIAQGLEWAATNNMHIANMSLGSDFPSSTLERAVNYATSRDVLVIAATGNNGSGSVGYPARYANAMAVGATDQNNRRANFSQYGTGIDIVAPGVNVQSTYPGNRYVSMNGTSMATPHVAGAAALVKQRYPSWNATQIRNHLKNTATNLGNSSQFGSGLVNAEAATR

SEQ ID NO: 86 - protein, 269 aa, Bacillus gibsonii
QQTVPWGITRVQAPAVHNRGVTGSGVRVAILDSGISTHSDLTIRGGASFVPGEPTTADLNGHGTHVAGTVAALNNSIGVIGVAPSADLYAVKVLGANGRGSVSGIAQGLEWAAANNMHIANMSLGSDAPSTTLERAVNYATSQGVLVIAATGNNGSGSVGYPARYANAMAVGATDQNNRRANFSQYGTGIDIVAPGVNVQSTYPGNRYASLNGTSMATPHVAGAAALVKQRYPSWNATQIRNHLKNTATNLGNSSQFGSGLVNAEAATR

SEQ ID NO: 87 - protein, 269 aa, Bacillus gibsonii
QQTVPWGITRVQAPAVHNRGITGSGVRVAILDSGISAHSDLNIRGGASFVPGEPTTADLNGHGTHVAGTVAALNNSIGVIGVAPNADLYAVKVLGANGSGSVSGIAQGLEWAATNNMHIANMSLGSDFPSSTLERAVNYATSRDVLVIAATGNNGSGSVGYPARYANAMAVGATDQNNRRANFSQYGTGIDIVAPGVNVQSTYPGNRYVSMNGTSMATPHVAGAAALVKQRYPSWNATQIRNHLKNTATNLGNSSQFGSGLVNAEAATR

SEQ ID NO: 88 - protein, 269 aa, Bacillus gibsonii
QQTVPWGITRVQAPAVHNRGVTGSGVRVAILDSGISTHSDLTIRGGASFVPGEPTTADLNGHGTHVAGTVAALNNSIGVIGVAPSADLYAVKVLGANGRGSVSGIAQGLEWAAANNMHIANMSLGSDAPSTTLERAVNYATSQGVLVIAATGNNGSGSVGYPARYANAMAVGATDQNNRRANFSQYGTGIDIVAPGVNVQSTYPGNRYVSMNGTSMATPHVAGAAALVKQRYPSWNATQIRNHLKNTATNLGNSSQFGSGLVNAEAATR

SEQ ID NO: 89 - protein, 269 aa, Bacillus gibsonii
QQTVPWGITRVQAPAVHNRGVTGSGVRVAILDSGISTHSDLTIRGGASFVPGEPTTADLNGHGTHVAGTVAALNNSIGVIGVAPSADLYAVKVLGANGRGSVSGIAQGLEWAAANNMHIANMSLGSDAPSTTLERAVNYATSQGVLVIAATGNNGSGSVGYPARYANAMAVGATDQNNRRANFSQYGSGIDIVAPGVNVQSTYPGNRYVSMNGTSMATPHVAGAAALVKQRYPSWNATQIRNHLKNTATNLGNSSQFGSGLVNAEAATR

SEQ ID NO: 90 - protein, 269 aa, Bacillus gibsonii
QQTVPWGITRVQAPAVHNRGVTGSGVRVAILDSGISTHSDLTIRGGASFVPGEPTTADLNGHGTHVAGTVAALNNSIGVIGVAPSADLYAVKVLGANGRGSVSGIAQGLEWAAANNMHIANMSLGSDAPSITLERAVNYATSQGVLVIAATGNNGSGSVGYPARYANAMAVGATDQNNRRANFSQYGTGIDIVAPGVNVQSTYPGNRYASLNGTSMATPHVAGAAALVKQRYPSWNATQIRNHLKNTATNLGNSSQFGSGLVNAEAATR

SEQ ID NO: 91 - protein, 269 aa, Bacillus gibsonii
QQTVPWGITRVQAPTVHNRGITGSGVKVAILDTGIAQHSDLTIRGGASFVPGESTTADLNGHGTHVAGTVAALNNSIGVIGVAPSADLYAVKVLGANGRGSVSGIAQGLEWAATNNMHIANMSLGSDAPSTTLERAVNYATSRGVLVIAATGNNGTGSIGYPARYANAMAVGATDQNNRRASFSQYGTGIDIVAPGVGIQSTYLNNSYASMPGTSMATPHVAGVAALVKQKNPSWNATQIRNHLKNTATNLGNSSQFGSGLVNADAATR

SEQ ID NO: 92 - protein, 269 aa, Bacillus gibsonii
QQTVPWGITRVQAPAVHNRGVTGSGVRVAILDSGISQHSDLTIRGGASFIPGEPTTADLNGHGTHVAGTVAALNNSIGVIGVAPSADLYAVKVLGANGRGSVSGIAQGLEWAAANNMHIANMSLGADAPSSTLERAVNYATSQGVLVIAATGNNGSGSVGYPARYANAMAVGATDQNNRRANFSQYGTGIDIVAPGVNVQSTYPGNRYASLNGTSMATPHVAGAAALVKQRNPSWNATQIRNHLKNTATNLGNSSQFGSGLVNAEAATR

SEQ ID NO: 93 - protein, 269 aa, Bacillus gibsonii
QQTVPWGITRVQAPAVHNRGVTGSGVRVAILDSGISQHSDLTIRGGASFVPGEPTTEDLNGHGTHVAGTVAALNNSFGVIGVAPSADLYAVKVLGAGGRGSVSGIAQGLEWAAANNMHIANMSLGADAPSSTLERAVNYATSRGVLVIAATGNNGSGSVGYPARYANAMAVGATDQNNRRANFSQYGTGIDIVAPGVNVQSTYPGNRYASLNGTSMATPHVAGVAALVKQRNPSWNATQIRNHLKNTATNLGNSSQFGSGLVNAEAAIR

SEQ ID NO: 94 - protein, 269 aa, synthetic construct
  REGION 1..269: note = Bacillus gibsonii-clade subtilisin
QQTVPWGITRVQAPAVHNRGVTGSGVRVAILDSGISAHSDLNIRGGASFVPGEPTTADLNGHGTHVAGTVAALNNSIGVVGVAPSADLYAVKVLGANGSGSVSGIAQGLEWAATNNIHIANMSLGSDAPSTTLERAVNYATSQGVLVIAATGNNGSGSVGYPARYANAMAVGATDQNNRRANFSQYGTGIDIVAPGVNVQSTYPGNRYVSMNGTSMATPHVAGVAALVKQRYPSWNATQIRNHLKNTATNLGNSSQFGSGLVNAEAATR

SEQ ID NO: 95 - protein, 269 aa, synthetic construct
  REGION 1..269: note = Bacillus gibsonii-clade subtilisin
QQTVPWGITRVQAPAVHNRGITGSGVRVAILDSGISAHSDLTIRGGASFVPGEPTTADLNGHGTHVAGTVAALNNSIGVVGVAPSADLYAVKVLGANGSGSVSGIAQGLEWAAANNMHIANMSLGSDAPSSTLERAVNYATSQGVLVIAATGNNGSGTISYPARYANAMAVGATDQNNRRANFSQYGTGIDIVAPGVGVQSTYPGNRYVSMNGTSMATPHVAGAAALVKQRYPSWNATQIRNHLKNTATNLGNSSQFGSGLVNAEAATR

SEQ ID NO: 96 - protein, 269 aa, synthetic construct
  REGION 1..269: note = Bacillus gibsonii-clade subtilisin
AQTVPWGITRVQAPAVHNRGITGSGVRVAILDSGISAHSDLNIRGGASFVPGEPTTADLNGHGTHVAGTVAALNNSIGVVGVAPNAELYAVKVLGANGSGSVSGIAQGLEWAATNNMHIANMSLGSDAPSTTLERAVNYATSAGVLVIAATGNNGSGSVGYPARYANALAVGATDQNNRRANFSQYGTGLDIVAPGVNVQSTYPGNRYVSMNGTSMATPHVAGAAALVKQRYPSWNATQIRNHLKNTATNLGNTNLYGSGLVNAEAATR

SEQ ID NO: 97 - protein, 269 aa, synthetic construct
  REGION 1..269: note = Bacillus gibsonii-clade subtilisin
QQTVPWGITRVQAPAVHNRGITGSGVRVAILDSGISAHSDLNIRGGASFVPGEPTTADLNGHGTHVAGTVAALDNSIGVVGVAPSADLYAVKVLGANGSGSVSGIAQGLEWAATNNMHIANMSLGSDFPSSTLERAVNYATSRDVLVIAATGNNGSGTVGYPARYANAMAVGATDQNNRRANFSQYGTGIDIVAPGVNVQSTYPGNRYVSMNGTSMATPHVAGAAALVKQRYPSWNATQIRDHLKNTATNLGNSSQFGSGLVNAEAATR

SEQ ID NO: 98 - protein, 269 aa, synthetic construct
  REGION 1..269: note = Bacillus gibsonii-clade subtilisin
QQTVPWGITRVQAPAVHNRGITGSGVRVAILDSGISAHSDLNIRGGASFVPGEPTTADLNGHGTHVAGTVAALDNSIGVVGVAPSADLYAVKVLGANGRGSISGIAQGLEWAATNNMHIANMSLGSDFPSSTLERAVNYATSRDVLVIAATGNNGSGTIGYPARYANAMAVGATDQNNRRANFSQYGTGIDIVAPGVNVQSTYPGNRYVSMNGTSMATPHVAGAAALVKQRYPSWNATQIRDHLKNTATNLGNSSQYGSGLVNAEAATR

SEQ ID NO: 99 - protein, 269 aa, synthetic construct
  REGION 1..269: note = Bacillus gibsonii-clade subtilisin
QQTVPWGITRVQAPAVHNRGITGSGVRVAILDSGISAHSDLNIRGGASFVPGEPTTADLNGHGTHVAGTVAALNNSIGVIGVAPSADLYAVKVLGANGRGSISGIAQGLEWAAQNNMHIANMSLGSDFPSSTLERAVNYATSRDVLVIAATGNNGSGTISYPARYANAMAVGATDQNNRRANFSQYGTGIDIVAPGVNVQSTYPGNRYVSMSGTSMATPHVAGAAALVKQRYPSWNATQIRDHLKNTATNLGNSSQYGSGLVNAEAATR

SEQ ID NO: 100 - protein, 269 aa, synthetic construct
  REGION 1..269: note = Bacillus gibsonii-clade subtilisin
QQTVPWGITRVQAPAVHNRGITGSGVRVAILDSGISAHSDLNIRGGASFVPGEPTTADLNGHGTHVAGTVAALDNSIGVVGVAPSADLYAVKVLGANGRGSISGIAQGLEWAATNNMHIANMSLGTDAPSSTLERAVNYATSADVLVIAATGNNGSGSVGYPARYANAMAVGATDQNNRRANFSQYGTGLDIVAPGVNVQSTYPGNRYVSMNGTSMATPHVAGAAALVKQRYPSWNATQIRDHLKNTATNLGNSSQYGSGLVNAEAATR

SEQ ID NO: 101 - protein, 269 aa, synthetic construct
  REGION 1..269: note = Bacillus gibsonii-clade subtilisin
QQTVPWGITRVQAPAVHNRGITGSGVRVAILDSGISAHSDLNIRGGASFVPGEPTTADLNGHGTHVAGTVAALDNSIGVVGVAPSADLYAVKVLGANGRGSISGIAQGLEWAAQNNMHIANMSLGSDFPSSTLERAVNYATSADVLVIAATGNNGSGSVGYPARYANAMAVGATDQNNRRANFSQYGTGLDIVAPGVNVQSTYPGNRYVSMNGTSMATPHVAGAAALVKQRYPSWNATQIRDHLKNTATNLGNSSQYGSGLVNAEAATR

SEQ ID NO: 102 - protein, 269 aa, synthetic construct
  REGION 1..269: note = Bacillus gibsonii-clade subtilisin
QQTVPWGITRVQAPAVHNRGITGSGVRVAILDSGISAHSDLNIRGGASFVPGEPTTADLNGHGTHVAGTVAALDNSIGVVGVAPSADLYAVKVLGANGRGSISGIAQGLEWAATNNMHIANMSLGSDFPSSTLERAVNYATSRDVLVIAATGNNGSGSVGYPARYANAMAVGATDQNNRRANFSQYGTGLDIVAPGVNVQSTYPGNRYVSMNGTSMATPHVAGAAALVKQRYPSWNATQIRDHLKNTATNLGNSSQYGSGLVNAEAATR

SEQ ID NO: 103 - protein, 269 aa, synthetic construct
  REGION 1..269: note = Bacillus gibsonii-clade subtilisin
QQTVPWGITRVQAPAVHNRGITGSGVRVAILDSGISAHSDLNIRGGASFVPGEPTTADLNGHGTHVAGTVAALDNSIGVIGVAPSADLYAVKVLGANGRGSISGIAQGLEWAATNNMHIANMSLGTDAPSSTLERAVNYATSRDVLVIAATGNNGSGSVGYPARYANAMAVGATDQNNRRANFSQYGTGLDIVAPGVNVQSTYPGNRYVSMNGTSMATPHVAGAAALVKQRYPSWNATQIRDHLKNTATNLGNSSQYGSGLVNAEAATR

SEQ ID NO: 104 - protein, 269 aa, synthetic construct
  REGION 1..269: note = Bacillus gibsonii-clade subtilisin
QQTVPWGITRVQAPAVHNRGITGSGVRVAILDSGISAHSDLNIRGGASFVPGEPTTADLNGHGTHVAGTVAALDNSIGVIGVAPNAELYAVKVLGANGRGSISGIAQGLEWAATNNMHIANMSLGTDAPSSTLERAVNYATSADVLVIAATGNNGSGTVGYPARYANAMAVGATDQNNRRANFSQYGTGIDIVAPGVNVQSTYPGNRYVSMSGTSMATPHVAGAAALVKQRYPSWNATQIRNHLKNTATNLGNSSQFGSGLVNAEAATR

SEQ ID NO: 105 - protein, 269 aa, synthetic construct
  REGION 1..269: note = Bacillus gibsonii-clade subtilisin
QQTVPWGITRVQAPAVHNRGITGSGVRVAILDSGISAHSDLNIRGGASFVPGEPTTADLNGHGTHVAGTVAALDNSIGVVGVAPNAELYAVKVLGANGRGSISGIAQGLEWAAQNNMHVANMSLGTDAPSSTLERAVNYATSRDVLVIAATGNNGSGTVSYPARYANAMAVGATDQNNRRANFSQYGTGIDIVAPGVNVQSTYPGNRYVSMSGTSMATPHVAGAAALVKQRYPSWNATQIRNHLKNTATNLGNSSQYGSGLVNAEAATR

SEQ ID NO: 106 - protein, 269 aa, synthetic construct
  REGION 1..269: note = Bacillus gibsonii-clade subtilisin
QQTVPWGITRVQAPAVHNRGITGSGVKVAILDSGISAHSDLNIRGGASFVPGEPTTADLNGHGTHVAGTVAALNNSIGVIGVAPNAELYAVKVLGANGRGSISGIAQGLEWAAQNNMHVANMSLGTDAPSSTLERAVNYATSRDVLVIAATGNNGSGTISYPARYANAMAVGATDQNNRRANFSQYGAGIDIVAPGVNVQSTYPGNRYVSMNGTSMATPHVAGAAALVKQRYPSWNATQIRDHLKNTATNLGNSSQFGSGLVNAEAATR

SEQ ID NO: 107 - protein, 269 aa, synthetic construct
  REGION 1..269: note = Bacillus gibsonii-clade subtilisin
QQTVPWGITRVQAPAVHNRGITGSGVKVAILDSGISAHSDLNIRGGASFVPGEPTTADLNGHGTHVAGTVAALNNSIGVIGVAPNAELYAVKVLGANGRGSISGIAQGLEWAATNNMHIANMSLGTDAPSSTLERAVNYATSADVLVIAATGNNGSGTISYPARYANAMAVGATDQNNRRANFSQYGTGIDIVAPGVNVQSTYPGNRYVSMNGTSMATPHVAGAAALVKQRYPSWNATQIRNHLKNTATNLGNSSQFGSGLVNAEAATR

SEQ ID NO: 108 - protein, 269 aa, synthetic construct
  REGION 1..269: note = Bacillus gibsonii-clade subtilisin
QQTVPWGITRVQAPAVHNRGITGSGVKVAILDSGISAHSDLNIRGGASFVPGEPTTADLNGHGTHVAGTVAALDNSIGVIGVAPNAELYAVKVLGANGRGSISGIAQGLEWAATNNMHVANMSLGTDAPSSTLERAVNYATSADVLVIAATGNNGSGSVSYPARYANAMAVGATDQNNRRANFSQYGTGIDIVAPGVNVQSTYPGNRYVSMSGTSMATPHVAGAAALVKQRYPSWNATQIRDHLKNTATNLGNSSQFGSGLVNAEAATR

SEQ ID NO: 109 - protein, 269 aa, synthetic construct
  REGION 1..269: note = Bacillus gibsonii-clade subtilisin
QQTVPWGITRVQAPAVHNRGITGSGVKVAILDSGISAHSDLNIRGGASFVPGEPTTADLNGHGTHVAGTVAALDNSIGVIGVAPNAELYAVKVLGANGRGSISGIAQGLEWAAQNNMHIANMSLGTDAPSSTLERAVNYATSRDVLVIAATGNNGSGSVGYPARYANAMAVGATDQNNRRANFSQYGAGLDIVAPGVNVQSTYPGNRYVSMSGTSMATPHVAGAAALVKQRYPSWNATQIRDHLKNTATNLGNSSQFGSGLVNAEAATR

SEQ ID NO: 110 - protein, 269 aa, synthetic construct
  REGION 1..269: note = Bacillus gibsonii-clade subtilisin
QQTVPWGITRVQAPAVHNRGITGSGVRVAILDSGISAHSDLNIRGGASFVPGEPTTADLNGHGTHVAGTVAALDNSIGVVGVAPNAELYAVKVLGANGRGSISGIAQGLEWAAQNNMHIANMSLGTDAPSSTLERAVNYATSADVLVIAATGNNGSGSVGYPARYANAMAVGATDQNNRRANFSQYGTGIDIVAPGVNVQSTYPGNRYVSMSGTSMATPHVAGAAALVKQRYPSWNATQIRDHLKNTATNLGNSSQFGSGLVNAEAATR

SEQ ID NO: 111 - protein, 269 aa, synthetic construct
  REGION 1..269: note = Bacillus gibsonii-clade subtilisin
QQTVPWGITRVQAPAVHNRGITGSGVRVAILDSGISAHSDLNIRGGASFVPGEPTTADLNGHGTHVAGTVAALNNSIGVIGVAPSADLYAVKVLGANGRGSVSGIAQGLEWAAQNNMHVANMSLGTDAPSSTLERAVNYATSADVLVIAATGNNGSGSVSYPARYANAMAVGATDQNNRRANFSQYGTGIDIVAPGVNVQSTYPGNRYVSMNGTSMATPHVAGAAALVKQRYPSWNATQIRNHLKNTATNLGNSSQYGSGLVNAEAATR

SEQ ID NO: 112 - protein, 269 aa, synthetic construct
  REGION 1..269: note = Bacillus gibsonii-clade subtilisin
QQTVPWGITRVQAPAVHNRGITGSGVRVAILDSGISAHSDLNIRGGASFVPGEPTTADLNGHGTHVAGTVAALNNSIGVVGVAPSADLYAVKVLGANGRGSISGIAQGLEWAAQNNMHVANMSLGTDAPSSTLERAVNYATSADVLVIAATGNNGSGSVGYPARYANAMAVGATDQNNRRANFSQYGTGIDIVAPGVNVQSTYPGNRYVSMNGTSMATPHVAGAAALVKQRYPSWNATQIRDHLKNTATNLGNSSQFGSGLVNAEAATR

SEQ ID NO: 113 - protein, 269 aa, synthetic construct
  REGION 1..269: note = Bacillus gibsonii-clade subtilisin
QQTVPWGITRVQAPAVHNRGITGSGVRVAILDSGISAHSDLNIRGGASFVPGEPTTADLNGHGTHVAGTVAALDNSIGVVGVAPSADLYAVKVLGANGRGSVSGIAQGLEWAAQNNMHIANMSLGTDAPSSTLERAVNYATSADVLVIAATGNNGSGSVGYPARYANAMAVGATDQNNRRANFSQYGTGIDIVAPGVNVQSTYPGNRYVSMNGTSMATPHVAGAAALVKQRYPSWNATQIRDHLKNTATNLGNSSQFGSGLVNAEAATR

SEQ ID NO: 114 - protein, 269 aa, synthetic construct
  REGION 1..269: note = Bacillus gibsonii-clade subtilisin
QQTVPWGITRVQAPAVHNRGITGSGVRVAILDSGISAHSDLNIRGGASFVPGEPTTADLNGHGTHVAGTVAALDNSIGVVGVAPSADLYAVKVLGANGRGSISGIAQGLEWAAQNNMHIANMSLGTDAPSSTLERAVNYATSRDVLVIAATGNNGSGSVGYPARYANAMAVGATDQNNRRANFSQYGTGIDIVAPGVNVQSTYPGNRYVSMNGTSMATPHVAGAAALVKQRYPSWNATQIRDHLKNTATNLGNSSQFGSGLVNAEAATR

SEQ ID NO: 115 - protein, 275 aa, synthetic construct
  REGION 1..275: note = Consensus sequence Figure 6
  SITE 37: note = MISC_FEATURE - Xaa can be any amino acid
  SITE 57: note = MISC_FEATURE - Xaa can be any amino acid
  SITE 82: note = MISC_FEATURE - Xaa can be any amino acid
  SITE 104: note = MISC_FEATURE - Xaa can be any amino acid
  SITE 116: note = MISC_FEATURE - Xaa can be any amino acid
  SITE 128: note = MISC_FEATURE - Xaa can be any amino acid
  REGION 145..146: note = MISC_FEATURE - Xaa can be any amino acid
  REGION 159..162: note = MISC_FEATURE - Xaa can be any amino acid
  SITE 248: note = MISC_FEATURE - Xaa can be any amino acid
QQTVPWGITRVQAPAVHNRGITGSGVRVAILDSGISXAHSDLNIRGGASFVPGEPTXTADLNGHGTHVAGTVAALNNSIGVXGVAPSADLYAVKVLGANGRGSXSGIAQGLEWAAXNNMHIANMSLGXDAPSSTLERAVNYATSXXVLVIAATGNNGSXXXXGSVGYPARYANAMAVGATDQNNRRANFSQYGTGIDIVAPGVNVQSTYPGNRYVSMNGTSMATPHVAGAAALVKQRYPSWNATQIRXHLKNTATNLGNSSQFGSGLVNAEAATR

SEQ ID NO: 116 - protein, 269 aa, synthetic construct
  REGION 1..269: note = Bacillus gibsonii-clade subtilisin
QQTVPWGITRVQAPAVHNRGITGSGVRVAILDSGISTHEDLNIRGGASFVPGEPTTADLNGHGTHVAGTVAALNNSIGVIGVAPNAELYAVKVLGANGRGSISGIAQGLEWAAQNNMHIANMSLGTDAPSSTLERAVNYATSADVLVIAATGNNGSGSVGYPARYANAMAVGATDQNNRRANFSQYGTGIDIVAPGVNVQSTYPGNRYVSMSGTSMATPHVAGAAALVKQRYPSWNATQIRDHLKNTATNLGNSSQFGSGLVNAEAATR

SEQ ID NO: 117 - protein, 269 aa, synthetic construct
  REGION 1..269: note = Bacillus gibsonii-clade subtilisin
QQTVPWGITRVQAPAVHNRGITGSGVRVAILDSGISTHEDLNIRGGASFVPGEPTTADLNGHGTHVAGTVAALDNSIGVIGVAPNAELYAVKVLGANGRGSISGIAQGLEWAATNNMHIANMSLGTDAPSSTLERAVNYATSADVLVIAATGNNGSGSVGYPARYANAMAVGATDQNNRRANFSQYGTGIDIVAPGVNVQSTYPGNRYVSMSGTSMATPHVAGAAALVKQRYPSWNATQIRDHLKNTATNLGNSSQFGSGLVNAEAATR

SEQ ID NO: 118 - protein, 269 aa, synthetic construct
  REGION 1..269: note = Bacillus gibsonii-clade subtilisin
QQTVPWGITRVQAPAVHNRGITGSGVRVAILDSGISTHEDLTIRGGASFVPGEPTTADLNGHGTHVAGTVAALNNSIGVIGVAPNAELYAVKVLGANGRGSISGIAQGLEWAAQNNMHIANMSLGTDAPSSTLERAVNYATSRDVLVIAATGNNGSGSVGYPARYANAMAVGATDQNNRRANFSQYGTGIDIVAPGVNVQSTYPGNRYVSMNGTSMATPHVAGAAALVKQRYPSWNATQIRDHLKNTATNLGNSSQFGSGLVNAEAATR

SEQ ID NO: 119 - protein, 269 aa, synthetic construct
  REGION 1..269: note = Bacillus gibsonii-clade subtilisin
QQTVPWGITRVQAPAVHNRGITGSGVRVAILDSGISTHEDLNIRGGVSFVPGEPTTADLNGHGTHVAGTVAALNNSIGVIGVAPNAELYAVKVLGANGRGSISGIAQGLEWAAANNMHVANMSLGTDAPSSTLERAVNYATSADVLVIAATGNNGSGTVSYPARYANAMAVGATDQNNRRANFSQYGTGIDIVAPGVNVQSTYPGNRYVSMSGTSMATPHVAGAAALVKQRYPSWNATQIRNHLKNTATNLGNSSQFGSGLVNAEAATR

SEQ ID NO: 120 - protein, 269 aa, synthetic construct
  REGION 1..269: note = Bacillus gibsonii-clade subtilisin
QQTVPWGITRVQAPAVHNRGITGSGVRVAILDSGISTHEDLNVRGGVSFVPGEPGTADLNGHGTHVAGTVAALNNSIGVIGVAPNAELYAVKVLGANGRGSISGIAQGLEWAAQNNMHIANMSLGTDAPSSTLERAVNYATSRDVLVIAATGNNGSGSVGYPARYANAMAVGATDQNNRRANFSQYGTGIDIVAPGVNVQSTYPGNRYVSMSGTSMATPHVAGAAALVKQRYPSWNATQIRNHLKNTATNLGNSSQFGSGLVNAEAATR

SEQ ID NO: 121 - protein, 269 aa, synthetic construct
  REGION 1..269: note = Bacillus gibsonii-clade subtilisin
QQTVPWGITRVQAPAVHNRGITGSGVRVAILDSGISTHEDLNIRGGASFVPGEPTTADLNGHGTHVAGTVAALDNSIGVVGVAPSADLYAVKVLGANGRGSISGIAQGLEWAAQNNMHIANMSLGTDAPSSTLERAVNYATSADVLVIAATGNNGSGSVGYPARYANAMAVGATDQNNRRANFSQYGTGIDIVAPGVNVQSTYPGNRYVSMNGTSMATPHVAGAAALVKQRYPSWNATQIRNHLKNTATNLGNSSQFGSGLVNAEAATR

SEQ ID NO: 122 - protein, 269 aa, synthetic construct
  REGION 1..269: note = Bacillus gibsonii-clade subtilisin
QQTVPWGITRVQAPAVHNRGITGFGVRVAILDSGISTHEDLNIRGGVSFVPGEPTTADLNGHGTHVAGTVAALDNSIGVVGVAPSADLYAVKVLGANGRGSVSGIAQGLEWAAQNNMHIANMSLGTDAPSSTLERAVNYATSRDVLVIAATGNNGSGSVGYPARYANAMAVGATDQNNRRANFSQYGTGIDIVAPGVNVQSTYPGNRYVSMSGTSMATPHVAGAAALVKQRYPSWNATQIRNHLKNTATNLGNSSQFGSGLVNAEAATR

SEQ ID NO: 123 - skipped

SEQ ID NO: 124 - protein, 269 aa, synthetic construct
  REGION 1..269: note = Bacillus gibsonii-clade subtilisin
QQTVPWGITRVQAPAVHNRGITGSGVRVAILDSGISTHEDLTIRGGVSFVPGEPTTADLNGHGTHVAGTVAALDNSIGVVGVAPSADLYAVKVLGANGRGSVSGIAQGLEWAAQNNMHIANMSLGSDAPSSTLERAVNYATSRDVLVIAATGNNGSGSVGYPARYANAMAVGATDQNNRRANFSQYGTGIDIVAPGVNVQSTYPGNRYVSMNGTSMATPHVAGAAALVKQRYPSWNATQIRDHLKNTATNLGNSSQFGSGLVNAEAATR

SEQ ID NO: 125 - protein, 269 aa, synthetic construct
  REGION 1..269: note = Bacillus gibsonii-clade subtilisin
QQTVPWGITRVQAPAVHNRGITGSGVRVAILDSGISTHEDLTIRGGVSFVPGEPTTADLNGHGTHVAGTVAALNNSIGVVGVAPSADLYAVKVLGANGRGSISGIAQGLEWAAQNNMHIANMSLGSDAPSSTLERAVNYATSRDVLVIAATGNNGSGSVGYPARYANAMAVGATDQNNRRANFSQYGTGIDIVAPGVNVQSTYPGNRYVSMNGTSMATPHVAGAAALVKQRYPSWNATQIRDHLKNTATNLGNSSQFGSGLVNAEAATR

SEQ ID NO: 126 - protein, 269 aa, synthetic construct
  REGION 1..269: note = Bacillus gibsonii-clade subtilisin
QQTVPWGITRVQAPAVHNRGITGSGVRVAILDSGISTHEDLTIRGGVSFVPGEPGTADLNGHGTHVAGTVAALNNSIGVVGVAPSADLYAVKVLGANGRGSVSGIAQGLEWAAQNNMHIANMSLGSDAPSSTLERAVNYATSRDVLVIAATGNNGSGSVGYPARYANAMAVGATDQNNRRANFSQYGTGIDIVAPGVNVQSTYPGNRYVSMSGTSMATPHVAGAAALVKQRYPSWNATQIRDHLKNTATNLGNSSQFGSGLVNAEAATR

SEQ ID NO: 127 - protein, 269 aa, synthetic construct
  REGION 1..269: note = Bacillus gibsonii-clade subtilisin
QQTVPWGITRVQAPAVHNRGITGSGVKVAILDSGISTHEDLNIRGGVSFVPGEPTTADLNGHGTHVAGTVAALNNSIGVVGVAPSADLYAVKVLGANGRGSVSGIAQGLEWAAQNNMHIANMSLGSDAPSSTLERAVNYATSRDVLVIAATGNNGSGSVGYPARYANAMAVGATDQNNRRANFSQYGTGIDIVAPGVNVQSTYPGNRYVSMSGTSMATPHVAGAAALVKQRYPSWNATQIRDHLKNTATNLGNSSQFGSGLVNAEAATR

SEQ ID NO: 128 - protein, 269 aa, synthetic construct
  REGION 1..269: note = Bacillus gibsonii-clade subtilisin
QQTVPWGITRVQAPAVHNRGITGSGVRVAILDSGISTHEDLNIRGGVSFVPGEPTTADLNGHGTHVAGTVAALNNSIGVVGVAPSADLYAVKVLGANGRGSVSGIAQGLEWAAQNNMHIANMSLGSDAPSSTLERAVNYATSADVLVIAATGNNGSGSVGYPARYANAMAVGATDQNNRRANFSQYGTGIDIVAPGVNVQSTYPGNRYVSMNGTSMATPHVAGAAALVKQRYPSWNATQIRDHLKNTATNLGNSSQFGSGLVNAEAATR

SEQ ID NO: 129 - protein, 269 aa, synthetic construct
  REGION 1..269: note = Bacillus gibsonii-clade subtilisin
QQTVPWGITRVQAPAVHNRGITGSGVRVAILDSGISTHEDLNIRGGVSFVPGEPTTADLNGHGTHVAGTVAALNNSIGVVGVAPSADLYAVKVLGANGRGSISGIAQGLEWAAQNNMHIANMSLGSDAPSSTLERAVNYATSADVLVIAATGNNGSGSVGYPARYANAMAVGATDQNNRRANFSQYGTGIDIVAPGVNVQSTYPGNRYVSMNGTSMATPHVAGAAALVKQRYPSWNATQIRDHLKNTATNLGNSSQFGSGLVNAEAATR

SEQ ID NO: 130 - protein, 269 aa, synthetic construct
  REGION 1..269: note = Bacillus gibsonii-clade subtilisin
QQTVPWGITRVQAPAVHNRGITGSGVRVAILDSGISTHEDLNVRGGVSFVPGEPTTADLNGHGTHVAGTVAALNNSIGVVGVAPSADLYAVKVLGANGRGSVSGIAQGLEWAAQNNMHIANMSLGTDAPSSTLERAVNYATSADVLVIAATGNNGSGSVGYPARYANAMAVGATDQNNRRANFSQYGTGIDIVAPGVNVQSTYPGNRYVSMNGTSMATPHVAGAAALVKQRYPSWNATQIRDHLKNTATNLGNSSQFGSGLVNAEAATR

SEQ ID NO: 131-132 - skipped (2 entries)

SEQ ID NO: 133 - protein, 269 aa, synthetic construct
  REGION 1..269: note = Bacillus gibsonii-clade subtilisin
QQTVPWGITRVQAPAVHNRGITGSGVRVAILDSGISTHEDLNVRGGVSFVPGEPTTADLNGHGTHVAGTVAALNNSIGVVGVAPSADLYAVKVLGANGRGSISGIAQGLEWAAQNNMHIANMSLGTDAPSSTLERAVNYATSRDVLVIAATGNNGSGSVGYPARYANAMAVGATDQNNRRANFSQYGTGIDIVAPGVNVQSTYPGNRYVSMNGTSMATPHVAGAAALVKQRYPSWNATQIRDHLKNTATNLGNSSQFGSGLVNAEAATR

SEQ ID NO: 134 - protein, 269 aa, synthetic construct
  REGION 1..269: note = Bacillus gibsonii-clade subtilisin
QQTVPWGITRVQAPAVHNRGITGSGVRVAILDSGISTHEDLNVRGGVSFVPGEPGTADLNGHGTHVAGTVAALNNSIGVVGVAPSADLYAVKVLGANGRGSISGIAQGLEWAAQNNMHIANMSLGSDAPSSTLERAVNYATSADVLVIAATGNNGSGSVGYPARYANAMAVGATDQNNRRANFSQYGTGIDIVAPGVNVQSTYPGNRYVSMNGTSMATPHVAGAAALVKQRYPSWNATQIRNHLKNTATNLGNSSQFGSGLVNAEAATR

SEQ ID NO: 135 - protein, 269 aa, synthetic construct
  REGION 1..269: note = Bacillus gibsonii-clade subtilisin
QQTVPWGITRVQAPAVHNRGITGSGVRVAILDSGISTHEDLNVRGGVSFVPGEPTTADLNGHGTHVAGTVAALNNSIGVVGVAPSADLYAVKVLGANGRGSISGIAQGLEWAAQNNMHIANMSLGSDAPSSTLERAVNYATSRDVLVIAATGNNGSGSVGYPARYANAMAVGATDQNNRRANFSQYGTGIDIVAPGVNVQSTYPGNRYVSMNGTSMATPHVAGAAALVKQRYPSWNATQIRNHLKNTATNLGNSSQFGSGLVNAEAATR

SEQ ID NO: 136 - protein, 269 aa, synthetic construct
  REGION 1..269: note = Bacillus gibsonii-clade subtilisin
QQTVPWGITRVQAPAVHNRGITGSGVRVAILDSGISTHEDLNVRGGVSFVPGEPTTADLNGHGTHVAGTVAALNNSIGVVGVAPSADLYAVKVLGANGRGSVSGIAQGLEWAAQNNMHIANMSLGSDAPSSTLERAVNYATSRDVLVIAATGNNGSGSVGYPARYANAMAVGATDQNNRRANFSQYGTGIDIVAPGVNVQSTYPGNRYVSMSGTSMATPHVAGAAALVKQRYPSWNATQIRDHLKNTATNLGNSSQFGSGLVNAEAATR

SEQ ID NO: 137 - protein, 269 aa, synthetic construct
  REGION 1..269: note = Bacillus gibsonii-clade subtilisin
QQTVPWGITRVQAPAVHNRGITGSGVRVAILDSGISTHEDLNVRGGVSFVPGEPTTADLNGHGTHVAGTVAALNNSIGVVGVAPSADLYAVKVLGANGRGSVSGIAQGLEWAAANNMHIANMSLGTDAPSSTLERAVNYATSRDVLVIAATGNNGSGSVGYPARYANAMAVGATDQNNRRANFSQYGTGIDIVAPGVNVQSTYPGNRYVSMNGTSMATPHVAGAAALVKQRYPSWNTTQIRDHLKNTATNLGNSSQFGSGLVNAEAATR

SEQ ID NO: 138 - protein, 269 aa, synthetic construct
  REGION 1..269: note = Bacillus gibsonii-clade subtilisin
QQTVPWGITRVQAPAVHNRGITGSGVRVAILDSGISTHEDLNVRGGVSFVPGEPTTADLNGHGTHVAGTVAALNNSIGVVGVAPSADLYAVKVLGANGRGSVSGIAQGLEWAAANNMHIANMSLGSDAPSSTLERAVNYATSRDVLVIAATGNNGSGSVGYPARYANAMAVGATDQNNRRANFSQYGTGIDIVAPGVNVQSTYPGNRYVSMNGTSMATPHVAGAAALVKQRYPSWNATQIRDHLKNTATNLGNSSQFGSGLVNAEAATR

SEQ ID NO: 139 - protein, 269 aa, synthetic construct
  REGION 1..269: note = Bacillus gibsonii-clade subtilisin
QQTVPWGITRVQAPAVHNRGITGSGVRVAILDSGISTHEDLNVRGGVSFVPGEPTYADLNGHGTHVAGTVAALNNSIGVVGVAPSADLYAVKVLGANGRGSVSGIAQGLEWAAANNMHIANMSLGSDAPSSTLERAVNYATSRDVLVIAATGNNGSGSVGYPARYANAMAVGATDQNNRRANFSQYGTGIDIVAPGVNVQSTYPGNRYVSMNGTSMATPHVAGAAALVKQRYPSWNATQIRDHLKNTATNLGNSSQFGSGLVNAEAATR

SEQ ID NO: 140 - protein, 269 aa, synthetic construct
  REGION 1..269: note = Bacillus gibsonii-clade subtilisin
QQTVPWGITRVQAPAVHNRGITGSGVRVAILDSGIAAHEDLNVRGGVSFVPGEPMTADLNGHGTHVAGTVAALNNSIGVVGVAPSADLYAVKVLGANGRGSVSGIAQGLEWAAANNMHIANMSLGSDAPSSTLERAVNYATSRDVLVIAATGNNGSGSVGYPARYANAMAVGATDQNNRRANFSQYGTGIDIVAPGVNVQSTYPGNRYVSMNGTSMATPHVAGAAALVKQRYPSWNATQIRNHLKNTATNLGNSSQFGSGLVNAEAATR

SEQ ID NO: 141 - protein, 269 aa, synthetic construct
  REGION 1..269: note = Bacillus gibsonii-clade subtilisin
QQTVPWGITRVQAPAVHNRGITGSGVRVAILDSGIAAHEDLNVRGGVSFVPGEPTTADLNGHGTHVAGTVAALNNSIGVVGVAPSADLYAVKVLGANGRGSVSGIAQGLEWAAANNMHIASMSLGSDAPSSTLERAVNYATSQDVLVIAATGNNGSGSVGYPARYANAMAVGATDQNNRRANFSQYGTGIDIVAPGVNVQSTYPGNRYVSMNGTSMATPHVAGAAALVKQRYPSWNATQIRNHLKNTATNLGNSSQFGSGLVNAEAATR

SEQ ID NO: 142 - protein, 269 aa, synthetic construct
  REGION 1..269: note = Bacillus gibsonii-clade subtilisin
QQTVPWGITRVQAPAVHNRGITGSGVRVAILDSGIAAHEDLNVRGGVSFVPGEPTTADLNGHGTHVAGTVAALNNSIGVVGVAPSADLYAVKVLGANGRGSVSGIAQGLEWAAANNMHIANMSLGSDAPSSTLERAVNYATSRDVLVIAATGNNGSGSVGYPARYANAMAVGATDQNNRRANFSQYGTGIDIVAPGVNVQSTYPGNRYVSMSGTSMATPHVAGAAALVKQRYPSWNATQIRNHLKNTATNLGNSNQFGSGLVNAEAATR

SEQ ID NO: 143 - protein, 269 aa, synthetic construct
  REGION 1..269: note = Bacillus gibsonii-clade subtilisin
QQTVPWGITRVQAPAVHNRGITGSGVRVAILDSGISAHEDLNVRGGVSFVPGEPTTADLNGHGTHVAGTVAALNNSIGVVGVAPSADLYAVKVLGANGRGSVSGIAQGLEWAAANNMHIANMSLGSDAPSSTLERAVNYATSRDVLVIAATGNNGSGSVGYPARYANAMAVGATDQNNRRANFSQYGTGIDIVAPGVNVQSTYPGNRYVSMSGTSMATPHVAGAAALVKQRYPSWNATQIRNHLLSTATNLGNSSQFGSGLVNAEAATR

SEQ ID NO: 144 - protein, 269 aa, synthetic construct
  REGION 1..269: note = Bacillus gibsonii-clade subtilisin
QQTVPWGITRVQAPAVHNRGITGSGVRVAILDSGISAHEDLNVRGGVSFVPGEPTTADLNGHGTHVAGTVAALNNSIGVVGVAPSADLYAVKVLGANGRGSVSGIAQGLEWAAANNMHIANMSLGSDAPSSTLERAVNYATSQDVLVIAATGNNGSGSVGYPARYANAMAVGATDQNNRRANFSQYGTGIDIVAPGVNVQSTYPGNRYVSMNGTSMATPHVAGAAALVKQRYPSWNATQIRDHLKKTATNLGNSSQFGSGLVNAEAATR

SEQ ID NO: 145 - protein, 269 aa, synthetic construct
  REGION 1..269: note = Bacillus gibsonii-clade subtilisin
QQTVPWGITRVQAPAVHNRGITGSGVRVAILDSGISTHEDLNVRGGVSFVPGEPTTADLNGHGTHVAGTVAALDNSIGVIGVAPSADLYAVKVLGANGRGSVSGIAQGLEWAAANNMHIANMSLGTDAPSSTLERAVNYATSRDVLVIAATGNNGSGTVGYPARYANAMAVGATDQNNRRANFSQYGTGIDIVAPGVNVQSTYPGNRYVSMSGTSMATPHVAGAAALVKQRYPSWNATQIRNHLKNTATNLGNSSQFGSGLVNAEAATR

SEQ ID NO: 146 - protein, 269 aa, synthetic construct
  REGION 1..269: note = Bacillus gibsonii-clade subtilisin
QQTVPWGITRVQAPAVHNRGITGSGVRVAILDSGISTHEDLNVRGGVSFVPGEPTTADLNGHGTHVAGTVAALDNSIGVIGVAPSADLYAVKVLGANGRGSVSGIAQGLEWAAANNMHIANMSLGSDAPSSTLERAVNYATSRDVLVIAATGNNGSGTVGYPARYANAMAVGATDQNNRRANFSQYGTGIDIVAPGVNVQSTYPGNRYVSMSGTSMATPHVAGAAALVKQRYPSWNATQIRNHLKNTATNLGNSSQFGSGLVNAEAATR

SEQ ID NO: 147 - protein, 269 aa, synthetic construct
  REGION 1..269: note = Bacillus gibsonii-clade subtilisin
QQTVPWGITRVQAPAVHNRGITGSGVRVAILDSGISTHEDLNVRGGVSFVPGEPTTADLNGHGTHVAGTVAALDNSIGVIGVAPSADLYAVKVLGANGRGSVSGIAQGLEWAAANNMHIANMSLGTDAPSSTLERAVNYATSRDVLVIAATGNNGSGSVGYPARYANAMAVGATDQNNRRANFSQYGTGIDIVAPGVNVQSTYPGNRYVSMSGTSMATPHVAGAAALVKQRYPSWNATQIRNHLKNTATNLGNSSQFGSGLVNAEAATR

SEQ ID NO: 148 - protein, 269 aa, synthetic construct
  REGION 1..269: note = Bacillus gibsonii-clade subtilisin
QQTVPWGITRVQAPAVHNRGITGSGVRVAILDSGISTHEDLNVRGGVSFVPGEPTTADLNGHGTHVAGTVAALDNSIGVVGVAPSADLYAVKVLGANGRGSVSGIAQGLEWAAANNMHIANMSLGSDAPSSTLERAVNYATSRDVLVIAATGNNGSGTVGYPARYANAMAVGATDQNNRRANFSQYGTGIDIVAPGVNVQSTYPGNRYVSMSGTSMATPHVAGAAALVKQRYPSWNATQIRNHLKNTATNLGNSSQFGSGLVNAEAATR

SEQ ID NO: 149 - protein, 269 aa, synthetic construct
  REGION 1..269: note = Bacillus gibsonii-clade subtilisin
QQTVPWGITRVQAPAVHNRGITGSGVRVAILDSGISTHEDLNVRGGVSFVPGEPTTADLNGHGTHVAGTVAALDNSIGVVGVAPSADLYAVKVLGANGRGSVSGIAQGLEWAAANNMHIANMSLGSDAPSSTLERAVNYATSRDVLVIAATGNNGSGSVGYPARYANAMAVGATDQNNRRANFSQYGTGIDIVAPGVNVQSTYPGNRYVSMSGTSMATPHVAGAAALVKQRYPSWNATQIRNHLKNTATNLGNSSQFGSGLVNAEAATR

SEQ ID NO: 150 - protein, 269 aa, synthetic construct
  REGION 1..269: note = Bacillus gibsonii-clade subtilisin
QQTVPWGITRVQAPAVHNRGITGSGVRVAILDSGISTHEDLNVRGGVSFVPGEPTTADLNGHGTHVAGTVAALDNSIGVIGVAPSADLYAVKVLGANGRGSVSGIAQGLEWAAANNMHIANMSLGSDAPSSTLERAVNYATSRDVLVIAATGNNGSGSVGYPARYANAMAVGATDQNNRRANFSQYGTGIDIVAPGVNVQSTYPGNRYVSMSGTSMATPHVAGAAALVKQRYPSWNATQIRQHLLNTATNLGNSSQFGSGLVNAEAATR

SEQ ID NO: 151 - protein, 269 aa, synthetic construct
  REGION 1..269: note = Bacillus gibsonii-clade subtilisin
QQTVPWGITRVQAPAVHNRGITGSGVRVAILDSGISTHEDLNVRGGVSFVPGEPTTADLNGHGTHVAGTVAALDNSIGVVGVAPSADLYAVKVLGANGRGSVSGIAQGLEWAAANNMHIANMSLGSDAPSSTLERAVNYATSRDVLVIAATGNNGSGSVGYPARYANAMAVGATDQNNRRANFSQYGTGIDIVAPGVNVQSTYPGNRYVSMSGTSMATPHVAGAAALVKQRYPSWNATQIRNHLLNTATNLGNSSQFGSGLVNAEAATR

SEQ ID NO: 152 - protein, 269 aa, synthetic construct
  REGION 1..269: note = Bacillus gibsonii-clade subtilisin
QQTVPWGITRVQAPAVHNRGITGSGVRVAILDSGISTHEDLNVRGGVSFVPGEPTTADLNGHGTHVAGTVAALDNSIGVVGVAPSADLYAVKVLGANGRGSVSGIAQGLEWAAANNMHIANMSLGSDAPSSTLERAVNYATSRDVLVIAATGNNGSGSVGYPARYANAMAVGATDQNNRRANFSQYGTGIDIVAPGVNVQSTYPGNRYVSMSGTSMATPHVAGAAALVKQRYPSWNATQIRQHLKNTATNLGNSSQFGSGLVNAEAATR

SEQ ID NO: 153 - protein, 269 aa, synthetic construct
  REGION 1..269: note = Bacillus gibsonii-clade subtilisin
QQTVPWGITRVQAPAVHNRGITGSGVRVAILDSGISTHEDLTIRGGVSFVPGEPTTADLNGHGTHVAGTVAALNNSIGVVGVAPSADLYAVKVLGANGRGSVSGIAQGLEWAAANNMHIANMSLGSDAPSSTLERAVNYATSRDVLVIAATGNNGSGTVGYPARYANAMAVGATDQNNRRANFSQYGTGIDIVAPGVNVQSTYPGNRYVSMSGTSMATPHVAGAAALVKQRYPSWNATQIRNHLKNTATNLGNSSQFGSGLVNAEAATR

SEQ ID NO: 154 - protein, 269 aa, synthetic construct
  REGION 1..269: note = Bacillus gibsonii-clade subtilisin
QQTVPWGITRVQAPAVHNRGITGSGVRVAILDSGISTHEDLNVRGGVSFVPGEPTTADLNGHGTHVAGTVAALNNSIGVVGVAPSADLYAVKVLGANGRGSISGIAQGLEWAAANNMHIANMSLGSDAPSSTLERAVNYATSRDVLVIAATGNNGSGSVGYPARYANAMAVGATDQNNRRANFSQYGTGIDIVAPGVNVQSTYPGNRYVSMSGTSMATPHVAGAAALVKQRYPSWNATQIRNHLKNTATNLGNSSQFGSGLVNAEAATR

SEQ ID NO: 155 - protein, 269 aa, synthetic construct
  REGION 1..269: note = Bacillus gibsonii-clade subtilisin
QQTVPWGITRVQAPAVHNRGITGSGVRVAILDSGISTHEDLNVRGGVSFVPGEPTTADLNGHGTHVAGTVAALNNSIGVVGVAPSADLYAVKVLGANGRGSVSGIAQGLEWAAANNMHIANMSLGTDAPSSTLERAVNYATSRDVLVIAATGNNGSGSVGYPARYANAMAVGATDQNNRRANFSQYGTGIDIVAPGVNVQSTYPGNRYVSMSGTSMATPHVAGAAALVKQRYPSWNATQIRNHLKNTATNLGNSSQFGSGLVNAEAATR

SEQ ID NO: 156 - protein, 269 aa, synthetic construct
  REGION 1..269: note = Bacillus gibsonii-clade subtilisin
QQTVPWGISRVQAPAVHNRGITGSGVRVAILDSGISTHEDLNVRGGVSFVPGEPTTADLNGHGTHVAGTVAALNNSIGVVGVAPSADLYAVKVLGANGRGSVSGIAQGLEWAAANNMHIANMSLGSDAPSSTLERAVNYATSRDVLVIAATGNNGSGSVGYPARYANAMAVGATDQNNRRANFSQYGTGIDIVAPGINVQSTYPGNRYVSMNGTSMATPHVAGAAALVKQRYPSWNATQIRNHLKNTATNLGNSSQFGSGLVNAEAATR

SEQ ID NO: 157 - protein, 269 aa, synthetic construct
  REGION 1..269: note = Bacillus gibsonii-clade subtilisin
QQTVPWGITRVQAPAVHNRGITGSGVRVAILDSGISTHEDLNVRGGVSFVPGEPTTADLNGHGTHVAGTVAALNNSIGVVGVAPSADLYAVKVLGANGRGSVSGIAQGLEWAAANNMHIANMSLGSDAPSSTLERAVNYATSRDVLVIAATGNNGSGSVGYPARYANAMAVGATDQNNRRANFSQYGTGLDIVAPGVNVQSTYPGNRYVSMNGTSMATPHVAGVAALVKQRYPSWNATQIRNHLKNTATNLGNSSQFGSGLVNAEAATR

SEQ ID NO: 158 - protein, 269 aa, synthetic construct
  REGION 1..269: note = Bacillus gibsonii-clade subtilisin
QQTIPWGITRVQAPAVHNRGITGSGVRVAILDSGISTHEDLNVRGGVSFVPGEPTTADLNGHGTHVAGTVAALNNSIGVVGVAPSADLYAVKVLGANGRGSVSGIAQGLEWAAANNMHIANMSLGSDAPSSTLERAVNYATSRDVLVIAATGNNGSGSVGYPARYANAMAVGATDQNNRRASFSQYGTGIDIVAPGVNVQSTYPGNRYVSMNGTSMATPHVAGAAALVKQRYPSWNATQIRNHLKNTATNLGNSSQFGSGLVNAEAATR

SEQ ID NO: 159 - protein, 275 aa, synthetic construct
  UNSURE 1..275: note = X can be any Amino Acid
QQTVPWGITRVQAPAVHNRGITGSGVRVAILDSGISTXHEDLNVRGGVSFVPGEPTXTADLNGHGTHVAGTVAALNNSIGVVGVAPSADLYAVKVLGANGRGSVSGIAQGLEWAAXNNMHIANMSLGSDAPSSTLERAVNYATSRDVLVIAATGNNGSXXXXGSVGYPARYANAMAVGATDQNNRRANFSQYGTGIDIVAPGVNVQSTYPGNRYVSMNGTSMATPHVAGAAALVKQRYPSWNATQIRNHLKNTATNLGNSSQFGSGLVNAEAATR

SEQ ID NO: 160 - protein, 269 aa, synthetic construct
  REGION 1..269: note = Bacillus gibsonii-clade subtilisin
QQTVPWGITRVQAPAVHNRGITGSGVRVAILDSGISTHEDLNVRGGVSFVPGEPTTADLNGHGTHVAGTVAALDNSIGVVGVAPSADLYAVKVLGANGRGSVSGIAQGLEWAAANNMHIANMSLGTDAPSSTLERAVNYATSRDVLVIAATGNNGSGTVGYPARYANAMAVGATDQNNRRANFSQYGTGIDIVAPGVNVQSTYPGNRYVSMSGTSMATPHVAGAAALVKQRYPSWNATQIRQHLKNTATNLGNSSQFGSGLVNAEAATR

SEQ ID NO: 161 - protein, 269 aa, synthetic construct
  REGION 1..269: note = Bacillus gibsonii-clade subtilisin
QQTVPWGITRVQAPAVHNRGITGSGVRVAILDSGISTHEDLNVRGGVSFVPGEPTTADLNGHGTHVAGTVAALDNSIGVIGVAPSADLYAVKVLGANGRGSVSGIAQGLEWAAANNMHIANMSLGTDAPSSTLERAVNYATSRDVLVIAATGNNGSGTVGYPARYANAMAVGATDQNNRRANFSQYGTGIDIVAPGVNVQSTYPGNRYVSMSGTSMATPHVAGAAALVKQRYPSWNATQIRQHLKNTATNLGNSSQFGSGLVNAEAATR

SEQ ID NO: 162 - protein, 269 aa, synthetic construct
  REGION 1..269: note = Bacillus gibsonii-clade subtilisin
QQTVPWGITRVQAPAVHNRGITGSGVRVAILDSGISTHEDLNVRGGVSFVPGEPTTADLNGHGTHVAGTVAALDNSIGVVGVAPSADLYAVKVLGANGRGSVSGIAQGLEWAAANNMHIANMSLGTDAPSSTLERAVNYATSRDVLVIAATGNNGSGTVGYPARYANAMAVGATDQNNRRANFSQYGTGIDIVAPGVNVQSTYPGNRYVSMSGTSMATPHVAGAAALVKQRYPSWNATQIRQHLLNTATNLGNSSQFGSGLVNAEAATR

SEQ ID NO: 163 - protein, 269 aa, synthetic construct; identical to SEQ ID NO: 148
  REGION 1..269: note = Bacillus gibsonii-clade subtilisin

SEQ ID NO: 164 - protein, 269 aa, synthetic construct
  REGION 1..269: note = Bacillus gibsonii-clade subtilisin
QQTVPWGITRVQAPAVHNRGITGSGVRVAILDSGISTHEDLNVRGGVSFVPGEPTTADLNGHGTHVAGTVAALDNSIGVIGVAPSADLYAVKVLGANGRGSVSGIAQGLEWAAANNMHIANMSLGTDAPSSTLERAVNYATSRDVLVIAATGNNGSGTVGYPARYANAMAVGATDQNNRRANFSQYGTGIDIVAPGVNVQSTYPGNRYVSMSGTSMATPHVAGAAALVKQRYPSWNATQIRQHLLNTATNLGNSSQFGSGLVNAEAATR

SEQ ID NO: 165 - protein, 269 aa, synthetic construct
  REGION 1..269: note = Bacillus gibsonii-clade subtilisin
QQTVPWGITRVQAPAVHNRGITGSGVRVAILDSGISTHEDLNVRGGVSFVPGEPTTADLNGHGTHVAGTVAALDNSIGVIGVAPSADLYAVKVLGANGRGSVSGIAQGLEWAAANNMHIANMSLGTDAPSSTLERAVNYATSRDVLVIAATGNNGSGTVGYPARYANAMAVGATDQNNRRANFSQYGTGIDIVAPGVNVQSTYPGNRYVSMSGTSMATPHVAGAAALVKQRYPSWNATQIRNHLLNTATNLGNSSQFGSGLVNAEAATR

SEQ ID NO: 166 - protein, 269 aa, synthetic construct
  REGION 1..269: note = Bacillus gibsonii-clade subtilisin
QQTVPWGITRVQAPAVHNRGITGSGVRVAILDSGISTHEDLNIRGGVSFVPGEPTYADLNGHGTHVAGTVAALNNSIGVVGVAPSADLYAVKVLGANGRGSVSGIAQGLEWAAQNNMHIANMSLGSDFPSSTLERAVNYATSADVLVIAATGNNGSGSVGYPARYANAMAVGATDQNNRRANFSQYGTGIDIVAPGVNVQSTYPGNRYVSMNGTSMATPHVAGAAALVKQRYPSWNATQIRDHLKNTATNLGNSSQFGSGLVNAEAATR

SEQ ID NO: 167 - protein, 269 aa, synthetic construct
  REGION 1..269: note = Bacillus gibsonii-clade subtilisin
QQTVPWGITRVQAPAVHNRGITGSGVRVAILDSGISTHEDLTVSGGVSFVPGEPTTADLNGHGTHVAGTVAALDNSIGVVGVAPSADLYAVKVLGANGRGSISGIAQGLEWAATNNMHIANMSLGTDAPSSTLERAVNYATSRDVLVIAATGNNGSGTVSYPARYANAMAVGATDQNNRRANFSQYGTGIDIVAPGVNVQSTYPGNRYVSMNGTSMATPHVAGAAALVKQRYPSWNATQIRDHLKNTATNLGNSSQFGSGLVNAEAATR

SEQ ID NO: 168 - protein, 269 aa, synthetic construct
  REGION 1..269: note = Bacillus gibsonii-clade subtilisin
QQTVPWGITRVQAPAVHNRGITGSGVRVAILDSGISTHEDLNVRGGVSFVPGEPGTADLNGHGTHVAGTVAALDNSIGVVGVAPSADLYAVKVLGANGRGSISGIAQGLEWAAQNNMHIANMSLGTDAPSSTLERAVNYATSRDVLVIAATGNNGSGTVGYPARYANAMAVGATDQNNRRANFSQYGTGIDIVAPGVNVQSTYPGNRYVSMNGTSMATPHVAGAAALVKQRYPSWNATQIRDHLKNTATNLGNSSQFGSGLVNAEAATR

SEQ ID NO: 169 - protein, 269 aa, synthetic construct
  REGION 1..269: note = Bacillus gibsonii-clade subtilisin
QQTVPWGITRVQAPAVHNRGITGSGVRVAILDSGISTHEDLNVRGGVSFVPGEPGTADLNGHGTHVAGTVAALDNSIGVVGVAPSADLYAVKVLGANGRGSVSGIAQGLEWAAQNNMHIANMSLGTDAPSSTLERAVNYATSADVLVIAATGNNGSGTVGYPARYANAMAVGATDQNNRRANFSQYGTGIDIVAPGVNVQSTYPGNRYVSMSGTSMATPHVAGAAALVKQRYPSWNATQIRNHLKNTATNLGNSSQFGSGLVNAEAATR

SEQ ID NO: 170 - protein, 269 aa, synthetic construct
  REGION 1..269: note = Bacillus gibsonii-clade subtilisin
QQTVPWGITRVQAPAVHNRGITGSGVRVAILDSGISTHEDLNVRGGVSFVPGEPGTADLNGHGTHVAGTVAALDNSIGVVGVAPSADLYAVKVLGANGRGSVSGIAQGLEWAAQNNMHIANMSLGTDAPSSTLERAVNYATSRDVLVIAATGNNGSGSVGYPARYANAMAVGATDQNNRRANFSQYGTGIDIVAPGVNVQSTYPGNRYVSMNGTSMATPHVAGAAALVKQRYPSWNATQIRDHLKNTATNLGNSSQFGSGLVNAEAATR

SEQ ID NO: 171 - protein, 269 aa, synthetic construct
  REGION 1..269: note = Bacillus gibsonii-clade subtilisin
QQTVPWGITRVQAPAVHNRGITGSGVRVAILDSGISTHEDLNVRGGVSFVPGEPGTADLNGHGTHVAGTVAALNNSIGVVGVAPSADLYAVKVLGANGRGSISGIAQGLEWAAQNNMHIANMSLGTDAPSSTLERAVNYATSADVLVIAATGNNGSGSVGYPARYANAMAVGATDQNNRRANFSQYGTGIDIVAPGVNVQSTYPGNRYVSMNGTSMATPHVAGAAALVKQRYPSWNATQIRNHLKNTATNLGNSSQFGSGLVNAEAATR

SEQ ID NO: 172 - protein, 269 aa, synthetic construct
  REGION 1..269: note = Bacillus gibsonii-clade subtilisin
QQTVPWGITRVQAPAVHNRGITGSGVRVAILDSGISTHEDLNVRGGVSFVPGEPTTADLNGHGTHVAGTVAALNNSIGVVGVAPSADLYAVKVLGANGRGSISGIAQGLEWAAQNNMHIANMSLGTDAPSSTLERAVNYATSADVLVIAATGNNGSGSVGYPARYANAMAVGATDQNNRRANFSQYGTGIDIVAPGVNVQSTYPGNRYVSMNGTSMATPHVAGAAALVKQRYPSWNATQIRDHLKNTATNLGNSSQFGSGLVNAEAATR

SEQ ID NO: 173 - protein, 269 aa, synthetic construct; identical to SEQ ID NO: 138
  REGION 1..269: note = Bacillus gibsonii-clade subtilisin

SEQ ID NO: 174 - protein, 269 aa, synthetic construct
  REGION 1..269: note = Bacillus gibsonii-clade subtilisin
QQTVPWGITRVQAPAVHNRGITGSGVRVAILDSGISTHEDLNVRGGVSFVPGEPTTADLNGHGTHVAGTVAALNNSIGVVGVAPSADLYAVKVLGANGRGSVSGIAQGLEWAAANNMHIANMSLGTDAPSSTLERAVNYATSADVLVIAATGNNGSGTVGYPARYANAMAVGATDQNNRRANFSQYGTGIDIVAPGVNVQSTYPGNRYVSMNGTSMATPHVAGAAALVKQRYPSWNATQIRDHLKNTATNLGNSSQFGSGLVNAEAATR

SEQ ID NO: 175 - protein, 269 aa, synthetic construct
  REGION 1..269: note = Bacillus gibsonii-clade subtilisin
QQTVPWGITRVQAPAVHNRGITGSGVRVAILDSGISTHEDLNVRGGVSFVPGEPTTADLNGHGTHVAGTVAALNNSIGVVGVAPSADLYAVKVLGANGRGSVSGIAQGLEWAAPNNMHIANMSLGTDAPSSTLERAVNYATSTDVLVIAATGNNGSGTVGYPARYANAMAVGATDQNNRRANFSQYGTGIDIVAPGVNVQSTYPGNRYVSMKGTSMATPHVAGAAALVKQRYPSWNATQIRDHLKNTATNLGNSSQFGSGLVNAEAATR

SEQ ID NO: 176 - protein, 269 aa, synthetic construct
  REGION 1..269: note = Bacillus gibsonii-clade subtilisin
QQTVPWGITRVQAPAVHNRGITGSGVRVAILDSGISTHEDLTIRGGVSFVPGEPTTADLNGHGTHVAGTVAALDNSIGVVGVAPSADLYAVKVLGANGRGSISGIAQGLEWAAQNNMHIANMSLGTDAPSSTLERAVNYATSRDVLVIAATGNNGSGSVGYPARYANAMAVGATDQNNRRANFSQYGTGIDIVAPGVNVQSTYPGNRYVSMNGTSMATPHVAGAAALVKQRYPSWNATQIRDHLKNTATNLGNSSQFGSGLVNAEAATR

SEQ ID NO: 177 - protein, 269 aa, synthetic construct
  REGION 1..269: note = Bacillus gibsonii-clade subtilisin
QQTVPWGITRVQAPAVHNRGITGSGVRVAILDSGISTHEDLNVRGGVSFVPGEPTYADLNGHGTHVAGTVAALDNSIGVVGVAPSADLYAVKVLGANGRGSVSGIAQGLEWAAQNNMHIANMSLGTDAPSSTLERAVNYATSADVLVIAATGNNGSGSVGYPARYANAMAVGATDQNNRRANFSQYGTGIDIVAPGVNVQSTYPGNRYVSMNGTSMATPHVAGAAALVKQRYPSWNATQIRDHLKNTATNLGNSSQFGSGLVNAEAATR

SEQ ID NO: 178 - protein, 269 aa, synthetic construct
  REGION 1..269: note = Bacillus gibsonii-clade subtilisin
QQTVPWGITRVQAPAVHNRGITGSGVRVAILDSGISTHEDLNVRGGVSFVPGEPTYADLNGHGTHVAGTVAALNNSIGVVGVAPSADLYAVKVLGANGRGSVSGIAQGLEWAAQNNMHVANMSLGTDAPSSTLERAVNYATSRDVLVIAATGNNGSGSVGYPARYANAMAVGATDQNNRRANFSQYGTGIDIVAPGVNVQSTYPGNRYVSMNGTSMATPHVAGAAALVKQRYPSWNATQIRNHLKNTATNLGNSSQYGSGLVNAEAATR

SEQ ID NO: 179 - protein, 269 aa, synthetic construct
  REGION 1..269: note = Bacillus gibsonii-clade subtilisin
QQTVPWGITRVQAPAVHNRGITGSGVRVAILDSGISTHEDLNVRGGVSFVPGEPGYADLNGHGTHVAGTVAALDNSIGVVGVAPSADLYAVKVLGANGRGSISGIAQGLEWAAQNNMHIANMSLGTDAPSSTLERAVNYATSRDVLVIAATGNNGSGSVGYPARYANAMAVGATDQNNRRANFSQYGTGIDIVAPGVNVQSTYPGNRYVSMNGTSMATPHVAGAAALVKQRYPSWNATQIRNHLKNTATNLGNSSQFGSGLVNAEAATR

SEQ ID NO: 180 - protein, 269 aa, synthetic construct
  REGION 1..269: note = Bacillus gibsonii-clade subtilisin
QQTVPWGITRVQAPAVHNRGITGSGVRVAILDSGISTHEDLNVRGGVSFVPGEPTTADLNGHGTHVAGTVAALNNSIGVVGVAPSADLYAVKVLGANGRGSVSGIAQGLEWAAANNMHVANMSLGTDAPSSTLERAVNYATSRDVLVIAATGNNGSGTVSYPARYANAMAVGATDQNNRRANFSQYGTGIDIVAPGVNVQSTYPGNRYVSMNGTSMATPHVAGAAALVKQRYPSWNATQIRDHLKNTATNLGNSSQYGSGLVNAEAATR

SEQ ID NO: 181 - protein, 269 aa, synthetic construct
  REGION 1..269: note = Bacillus gibsonii-clade subtilisin
QQTVPWGITRVQAPAVHNRGITGSGVKVAILDSGISTHEDLNVRGGVSFVPGEPTYADLNGHGTHVAGTVAALNNSIGVVGVAPSADLYAVKVLGANGRGSISGIAQGLEWAAQNNMHIANMSLGTDAPSSTLERAVNYATSRDVLVIAATGNNGSGSVGYPARYANAMAVGATDQNNRRANFSQYGAGLDIVAPGVNVQSTYPGNRYVSMNGTSMATPHVAGAAALVKQRYPSWNATQIRNHLKNTATNLGNSSQYGSGLVNAEAATR

SEQ ID NO: 182 - protein, 269 aa, synthetic construct
  REGION 1..269: note = Bacillus gibsonii-clade subtilisin
QQTVPWGITRVQAPAVHNRGITGSGVKVAILDSGISTHEDLNVRGGVSFVPGEPTNADLNGHGTHVAGTVAALNNSIGVVGVAPSADLYAVKVLGANGRGSVSGIAQGLEWAAPNNMHIANMSLGTDAPSSTLERAVNYATSTDVLVIAATGNNGSGSIGYPARYANAMAVGATDQNNRRANFSQYGTGIDIVAPGVNVQSTYPGNRYVSMTGTSMATPHVAGAAALVKQRYPSWNATQIRNHLKNTATNLGNSSQYGSGLVNAEAATR

SEQ ID NO: 183 - protein, 269 aa, synthetic construct
  REGION 1..269: note = Bacillus gibsonii-clade subtilisin
QQTVPWGITRVQAPAVHNRGITGSGVRVAILDSGISTHEDLNVRGGVSFVPGEPTTADLNGHGTHVAGTVAALNNSIGVVGVAPSADLYAVKVLGANGRGSISGIAQGLEWAAANNMHVANMSLGTDAPSSTLERAVNYATSRDVLVIAATGNNGSGSVGYPARYANAMAVGATDQNNRRANFSQYGTGIDIVAPGVNVQSTYPGNRYVSMNGTSMATPHVAGAAALVKQRYPSWNATQIRNHLKNTATNLGNSSQYGSGLVNAEAATR

SEQ ID NO: 184 - protein, 269 aa, synthetic construct
  REGION 1..269: note = Bacillus gibsonii-clade subtilisin
QQTVPWGITRVQAPAVHNRGITGSGVRVAILDSGISTHEDLNVRGGVSFVPGEPTYADLNGHGTHVAGTVAALNNSIGVVGVAPSADLYAVKVLGANGRGSVSGIAQGLEWAAQNNMHVANMSLGTDAPSSTLERAVNYATSRDVLVIAATGNNGSGTISYPARYANAMAVGATDQNNRRANFSQYGTGIDIVAPGVNVQSTYPGNRYVSMNGTSMATPHVAGAAALVKQRYPSWNATQIRNHLKNTATNLGNSSQYGSGLVNAEAATR

SEQ ID NO: 185 - protein, 269 aa, synthetic construct
  REGION 1..269: note = Bacillus gibsonii-clade subtilisin
QQTVPWGITRVQAPAVHNRGITGSGVKVAILDSGISTHEDLNIRGGVSFVPGEPTTADLNGHGTHVAGTVAALNNSIGVVGVAPSADLYAVKVLGANGRGSVSGIAQGLEWAAQNNMHVANMSLGTDAPSSTLERAVNYATSRDVLVIAATGNNGSGSVSYPARYANAMAVGATDQNNRRANFSQYGTGIDIVAPGVNVQSTYPGNRYVSMNGTSMATPHVAGAAALVKQRYPSWNATQIRDHLKNTATNLGNSSQYGSGLVNAEAATR

SEQ ID NO: 186 - protein, 269 aa, synthetic construct
  REGION 1..269: note = Bacillus gibsonii-clade subtilisin
QQTVPWGITRVQAPAVHNRGITGSGVRVAILDSGISTHEDLNVRGGVSFVPGEPTTADLNGHGTHVAGTVAALDNSIGVVGVAPSADLYAVKVLGANGRGSVSGIAQGLEWAAANNMHIANMSLGTDAPSSTLERAVNYATSRDVLVIAATGNNGSGSVGYPARYANAMAVGATDQNNRRANFSQYGAGLDIVAPGVNVQSTYPGNRYVSMNGTSMATPHVAGAAALVKQRYPSWNATQIRNHLKNTATNLGNSSQYGSGLVNAEAATR

SEQ ID NO: 187 - protein, 269 aa, synthetic construct
  REGION 1..269: note = Bacillus gibsonii-clade subtilisin
QQTVPWGITRVQAPAVHNRGITGSGVRVAILDSGIANHEDLNVRGGVSFVPGEPTTADLNGHGTHVAGTVAALNNSIGVVGVAPSADLYAVKVLGANGRGSVSGIAQGLEWAAANNMHIANMSLGSDAPSSTLERAVNYATSRDVLVIAATGNNGSGSVGYPARYANAMAVGATDQNNRRANFSQYGTGIDIVAPGVNVQSTYPGNRYVSMNGTSMATPHVAGAAALVKQRYPSWNATQIRDHLLSTATNLGNSNQFGSGLVNAEAATR

SEQ ID NO: 188 - protein, 269 aa, synthetic construct
  REGION 1..269: note = Bacillus gibsonii-clade subtilisin
QQTVPWGITRVQAPAVHNRGITGSGVRVAILDSGIAAHEDLNVRGGVSFVPGEPTTADLNGHGTHVAGTVAALDNSIGVVGVAPSADLYAVKVLGANGRGSVSGIAQGLEWAAANNMHIANMSLGSDAPSSTLERAVNYATSRDVLVIAATGNNGSGSVGYPARYANAMAVGATDQNNRRANFSQYGTGIDIVAPGVNVQSTYPGNRYVSMNGTSMATPHVAGVAALVKQRYPSWNATQIRNHLKNTATNLGNSNQFGSGLVNAEAATR

SEQ ID NO: 189 - protein, 269 aa, synthetic construct
  REGION 1..269: note = Bacillus gibsonii-clade subtilisin
QQTVPWGISRVQAPAVHNRGITGSGVRVAILDSGIAAHEDLNVRGGVSFVPGEPTTADLNGHGTHVAGTVAALNNSIGVVGVAPSADLYAVKVLGANGRGSVSGIAQGLEWAAANNMHIANMSLGSDAPSSTLERAVNYATSQDVLVIAATGNNGSGSVGYPARYANAMAVGATDQNNRRASFSQYGAGLDIVAPGVNVQSTYPGNRYVSMNGTSMATPHVAGAAALVKQRYPSWNATQIRNHLKNTATNLGNSSQFGSGLVNAEAATR

SEQ ID NO: 190 - protein, 269 aa, synthetic construct
  REGION 1..269: note = Bacillus gibsonii-clade subtilisin
QQTVPWGITRVQAPAVHNRGITGSGVRVAILDSGISTHEDLNVRGGVSFVPGESTYQDLNGHGTHVAGTVAALDNSIGVVGVAPSADLYAVKVLGANGRGSVSGIAQGLEWAAANNMHIASMSLGSDAPSSTLERAVNYATSRDVLVIAATGNNGSGTVGYPARYANAMAVGATDQNNRRANFSQYGTGIDIVAPGVNVQSTYPGNRYVSMNGTSMATPHVAGVAALVKQRYPSWNATQIRNHLKNTATNLGNSNQFGSGLVNAEAATR

SEQ ID NO: 191 - protein, 269 aa, synthetic construct
  REGION 1..269: note = Bacillus gibsonii-clade subtilisin
QQTVPWGITRVQAPAVHNRGITGSGVRVAILDSGISTHEDLNVRGGVSFVPGEPTTADLNGHGTHVAGTVAALNNSIGVVGVAPSADLYAVKVLGANGRGSVSGIAQGLEWAAANNMHIANMSLGSDAPSSTLERAVNYATSQDVLVIAATGNNGSGSVGYPARYANAMAVGATDQNNRRANFSQYGTGIDIVAPGVNVQSTYPGNRYVSMSGTSMATPHVAGVAALVKQRNPSWNATQIRNHLLSTATNLGNSNQFGSGLVNAEAATR

SEQ ID NO: 192 - protein, 269 aa, synthetic construct
  REGION 1..269: note = Bacillus gibsonii-clade subtilisin
QQTVPWGITRVQAPAVHNRGITGSGVRVAILDSGISAHEDLNVRGGVSFVPGEPTTADLNGHGTHVAGTVAALNNSIGVVGVAPSADLYAVKVLGANGRGSVSGIAQGLEWAAANNMHIANMSLGSDAPSSTLERAVNYATSRDVLVIAATGNNGSGSVGYPARYANAMAVGATDQNNRRANFSQYGTGIDIVAPGVNVQSTYPGNRYVSMNGTSMATPHVAGVAALVKQRYPSWNATQIRDHLKSTATNLGNSNQFGSGLVNAEAATR

SEQ ID NO: 193 - protein, 269 aa, synthetic construct
  REGION 1..269: note = Bacillus gibsonii-clade subtilisin
QQTVPWGITRVQAPAVHNRGITGSGVRVAILDSGISTHEDLNVRGGVSFVPGEPTTADLNGHGTHVAGTVAALNNSIGVVGVAPSADLYAVKVLGANGRGSVSGIAQGLEWAAANNMHIASMSLGSDAPSSTLERAVNYATSRDVLVIAATGNNGSGSVGYPARYANAMAVGATDQNNRRASFSQYGTGLDIVAPGVNVQSTYPGNRYVSMNGTSMATPHVAGAAALVKQRYPSWNATQIRNHLLSTATNLGNSNQFGSGLVNAEAATR

SEQ ID NO: 194 - protein, 269 aa, synthetic construct
  REGION 1..269: note = Bacillus gibsonii-clade subtilisin
QQTIPWGISRVQAPAVHNRGITGSGVRVAILDSGISTHEDLNVRGGVSFVPGEPTTADLNGHGTHVAGTVAALNNSIGVVGVAPSADLYAVKVLGANGRGSVSGIAQGLEWAAANNMHIASMSLGSDAPSSTLERAVNYATSQDVLVIAATGNNGSGSVGYPARYANAMAVGATDQNNRRANFSQYGTGIDIVAPGVNVQSTYPGNRYVSMNGTSMATPHVAGIAALVKQKYPSWNATQIRNHLLNTATNLGNSSQFGSGLVNAEAATR

SEQ ID NO: 195 - protein, 269 aa, synthetic construct
  REGION 1..269: note = Bacillus gibsonii-clade subtilisin
QQTVPWGITRVQAPAVHNRGITGSGVRVAILDSGISTHEDLNVRGGVSFVPGEPTTADLNGHGTHVAGTVAALDNSIGVVGVAPSADLYAVKVLGANGRGSISGIAQGLEWAAANNMHIASMSLGSDAPSSTLERAVNYATSQDVLVIAATGNNGSGSVGYPARYANAMAVGATDQNNRRANFSQYGTGIDIVAPGVNVQSTYPGNRYVSMNGTSMATPHVAGAAALVKQRYPSWNATQIRNHLKNTATNLGNSSQFGSGLVNAEAATR

SEQ ID NO: 196 - protein, 269 aa, synthetic construct
  REGION 1..269: note = Bacillus gibsonii-clade subtilisin
QQTVPWGITRVQAPAVHNRGITGSGVRVAILDSGISTHEDLNVRGGVSFVPGEPTTADLNGHGTHVAGTVAALNNSIGVVGVAPSADLYAVKVLGANGRGSVSGIAQGLEWAAANNMHIASMSLGSDAPSSTLERAVNYATSRDVLVIAATGNNGSGSVGYPARYANAMAVGATDQNNRRANFSQYGTGIDIVAPGVNVQSTYPGNRYVSMNGTSMATPHVAGAAALVKQRYPSWNATQIRNHLKSTATNLGNSNQFGSGLVNAEAATR

SEQ ID NO: 197 - protein, 269 aa, synthetic construct
  REGION 1..269: note = Bacillus gibsonii-clade subtilisin
QQTVPWGITRVQAPAVHNRGITGSGVRVAILDSGISTHEDLNVRGGVSFVPGEPTTADLNGHGTHVAGTVAALDNSIGVVGVAPSADLYAVKVLGANGRGSVSGIAQGLEWAAQNNMHVANMSLGTDAPSSTLERAVNYATSRDVLVIAATGNNGSGSVGYPARYANAMAVGATDQNNRRANFSQYGTGIDIVAPGVNVQSTYPGNRYVSMNGTSMATPHVAGAAALVKQRYPSWNATQIRNHLKNTATNLGNSSQFGSGLVNAEAATR

SEQ ID NO: 198 - protein, 275 aa, synthetic construct
  REGION 1..275: note = Consensus sequence Figure 8
  SITE 37: note = MISC_FEATURE - Xaa can be any amino acid
  SITE 57: note = MISC_FEATURE - Xaa can be any amino acid
  SITE 116: note = MISC_FEATURE - Xaa can be any amino acid
  REGION 161..164: note = MISC_FEATURE - Xaa can be any amino acid
  SITE 248: note = MISC_FEATURE - Xaa can be any amino acid
QQTVPWGITRVQAPAVHNRGITGSGVRVAILDSGISXTHEDLNVRGGVSFVPGEPTXTADLNGHGTHVAGTVAALNNSIGVVGVAPSADLYAVKVLGANGRGSVSGIAQGLEWAAXNNMHIANMSLGTDAPSSTLERAVNYATSRDVLVIAATGNNGSGSXXXXVGYPARYANAMAVGATDQNNRRANFSQYGTGIDIVAPGVNVQSTYPGNRYVSMNGTSMATPHVAGAAALVKQRYPSWNATQIRXHLKNTATNLGNSSQFGSGLVNAEAATR

SEQ ID NO: 199 - protein, 269 aa, synthetic construct
  UNSURE 1..269: note = X can be any Amino Acid
QQTVPWGITRVQAPAVHNRGITGSGVRVAILDSGISAHSDLNIRGGASFVPGEPTTADLNGHGTHVAGTVAALDNSIGVVGVAPSADLYAVKVLGANGRGSISGIAQGLEWAATNNMHIANMSLGTDAPSSTLERAVNYATSXDVLVIAATGNNGSGSVGYPARYANAMAVGATDQNNRRANFSQYGTGIDIVAPGVNVQSTYPGNRYVSMNGTSMATPHVAGAAALVKQRYPSWNATQIRDHLKNTATNLGNSSQFGSGLVNAEAATR

SEQ ID NO: 200 - protein, 269 aa, synthetic construct
  REGION 1..269: note = Bacillus gibsonii-clade subtilisin
QQTVPWGITRVQAPAVHNRGITGSGVRVAILDSGISTHSDLNIRGGASFVPGEPTTADLNGHGTHVAGTVAALNNSIGVIGVAPNAELYAVKVLGANGSGSVSGIAQGLEWAATNNMHIANMSLGSDFPSSTLERAVNYATSRDVLVIAATGNNGSGSVGYPARYANAMAVGATDQNNRRANFSQYGTGIDIVAPGVNVQSTYPGNRYVSMNGTSMATPHVAGAAALVKQRYPSWNATQIRNHLKNTATNLGNSSQFGSGLVNAEAATR

SEQ ID NO: 201 - protein, 269 aa, synthetic construct
  REGION 1..269: note = Bacillus gibsonii-clade subtilisin
QQTVPWGITRVQAPAVHNRGITGSGVRVAILDSGISAHEDLNIRGGASFVPGEPTTADLNGHGTHVAGTVAALNNSIGVIGVAPNAELYAVKVLGANGSGSVSGIAQGLEWAATNNMHIANMSLGSDFPSSTLERAVNYATSRDVLVIAATGNNGSGSVGYPARYANAMAVGATDQNNRRANFSQYGTGIDIVAPGVNVQSTYPGNRYVSMNGTSMATPHVAGAAALVKQRYPSWNATQIRNHLKNTATNLGNSSQFGSGLVNAEAATR

SEQ ID NO: 202 - protein, 269 aa, synthetic construct
  REGION 1..269: note = Bacillus gibsonii-clade subtilisin
QQTVPWGITRVQAPAVHNRGITGSGVRVAILDSGISAHSDLNIRGGVSFVPGEPTTADLNGHGTHVAGTVAALNNSIGVIGVAPNAELYAVKVLGANGSGSVSGIAQGLEWAATNNMHIANMSLGSDFPSSTLERAVNYATSRDVLVIAATGNNGSGSVGYPARYANAMAVGATDQNNRRANFSQYGTGIDIVAPGVNVQSTYPGNRYVSMNGTSMATPHVAGAAALVKQRYPSWNATQIRNHLKNTATNLGNSSQFGSGLVNAEAATR

SEQ ID NO: 203 - protein, 269 aa, synthetic construct
  REGION 1..269: note = Bacillus gibsonii-clade subtilisin
QQTVPWGITRVQAPAVHNRGITGSGVRVAILDSGISAHSDLNIRGGASFVPGEPTYADLNGHGTHVAGTVAALNNSIGVIGVAPNAELYAVKVLGANGSGSVSGIAQGLEWAATNNMHIANMSLGSDFPSSTLERAVNYATSRDVLVIAATGNNGSGSVGYPARYANAMAVGATDQNNRRANFSQYGTGIDIVAPGVNVQSTYPGNRYVSMNGTSMATPHVAGAAALVKQRYPSWNATQIRNHLKNTATNLGNSSQFGSGLVNAEAATR

SEQ ID NO: 204 - protein, 269 aa, synthetic construct; identical to SEQ ID NO: 87
  REGION 1..269: note = Bacillus gibsonii-clade subtilisin

SEQ ID NO: 205 - protein, 269 aa, synthetic construct
  REGION 1..269: note = Bacillus gibsonii-clade subtilisin
QQTVPWGITRVQAPAVHNRGITGSGVRVAILDSGISAHSDLNIRGGASFVPGEPTTADLNGHGTHVAGTVAALNNSIGVIGVAPNAELYAVKVLGANGRGSVSGIAQGLEWAATNNMHIANMSLGSDFPSSTLERAVNYATSRDVLVIAATGNNGSGSVGYPARYANAMAVGATDQNNRRANFSQYGTGIDIVAPGVNVQSTYPGNRYVSMNGTSMATPHVAGAAALVKQRYPSWNATQIRNHLKNTATNLGNSSQFGSGLVNAEAATR

SEQ ID NO: 206 - protein, 269 aa, synthetic construct
  REGION 1..269: note = Bacillus gibsonii-clade subtilisin
QQTVPWGITRVQAPAVHNRGITGSGVRVAILDSGISAHSDLNIRGGASFVPGEPTTADLNGHGTHVAGTVAALNNSIGVIGVAPNAELYAVKVLGANGSGSVSGIAQGLEWAAANNMHIANMSLGSDFPSSTLERAVNYATSRDVLVIAATGNNGSGSVGYPARYANAMAVGATDQNNRRANFSQYGTGIDIVAPGVNVQSTYPGNRYVSMNGTSMATPHVAGAAALVKQRYPSWNATQIRNHLKNTATNLGNSSQFGSGLVNAEAATR

SEQ ID NO: 207 - protein, 269 aa, synthetic construct
  REGION 1..269: note = Bacillus gibsonii-clade subtilisin
QQTVPWGITRVQAPAVHNRGITGSGVRVAILDSGISAHSDLNIRGGASFVPGEPTTADLNGHGTHVAGTVAALNNSIGVIGVAPNAELYAVKVLGANGSGSVSGIAQGLEWAATNNMHIANMSLGTDFPSSTLERAVNYATSRDVLVIAATGNNGSGSVGYPARYANAMAVGATDQNNRRANFSQYGTGIDIVAPGVNVQSTYPGNRYVSMNGTSMATPHVAGAAALVKQRYPSWNATQIRNHLKNTATNLGNSSQFGSGLVNAEAATR

SEQ ID NO: 208 - protein, 269 aa, synthetic construct
  REGION 1..269: note = Bacillus gibsonii-clade subtilisin
QQTVPWGITRVQAPAVHNRGITGSGVRVAILDSGISAHSDLNIRGGASFVPGEPTTADLNGHGTHVAGTVAALNNSIGVIGVAPNAELYAVKVLGANGSGSVSGIAQGLEWAATNNMHIANMSLGSDAPSSTLERAVNYATSRDVLVIAATGNNGSGSVGYPARYANAMAVGATDQNNRRANFSQYGTGIDIVAPGVNVQSTYPGNRYVSMNGTSMATPHVAGAAALVKQRYPSWNATQIRNHLKNTATNLGNSSQFGSGLVNAEAATR

SEQ ID NO: 209 - protein, 269 aa, synthetic construct
  REGION 1..269: note = Bacillus gibsonii-clade subtilisin
QQTVPWGITRVQAPAVHNRGITGSGVRVAILDSGISAHSDLNIRGGASFVPGEPTTADLNGHGTHVAGTVAALNNSIGVIGVAPNAELYAVKVLGANGSGSVSGIAQGLEWAATNNMHIANMSLGSDFPSSTLERAVNYATSRDVLVIAATGNNGSGSVGYPARYANAMAVGATDQNNRRANFSQYGTGIDIVAPGVNVQSTYPGNRYVSMNGTSMATPHVAGAAALVKQRYPSWNATQIRDHLKNTATNLGNSSQFGSGLVNAEAATR

SEQ ID NO: 210 - protein, 269 aa, synthetic construct
  REGION 1..269: note = Bacillus gibsonii-clade subtilisin
QQTVPWGITRVQAPAVHNRGITGSGVRVAILDSGISAHSDLNIRGGASFVPGEPTTADLNGHGTHVAGTVAALNNSIGVIGVAPNAELYAVKVLGANGSGSVSGIAQGLEWAAQNNMHIANMSLGSDFPSSTLERAVNYATSRDVLVIAATGNNGSGSVGYPARYANAMAVGATDQNNRRANFSQYGTGIDIVAPGVNVQSTYPGNRYVSMNGTSMATPHVAGAAALVKQRYPSWNATQIRNHLKNTATNLGNSSQFGSGLVNAEAATR

SEQ ID NO: 211 - protein, 269 aa, synthetic construct
  REGION 1..269: note = Bacillus gibsonii-clade subtilisin
QQTVPWGITRVQAPAVHNRGITGSGVRVAILDSGISAHSDLNIRGGASFVPGEPTTADLNGHGTHVAGTVAALNNSIGVIGVAPSAELYAVKVLGANGSGSVSGIAQGLEWAATNNMHIANMSLGSDFPSSTLERAVNYATSRDVLVIAATGNNGSGSVGYPARYANAMAVGATDQNNRRANFSQYGTGIDIVAPGVNVQSTYPGNRYVSMNGTSMATPHVAGAAALVKQRYPSWNATQIRNHLKNTATNLGNSSQFGSGLVNAEAATR

SEQ ID NO: 212 - protein, 269 aa, synthetic construct
  REGION 1..269: note = Bacillus gibsonii-clade subtilisin
QQTVPWGITRVQAPAVHNRGITGSGVRVAILDSGISAHSDLNIRGGASFVPGEPTTADLNGHGTHVAGTVAALNNSIGVVGVAPNAELYAVKVLGANGSGSVSGIAQGLEWAATNNMHIANMSLGSDFPSSTLERAVNYATSRDVLVIAATGNNGSGSVGYPARYANAMAVGATDQNNRRANFSQYGTGIDIVAPGVNVQSTYPGNRYVSMNGTSMATPHVAGAAALVKQRYPSWNATQIRNHLKNTATNLGNSSQFGSGLVNAEAATR

SEQ ID NO: 213 - protein, 269 aa, synthetic construct
  REGION 1..269: note = Bacillus gibsonii-clade subtilisin
QQTVPWGITRVQAPAVHNRGITGSGVRVAILDSGISAHEDLNIRGGASFVPGEPTTADLNGHGTHVAGTVAALNNSIGVIGVAPNADLYAVKVLGANGSGSVSGIAQGLEWAATNNMHIANMSLGSDFPSSTLERAVNYATSRDVLVIAATGNNGSGSVGYPARYANAMAVGATDQNNRRANFSQYGTGIDIVAPGVNVQSTYPGNRYVSMNGTSMATPHVAGAAALVKQRYPSWNATQIRNHLKNTATNLGNSSQFGSGLVNAEAATR

SEQ ID NO: 214 - protein, 269 aa, synthetic construct
  REGION 1..269: note = Bacillus gibsonii-clade subtilisin
QQTVPWGITRVQAPAVHNRGITGSGVRVAILDSGISAHEDLNIRGGASFVPGEPTTADLNGHGTHVAGTVAALNNSIGVIGVAPNADLYAVKVLGANGSGSVSGIAQGLEWAATNNMHIANMSLGSDFPSSTLERAVNYATSRDVLVIAATGNNGSGSVGYPARYANAMAVGATDQNNRRANFSQYGTGIDIVAPGVNVQSTYPGNRYVSMNGTSMATPHVAGAAALVKQRYPSWNATQIRDHLKNTATNLGNSSQFGSGLVNAEAATR

SEQ ID NO: 215 - protein, 269 aa, synthetic construct
  REGION 1..269: note = Bacillus gibsonii-clade subtilisin
QQTVPWGITRVQAPAVHNRGITGSGVRVAILDSGISAHEDLNIRGGASFVPGEPTTADLNGHGTHVAGTVAALNNSIGVIGVAPNADLYAVKVLGANGRGSVSGIAQGLEWAATNNMHIANMSLGSDAPSSTLERAVNYATSRDVLVIAATGNNGSGSVGYPARYANAMAVGATDQNNRRANFSQYGTGIDIVAPGVNVQSTYPGNRYVSMNGTSMATPHVAGAAALVKQRYPSWNATQIRNHLKNTATNLGNSSQFGSGLVNAEAATR

SEQ ID NO: 216 - protein, 269 aa, synthetic construct
  REGION 1..269: note = Bacillus gibsonii-clade subtilisin
QQTVPWGITRVQAPAVHNRGITGSGVRVAILDSGISAHEDLNIRGGASFVPGEPTTADLNGHGTHVAGTVAALNNSIGVIGVAPNADLYAVKVLGANGRGSVSGIAQGLEWAATNNMHIANMSLGSDAPSSTLERAVNYATSRDVLVIAATGNNGSGSVGYPARYANAMAVGATDQNNRRANFSQYGTGIDIVAPGVNVQSTYPGNRYVSMNGTSMATPHVAGAAALVKQRYPSWNATQIRDHLKNTATNLGNSSQFGSGLVNAEAATR

SEQ ID NO: 217 - protein, 269 aa, synthetic construct
  REGION 1..269: note = Bacillus gibsonii-clade subtilisin
QQTVPWGITRVQAPAVHNRGITGSGVRVAILDSGISAHSDLNIRGGASFVPGEPTYADLNGHGTHVAGTVAALNNSIGVIGVAPNAELYAVKVLGANGRGSVSGIAQGLEWAAQNNMHIANMSLGSDAPSSTLERAVNYATSRDVLVIAATGNNGSGSVGYPARYANAMAVGATDQNNRRANFSQYGTGIDIVAPGVNVQSTYPGNRYVSMNGTSMATPHVAGAAALVKQRYPSWNATQIRNHLKNTATNLGNSSQFGSGLVNAEAATR

SEQ ID NO: 218 - protein, 269 aa, synthetic construct
  REGION 1..269: note = Bacillus gibsonii-clade subtilisin
QQTVPWGITRVQAPAVHNRGITGSGVRVAILDSGISAHEDLNIRGGASFVPGEPTTADLNGHGTHVAGTVAALNNSIGVIGVAPNADLYAVKVLGANGRGSVSGIAQGLEWAATNNMHIANMSLGTDFPSSTLERAVNYATSRDVLVIAATGNNGSGSVGYPARYANAMAVGATDQNNRRANFSQYGTGIDIVAPGVNVQSTYPGNRYVSMNGTSMATPHVAGAAALVKQRYPSWNATQIRNHLKNTATNLGNSSQFGSGLVNAEAATR

SEQ ID NO: 219 - protein, 269 aa, synthetic construct
  REGION 1..269: note = Bacillus gibsonii-clade subtilisin
QQTVPWGITRVQAPAVHNRGITGSGVRVAILDSGISAHSDLNIRGGASFVPGEPTTADLNGHGTHVAGTVAALNNSIGVIGVAPNAELYAVKVLGANGRGSVSGIAQGLEWAATNNMHIANMSLGTDFPSSTLERAVNYATSRDVLVIAATGNNGSGSVGYPARYANAMAVGATDQNNRRANFSQYGTGIDIVAPGVNVQSTYPGNRYVSMNGTSMATPHVAGAAALVKQRYPSWNATQIRNHLKNTATNLGNSSQFGSGLVNAEAATR

SEQ ID NO: 220 - protein, 269 aa, synthetic construct
  REGION 1..269: note = Bacillus gibsonii-clade subtilisin
QQTVPWGITRVQAPAVHNRGITGSGVRVAILDSGISAHSDLNIRGGASFVPGEPTTADLNGHGTHVAGTVAALNNSIGVIGVAPNAELYAVKVLGANGRGSVSGIAQGLEWAATNNMHIANMSLGTDFPSSTLERAVNYATSRDVLVIAATGNNGSGSVGYPARYANAMAVGATDQNNRRANFSQYGTGIDIVAPGVNVQSTYPGNRYVSMNGTSMATPHVAGAAALVKQRYPSWNATQIRDHLKNTATNLGNSSQFGSGLVNAEAATR

SEQ ID NO: 221 - protein, 269 aa, synthetic construct
  REGION 1..269: note = Bacillus gibsonii-clade subtilisin
QQTVPWGITRVQAPAVHNRGITGSGVRVAILDSGISAHEDLNIRGGASFVPGEPTYADLNGHGTHVAGTVAALNNSIGVIGVAPNADLYAVKVLGANGSGSVSGIAQGLEWAAQNNMHIANMSLGSDFPSSTLERAVNYATSRDVLVIAATGNNGSGSVGYPARYANAMAVGATDQNNRRANFSQYGTGIDIVAPGVNVQSTYPGNRYVSMNGTSMATPHVAGAAALVKQRYPSWNATQIRNHLKNTATNLGNSSQFGSGLVNAEAATR

SEQ ID NO: 222 - protein, 269 aa, synthetic construct
  REGION 1..269: note = Bacillus gibsonii-clade subtilisin
QQTVPWGITRVQAPAVHNRGITGSGVRVAILDSGISAHSDLNIRGGASFVPGEPTYADLNGHGTHVAGTVAALNNSIGVIGVAPNAELYAVKVLGANGSGSVSGIAQGLEWAAQNNMHIANMSLGSDFPSSTLERAVNYATSRDVLVIAATGNNGSGSVGYPARYANAMAVGATDQNNRRANFSQYGTGIDIVAPGVNVQSTYPGNRYVSMNGTSMATPHVAGAAALVKQRYPSWNATQIRDHLKNTATNLGNSSQFGSGLVNAEAATR

SEQ ID NO: 223 - protein, 269 aa, synthetic construct
  REGION 1..269: note = Bacillus gibsonii-clade subtilisin
QQTVPWGITRVQAPAVHNRGITGSGVRVAILDSGISAHSDLNIRGGASFVPGEPTTADLNGHGTHVAGTVAALNNSIGVIGVAPNAELYAVKVLGANGRGSVSGIAQGLEWAATNNMHIANMSLGSDAPSSTLERAVNYATSRDVLVIAATGNNGSGSVGYPARYANAMAVGATDQNNRRANFSQYGTGIDIVAPGVNVQSTYPGNRYVSMNGTSMATPHVAGAAALVKQRYPSWNATQIRNHLKNTATNLGNSSQFGSGLVNAEAATR

SEQ ID NO: 224 - protein, 269 aa, synthetic construct
  REGION 1..269: note = Bacillus gibsonii-clade subtilisin
QQTVPWGITRVQAPAVHNRGITGSGVRVAILDSGISAHSDLNIRGGASFVPGEPTYADLNGHGTHVAGTVAALNNSIGVIGVAPNAELYAVKVLGANGSGSVSGIAQGLEWAAQNNMHIANMSLGSDFPSSTLERAVNYATSRDVLVIAATGNNGSGSVGYPARYANAMAVGATDQNNRRANFSQYGTGIDIVAPGVNVQSTYPGNRYVSMNGTSMATPHVAGAAALVKQRYPSWNATQIRNHLKNTATNLGNSSQFGSGLVNAEAATR

SEQ ID NO: 225 - protein, 269 aa, synthetic construct
  REGION 1..269: note = Bacillus gibsonii-clade subtilisin
QQTVPWGITRVQAPAVHNRGITGSGVRVAILDSGISAHSDLNIRGGASFVPGEPTYADLNGHGTHVAGTVAALNNSIGVIGVAPNAELYAVKVLGANGRGSVSGIAQGLEWAAQNNMHIANMSLGTDFPSSTLERAVNYATSRDVLVIAATGNNGSGSVGYPARYANAMAVGATDQNNRRANFSQYGTGIDIVAPGVNVQSTYPGNRYVSMNGTSMATPHVAGAAALVKQRYPSWNATQIRNHLKNTATNLGNSSQFGSGLVNAEAATR

SEQ ID NO: 226 - protein, 269 aa, synthetic construct
  REGION 1..269: note = Bacillus gibsonii-clade subtilisin
QQTVPWGITRVQAPAVHNRGITGSGVRVAILDSGISAHSDLNIRGGASFVPGEPTTADLNGHGTHVAGTVAALNNSIGVIGVAPNAELYAVKVLGANGRGSVSGIAQGLEWAATNNMHIANMSLGSDAPSSTLERAVNYATSRDVLVIAATGNNGSGSVGYPARYANAMAVGATDQNNRRANFSQYGTGIDIVAPGVNVQSTYPGNRYVSMNGTSMATPHVAGAAALVKQRYPSWNATQIRDHLKNTATNLGNSSQFGSGLVNAEAATR

SEQ ID NO: 227 - protein, 269 aa, synthetic construct
  REGION 1..269: note = Bacillus gibsonii-clade subtilisin
QQTVPWGITRVQAPAVHNRGITGSGVRVAILDSGISAHEDLNIRGGASFVPGEPTTADLNGHGTHVAGTVAALNNSIGVIGVAPNAELYAVKVLGANGRGSVSGIAQGLEWAATNNMHIANMSLGTDAPSSTLERAVNYATSRDVLVIAATGNNGSGSVGYPARYANAMAVGATDQNNRRANFSQYGTGIDIVAPGVNVQSTYPGNRYVSMNGTSMATPHVAGAAALVKQRYPSWNATQIRNHLKNTATNLGNSSQFGSGLVNAEAATR

SEQ ID NO: 228 - protein, 269 aa, synthetic construct
  REGION 1..269: note = Bacillus gibsonii-clade subtilisin
QQTVPWGITRVQAPAVHNRGITGSGVRVAILDSGISAHEDLNIRGGASFVPGEPTYADLNGHGTHVAGTVAALNNSIGVIGVAPNAELYAVKVLGANGRGSVSGIAQGLEWAATNNMHIANMSLGTDAPSSTLERAVNYATSRDVLVIAATGNNGSGSVGYPARYANAMAVGATDQNNRRANFSQYGTGIDIVAPGVNVQSTYPGNRYVSMNGTSMATPHVAGAAALVKQRYPSWNATQIRNHLKNTATNLGNSSQFGSGLVNAEAATR

SEQ ID NO: 229 - protein, 269 aa, synthetic construct
  REGION 1..269: note = Bacillus gibsonii-clade subtilisin
QQTVPWGITRVQAPAVHNRGITGSGVRVAILDSGISAHSDLNIRGGASFVPGEPTTADLNGHGTHVAGTVAALNNSIGVIGVAPNAELYAVKVLGANGSGSVSGIAQGLEWAATNNMHIANMSLGTDAPSSTLERAVNYATSRDVLVIAATGNNGSGSVGYPARYANAMAVGATDQNNRRANFSQYGTGIDIVAPGVNVQSTYPGNRYVSMNGTSMATPHVAGAAALVKQRYPSWNATQIRNHLKNTATNLGNSSQFGSGLVNAEAATR

SEQ ID NO: 230 - protein, 269 aa, synthetic construct
  REGION 1..269: note = Bacillus gibsonii-clade subtilisin
QQTVPWGITRVQAPAVHNRGITGSGVRVAILDSGISAHEDLNIRGGASFVPGEPTTADLNGHGTHVAGTVAALNNSIGVIGVAPNAELYAVKVLGANGRGSVSGIAQGLEWAATNNMHIANMSLGTDFPSSTLERAVNYATSRDVLVIAATGNNGSGSVGYPARYANAMAVGATDQNNRRANFSQYGTGIDIVAPGVNVQSTYPGNRYVSMNGTSMATPHVAGAAALVKQRYPSWNATQIRNHLKNTATNLGNSSQFGSGLVNAEAATR

SEQ ID NO: 231 - protein, 269 aa, synthetic construct
  REGION 1..269: note = Bacillus gibsonii-clade subtilisin
QQTVPWGITRVQAPAVHNRGITGSGVRVAILDSGISAHEDLNIRGGASFVPGEPTTADLNGHGTHVAGTVAALNNSIGVIGVAPNAELYAVKVLGANGRGSVSGIAQGLEWAATNNMHIANMSLGSDAPSSTLERAVNYATSRDVLVIAATGNNGSGSVGYPARYANAMAVGATDQNNRRANFSQYGTGIDIVAPGVNVQSTYPGNRYVSMNGTSMATPHVAGAAALVKQRYPSWNATQIRNHLKNTATNLGNSSQFGSGLVNAEAATR

SEQ ID NO: 232 - protein, 269 aa, synthetic construct
  REGION 1..269: note = Bacillus gibsonii-clade subtilisin
QQTVPWGITRVQAPAVHNRGITGSGVRVAILDSGISAHSDLNIRGGASFVPGEPTTADLNGHGTHVAGTVAALNNSIGVIGVAPNAELYAVKVLGANGRGSVSGIAQGLEWAATNNMHIANMSLGTDAPSSTLERAVNYATSRDVLVIAATGNNGSGSVGYPARYANAMAVGATDQNNRRANFSQYGTGIDIVAPGVNVQSTYPGNRYVSMNGTSMATPHVAGAAALVKQRYPSWNATQIRNHLKNTATNLGNSSQFGSGLVNAEAATR

SEQ ID NO: 233 - protein, 269 aa, synthetic construct
  REGION 1..269: note = Bacillus gibsonii-clade subtilisin
QQTVPWGITRVQAPAVHNRGITGSGVRVAILDSGISAHEDLNIRGGVSFVPGEPTYADLNGHGTHVAGTVAALNNSIGVIGVAPNADLYAVKVLGANGRGSVSGIAQGLEWAAQNNMHIANMSLGTDAPSSTLERAVNYATSRDVLVIAATGNNGSGSVGYPARYANAMAVGATDQNNRRANFSQYGTGIDIVAPGVNVQSTYPGNRYVSMNGTSMATPHVAGAAALVKQRYPSWNATQIRDHLKNTATNLGNSSQFGSGLVNAEAATR

SEQ ID NO: 234 - protein, 269 aa, synthetic construct
  REGION 1..269: note = Bacillus gibsonii-clade subtilisin
QQTVPWGITRVQAPAVHNRGITGSGVRVAILDSGISTHEDLNIRGGASFVPGEPTTADLNGHGTHVAGTVAALNNSIGVIGVAPNAELYAVKVLGANGRGSVSGIAQGLEWAAQNNMHIANMSLGTDAPSSTLERAVNYATSRDVLVIAATGNNGSGSVGYPARYANAMAVGATDQNNRRANFSQYGTGIDIVAPGVNVQSTYPGNRYVSMNGTSMATPHVAGAAALVKQRYPSWNATQIRDHLKNTATNLGNSSQFGSGLVNAEAATR

SEQ ID NO: 235 - protein, 269 aa, synthetic construct
  REGION 1..269: note = Bacillus gibsonii-clade subtilisin
QQTVPWGITRVQAPAVHNRGITGSGVRVAILDSGISAHEDLNIRGGASFVPGEPTTADLNGHGTHVAGTVAALNNSIGVIGVAPNADLYAVKVLGANGRGSVSGIAQGLEWAATNNMHIANMSLGTDAPSSTLERAVNYATSRDVLVIAATGNNGSGSVGYPARYANAMAVGATDQNNRRANFSQYGTGIDIVAPGVNVQSTYPGNRYVSMNGTSMATPHVAGAAALVKQRYPSWNATQIRNHLKNTATNLGNSSQFGSGLVNAEAATR

SEQ ID NO: 236 - protein, 269 aa, synthetic construct
  REGION 1..269: note = Bacillus gibsonii-clade subtilisin
QQTVPWGITRVQAPAVHNRGITGSGVRVAILDSGISAHEDLNIRGGASFVPGEPTYADLNGHGTHVAGTVAALNNSIGVIGVAPNADLYAVKVLGANGRGSVSGIAQGLEWAATNNMHIANMSLGTDAPSSTLERAVNYATSRDVLVIAATGNNGSGSVGYPARYANAMAVGATDQNNRRANFSQYGTGIDIVAPGVNVQSTYPGNRYVSMNGTSMATPHVAGAAALVKQRYPSWNATQIRNHLKNTATNLGNSSQFGSGLVNAEAATR

SEQ ID NO: 237 - protein, 269 aa, synthetic construct
  REGION 1..269: note = Bacillus gibsonii-clade subtilisin
QQTVPWGITRVQAPAVHNRGITGSGVRVAILDSGISTHEDLNIRGGASFVPGEPTTADLNGHGTHVAGTVAALNNSIGVVGVAPSADLYAVKVLGANGRGSVSGIAQGLEWAAQNNMHIANMSLGTDAPSSTLERAVNYATSRDVLVIAATGNNGSGSVGYPARYANAMAVGATDQNNRRANFSQYGTGIDIVAPGVNVQSTYPGNRYVSMNGTSMATPHVAGAAALVKQRYPSWNATQIRDHLKNTATNLGNSSQFGSGLVNAEAATR

SEQ ID NO: 238 - protein, 269 aa, synthetic construct
  REGION 1..269: note = Bacillus gibsonii-clade subtilisin
QQTVPWGITRVQAPAVHNRGITGSGVRVAILDSGISTHEDLNIRGGASFVPGEPTYADLNGHGTHVAGTVAALNNSIGVIGVAPNAELYAVKVLGANGRGSVSGIAQGLEWAATNNMHIANMSLGTDAPSSTLERAVNYATSRDVLVIAATGNNGSGSVGYPARYANAMAVGATDQNNRRANFSQYGTGIDIVAPGVNVQSTYPGNRYVSMNGTSMATPHVAGAAALVKQRYPSWNATQIRDHLKNTATNLGNSSQFGSGLVNAEAATR

SEQ ID NO: 239 - protein, 269 aa, synthetic construct
  REGION 1..269: note = Bacillus gibsonii-clade subtilisin
QQTVPWGITRVQAPAVHNRGITGSGVRVAILDSGISTHEDLNIRGGVSFVPGEPTTADLNGHGTHVAGTVAALNNSIGVVGVAPSADLYAVKVLGANGRGSVSGIAQGLEWAAANNMHIANMSLGTDAPSSTLERAVNYATSRDVLVIAATGNNGSGSVGYPARYANAMAVGATDQNNRRANFSQYGTGIDIVAPGVNVQSTYPGNRYVSMNGTSMATPHVAGAAALVKQRYPSWNATQIRNHLKNTATNLGNSSQFGSGLVNAEAATR

SEQ ID NO: 240 - protein, 269 aa, synthetic construct
  REGION 1..269: note = Bacillus gibsonii-clade subtilisin
QQTVPWGITRVQAPAVHNRGITGSGVRVAILDSGISTHEDLNIRGGASFVPGEPTTADLNGHGTHVAGTVAALNNSIGVIGVAPNADLYAVKVLGANGRGSVSGIAQGLEWAAANNMHIANMSLGSDAPSSTLERAVNYATSRDVLVIAATGNNGSGSVGYPARYANAMAVGATDQNNRRANFSQYGTGIDIVAPGVNVQSTYPGNRYVSMNGTSMATPHVAGAAALVKQRYPSWNATQIRNHLKNTATNLGNSSQFGSGLVNAEAATR

SEQ ID NO: 241 - protein, 269 aa, synthetic construct
  REGION 1..269: note = Bacillus gibsonii-clade subtilisin
QQTVPWGITRVQAPAVHNRGITGSGVRVAILDSGISTHEDLNIRGGVSFVPGEPTYADLNGHGTHVAGTVAALNNSIGVIGVAPNAELYAVKVLGANGRGSVSGIAQGLEWAAQNNMHIANMSLGTDAPSSTLERAVNYATSRDVLVIAATGNNGSGSVGYPARYANAMAVGATDQNNRRANFSQYGTGIDIVAPGVNVQSTYPGNRYVSMNGTSMATPHVAGAAALVKQRYPSWNATQIRNHLKNTATNLGNSSQFGSGLVNAEAATR

SEQ ID NO: 242 - protein, 269 aa, synthetic construct
  REGION 1..269: note = Bacillus gibsonii-clade subtilisin
QQTVPWGITRVQAPAVHNRGITGSGVRVAILDSGISAHEDLNIRGGVSFVPGEPTTADLNGHGTHVAGTVAALNNSIGVIGVAPNADLYAVKVLGANGRGSVSGIAQGLEWAAQNNMHIANMSLGTDAPSSTLERAVNYATSRDVLVIAATGNNGSGSVGYPARYANAMAVGATDQNNRRANFSQYGTGIDIVAPGVNVQSTYPGNRYVSMNGTSMATPHVAGAAALVKQRYPSWNATQIRDHLKNTATNLGNSSQFGSGLVNAEAATR

SEQ ID NO: 243 - protein, 269 aa, synthetic construct
  REGION 1..269: note = Bacillus gibsonii-clade subtilisin
QQTVPWGITRVQAPAVHNRGITGSGVRVAILDSGISAHEDLNIRGGASFVPGEPTTADLNGHGTHVAGTVAALNNSIGVIGVAPSAELYAVKVLGANGRGSVSGIAQGLEWAATNNMHIANMSLGSDAPSSTLERAVNYATSRDVLVIAATGNNGSGSVGYPARYANAMAVGATDQNNRRANFSQYGTGIDIVAPGVNVQSTYPGNRYVSMNGTSMATPHVAGAAALVKQRYPSWNATQIRNHLKNTATNLGNSSQFGSGLVNAEAATR

SEQ ID NO: 244 - protein, 269 aa, synthetic construct
  REGION 1..269: note = Bacillus gibsonii-clade subtilisin
QQTVPWGITRVQAPAVHNRGITGSGVRVAILDSGISAHEDLNIRGGASFVPGEPTTADLNGHGTHVAGTVAALNNSIGVIGVAPNAELYAVKVLGANGRGSVSGIAQGLEWAAQNNMHIANMSLGSDAPSSTLERAVNYATSRDVLVIAATGNNGSGSVGYPARYANAMAVGATDQNNRRANFSQYGTGIDIVAPGVNVQSTYPGNRYVSMNGTSMATPHVAGAAALVKQRYPSWNATQIRNHLKNTATNLGNSSQFGSGLVNAEAATR

SEQ ID NO: 245 - protein, 269 aa, synthetic construct
  REGION 1..269: note = Bacillus gibsonii-clade subtilisin
QQTVPWGITRVQAPAVHNRGITGSGVRVAILDSGISTHEDLNIRGGASFVPGEPTYADLNGHGTHVAGTVAALNNSIGVIGVAPNAELYAVKVLGANGRGSVSGIAQGLEWAAQNNMHIANMSLGTDAPSSTLERAVNYATSRDVLVIAATGNNGSGSVGYPARYANAMAVGATDQNNRRANFSQYGTGIDIVAPGVNVQSTYPGNRYVSMNGTSMATPHVAGAAALVKQRYPSWNATQIRDHLKNTATNLGNSSQFGSGLVNAEAATR

SEQ ID NO: 246 - protein, 269 aa, synthetic construct
  REGION 1..269: note = Bacillus gibsonii-clade subtilisin
QQTVPWGITRVQAPAVHNRGITGSGVRVAILDSGISTHEDLNIRGGVSFVPGEPTYADLNGHGTHVAGTVAALNNSIGVIGVAPNAELYAVKVLGANGRGSVSGIAQGLEWAAANNMHIANMSLGTDAPSSTLERAVNYATSRDVLVIAATGNNGSGSVGYPARYANAMAVGATDQNNRRANFSQYGTGIDIVAPGVNVQSTYPGNRYVSMNGTSMATPHVAGAAALVKQRYPSWNATQIRNHLKNTATNLGNSSQFGSGLVNAEAATR

SEQ ID NO: 247 - protein, 269 aa, synthetic construct
  REGION 1..269: note = Bacillus gibsonii-clade subtilisin
QQTVPWGITRVQAPAVHNRGITGSGVRVAILDSGISAHEDLNIRGGVSFVPGEPTYADLNGHGTHVAGTVAALNNSIGVIGVAPNADLYAVKVLGANGRGSVSGIAQGLEWAAANNMHIANMSLGTDAPSSTLERAVNYATSRDVLVIAATGNNGSGSVGYPARYANAMAVGATDQNNRRANFSQYGTGIDIVAPGVNVQSTYPGNRYVSMNGTSMATPHVAGAAALVKQRYPSWNATQIRDHLKNTATNLGNSSQFGSGLVNAEAATR

SEQ ID NO: 248 - protein, 269 aa, synthetic construct
  REGION 1..269: note = Bacillus gibsonii-clade subtilisin
QQTVPWGITRVQAPAVHNRGITGSGVRVAILDSGISTHEDLNIRGGASFVPGEPTYADLNGHGTHVAGTVAALNNSIGVIGVAPSAELYAVKVLGANGRGSVSGIAQGLEWAAQNNMHIANMSLGTDAPSSTLERAVNYATSRDVLVIAATGNNGSGSVGYPARYANAMAVGATDQNNRRANFSQYGTGIDIVAPGVNVQSTYPGNRYVSMNGTSMATPHVAGAAALVKQRYPSWNATQIRDHLKNTATNLGNSSQFGSGLVNAEAATR

SEQ ID NO: 249 - protein, 269 aa, synthetic construct
  REGION 1..269: note = Bacillus gibsonii-clade subtilisin
QQTVPWGITRVQAPAVHNRGITGSGVRVAILDSGISTHEDLNIRGGVSFVPGEPTYADLNGHGTHVAGTVAALNNSIGVIGVAPNADLYAVKVLGANGRGSVSGIAQGLEWAAANNMHIANMSLGTDAPSSTLERAVNYATSRDVLVIAATGNNGSGSVGYPARYANAMAVGATDQNNRRANFSQYGTGIDIVAPGVNVQSTYPGNRYVSMNGTSMATPHVAGAAALVKQRYPSWNATQIRDHLKNTATNLGNSSQFGSGLVNAEAATR

SEQ ID NO: 250 - protein, 269 aa, synthetic construct
  REGION 1..269: note = Bacillus gibsonii-clade subtilisin
QQTVPWGITRVQAPAVHNRGITGSGVRVAILDSGISAHEDLNIRGGVSFVPGEPTYADLNGHGTHVAGTVAALNNSIGVIGVAPSADLYAVKVLGANGRGSVSGIAQGLEWAAQNNMHIANMSLGTDAPSSTLERAVNYATSRDVLVIAATGNNGSGSVGYPARYANAMAVGATDQNNRRANFSQYGTGIDIVAPGVNVQSTYPGNRYVSMNGTSMATPHVAGAAALVKQRYPSWNATQIRNHLKNTATNLGNSSQFGSGLVNAEAATR

SEQ ID NO: 251 - protein, 269 aa, synthetic construct
  REGION 1..269: note = Bacillus gibsonii-clade subtilisin
QQTVPWGITRVQAPAVHNRGITGSGVRVAILDSGISTHEDLNIRGGASFVPGEPTYADLNGHGTHVAGTVAALNNSIGVIGVAPSADLYAVKVLGANGRGSVSGIAQGLEWAAQNNMHIANMSLGSDAPSSTLERAVNYATSRDVLVIAATGNNGSGSVGYPARYANAMAVGATDQNNRRANFSQYGTGIDIVAPGVNVQSTYPGNRYVSMNGTSMATPHVAGAAALVKQRYPSWNATQIRNHLKNTATNLGNSSQFGSGLVNAEAATR

SEQ ID NO: 252 - protein, 269 aa, synthetic construct
  REGION 1..269: note = Bacillus gibsonii-clade subtilisin
QQTVPWGITRVQAPAVHNRGITGSGVRVAILDSGISAHEDLNIRGGASFVPGEPTYADLNGHGTHVAGTVAALNNSIGVIGVAPNAELYAVKVLGANGRGSVSGIAQGLEWAATNNMHIANMSLGSDAPSSTLERAVNYATSRDVLVIAATGNNGSGSVGYPARYANAMAVGATDQNNRRANFSQYGTGIDIVAPGVNVQSTYPGNRYVSMNGTSMATPHVAGAAALVKQRYPSWNATQIRNHLKNTATNLGNSSQFGSGLVNAEAATR

SEQ ID NO: 253 - protein, 269 aa, synthetic construct
  REGION 1..269: note = Bacillus gibsonii-clade subtilisin
QQTVPWGITRVQAPAVHNRGITGSGVRVAILDSGISAHEDLNIRGGVSFVPGEPTTADLNGHGTHVAGTVAALNNSIGVVGVAPNADLYAVKVLGANGRGSVSGIAQGLEWAAANNMHIANMSLGTDAPSSTLERAVNYATSRDVLVIAATGNNGSGSVGYPARYANAMAVGATDQNNRRANFSQYGTGIDIVAPGVNVQSTYPGNRYVSMNGTSMATPHVAGAAALVKQRYPSWNATQIRDHLKNTATNLGNSSQFGSGLVNAEAATR

SEQ ID NO: 254 - protein, 269 aa, synthetic construct
  REGION 1..269: note = Bacillus gibsonii-clade subtilisin
QQTVPWGITRVQAPAVHNRGITGSGVRVAILDSGISTHEDLNIRGGASFVPGEPTYADLNGHGTHVAGTVAALNNSIGVIGVAPSADLYAVKVLGANGRGSVSGIAQGLEWAAANNMHIANMSLGTDAPSSTLERAVNYATSRDVLVIAATGNNGSGSVGYPARYANAMAVGATDQNNRRANFSQYGTGIDIVAPGVNVQSTYPGNRYVSMNGTSMATPHVAGAAALVKQRYPSWNATQIRDHLKNTATNLGNSSQFGSGLVNAEAATR

SEQ ID NO: 255 - protein, 269 aa, synthetic construct
  REGION 1..269: note = Bacillus gibsonii-clade subtilisin
QQTVPWGITRVQAPAVHNRGITGSGVRVAILDSGISAHEDLNIRGGASFVPGEPTTADLNGHGTHVAGTVAALNNSIGVIGVAPNAELYAVKVLGANGRGSVSGIAQGLEWAAANNMHIANMSLGTDFPSSTLERAVNYATSRDVLVIAATGNNGSGSVGYPARYANAMAVGATDQNNRRANFSQYGTGIDIVAPGVNVQSTYPGNRYVSMNGTSMATPHVAGAAALVKQRYPSWNATQIRNHLKNTATNLGNSSQFGSGLVNAEAATR

SEQ ID NO: 256 - protein, 269 aa, synthetic construct
  REGION 1..269: note = Bacillus gibsonii-clade subtilisin
QQTVPWGITRVQAPAVHNRGITGSGVRVAILDSGISAHEDLNIRGGASFVPGEPTTADLNGHGTHVAGTVAALNNSIGVIGVAPNAELYAVKVLGANGRGSVSGIAQGLEWAAQNNMHIANMSLGTDAPSSTLERAVNYATSRDVLVIAATGNNGSGSVGYPARYANAMAVGATDQNNRRANFSQYGTGIDIVAPGVNVQSTYPGNRYVSMNGTSMATPHVAGAAALVKQRYPSWNATQIRDHLKNTATNLGNSSQFGSGLVNAEAATR

SEQ ID NO: 257 - protein, 269 aa, synthetic construct
  UNSURE 1..269: note = X can be any Amino Acid
QQTVPWGITRVQAPAVHNRGITGSGVRVAILDSGISAHEDLNIRGGASFVPGEPTTADLNGHGTHVAGTVAALNNSIGVIGVAPNAELYAVKVLGANGRGSVSGIAQGLEWAAXNNMHIANMSLGTDAPSSTLERAVNYATSRDVLVIAATGNNGSGSVGYPARYANAMAVGATDQNNRRANFSQYGTGIDIVAPGVNVQSTYPGNRYVSMNGTSMATPHVAGAAALVKQRYPSWNATQIRNHLKNTATNLGNSSQFGSGLVNAEAATR

(19) United States
(12) Patent Application Publication
(10) Pub. No.: US 2024/0368573 A1
(43) Pub. Date: Nov. 7, 2024

(54) BACILLUS GIBSONII-CLADE SERINE PROTEASES

(71) Applicant: Danisco US Inc., Palo Alto, CA (US)

(72) Inventors: Lilia Maria Babe, Emerald Mills, CA (US); Richard R. Bott, Kirkland, WA (US); David A. Estell, San Mateo, CA (US); Harm Mulder, Voorhout (NL); Frits Goedegebuur, Vlaardingen (NL); Jian Yao, Sunnyvale, CA (US); Sina Pricelius, Leiden (NL); Miles Scotcher, San Ramon, CA (US)

(21) Appl. No.: 17/961,007

(22) Filed: Oct. 6, 2022

Related U.S. Application Data
(62) Division of application No. 15/736,401, filed on Dec. 14, 2017, now Pat. No. 11,499,146.
(60) Provisional application No. 62/180,673, filed on Jun. 17, 2015.

(51) Int. Cl. C12N 9/54; C11D 3/386

(52) U.S. Cl. CPC C12N 9/54; C11D 3/386; C11D 3/38663; C12Y 304/21062

(57) ABSTRACT

Disclosed herein is one or more subtilisin variant, nucleic acid encoding same, and compositions and methods related to the production and use thereof, including one or more Bacillus gibsonii-clade subtilisin variant that has improved stability and/or soil removal compared to one or more reference subtilisin.

### CROSS REFERENCE TO RELATED APPLICATIONS

This application is a Divisional of U.S. application Ser. No. 15/736,401, filed Dec. 14, 2017, which is a 371 of International Application No. PCT/US16/38245, filed Jun. 17, 2016, and claims the benefit of U.S. Provisional Application No. 62/180,673, filed Jun. 17, 2015, all of which are hereby incorporated by reference in their entirety.

### REFERENCE TO SEQUENCE LISTING SUBMITTED ELECTRONICALLY

The content of the sequence listing electronically submitted with the application as an XML file (20240729_NB40848USPCD_SeqLst); Size: 397,978 bytes; Created: Jul. 29, 2024) forms part of the application and is hereby incorporated herein by reference in its entirety.

Disclosed herein is one or more subtilisin variant, nucleic acid encoding same, and compositions and methods related to the production and use thereof, including one or more Bacillus gibsonii-clade subtilisin variant that has improved stability and/or soil removal compared to one or more reference subtilisin. Compositions containing the serine proteases are suitable for use in cleaning fabrics and hard surfaces, as well as in a variety of industrial applications.

Serine proteases are enzymes (EC No. 3.4.21) possessing an active site serine that initiates hydrolysis of peptide bonds of proteins. There are two broad categories of serine proteases, based on their structure: chymotrypsin-like (trypsin-like) and subtilisin-like. The prototypical subtilisin (EC No. 3.4.21.62) was initially obtained from B. subtilis. Subtilisins and their homologues are members of the S8 peptidase family of the MEROPS classification scheme. Members of family S8 have a catalytic triad in the order Asp, His and Ser in their amino acid sequence.

Although serine proteases have long been known in the art of industrial enzymes, there remains a need for further serine proteases that are suitable for particular conditions and uses.

The present variants, compositions and methods relate to recombinant serine proteases generated through conventional molecular biology techniques (see, e.g., Sambrook et al, Molecular Cloning: Cold Spring Harbor Laboratory Press). Compositions containing the B. gibsonii-clade serine proteases disclosed herein are suitable for use in cleaning fabrics and hard surfaces, as well as in a variety of industrial applications.

Some embodiments are directed to a subtilisin variant comprising an amino acid sequence comprising two, three, or four or more variations versus SEQ ID NO:85 at positions selected from: (i) 1, 4, 9, 21, 24, 27, 36, 37, 39, 42, 43, 44, 47, 54, 55, 56, 74, 80, 85, 87, 99, 102, 114, 117, 119, 121, 126, 128, 131, 143, 144, 158, 159, 160, 169, 182, 188, 190, 197, 198, 212, 224, 231, 232, 237, 242, 245, 246, 254, 255, 256, and 257; (ii) 37, 39, 47, 56, 80, 85, 87, 99, 114, 126, 128, and 242; (iii) 39, 99, 126, and 128; (iv) 39 in combination with one or more variation at a position selected from 37, 47, 56, 80, 85, 87, 99, 114, 126, 128, and 242; (v) 56 in combination with one or more variation at a position selected from 37, 39, 47, 80, 85, 87, 99, 114, 126, 128, and 242; (vi) 114 in combination with one or more variation at a position selected from 37, 39, 47, 56, 80, 85, 87, 99, 126, 128, and 242; (vii) 126 in combination with one or more variation at a position selected from 37, 39, 47, 56, 80, 85, 87, 99, 114, 128, and 242; (viii) 242 in combination with one or more variation at a position selected from 37, 39, 47, 56, 80, 85, 87, 99, 114, 126, and 128; (ix) 99+128 in combination with one or more variation at a position selected from 39, 56, 114, 126 and 242; (x) 39+242 in combination with one or more variation at a position selected from 37, 47, 56, 80, 85, 87, 99, 114, 126, and 128; or (xi) 39+99+128 in combination with one or more variation at a position selected from 37, 47, 56, 80, 85, 87, 114, 126, and 242; with the proviso that one or more of said two, three, or four or more variations is non-naturally occurring; and wherein the amino acid positions of the variant are numbered by correspondence with the amino acid sequence of SEQ ID NO:85.

Other embodiments are directed to a subtilisin variant comprising an amino acid sequence comprising two, three, or four or more amino acid substitutions at positions selected from: (i) 1, 4, 9, 21, 24, 27, 36, 37, 39, 42, 43, 44, 47, 54, 55, 56, 74, 80, 85, 87, 99, 102, 114, 117, 119, 121, 126, 128, 131, 143, 144, 158, 159, 160, 169, 182, 188, 190, 197, 198,212, 224, 231, 232, 237, 242, 245, 246, 254, 255, 256, and 257; (ii) 37, 39, 47, 56, 80, 85, 87, 99, 114, 126, 128, and 242; (iii) 39, 99, 126, and 128; (iv) 39 in combination with one or more amino acid substitution at a position selected from 37, 47, 56, 80, 85, 87, 99, 114, 126, 128, and 242; (v) 56 in combination with one or more amino acid substitution at a position selected from 37, 39, 47, 80, 85, 87, 99, 114, 126, 128, and 242; (vi) 114 in combination with one or more amino acid substitution at a position selected from 37, 39, 47, 56, 80, 85, 87, 99, 126, 128, and 242; (vii) A/S126 in combination with one or more amino acid substitution at a position selected from 37, 39, 47, 56, 80, 85, 87, 99, 114, 128, and 242; (viii) N242 in combination with one or more amino acid substitution at a position selected from 37, 39, 47, 56, 80, 85, 87, 99, 114, 126, and 128; (ix) 99+128 in combination with one or more amino acid substitution at a position selected from 39, 56, 114, 126 and 242; (x) 39+242 in combination with one or more amino acid substitution at a position selected from 37, 47, 56, 80, 85, 87, 99, 114, 126, and 128; or (xi) 39+99+128 in combination with one or more amino acid substitution at a position selected from 37, 47, 56, 80, 85, 87, 114, 126, and 242; with the proviso that one or more of said two, three, or four or more substitutions is non-naturally occurring; and wherein the amino acid positions of the variant are numbered by correspondence with the amino acid sequence of SEQ ID NO:85.

A further embodiment is directed to a subtilisin variant comprising an amino acid sequence comprising: (i) one or more variation versus SEQ ID NO:85 at positions selected from 56, 114, and 126; (ii) one or more variation versus SEQ ID NO:85 at positions selected from 56N/Y, 114A/P/Q, and 126T; (iii) one or more amino acid substitution at positions selected from 56, 114, and 126; (iv) one or more amino acid substitution at positions selected from T56, A/T114, and A/S126; (v) one or more amino acids substitutions selected from: 56N/Y, 114A/P/Q, and 126T; or (vi) one or more amino acids substitutions selected from: T56N/Y, A/T114A/P/Q, and A/S126T; with the proviso that one or more of said two, three, or four or more substitutions or variations is non-naturally occurring; and wherein the amino acid positions of the variant are numbered by correspondence with the amino acid sequence of SEQ ID NO:85.

In another embodiment, one or more subtilisin variant described herein is a member of the B. gibsonii-clade. In a still further embodiment, one or more subtilisin variant described herein has 70%, 75%, 80%, 85%, 86%, 87%, 88%, 89%, 90%, 91%, 92%, 93%, 94%, 95%, 96%, 97%, 98%, 99% or less than 100% amino acid sequence identity to the amino acid sequence of SEQ ID NO:18 or 85. In yet a still further embodiment, at least one variant described herein has proteolytic activity or an increase in proteolytic or cleaning activity compared to the proteolytic or cleaning activity of the protease having the sequence of SEQ ID NO:18 or 85. In another embodiment, the proteolytic activity is casein hydrolysis.

Another embodiment is directed to a composition comprising a surfactant and at least one variant described herein. In some embodiments, the composition further comprises at least one calcium ion and/or zinc ion, at least one stabilizer, at least one bleaching agent, phosphate, or boron. In some embodiments, the composition is phosphate-free and/or boron-free. In some embodiments, the composition is a granular, powder, solid, bar, liquid, tablet, gel, paste or unit dose composition. In some embodiments, the composition further comprises one or more additional enzymes or enzyme derivatives.

Some embodiments are directed to a method of cleaning, comprising contacting a surface or an item with at least one composition described herein. Other embodiments are directed to a method for producing a variant described herein, comprising stably transforming a host cell with an expression vector comprising a polynucleotide encoding at least one of the variants described herein. Still further embodiments are directed to a polynucleotide comprising a nucleic acid sequence encoding at least one variant described herein.

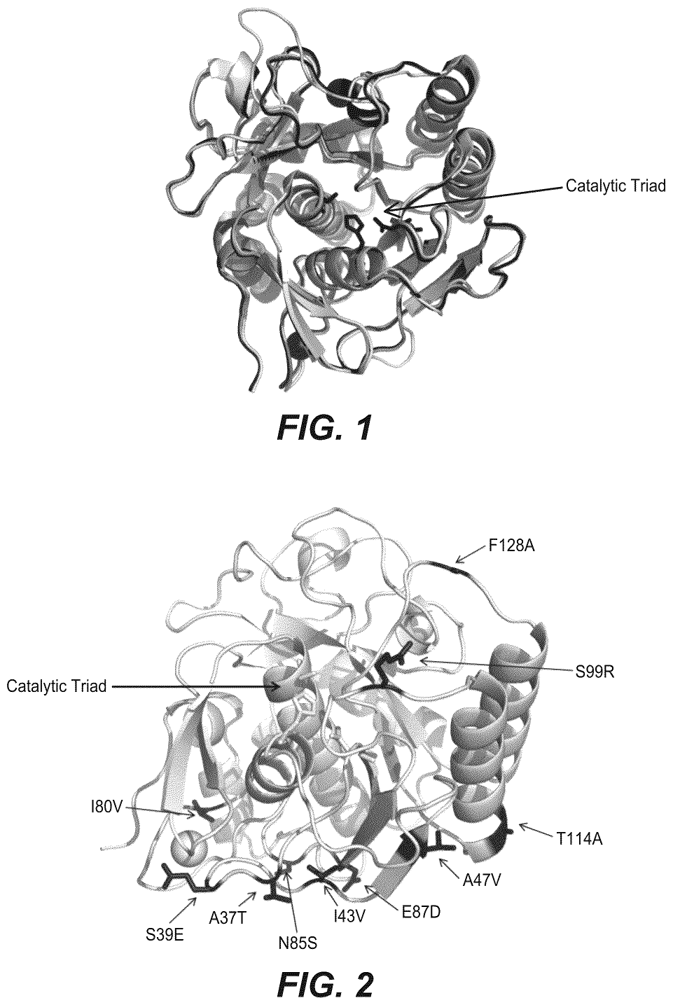

FIG. 1 provides a comparison of the main chain folding of BSP-00801 (black lines) with B. lentus subtilisin (medium gray lines) and subtilisin BPN′ (light gray lines). The side chains of the catalytic triad in BSP-00801 are shown for reference. Apart from a few external loops, the folding pattern in all three subtilisins is conserved.

FIG. 2 provides the structural location of substitutions in B. gibsonii-clade subtilisin BSP-00801. Eight of the ten substitutions (A37T/N, S39E, I43V, A47V, I80V, N85S, E87D, and T114A) in BSP-00801 relative to the parent protease are found along one surface of the molecule near the first calcium binding site.

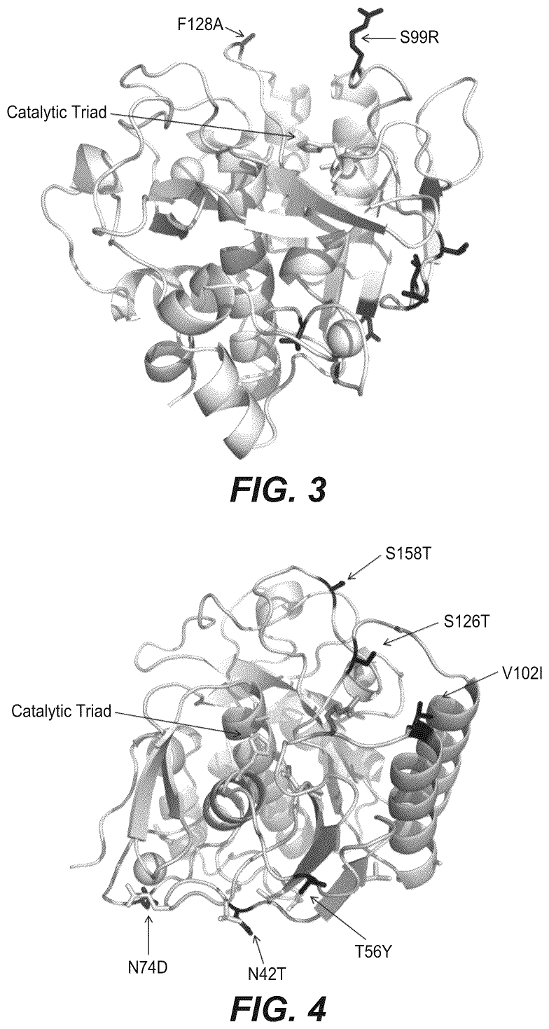

FIG. 3 provides a second view of the BSP-00801 structure with the catalytic triad shown as light sticks and the side chains of the ten substitutions shown as black sticks. This view looks down on the substrate binding surface and it is possible to see that S99R and F128A are located on two of the three loops that form the substrate binding surface corresponding to sub-sites S4-S1.

FIG. 4 provides the location of additional substitutions on BSP-00801. Six additional substitutions are found to enhance the performance of this variant. Three of these (N42T, S56Y and N74D) are found along one surface of the molecule and the other three (V102I, S126T and S158T) are found on loops that form the substrate binding region.

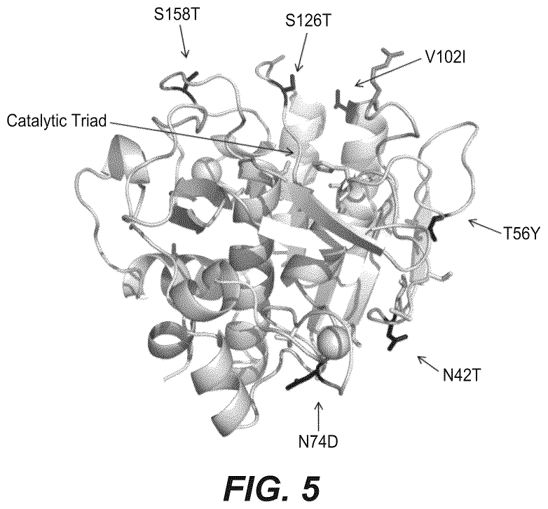

FIG. 5 provides a second view of the BSP-00801 structure with the catalytic triad, and the side chains of the 10 substitutions shown as light gray sticks. The additional sites that further enhance the performance are shown as black sticks. This view looks down the substrate binding surface and it is possible to see the location of three of the additional sites (V102I, S126T and S158T) one on each of the three loops that form the sub-sites S4-S1.

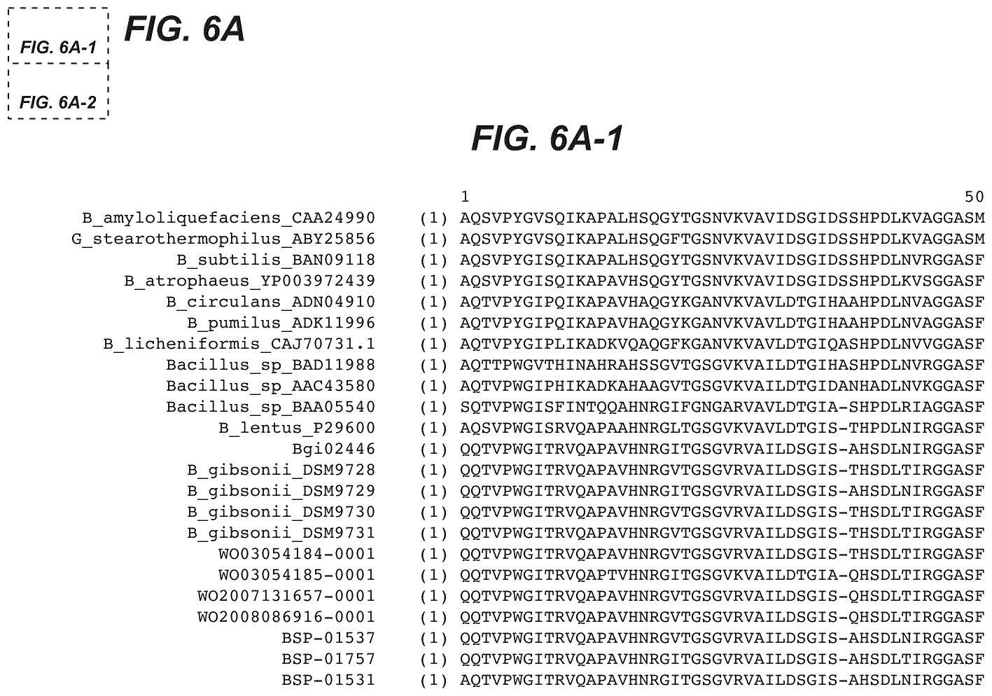

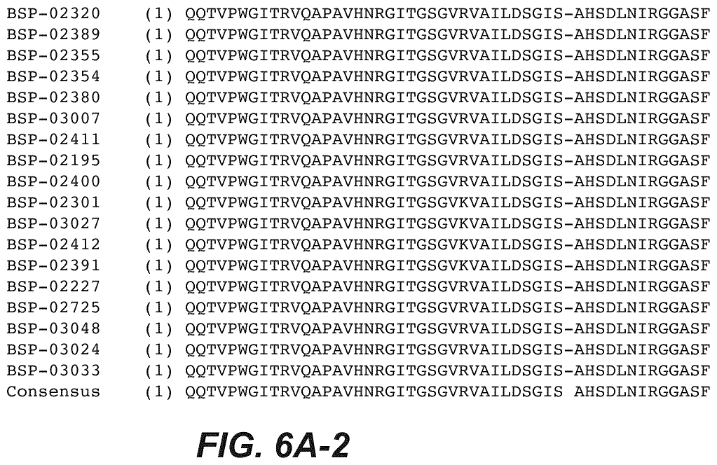

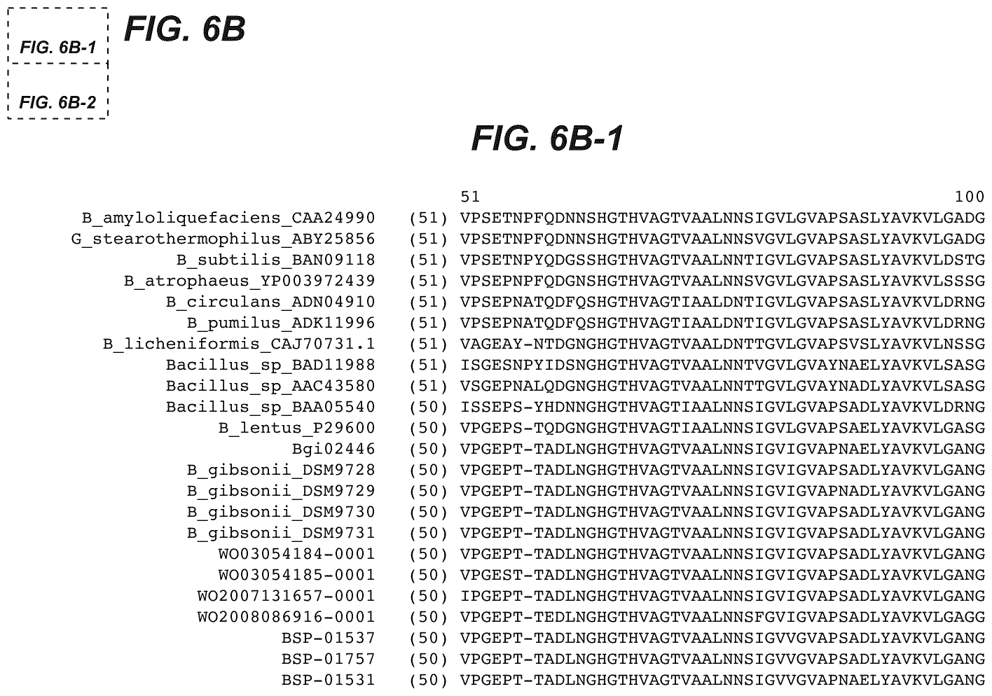

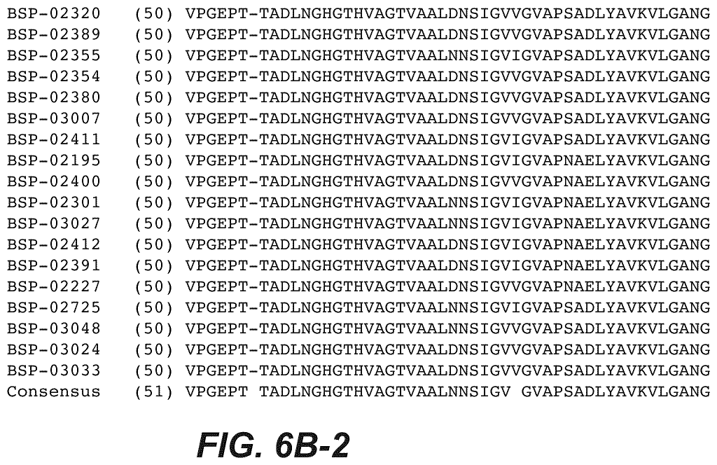

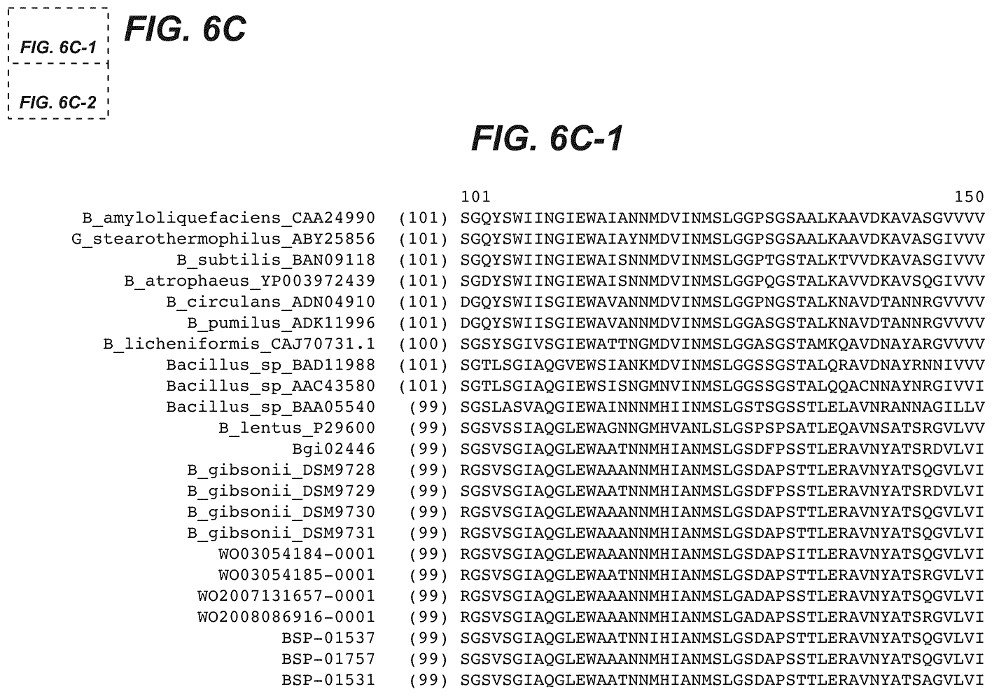

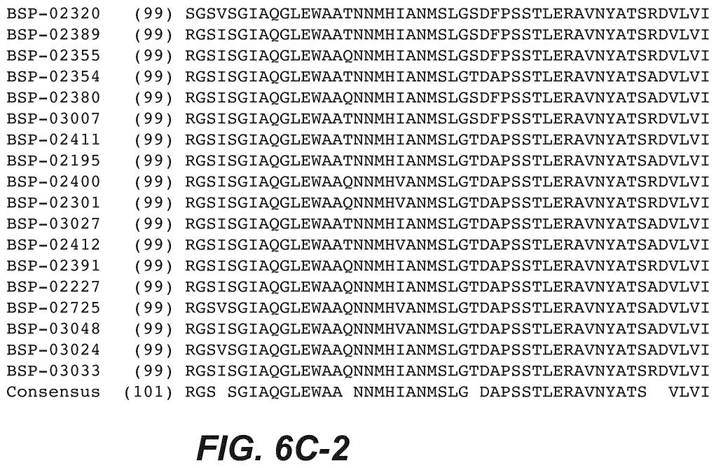

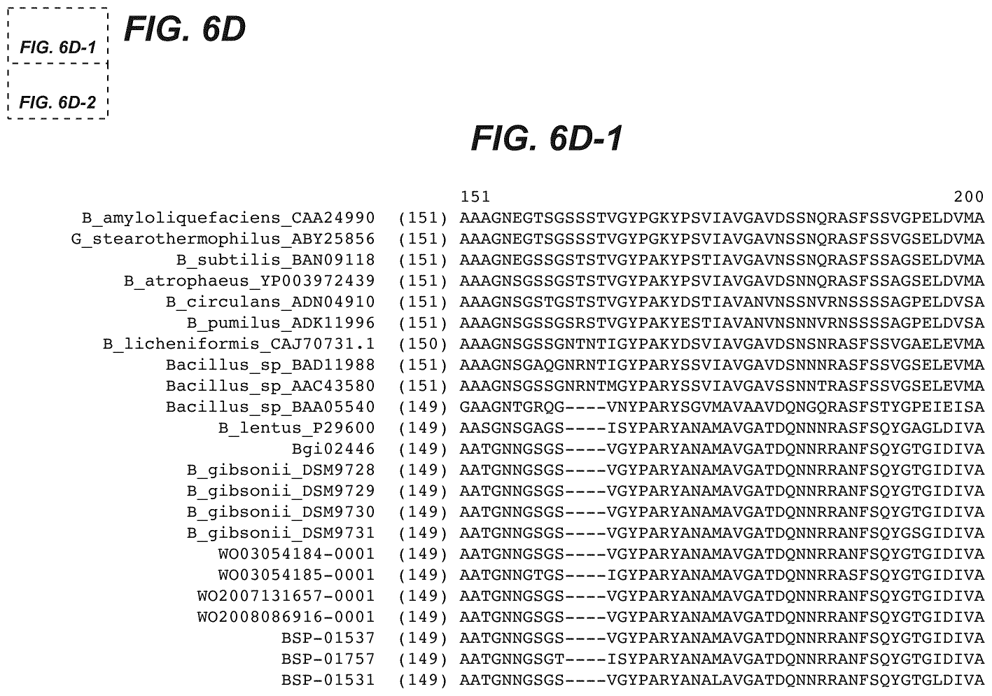

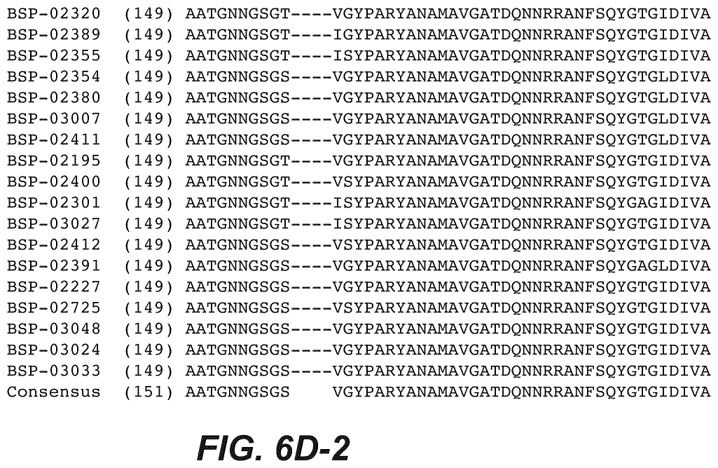

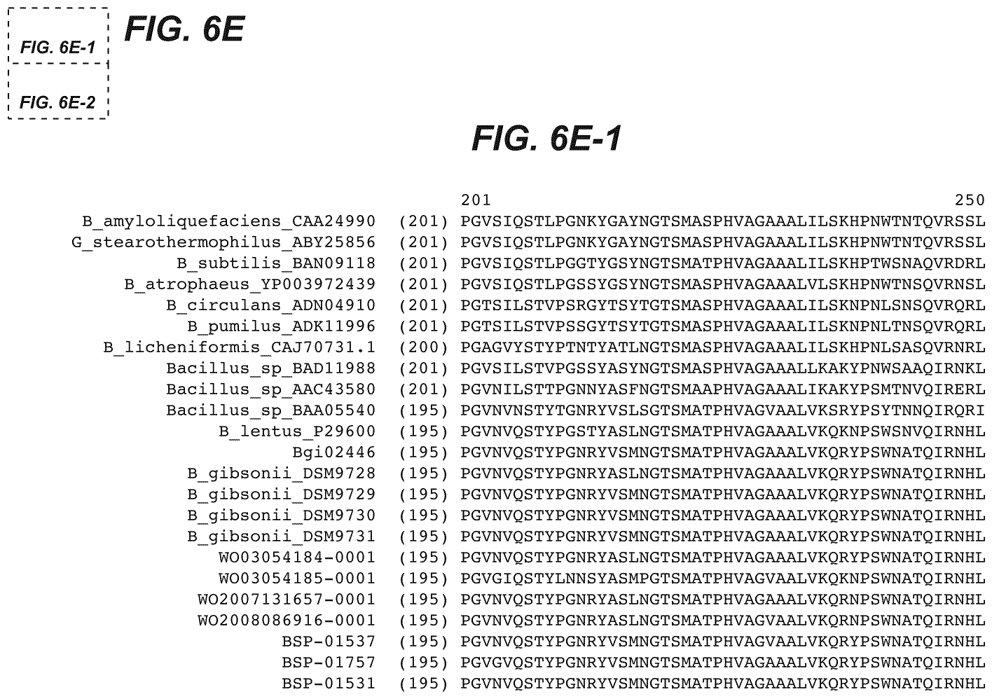

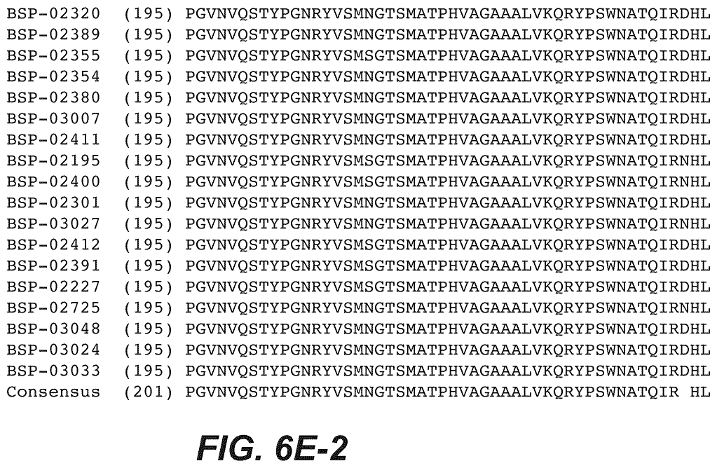

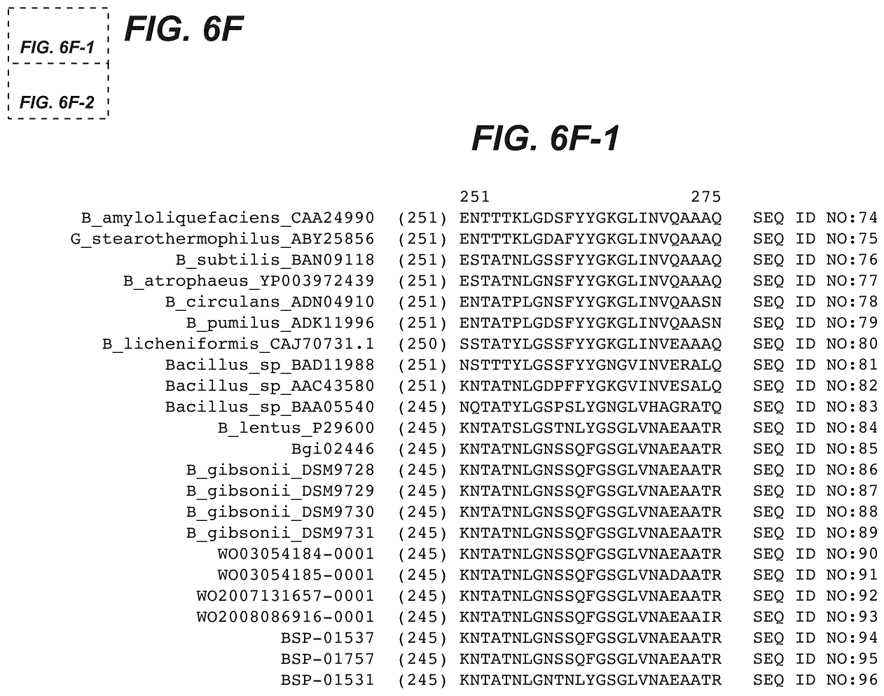

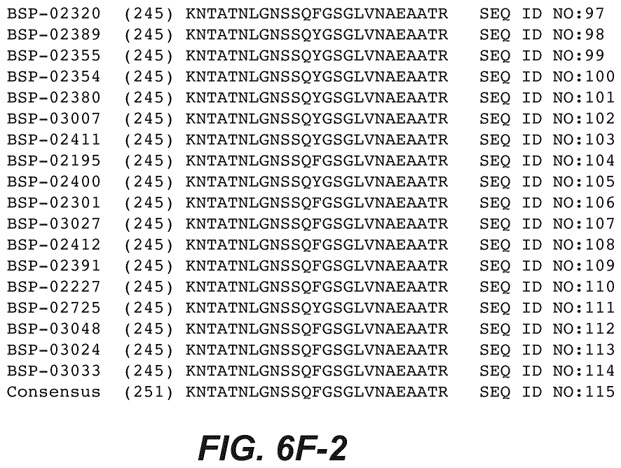

FIG. 6A-F provides an alignment of the amino acid sequences of the predicted mature forms of the B. gibsonii-clade subtilisins of Table 3; B. gibsonii-clade subtilisins DSM 9728, DSM 9729, DSM 9730, DSM 9731 and Bgi02446; and the amino acid sequences of multiple proteases listed in Tables 6-1 and 6-2. A consensus sequence is shown below the alignment.

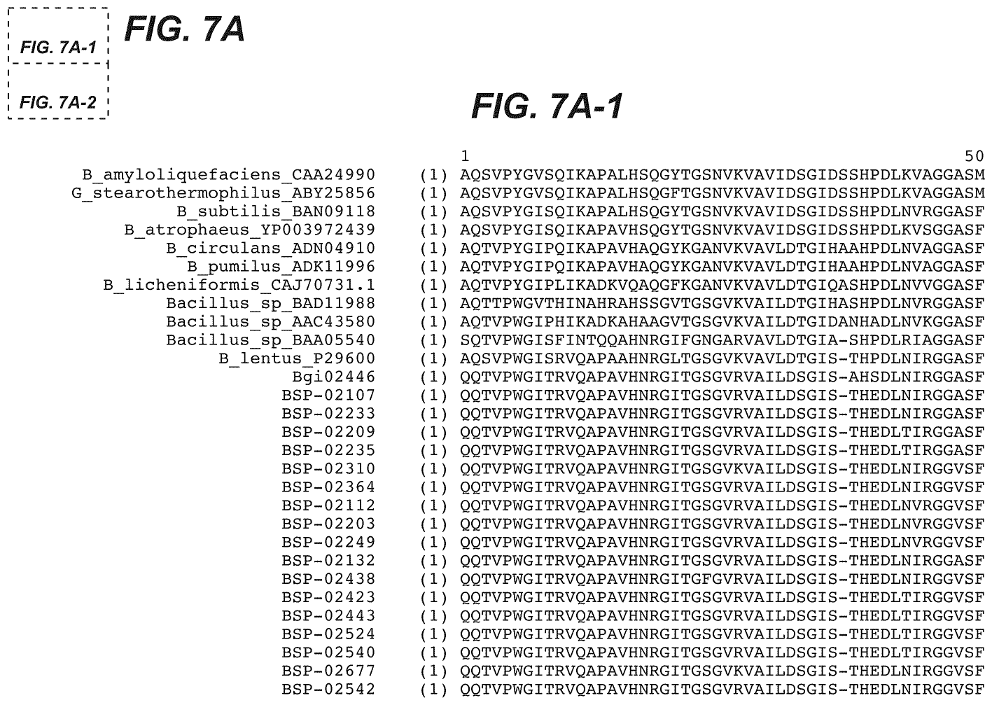

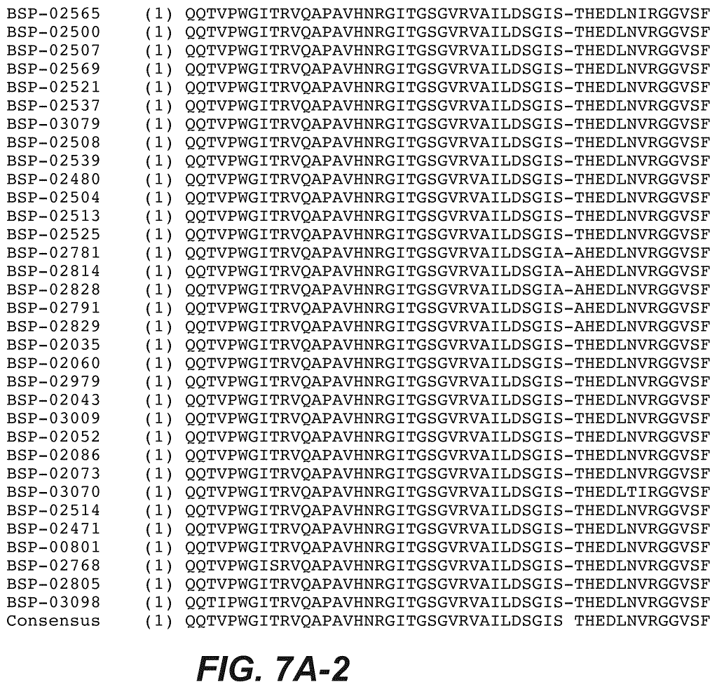

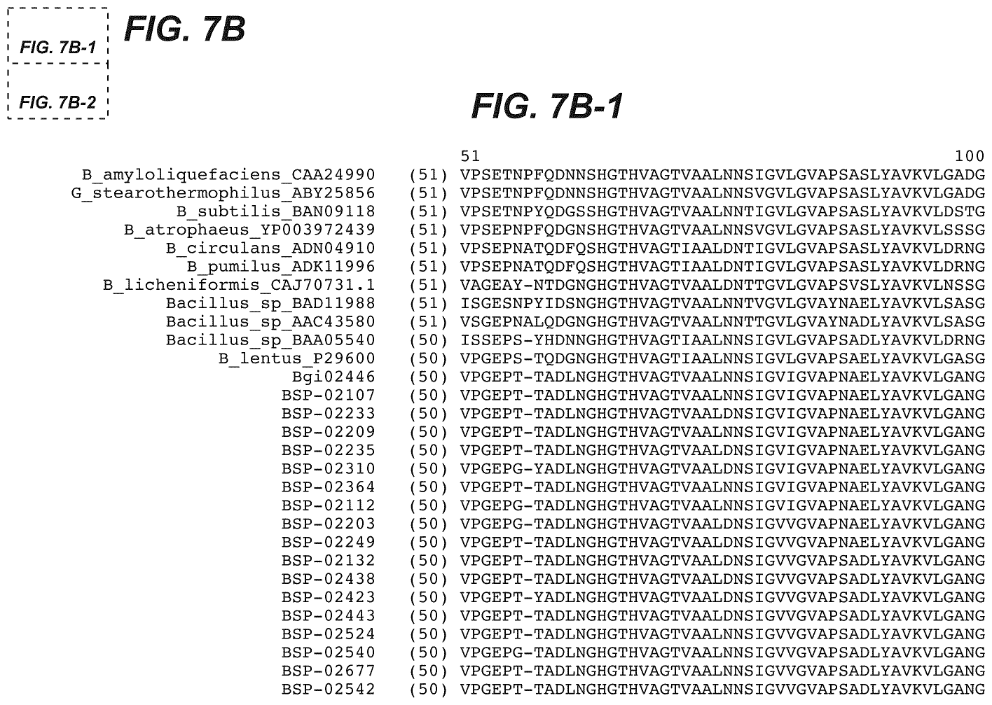

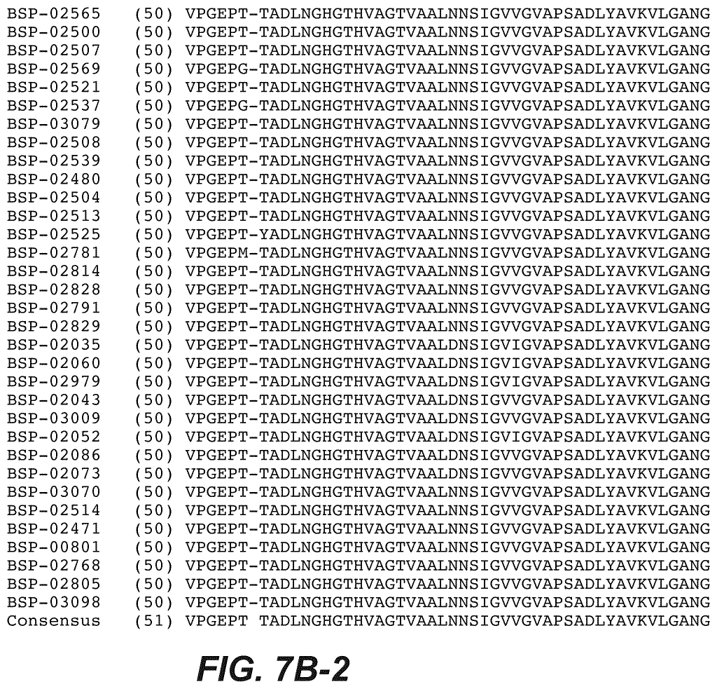

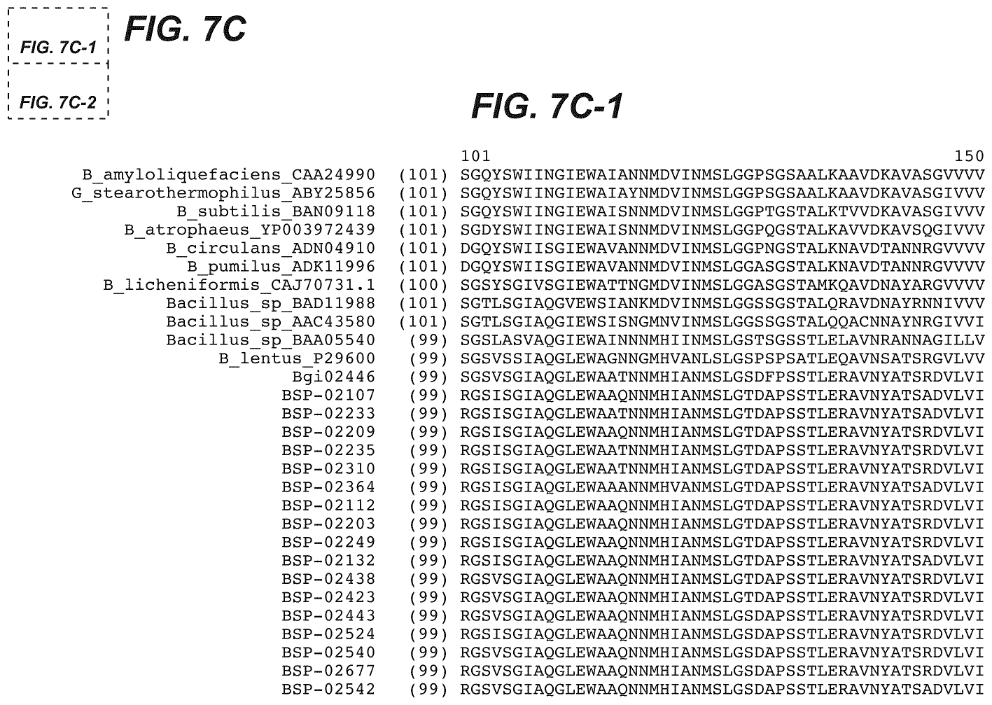

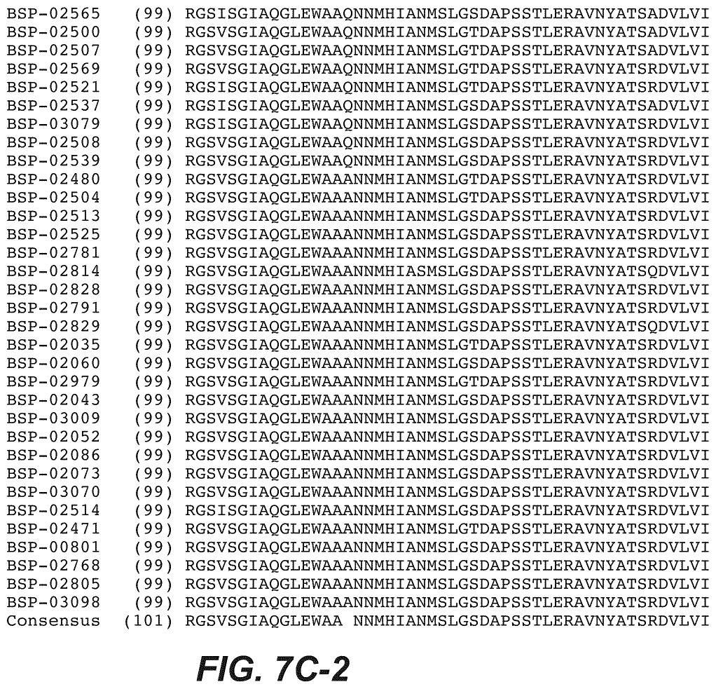

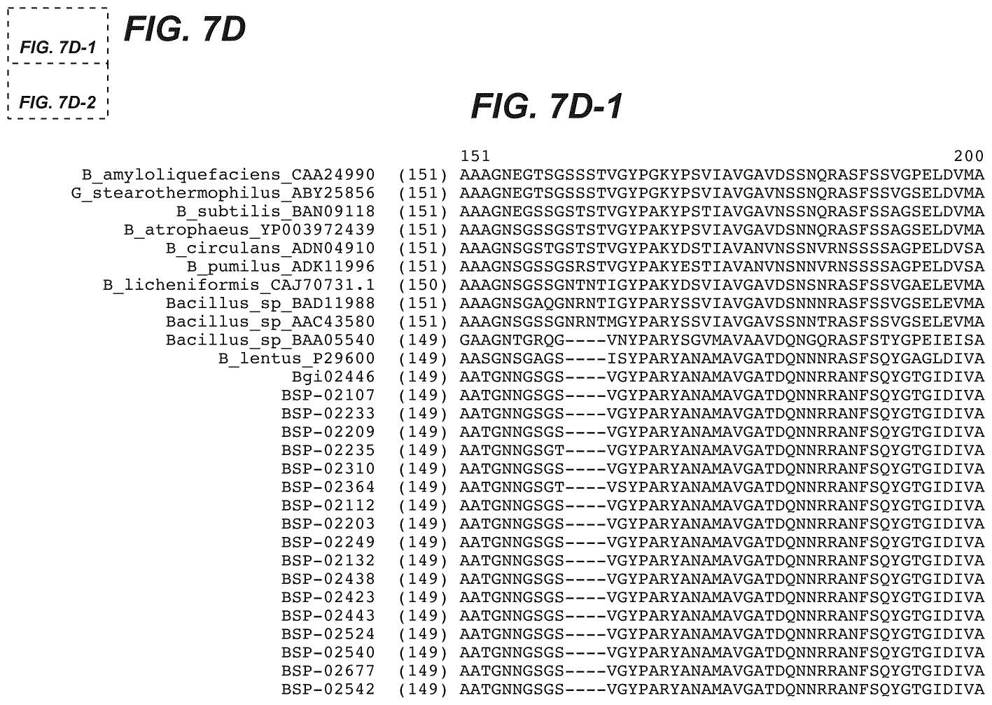

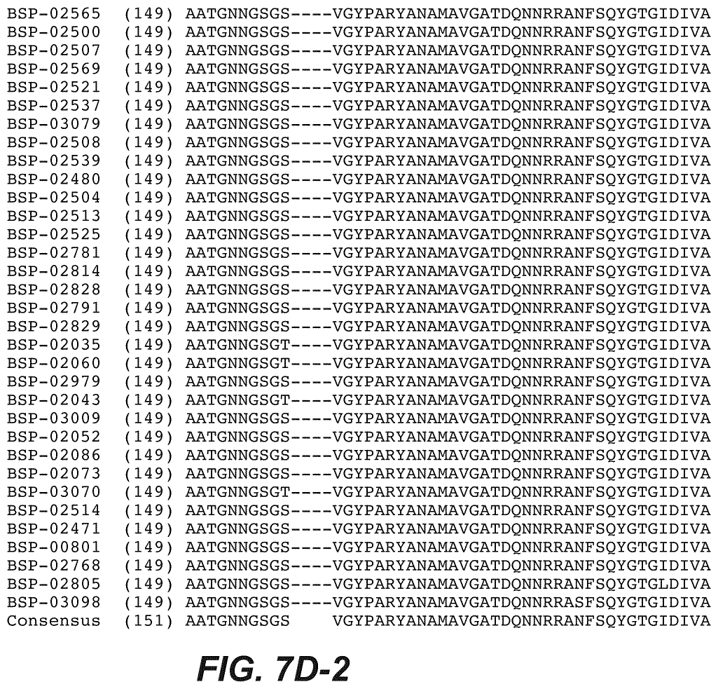

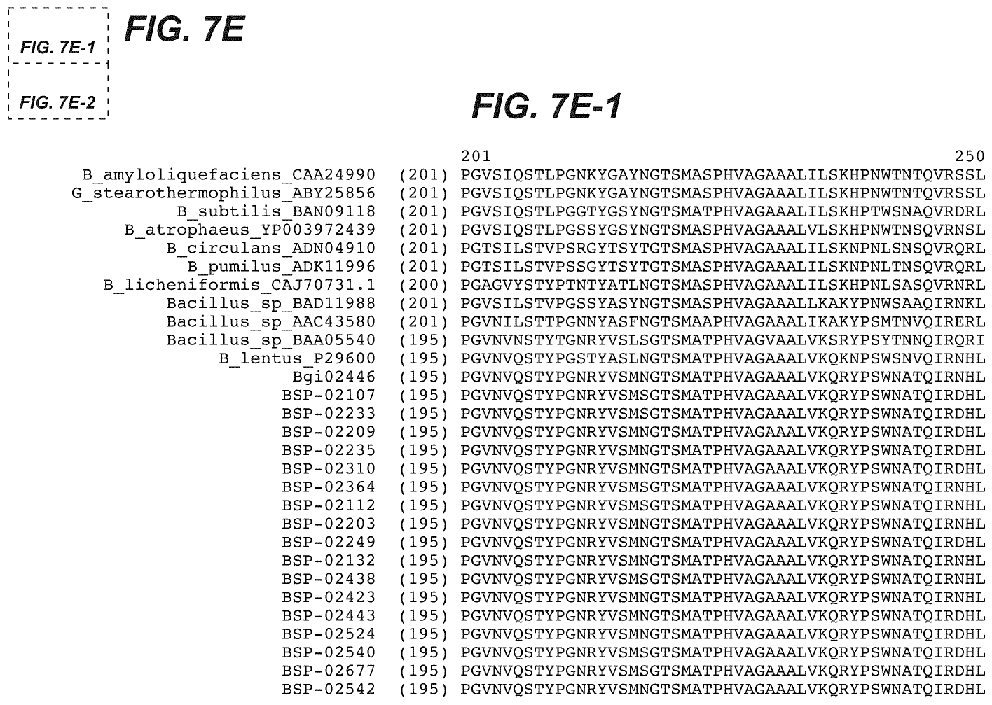

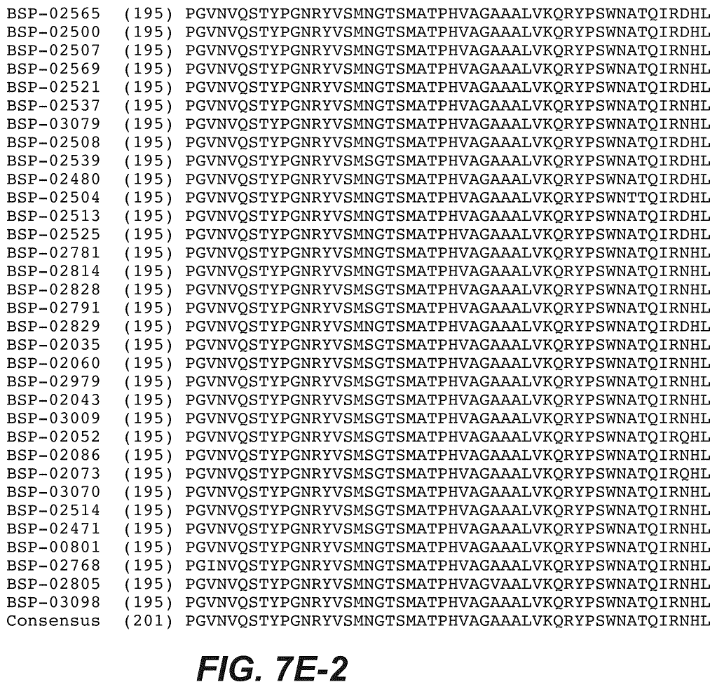

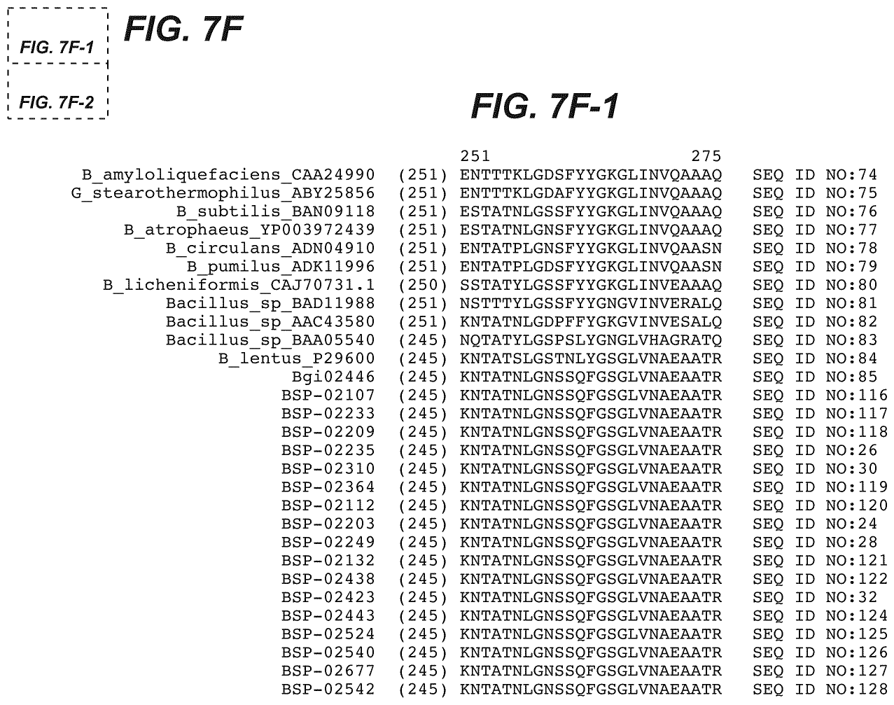

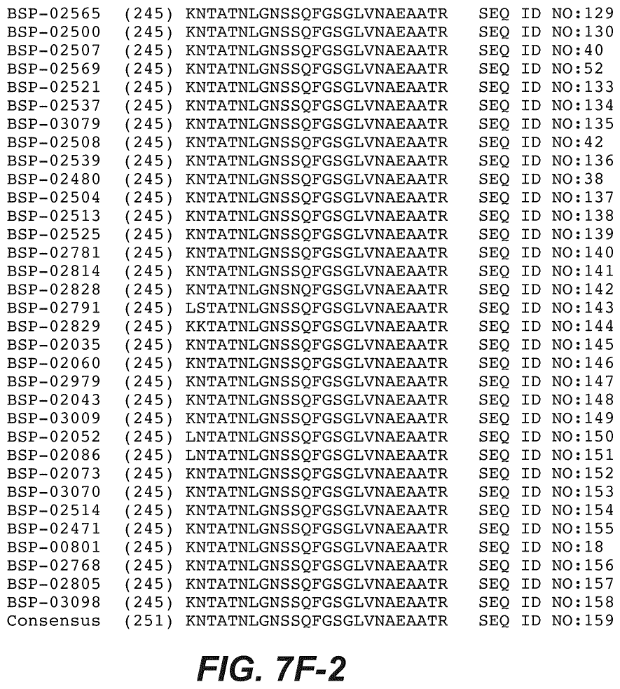

FIG. 7A-F provides an alignment of the amino acid sequences of the predicted mature forms of the B. gibsonii-clade subtilisins of Table 4; B. gibsonii-clade subtilisin Bgi02446; and the amino acid sequences of multiple proteases listed in Table 6-1. A consensus sequence is shown below the alignment.

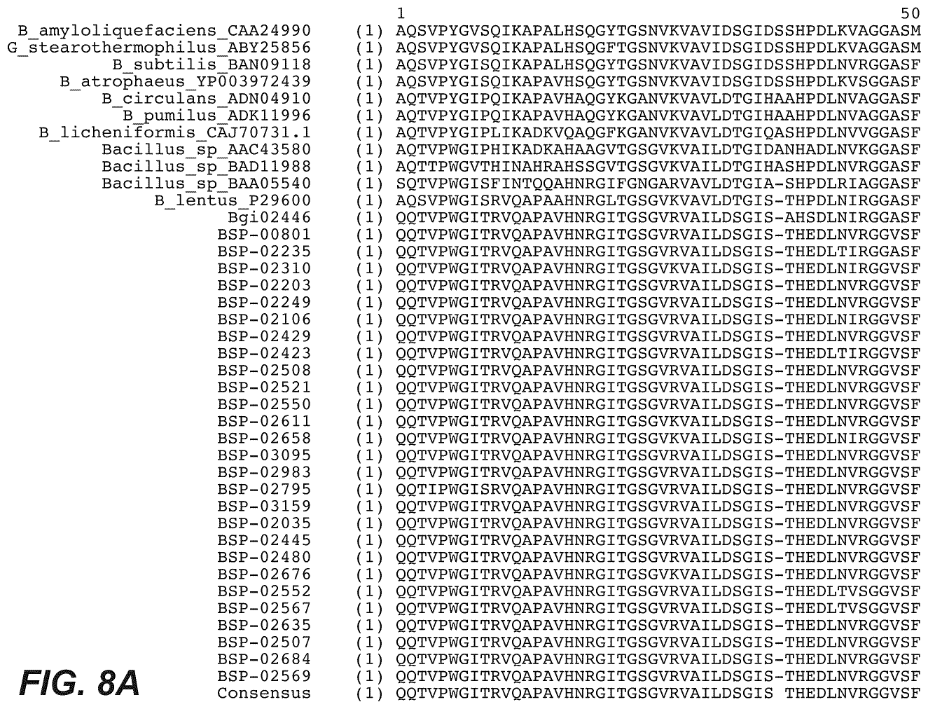

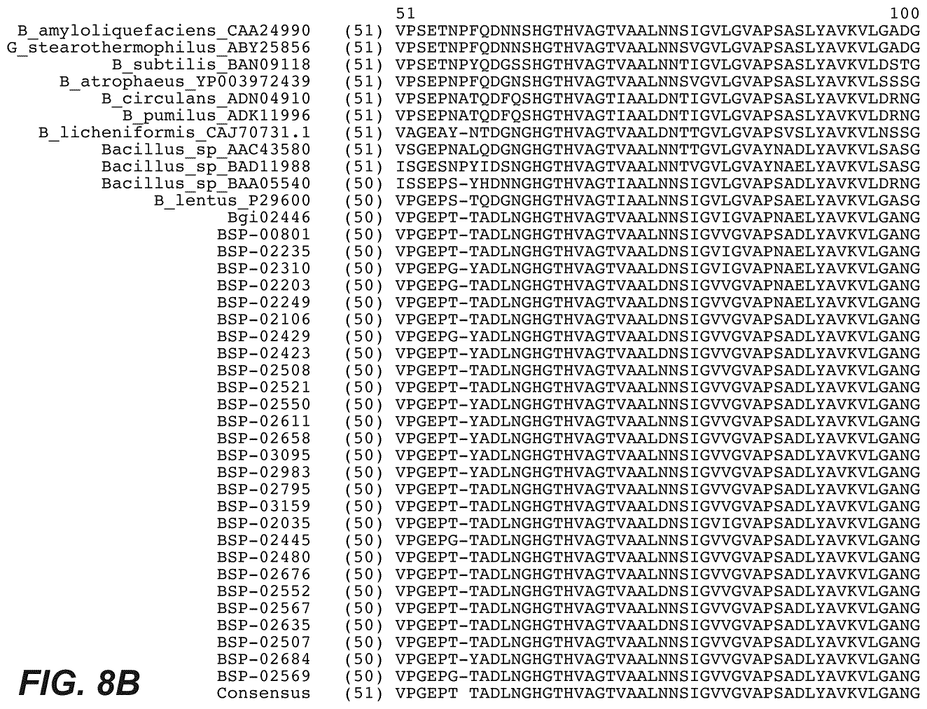

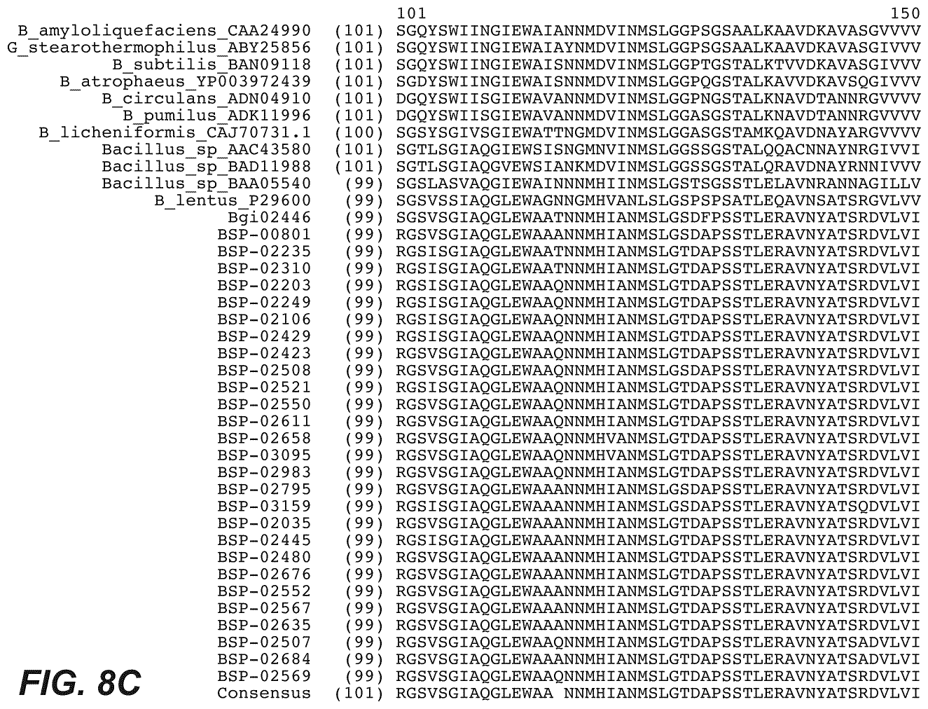

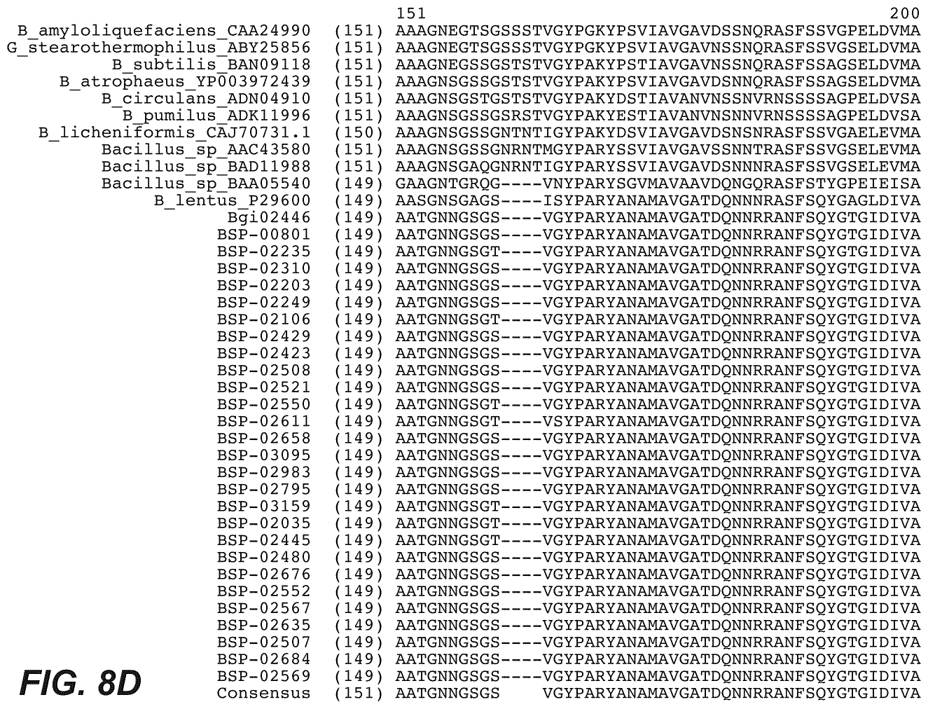

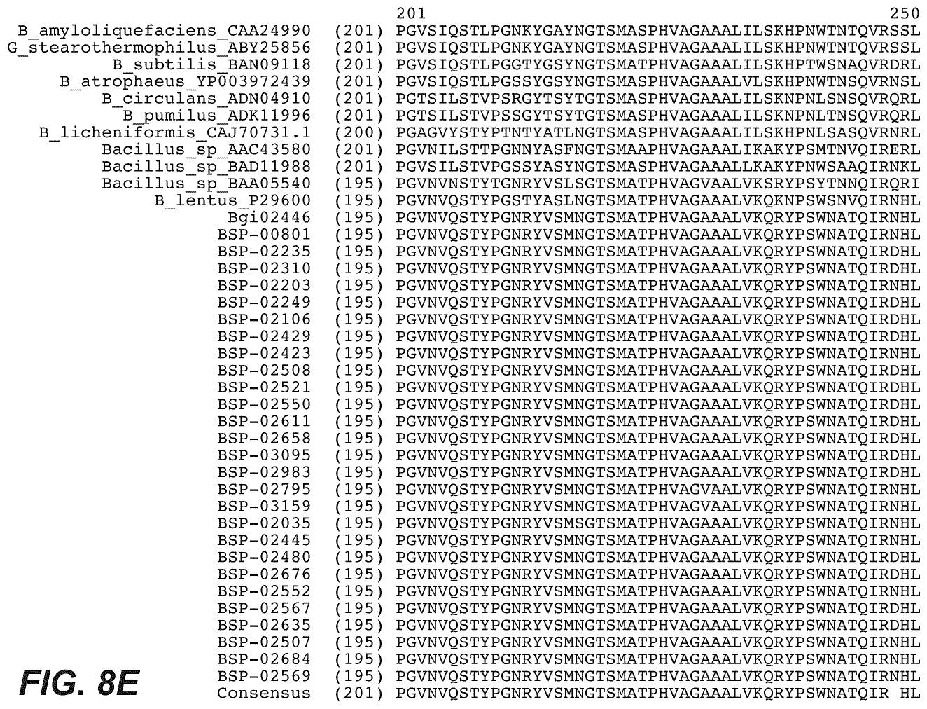

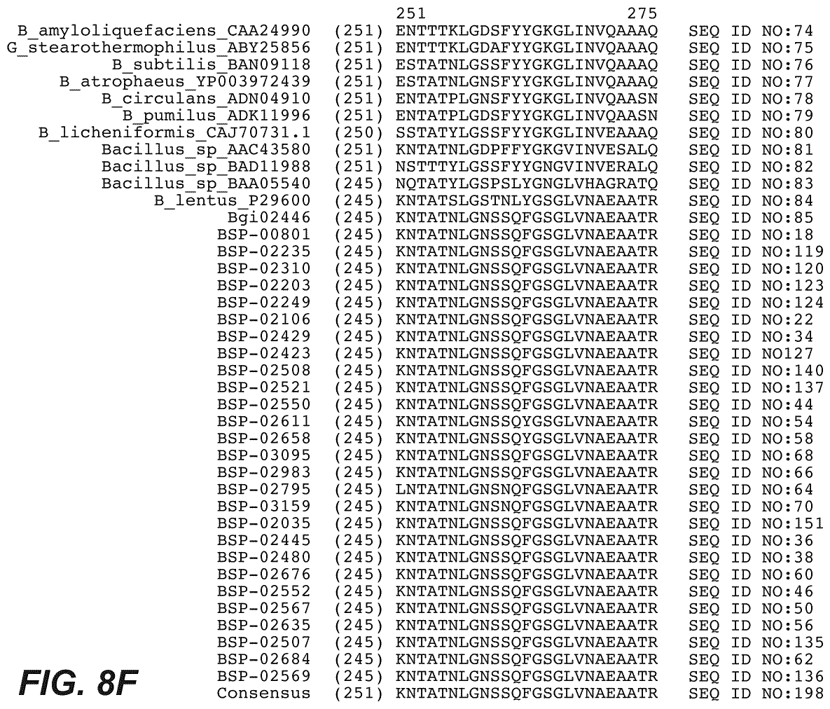

FIG. 8A-F provides an alignment of the amino acid sequences of the predicted mature forms of multiple B. gibsonii-clade BSP-00801 variant subtilisins of Table 5; B. gibsonii-clade subtilisin Bgi02446; and the amino acid sequences of multiple proteases listed in Table 6-1. A consensus sequence is shown below the alignment.

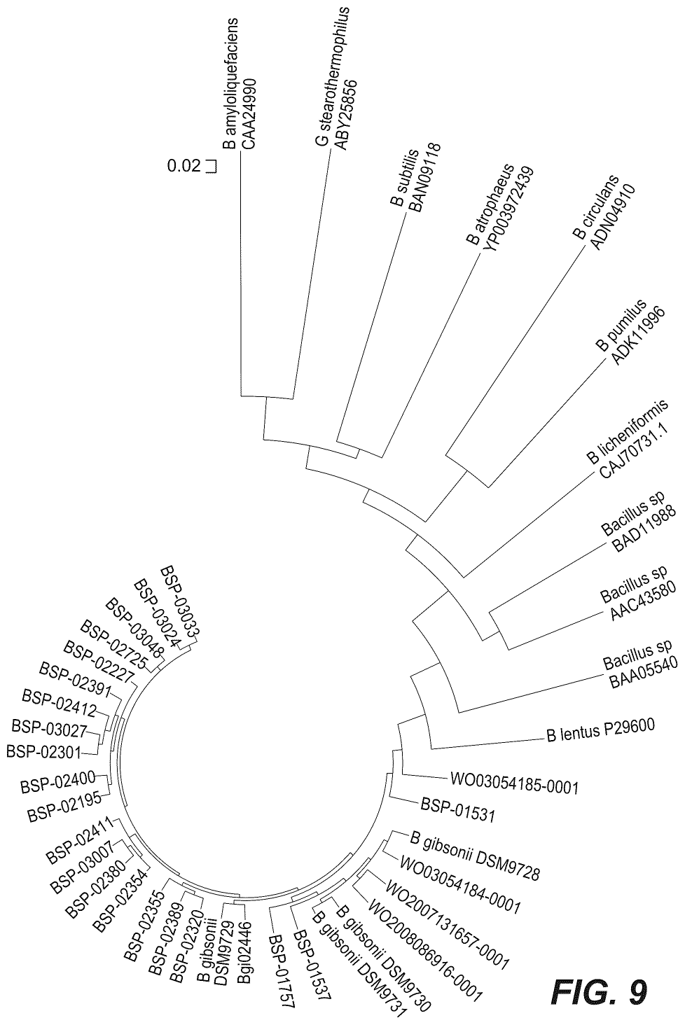

FIG. 9 provides a phylogenetic tree of the B. gibsonii-clade subtilisins of Table 3, the B. gibsonii-clade subtilisins DSM 9728, DSM 9729, DSM 9730, DSM 9731 and Bgi02446, and various other bacterial serine proteases.

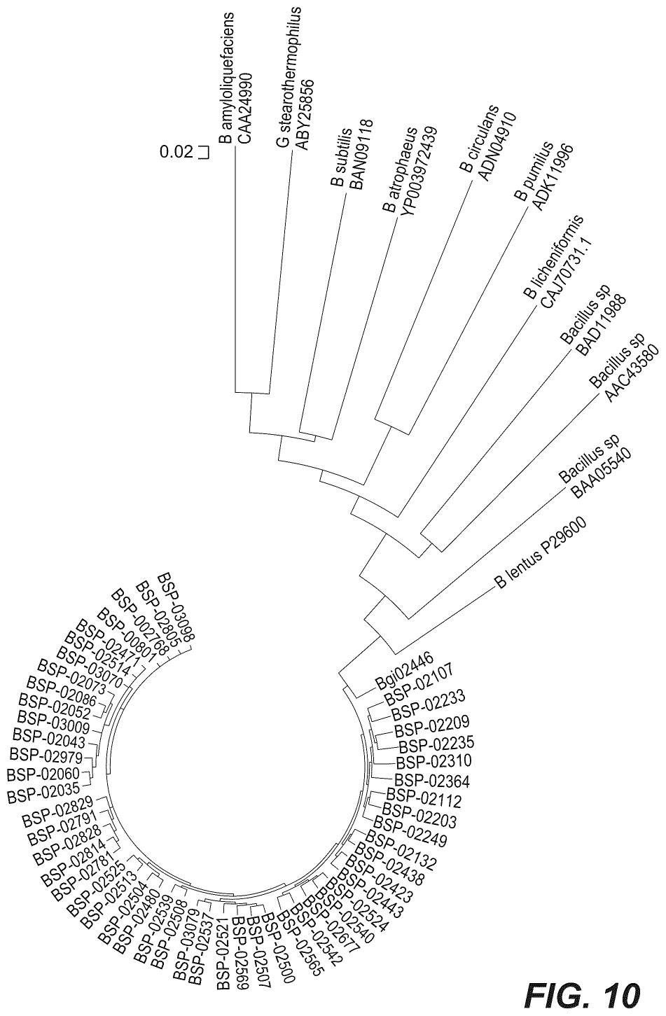

FIG. 10 provides a phylogenetic tree of the B. gibsonii-clade subtilisins of Table 4, B. gibsonii-clade subtilisin Bgi02446; and various other bacterial serine proteases.

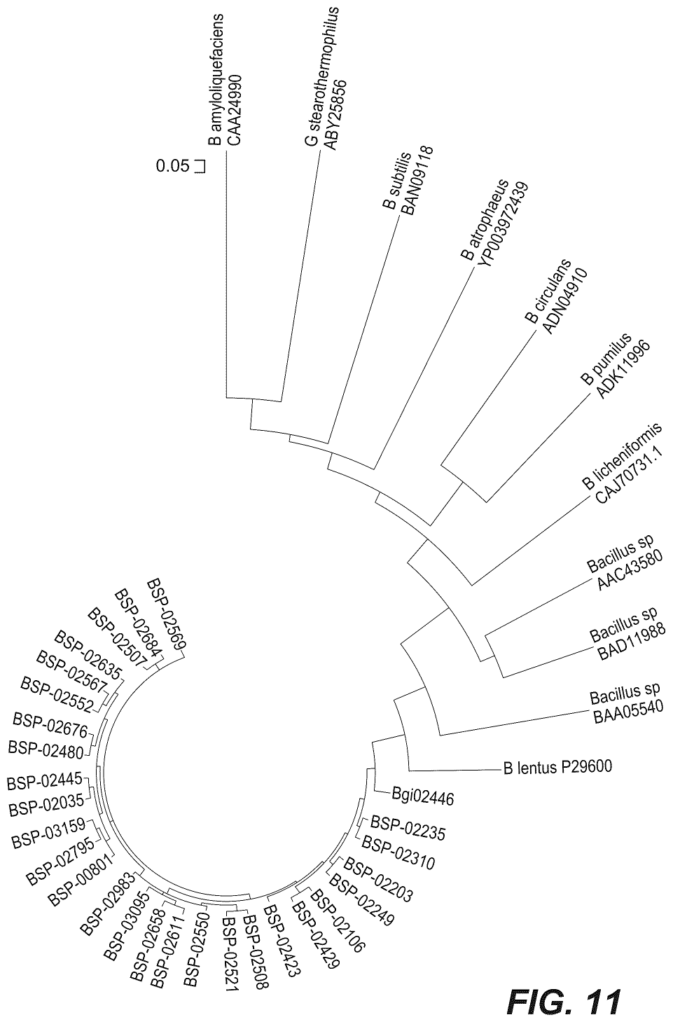

FIG. 11 provides a phylogenetic tree of multiple B. gibsonii-clade subtilisins of Table 5, B. gibsonii-clade subtilisin Bgi02446; and various other bacterial serine proteases.

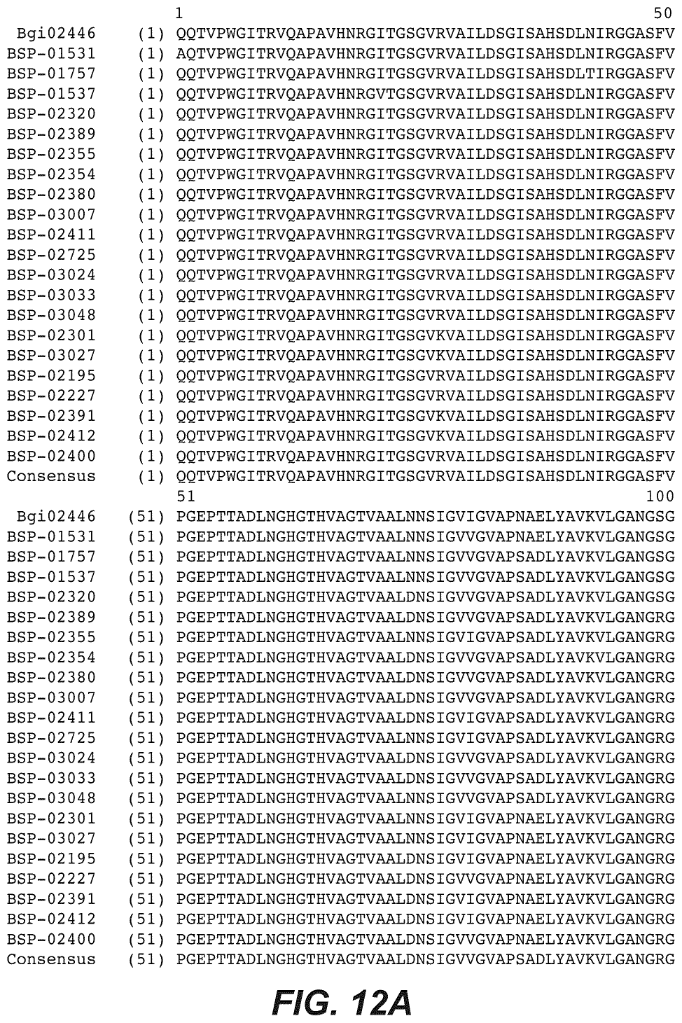

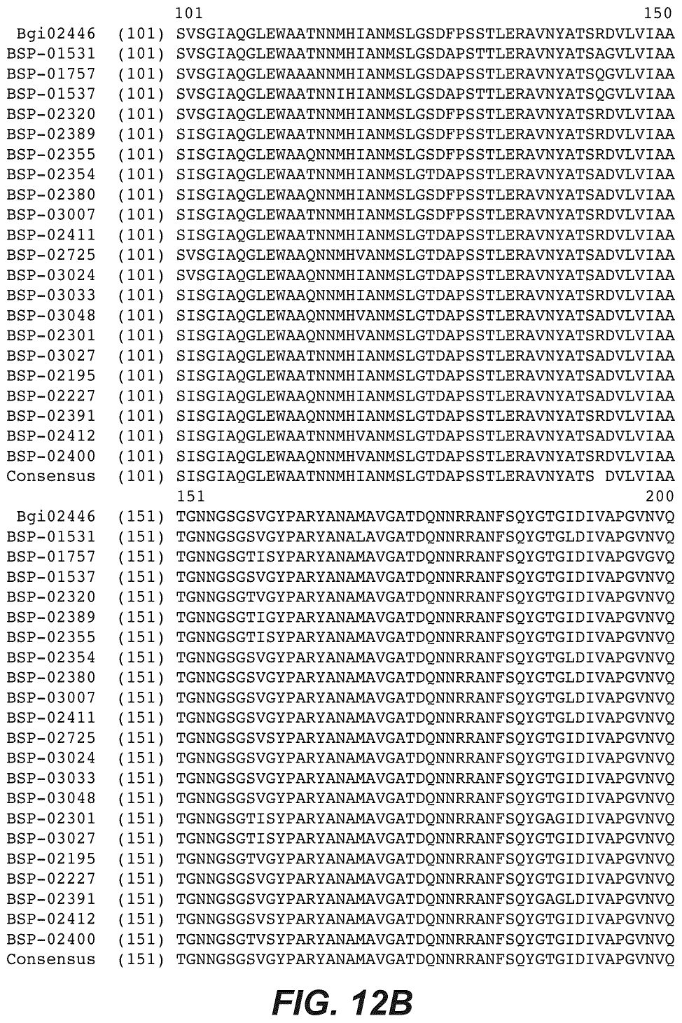

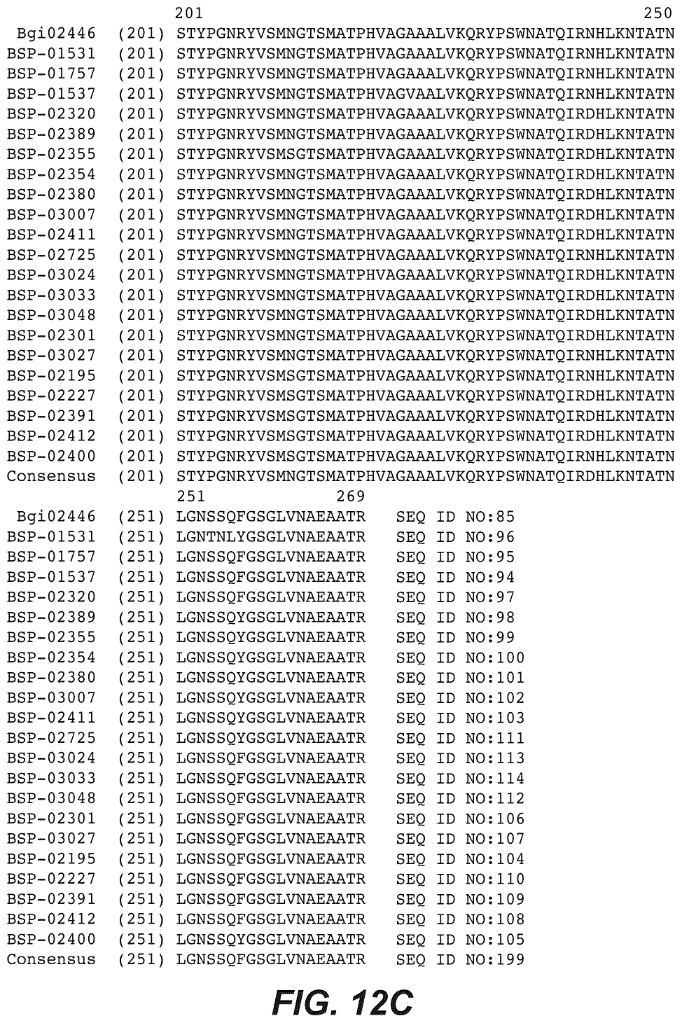

FIG. 12A-C provides the structural alignment of the amino acid sequences of the predicted mature forms of the B. gibsonii-clade subtilisins of Table 3 and Bgi02446, wherein these sequences are shown to share a common motif extending between Asp (D)32 and His (H)65.

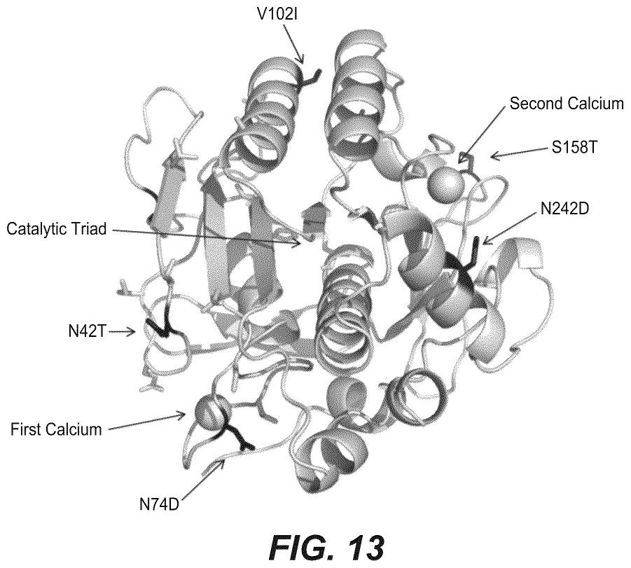

FIG. 13 provides the location of the N242D substitution relative to some of the other sites in BSP-00801 which are visible in this orientation. N242D is situated far removed from the substrate binding surface and the surface near the first calcium binding site. It is found closer to the second calcium binding site.

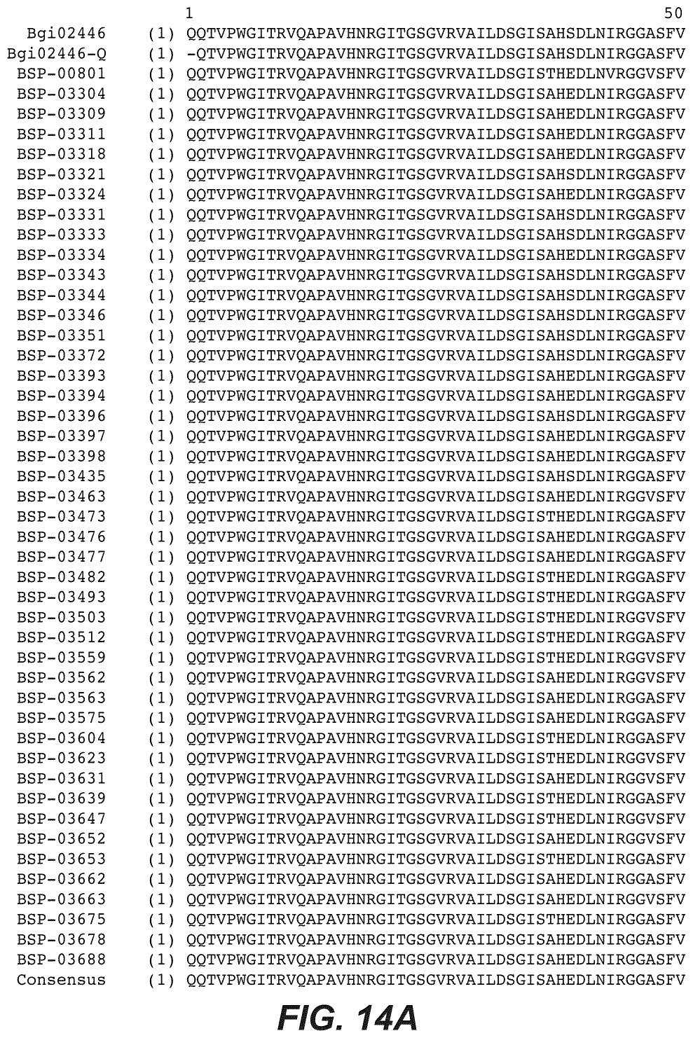

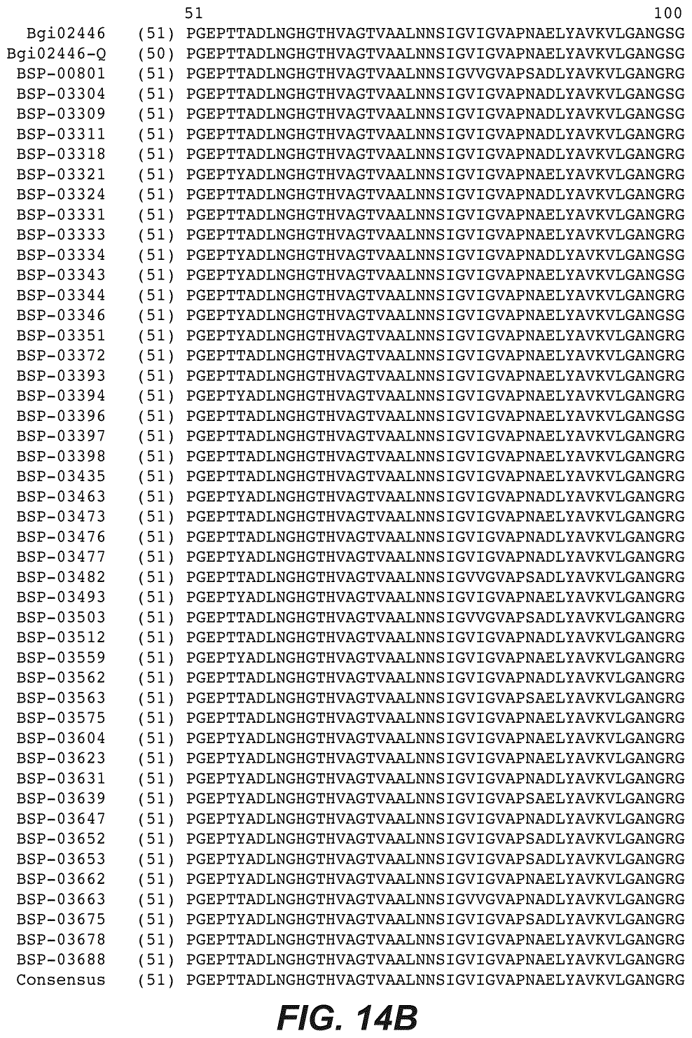

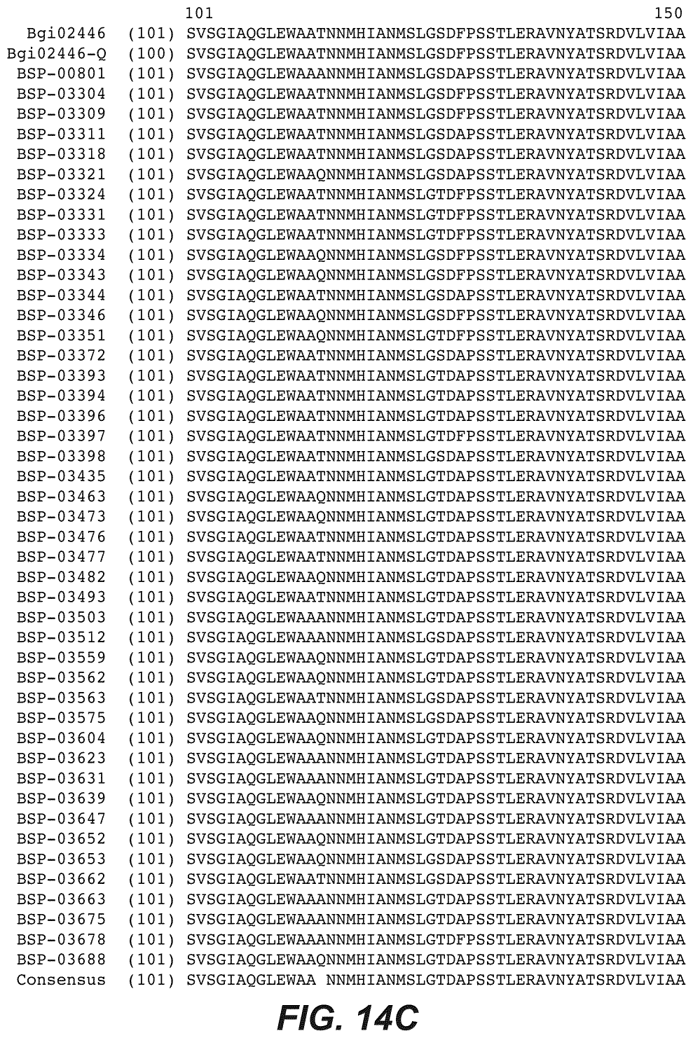

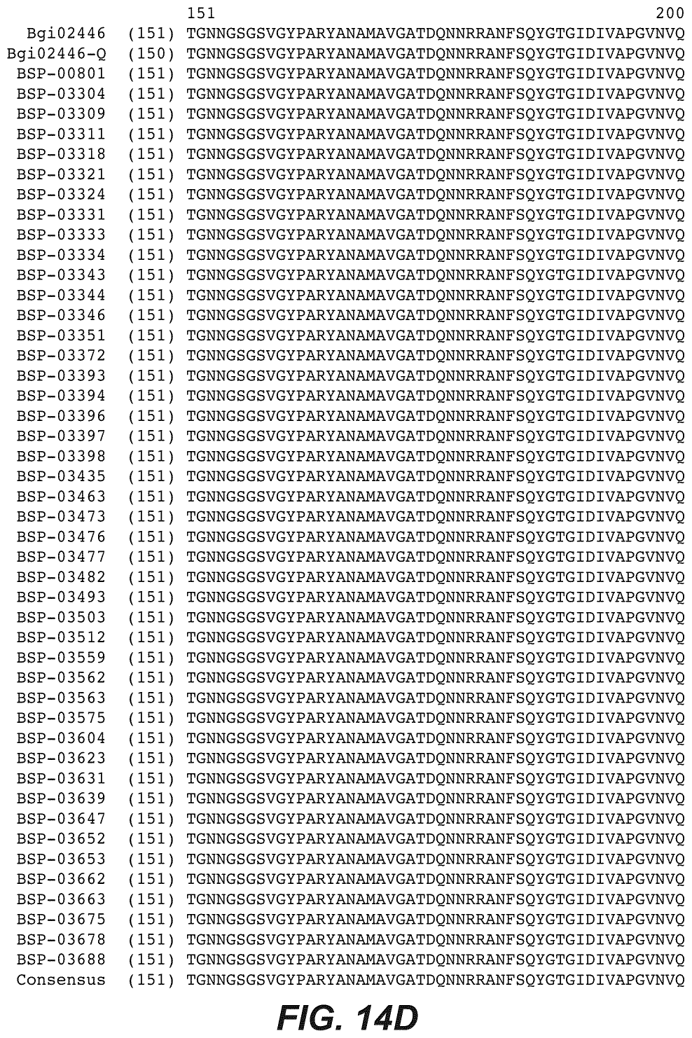

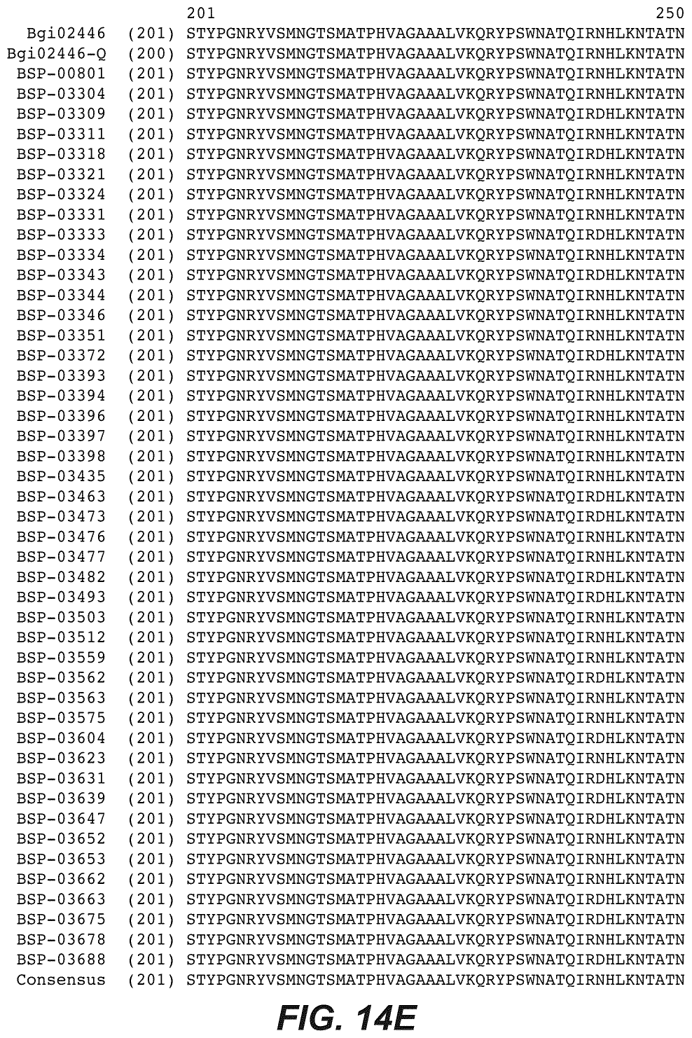

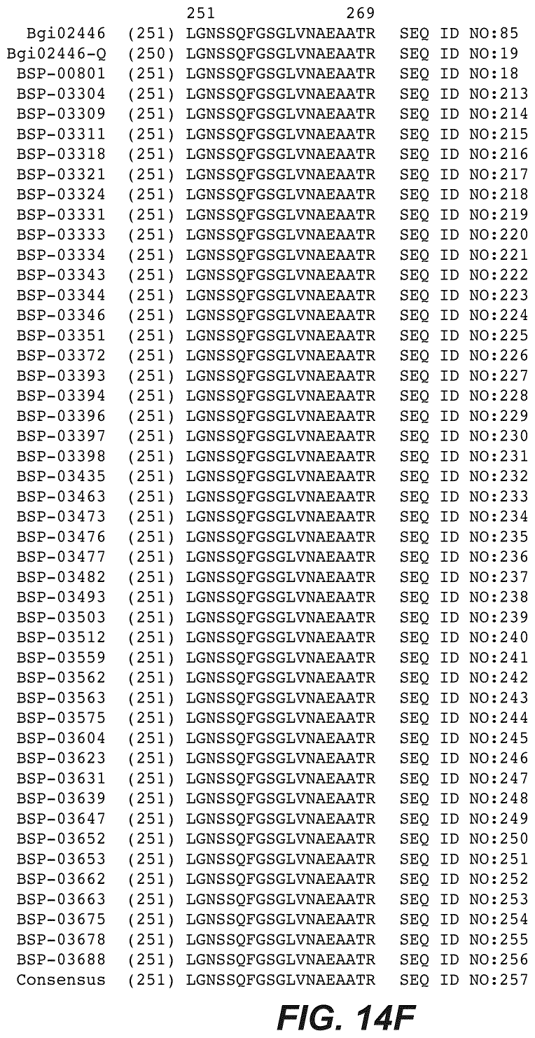

FIG. 14A-F provides an alignment of the amino acid sequences of Bgi02446, B. gibsonii-clade BSP-00801 variant subtilisin and mature forms of multiple B. gibsonii-clade variant subtilisins of Table 7.

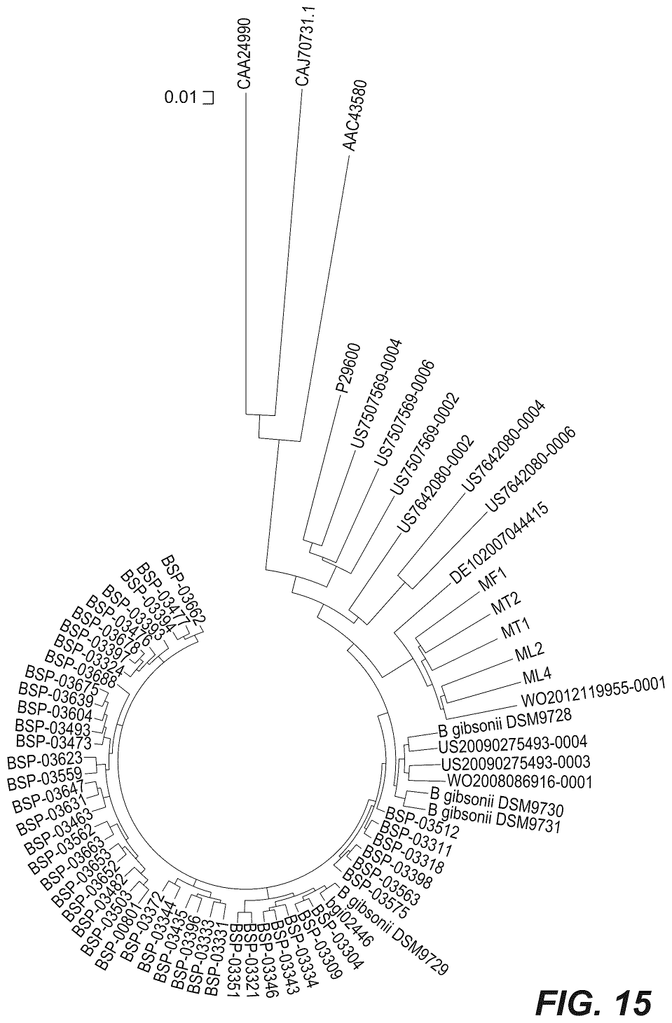

FIG. 15 provides a phylogenetic tree of the B. gibsonii-clade subtilisins of Table 7, the B. gibsonii-clade subtilisins DSM 9728, DSM 9729, DSM 9730, DSM 9731 and Bgi02446; the amino acid sequences of multiple proteases listed in Tables 6-1 and 6-2, and the amino acid sequences of BgAP variants ML2, ML4, MT1, MT2, MF1.

Described herein are variants, compositions and methods relating to recombinant serine proteases generated through conventional molecular biology techniques (see, e.g., Sambrook et al, Molecular Cloning: Cold Spring Harbor Laboratory Press). Compositions containing the serine proteases disclosed herein are suitable for use in cleaning fabrics and hard surfaces, as well as in a variety of industrial applications, such as, for example, textile, leather and feather processing. At least one B. gibsonii-clade serine proteases disclosed herein is also well suited for inclusion in compositions for protein degradation, including but not limited to laundry and dishwashing detergents; personal care compositions; and human food and animal feed.

Prior to describing the present compositions and methods in detail, the following terms are defined for clarity. Terms and abbreviations not defined should be accorded their ordinary meaning as used in the art. Unless defined otherwise herein, all technical and scientific terms used herein have the same meaning as commonly understood by one of ordinary skill in the art. Unless otherwise indicated, the practice of the present disclosure involves conventional techniques commonly used in molecular biology, protein engineering, and microbiology. Although any methods and materials similar or equivalent to those described herein find use in the practice of the present disclosure, some suitable methods and materials are described herein. The terms defined immediately below are more fully described by reference to the Specification as a whole.

As used herein, the singular “a,” “an” and “the” includes the plural unless the context clearly indicates otherwise. Unless otherwise indicated, nucleic acid sequences are written left to right in 5′ to 3′ orientation; and amino acid sequences are written left to right in amino to carboxy orientation. It is to be understood that this disclosure is not limited to the particular methodology, protocols, and reagents described herein, absent an indication to the contrary.

It is intended that every maximum numerical limitation given throughout this Specification includes every lower numerical limitation, as if such lower numerical limitations were expressly written herein. Every minimum numerical limitation given throughout this Specification will include every higher numerical limitation, as if such higher numerical limitations were expressly written herein. Every numerical range given throughout this Specification will include every narrower numerical range that falls within such broader numerical range, as if such narrower numerical ranges were all expressly written herein.

As used herein in connection with a numerical value, the term “about” refers to a range of +/−0.5 of the numerical value, unless the term is otherwise specifically defined in context. For instance, the phrase a “pH value of about 6” refers to pH values of from 5.5 to 6.5, unless the pH value is specifically defined otherwise.

The nomenclature of the amino acid substitution(s) or variation(s) of one or more subtilisin variant described herein uses one or more of the following: position; position:amino acid substitution(s) or variation(s); or starting amino acid(s):position:amino acid substitution(s). Reference to a “position” (i.e. 5, 8, 17, 22, etc) encompasses any starting amino acid that may be present at such position, and any variation from the starting amino acid or any substitution that may be present at such position. Reference to a “position: amino acid substitution(s) and/or variation(s)” (i.e. 1S/T/G, 3G, 17T, etc) encompasses any starting amino acid that may be present at such position and the one or more amino acid(s) that may vary from the starting amino acid and/or with which such starting amino acid may be substituted. An amino acid substitution will exclude the starting amino acid, where the substituted amino acid and starting amino acid are the same. Reference to a starting amino acid or an amino acid substitution or an amino acid variation may be further expressed as several starting, substituted, or varied amino acids separated by a foreslash (“/”). For example, D275S/K indicates position 275 is substituted with serine (S) or lysine (K) and P/S197K indicates that starting amino acid proline (P) or serine (S) at position 197 is substituted with lysine (K).

The position of an amino acid residue in a given amino acid sequence is numbered by correspondence with the amino acid sequence of SEQ ID NO:85. That is, the amino acid sequence of SEQ ID NO:85 serves as a reference sequence. For example, the amino acid sequence of a BG46-clade subtilisin or one or more subtilisin variant described herein is aligned with the amino acid sequence of SEQ ID NO:85 using an alignment algorithm as described herein, and each amino acid residue in the given amino acid sequence that aligns (preferably optimally aligns) with an amino acid residue in SEQ ID NO:85 is conveniently numbered by reference to the numerical position of that corresponding amino acid residue. Sequence alignment algorithms, such as, for example, described herein will identify the location where insertions or deletions occur in a subject sequence when compared to a query sequence. Additionally, members of the B. Gibsonii-clade with a QTVP at the N-terminus, such as, for example, SEQ ID NO:19, are for numbering purposes to be aligned with SEQ ID NO:85 as set forth in FIG. 14.

The term “variation(s)” when used in the phrase “two, three, or four or more amino acid variations versus SEQ ID NO:85” encompasses each amino acid that is different from the amino acid present at the corresponding position in SEQ ID NO:85. For example, the sequence of the variant of interest is aligned with SEQ ID NO:85 and each position in the variant compared to SEQ ID NO:85 to identify the amino acids at each position that are different from the amino acid present at the corresponding positions in SEQ ID NO:85 and each amino acid that is different from the corresponding amino acid in SEQ ID NO:85 is a variation.

As used herein, the terms “protease” and “proteinase” refer to an enzyme that has the ability to break down proteins and peptides. A protease has the ability to conduct “proteolysis,” by hydrolysis of peptide bonds that link amino acids together in a peptide or polypeptide chain forming the protein. This activity of a protease as a protein-digesting enzyme is referred to as “proteolytic activity.” Many well-known procedures exist for measuring proteolytic activity. For example, proteolytic activity may be ascertained by comparative assays that analyze the respective protease's ability to hydrolyze a suitable substrate. Exemplary substrates useful in the analysis of protease or proteolytic activity, include, but are not limited to, di-methyl casein (Sigma C-9801), bovine collagen (Sigma C-9879), bovine elastin (Sigma E-1625), and bovine keratin (ICN Biomedical 902111). Colorimetric assays utilizing these substrates are well known in the art (See e.g., WO99/34011 and U.S. Pat. No. 6,376,450). The pNA peptidyl assay (See e.g., Del Mar et al., Anal Biochem, 99:316-320, 1979) also finds use in determining the active enzyme concentration. This assay measures the rate at which p-nitroaniline is released as the enzyme hydrolyzes a soluble synthetic substrate, such as succinyl-alanine-alanine-proline-phenylalanine-p-nitroanilide (suc-AAPF-pNA). The rate of production of yellow color from the hydrolysis reaction is measured at 410 nm on a spectrophotometer and is proportional to the active enzyme concentration. In addition, absorbance measurements at 280 nanometers (nm) can be used to determine the total protein concentration in a sample of purified protein. The activity on substrate/protein concentration gives the enzyme specific activity.

The term “variant,” with respect to a polypeptide, refers to a polypeptide that differs from a specified wild-type, parental, or reference polypeptide in that it includes one or more naturally-occurring or man-made substitutions, insertions, or deletions of an amino acid. Similarly, the term “variant,” with respect to a polynucleotide, refers to a polynucleotide that differs in nucleotide sequence from a specified wild-type, parental, or reference polynucleotide. The identity of the wild-type, parental, or reference polypeptide or polynucleotide will be apparent from context.

As used herein, “the genus Bacillus” includes all species within the genus “Bacillus,” as known to those of skill in the art, including but not limited to B. subtilis, B. lichenformis, B. lentus, B. brevis, B. stearothermophilus, B. alkalophilus, B. amyloliquefaciens, B. clausii, B. halodurans, B. megaterium, B. coagulans, B. circulans, B. lautus, B. gibsonii, B. pabuli, B. cereus, B. agaradhaerens, B akibai, B. clarkii, and B. thuringiensis. It is recognized that the genus Bacillus continues to undergo taxonomical reorganization. Thus, it is intended that the genus include species that have been reclassified, including but not limited to such organisms as B. stearothermophilus, which is now named “Geobacillus stearothermophilus.” The production of resistant endospores under stressful environmental conditions is considered the defining feature of the genus Bacillus, although this characteristic also applies to the recently named Alicyclobacillus, Amphibacillus, Aneurinibacillus, Anoxybacillus, Brevibacillus, Filobacillus, Gracilibacillus, Halobacillus, Paenibacillus, Salibacillus, Thermobacillus, Ureibacillus, and Virgibacillus.

As used herein, the term “mutation” refers to changes made to a reference amino acid or nucleic acid sequence. It is intended that the term encompass substitutions, insertions and deletions.

As used herein, the term “vector” refers to a nucleic acid construct used to introduce or transfer nucleic acid(s) into a target cell or tissue. A vector is typically used to introduce foreign DNA into a cell or tissue. Vectors include plasmids, cloning vectors, bacteriophages, viruses (e.g., viral vector), cosmids, expression vectors, shuttle vectors, and the like. A vector typically includes an origin of replication, a multicloning site, and a selectable marker. The process of inserting a vector into a target cell is typically referred to as transformation. The present invention includes, in some embodiments, a vector that comprises a DNA sequence encoding a serine protease polypeptide (e.g., precursor or mature serine protease polypeptide) that is operably linked to a suitable prosequence (e.g., secretory, signal peptide sequence, etc.) capable of effecting the expression of the DNA sequence in a suitable host, and the folding and translocation of the recombinant polypeptide chain.

As used herein, the term “expression cassette,” “expression plasmid” or “expression vector” refers to a nucleic acid construct or vector generated recombinantly or synthetically for the expression of a nucleic acid of interest in a target cell. An expression vector or expression cassette typically comprises a promoter nucleotide sequence that drives expression of the foreign nucleic acid. The expression vector or cassette also typically includes any other specified nucleic acid elements that permit transcription of a particular nucleic acid in a target cell. A recombinant expression cassette can be incorporated into a plasmid, chromosome, mitochondrial DNA, plastid DNA, virus, or nucleic acid fragment. Many prokaryotic and eukaryotic expression vectors are commercially available.

As used herein, a “plasmid” refers to an extrachromosomal DNA molecule which is capable of replicating independently from the chromosomal DNA. A plasmid is double stranded (ds) and may be circular and is typically used as a cloning vector.

As used herein in the context of introducing a nucleic acid sequence into a cell, the term “introduced” refers to any method suitable for transferring the nucleic acid sequence into the cell. Such methods for introduction include but are not limited to protoplast fusion, transfection, transformation, electroporation, conjugation, and transduction. Transformation refers to the genetic alteration of a cell which results from the uptake, optional genomic incorporation, and expression of genetic material (e.g., DNA).

As used herein, a nucleic acid is “operably linked” with another nucleic acid sequence when it is placed into a functional relationship with another nucleic acid sequence. For example, a promoter or enhancer is operably linked to a nucleotide coding sequence if the promoter affects the transcription of the coding sequence. A ribosome binding site may be operably linked to a coding sequence if it is positioned so as to facilitate translation of the coding sequence. Typically, “operably linked” DNA sequences are contiguous. However, enhancers do not have to be contiguous. Linking is accomplished by ligation at convenient restriction sites. If such sites do not exist, synthetic oligonucleotide adaptors or linkers may be used in accordance with conventional practice.

As used herein the term “gene” refers to a polynucleotide (e.g., a DNA segment), that encodes a polypeptide and includes regions preceding and following the coding regions. In some instances a gene includes intervening sequences (introns) between individual coding segments (exons).

As used herein, “recombinant” when used with reference to a cell typically indicates that the cell has been modified by the introduction of a foreign nucleic acid sequence or that the cell is derived from a cell so modified. For example, a recombinant cell may comprise a gene not found in identical form within the native (non-recombinant) form of the cell, or a recombinant cell may comprise a native gene (found in the native form of the cell) that has been modified and re-introduced into the cell. A recombinant cell may comprise a nucleic acid endogenous to the cell that has been modified without removing the nucleic acid from the cell; such modifications include those obtained by gene replacement, site-specific mutation, and related techniques known to those of ordinary skill in the art. Recombinant DNA technology includes techniques for the production of recombinant DNA in vitro and transfer of the recombinant DNA into cells where it may be expressed or propagated, thereby producing a recombinant polypeptide. “Recombination” and “recombining” of polynucleotides or nucleic acids refer generally to the assembly or combining of two or more nucleic acid or polynucleotide strands or fragments to generate a new polynucleotide or nucleic acid.

A nucleic acid or polynucleotide is said to “encode” a polypeptide if, in its native state or when manipulated by methods known to those of skill in the art, it can be transcribed and/or translated to produce the polypeptide or a fragment thereof. The anti-sense strand of such a nucleic acid is also said to encode the sequence.

The terms “host strain” and “host cell” refer to a suitable host for an expression vector comprising a DNA sequence of interest.

A “protein” or “polypeptide” comprises a polymeric sequence of amino acid residues. The terms “protein” and “polypeptide” are used interchangeably herein. The single and 3-letter code for amino acids as defined in conformity with the IUPAC-IUB Joint Commission on Biochemical Nomenclature (JCBN) is used throughout this disclosure. The single letter X refers to any of the twenty amino acids. It is also understood that a polypeptide may be coded for by more than one nucleotide sequence due to the degeneracy of the genetic code. Mutations can be named by the one letter code for the parent amino acid, followed by a position number and then the one letter code for the variant amino acid. For example, mutating glycine (G) at position 87 to serine (S) is represented as “G087S” or “G87S.

A “prosequence” or “propeptide sequence” refers to an amino acid sequence between the signal peptide sequence and mature protease sequence that is necessary for the proper folding and secretion of the protease; they are sometimes referred to as intramolecular chaperones. Cleavage of the prosequence or propeptide sequence results in a mature active protease. Bacterial serine proteases are often expressed as pro-enzymes.

The terms “signal sequence” and “signal peptide” refer to a sequence of amino acid residues that may participate in the secretion or direct transport of the mature or precursor form of a protein. The signal sequence is typically located N-terminal to the precursor or mature protein sequence. The signal sequence may be endogenous or exogenous. A signal sequence is normally absent from the mature protein. A signal sequence is typically cleaved from the protein by a signal peptidase after the protein is transported.

The term “mature” form of a protein, polypeptide, or peptide refers to the functional form of the protein, polypeptide, or peptide without the signal peptide sequence and propeptide sequence.

The term “precursor” form of a protein or peptide refers to a mature form of the protein having a prosequence operably linked to the amino or carbonyl terminus of the protein. The precursor may also have a “signal” sequence operably linked to the amino terminus of the prosequence. The precursor may also have additional polypeptides that are involved in post-translational activity (e.g., polypeptides cleaved therefrom to leave the mature form of a protein or peptide).

The term “wild-type” in reference to an amino acid sequence or nucleic acid sequence indicates that the amino acid sequence or nucleic acid sequence is a native or naturally-occurring sequence. As used herein, the term “naturally-occurring” refers to anything (e.g., proteinsor nucleic acid sequences) that is found in nature. Conversely, the term “non-naturally occurring” refers to anything that is not found in nature (e.g., recombinant nucleic acids and protein sequences produced in the laboratory or modification of the wild-type sequence).

As used herein with regard to amino acid residue positions, “corresponding to” or “corresponds to” or “corresponds” refers to an amino acid residue at the enumerated position in a protein or peptide, or an amino acid residue that is analogous, homologous, or equivalent to an enumerated residue in a protein or peptide. As used herein, “corresponding region” generally refers to an analogous position in a related proteins or a reference protein.

The terms “derived from” and “obtained from” refer to not only a protein produced or producible by a strain of the organism in question, but also a protein encoded by a DNA sequence isolated from such strain and produced in a host organism containing such DNA sequence. Additionally, the term refers to a protein which is encoded by a DNA sequence of synthetic and/or cDNA origin and which has the identifying characteristics of the protein in question. To exemplify, “proteases derived from Bacillus” refers to those enzymes having proteolytic activity that are naturally produced by Bacillus, as well as to serine proteases like those produced by Bacillus sources but which through the use of genetic engineering techniques are produced by other host cells transformed with a nucleic acid encoding the serine proteases.

The term “identical” in the context of two polynucleotide or polypeptide sequences refers to the nucleic acids or amino acids in the two sequences that are the same when aligned for maximum correspondence, as measured using sequence comparison or analysis algorithms.

As used herein, “% identity” or percent identity” or “PID” refers to protein sequence identity. Percent identity may be determined using standard techniques known in the art. Useful algorithms include the BLAST algorithms (See, Altschul et al., J Mol Biol, 215:403-410, 1990; and Karlin and Altschul, Proc Natl Acad Sci USA, 90:5873-5787, 1993). The BLAST program uses several search parameters, most of which are set to the default values. The NCBI BLAST algorithm finds the most relevant sequences in terms of biological similarity but is not recommended for query sequences of less than 20 residues (Altschul et al., Nucleic Acids Res, 25:3389-3402, 1997; and Schaffer et al., Nucleic Acids Res, 29:2994-3005, 2001). Exemplary default BLAST parameters for a nucleic acid sequence searches include: Neighboring words threshold=11; E-value cutoff=10; Scoring Matrix=NUC.3.1 (match=1, mismatch=−3); Gap Opening=5; and Gap Extension=2. Exemplary default BLAST parameters for amino acid sequence searches include: Word size=3; E-value cutoff=10; Scoring Matrix=BLOSUM62; Gap Opening=11; and Gap extension=1. A percent (%) amino acid sequence identity value is determined by the number of matching identical residues divided by the total number of residues of the “reference” sequence including any gaps created by the program for optimal/maximum alignment. BLAST algorithms refer to the “reference” sequence as the “query” sequence.

As used herein, “homologous proteins” or “homologous proteases” refers to proteins that have distinct similarity in primary, secondary, and/or tertiary structure. Protein homology can refer to the similarity in linear amino acid sequence when proteins are aligned. Homologous search of protein sequences can be done using BLASTP and PSI-BLAST from NCBI BLAST with threshold (E-value cut-off) at 0.001. (Altschul S F, Madde T L, Shaffer A A, Zhang J, Zhang Z, Miller W, Lipman D J. Gapped BLAST and PSI BLAST a new generation of protein database search programs. Nucleic Acids Res 1997 Set 1; 25(17):3389-402). Using this information, proteins sequences can be grouped. A phylogenetic tree can be built using the amino acid sequences. Amino acid sequences can be entered in a program such as the Vector NTI Advance suite and a Guide Tree can be created using the Neighbor Joining (NJ) method (Saitou and Nei, Mol Biol Evol, 4:406-425, 1987). The tree construction can be calculated using Kimura's correction for sequence distance and ignoring positions with gaps. A program such as AlignX can display the calculated distance values in parenthesis following the molecule name displayed on the phylogenetic tree.

Understanding the homology between molecules can reveal the evolutionary history of the molecules as well as information about their function; if a newly sequenced protein is homologous to an already characterized protein, there is a strong indication of the new protein's biochemical function. The most fundamental relationship between two entities is homology; two molecules are said to be homologous if they have been derived from a common ancestor. Homologous molecules, or homologs, can be divided into two classes, paralogs and orthologs. Paralogs are homologs that are present within one species. Paralogs often differ in their detailed biochemical functions. Orthologs are homologs that are present within different species and have very similar or identical functions. A protein superfamily is the largest grouping (clade) of proteins for which common ancestry can be inferred. Usually this common ancestry is based on sequence alignment and mechanistic similarity. Superfamilies typically contain several protein families which show sequence similarity within the family. The term “protein clan” is commonly used for protease superfamilies based on the MEROPS protease classification system.

The CLUSTAL W algorithm is another example of a sequence alignment algorithm (See, Thompson et al., Nucleic Acids Res, 22:4673-4680, 1994). Default parameters for the CLUSTAL W algorithm include: Gap opening penalty=10.0; Gap extension penalty=0.05; Protein weight matrix=BLOSUM series; DNA weight matrix=IUB; Delay divergent sequences %=40; Gap separation distance=8; DNA transitions weight=0.50; List hydrophilic residues=GPSNDQEKR; Use negative matrix=OFF; Toggle Residue specific penalties=ON; Toggle hydrophilic penalties=ON; and Toggle end gap separation penalty=OFF. In CLUSTAL algorithms, deletions occurring at either terminus are included. For example, a variant with a five amino acid deletion at either terminus (or within the polypeptide) of a polypeptide of 500 amino acids would have a percent sequence identity of 99% (495/500 identical residues×100) relative to the “reference” polypeptide. Such a variant would be encompassed by a variant having “at least 99% sequence identity” to the polypeptide.

A nucleic acid or polynucleotide is “isolated” when it is at least partially or completely separated from other components, including but not limited to for example, other proteins, nucleic acids, cells, etc. Similarly, a polypeptide, protein or peptide is “isolated” when it is at least partially or completely separated from other components, including but not limited to for example, other proteins, nucleic acids, cells, etc. On a molar basis, an isolated species is more abundant than are other species in a composition. For example, an isolated species may comprise at least about 60%, about 65%, about 70%, about 75%, about 80%, about 85%, about 90%, about 91%, about 92%, about 93%, about 94%, about 95%, about 96%, about 97%, about 98%, about 99%, or about 100% (on a molar basis) of all macromolecular species present. Preferably, the species of interest is purified to essential homogeneity (i.e., contaminant species cannot be detected in the composition by conventional detection methods). Purity and homogeneity can be determined using a number of techniques well known in the art, such as agarose or polyacrylamide gel electrophoresis of a nucleic acid or a protein sample, respectively, followed by visualization upon staining. If desired, a high-resolution technique, such as high performance liquid chromatography (HPLC) or a similar means can be utilized for purification of the material.

The term “purified” as applied to nucleic acids or polypeptides generally denotes a nucleic acid or polypeptide that is essentially free from other components as determined by analytical techniques well known in the art (e.g., a purified polypeptide or polynucleotide forms a discrete band in an electrophoretic gel, chromatographic eluate, and/or a media subjected to density gradient centrifugation). For example, a nucleic acid or polypeptide that gives rise to essentially one band in an electrophoretic gel is “purified.” A purified nucleic acid or polypeptide is at least about 50% pure, usually at least about 60%, about 65%, about 70%, about 75%, about 80%, about 85%, about 90%, about 91%, about 92%, about 93%, about 94%, about 95%, about 96%, about 97%, about 98%, about 99%, about 99.5%, about 99.6%, about 99.7%, about 99.8% or more pure (e.g., percent by weight on a molar basis). In a related sense, a composition is enriched for a molecule when there is a substantial increase in the concentration of the molecule after application of a purification or enrichment technique. The term “enriched” refers to a compound, polypeptide, cell, nucleic acid, amino acid, or other specified material or component that is present in a composition at a relative or absolute concentration that is higher than a starting composition.

The phrase “composition(s) substantially-free of boron” or “detergent(s) substantially-free of boron” refers to composition(s) or detergent(s), respectively, that contain trace amounts of boron, for example, less than about 1000 ppm (1 mg/kg or liter equals 1 ppm), less than about 100 ppm, less than about 50 ppm, less than about 10 ppm, or less than about 5 ppm, or less than about 1 ppm, perhaps from other compositions or detergent constituents.

As used herein, the term “functional assay” refers to an assay that provides an indication of a protein's activity. In some embodiments, the term refers to assay systems in which a protein is analyzed for its ability to function in its usual capacity. For example, in the case of a protease, a functional assay involves determining the effectiveness of the protease to hydrolyze a proteinaceous substrate.

The term “cleaning activity” refers to a cleaning performance achieved by a serine protease polypeptide or reference protease under conditions prevailing during the proteolytic, hydrolyzing, cleaning, or other process of the disclosure. In some embodiments, cleaning performance of a serine protease polypeptide or reference protease may be determined by using various assays for cleaning one or more various enzyme sensitive stains on an item or surface (e.g., a stain resulting from food, grass, blood, ink, milk, oil, and/or egg protein). Cleaning performance of a variant or reference protease can be determined by subjecting the stain on the item or surface to standard wash condition(s) and assessing the degree to which the stain is removed by using various chromatographic, spectrophotometric, or other quantitative methodologies. Exemplary cleaning assays and methods are known in the art and include, but are not limited to those described in WO99/34011 and U.S. Pat. No. 6,605,458, both of which are herein incorporated by reference, as well as those cleaning assays and methods included in the Examples provided below.

The term “cleaning effective amount” of a serine protease polypeptide or reference protease refers to the amount of protease that achieves a desired level of enzymatic activity in a specific cleaning composition. Such effective amounts are readily ascertained by one of ordinary skill in the art and are based on many factors, such as the particular protease used, the cleaning application, the specific composition of the cleaning composition, and whether a liquid or dry (e.g., granular, tablet, bar) composition is required, etc.

The term “cleaning adjunct material” refers to any liquid, solid, or gaseous material included in cleaning composition other than a serine protease polypeptide of the disclosure. In some embodiments, the cleaning compositions of the present disclosure include one or more cleaning adjunct materials. Each cleaning adjunct material is typically selected depending on the particular type and form of cleaning composition (e.g., liquid, granule, powder, bar, paste, spray, tablet, gel, foam, or other composition). Preferably, each cleaning adjunct material is compatible with the protease enzyme used in the composition.

Cleaning compositions and cleaning formulations include any composition that is suited for cleaning, bleaching, disinfecting, and/or sterilizing any object, item, and/or surface. Such compositions and formulations include, but are not limited to for example, liquid and/or solid compositions, including cleaning or detergent compositions (e.g., liquid, tablet, gel, bar, granule, and/or solid laundry cleaning or detergent compositions and fine fabric detergent compositions; hard surface cleaning compositions and formulations, such as for glass, wood, ceramic and metal counter tops and windows; carpet cleaners; oven cleaners; fabric fresheners; fabric softeners; and textile, laundry booster cleaning or detergent compositions, laundry additive cleaning compositions, and laundry pre-spotter cleaning compositions; dishwashing compositions, including hand or manual dishwashing compositions (e.g., “hand” or “manual” dishwashing detergents) and automatic dishwashing compositions (e.g., “automatic dishwashing detergents”). Single dosage unit forms also find use with the present invention, including but not limited to pills, tablets, gelcaps, or other single dosage units such as pre-measured powders or liquids.

Cleaning composition or cleaning formulations, as used herein, include, unless otherwise indicated, granular or powder-form all-purpose or heavy-duty washing agents, especially cleaning detergents; liquid, granular, gel, solid, tablet, paste, or unit dosage form all-purpose washing agents, especially the so-called heavy-duty liquid (HDL) detergent or heavy-duty dry (HDD) detergent types; liquid fine-fabric detergents; hand or manual dishwashing agents, including those of the high-foaming type; hand or manual dishwashing, automatic dishwashing, or dishware or tableware washing agents, including the various tablet, powder, solid, granular, liquid, gel, and rinse-aid types for household and institutional use; liquid cleaning and disinfecting agents, including antibacterial hand-wash types, cleaning bars, mouthwashes, denture cleaners, car shampoos, carpet shampoos, bathroom cleaners; hair shampoos and/or hair-rinses for humans and other animals; shower gels and foam baths and metal cleaners; as well as cleaning auxiliaries, such as bleach additives and “stain-stick” or pre-treat types. In some embodiments, granular compositions are in “compact” form; in some embodiments, liquid compositions are in a “concentrated” form.

As used herein, “fabric cleaning compositions” include hand and machine laundry detergent compositions including laundry additive compositions and compositions suitable for use in the soaking and/or pretreatment of stained fabrics (e.g., clothes, linens, and other textile materials).

As used herein, “non-fabric cleaning compositions” include non-textile (i.e., non-fabric) surface cleaning compositions, including, but not limited to for example, hand or manual or automatic dishwashing detergent compositions, oral cleaning compositions, denture cleaning compositions, contact lens cleaning compositions, wound debridement compositions, and personal cleansing compositions.

As used herein, the term “detergent composition” or “detergent formulation” is used in reference to a composition intended for use in a wash medium for the cleaning of soiled or dirty objects, including particular fabric and/or non-fabric objects or items. Such compositions of the present disclosure are not limited to any particular detergent composition or formulation. Indeed, in some embodiments, the detergents of the disclosure comprise at least one serine protease polypeptide of the disclosure and, in addition, one or more surfactants, transferase(s), hydrolytic enzymes, oxido reductases, builders (e.g., a builder salt), bleaching agents, bleach activators, bluing agents, fluorescent dyes, caking inhibitors, masking agents, enzyme activators, antioxidants, and/or solubilizers. In some instances, a builder salt is a mixture of a silicate salt and a phosphate salt, preferably with more silicate (e.g., sodium metasilicate) than phosphate (e.g., sodium tripolyphosphate). Some compositions of the disclosure, such as, but not limited to, cleaning compositions or detergent compositions, do not contain any phosphate (e.g., phosphate salt or phosphate builder).

As used herein, the term “bleaching” refers to the treatment of a material (e.g., fabric, laundry, pulp, etc.) or surface for a sufficient length of time and/or under appropriate pH and/or temperature conditions to effect a brightening (i.e., whitening) and/or cleaning of the material. Examples of chemicals suitable for bleaching include, but are not limited to, for example, ClO₂, H₂O₂, peracids, NO₂, etc.

As used herein, “wash performance” of a protease (e.g., a serine protease polypeptide of the disclosure) refers to the contribution of a serine protease polypeptide to washing that provides additional cleaning performance to the detergent as compared to the detergent without the addition of the serine protease polypeptide to the composition. Wash performance is compared under relevant washing conditions. In some test systems, other relevant factors, such as detergent composition, sud concentration, water hardness, washing mechanics, time, pH, and/or temperature, can be controlled in such a way that condition(s) typical for household application in a certain market segment (e.g., hand or manual dishwashing, automatic dishwashing, dishware cleaning, tableware cleaning, fabric cleaning, etc.) are imitated.

The term “relevant washing conditions” is used herein to indicate the conditions, particularly washing temperature, time, washing mechanics, sud concentration, type of detergent and water hardness, actually used in households in a hand dishwashing, automatic dishwashing, or laundry detergent market segment.

As used herein, the term “disinfecting” refers to the removal of contaminants from the surfaces, as well as the inhibition or killing of microbes on the surfaces of items. It is not intended that the present disclosure be limited to any particular surface, item, or contaminant(s) or microbes to be removed.

The “compact” form of the cleaning compositions herein is best reflected by density and, in terms of composition, by the amount of inorganic filler salt. Inorganic filler salts are conventional ingredients of detergent compositions in powder form. In conventional detergent compositions, the filler salts are present in substantial amounts, typically about 17 to about 35% by weight of the total composition. In contrast, in compact compositions, the filler salt is present in amounts not exceeding about 15% of the total composition. In some embodiments, the filler salt is present in amounts that do not exceed about 10%, or more preferably, about 5%, by weight of the composition. In some embodiments, the inorganic filler salts are selected from the alkali and alkaline-earth-metal salts of sulfates and chlorides. In some embodiments, the filler salt is sodium sulfate.

Disclosed herein is one or more subtilisin variant useful for cleaning applications and in methods of cleaning, as well as in a variety of industrial applications. In one embodiment, one or more serine protease or subtilisin variant described herein is a member of the B. gibsonii-clade. In another embodiment, one or more subtilisin variant described herein is an isolated, recombinant, substantially pure, and/or non-naturally occurring polypeptide. In some embodiments, one or more subtilisin variant described herein can be incorporated into one or more cleaning composition useful in one or more method of cleaning an item or a surface in need thereof.

Some embodiments are directed to a subtilisin variant comprising an amino acid sequence comprising two, three, or four or more variations versus SEQ ID NO:85 at positions selected from: (i) 1, 4, 9, 21, 24, 27, 36, 37, 39, 42, 43, 44, 47, 54, 55, 56, 74, 80, 85, 87, 99, 102, 114, 117, 119, 121, 126, 128, 131, 143, 144, 158, 159, 160, 169, 182, 188, 190, 197, 198, 212, 224, 231, 232, 237, 242, 245, 246, 254, 255, 256, and 257; (ii) 37, 39, 47, 56, 80, 85, 87, 99, 114, 126, 128, and 242; (iii) 39, 99, 126, and 128; (iv) 39 in combination with one or more variation at a position selected from 37, 47, 56, 80, 85, 87, 99, 114, 126, 128, and 242; (v) 56 in combination with one or more variation at a position selected from 37, 39, 47, 80, 85, 87, 99, 114, 126, 128, and 242; (vi) 114 in combination with one or more variation at a position selected from 37, 39, 47, 56, 80, 85, 87, 99, 126, 128, and 242; (vii) 126 in combination with one or more variation at a position selected from 37, 39, 47, 56, 80, 85, 87, 99, 114, 128, and 242; (viii) 242 in combination with one or more variation at a position selected from 37, 39, 47, 56, 80, 85, 87, 99, 114, 126, and 128; (ix) 99+128 in combination with one or more variation at a position selected from 39, 56, 114, 126 and 242; (x) 39+242 in combination with one or more variation at a position selected from 37, 47, 56, 80, 85, 87, 99, 114, 126, and 128; or (xi) 39+99+128 in combination with one or more variation at a position selected from 37, 47, 56, 80, 85, 87, 114, 126, and 242; with the proviso that one or more of said two, three, or four or more variations is non-naturally occurring; and wherein the amino acid positions of the variant are numbered by correspondence with the amino acid sequence of SEQ ID NO:85. Yet other embodiments are directed to a subtilisin variant comprising an amino acid sequence comprising two, three, or four or more variations versus SEQ ID NO:85 at positions selected from: (i) 1A, 4I, 9S, 21V, 24F, 27K, 36A, 37T/N, 39E, 42T, 43V, 44S, 47V, 54S, 55M/G, 56N/Y, 74D, 80V, 85S, 87D, 99R, 102I, 114A/P/Q, 117I, 119V, 121S, 126T, 128A, 131T, 143A/T/Q, 144G, 158T, 159I, 160S, 169L, 182S, 188A, 190L, 197I, 198G, 212S/T/K, 224I/V, 231K, 232N, 237T, 242D/Q, 245L, 246S/K, 254T, 255N, 256L, and 257Y; (ii) 37T/N, 39E, 47V, 56N/Y, 80V, 85S, 87D, 99R, 114A/P/Q, 126T, 128A, and 242D/Q; or (iii) 39E, 99R, 126T, and 128A; (iv) 39E in combination with one or more variation at a position selected from 37T/N, 47V, 56N/Y, 80V, 85S, 87D, 99R, 114A/P/Q, 126T, 128A, and 242D/Q; (v) 56N/Y in combination with one or more variation at a position selected from 37T/N, 39E, 47V, 80V, 85S, 87D, 99R, 114A/P/Q, 126T, 128A, and 242D/Q; (vi) 114A/P/Q in combination with one or more variation at a position selected from 37T/N, 39E, 47V, 56N/Y, 80V, 85S, 87D, 99R, 126T, 128A, and 242D/Q; (vii) 126T in combination with one or more variation at a position selected from 37T/N, 39E, 47V, 56N/Y, 80V, 85S, 87D, 99R, 114A/P/Q, 128A, and 242D/Q; (viii) 242D/Q in combination with one or more variation at a position selected from 37T/N, 39E, 47V, 56N/Y, 80V, 85S, 87D, 99R, 114A/P/Q, 126T, and 128A; (ix) 99R+128A in combination with one or more variation at a position selected from 39E, 56N/Y, 114A/P/Q, 126T, and 242D/Q; (x) 39E+242D/Q in combination with one or more variation at a position selected from 37T/N, 47V, 56N/Y, 80V, 85S, 87D, 99R, 114A/P/Q, 126T, and 128A; or (xi) 39E+99R+128A in combination with one or more variation at a position selected from 37T/N, 47V, 56N/Y, 80V, 85S, 87D, 114A/P/Q, 126T, and 242D/Q; with the proviso that one or more of said two, three, or four or more variations is non-naturally occurring; and wherein the amino acid positions of the variant are numbered by correspondence with the amino acid sequence of SEQ ID NO:85.

Other embodiments are directed to a subtilisin variant comprising an amino acid sequence comprising two, three, or four or more amino acid substitutions at positions selected from: (i) 1, 4, 9, 21, 24, 27, 36, 37, 39, 42, 43, 44, 47, 54, 55, 56, 74, 80, 85, 87, 99, 102, 114, 117, 119, 121, 126, 128, 131, 143, 144, 158, 159, 160, 169, 182, 188, 190, 197, 198,212, 224, 231, 232, 237, 242, 245, 246, 254, 255, 256, and 257; (ii) 37, 39, 47, 56, 80, 85, 87, 99, 114, 126, 128, and 242; (iii) 39, 99, 126, and 128; (iv) 39 in combination with one or more amino acid substitution at a position selected from 37, 47, 56, 80, 85, 87, 99, 114, 126, 128, and 242; (v) 56 in combination with one or more amino acid substitution at a position selected from 37, 39, 47, 80, 85, 87, 99, 114, 126, 128, and 242; (vi) 114 in combination with one or more amino acid substitution at a position selected from 37, 39, 47, 56, 80, 85, 87, 99, 126, 128, and 242; (vii) A/S126 in combination with one or more amino acid substitution at a position selected from 37, 39, 47, 56, 80, 85, 87, 99, 114, 128, and 242; (viii) N242 in combination with one or more amino acid substitution at a position selected from 37, 39, 47, 56, 80, 85, 87, 99, 114, 126, and 128; (ix) 99+128 in combination with one or more amino acid substitution at a position selected from 39, 56, 114, 126 and 242; (x) 39+242 in combination with one or more amino acid substitution at a position selected from 37, 47, 56, 80, 85, 87, 99, 114, 126, and 128; or (xi) 39+99+128 in combination with one or more amino acid substitution at a position selected from 37, 47, 56, 80, 85, 87, 114, 126, and 242; with the proviso that one or more of said two, three, or four or more substitutions is non-naturally occurring; and wherein the amino acid positions of the variant are numbered by correspondence with the amino acid sequence of SEQ ID NO:85.

Still other embodiments are directed to a subtilisin variant comprising an amino acid sequence comprising two, three, or four or more amino acid substitutions at positions selected from: (i) Q1, V4, S/T9, I/V21, S24, K/R27, S36, Q/T/S/A37, S/P/T39, N/T42, I43, R/S44, A/V47, P/S54, S/T55, T56, N74, I/V80, N/S85, D/E/Q87, S/R99, I/V102, A/T114, M117, I119, N121, A/S126, A/F128, I/S/T131, Q/R143, D/G144, N/S158, I/V159, G160, M169, N/S182, S/T188, I190, V197, G/N198, N/P212, A/V224, K/R231, N/Y232, A/N237, N242, K245, N246, S254, S255, Q256, and F257; (ii) Q/T/S/A37, S/P/T39, A/V47, T56, I/V80, N/S85, D/E/Q87, S/R99, A/T114, A/S126, A/F128, and N242; (iii) S/P/T39, S/R99, A/S126, and A/F128; (iv) S/P/T39 in combination with one or more amino acid substitution at a position selected from Q/T/S/A37, A/V47, T56, I/V80, N/S85, D/E/Q87, S/R99, A/T114, A/S126, A/F128, and N242; (v) T56 in combination with one or more amino acid substitution at a position selected from Q/T/S/A37, S/P/T39, A/V47, I/V80, N/S85, D/E/Q87, S/R99, A/T114, A/S126, A/F128, and N242; (vi) A/T114 in combination with one or more amino acid substitution at a position selected from Q/T/S/A37, S/P/T39, A/V47, T56, I/V80, N/S85, D/E/Q87, S/R99, A/S126, A/F128, and N242; (vii) A/S126 in combination with one or more amino acid substitution at a position selected from Q/T/S/A37, S/P/T39, A/V47, T56, I/V80, N/S85, D/E/Q87, S/R99, A/T114, A/F128, and N242; (viii) N242 in combination with one or more amino acid substitution at a position selected from Q/T/S/A37, S/P/T39, A/V47, T56, I/V80, N/S85, D/E/Q87, S/R99, A/T114, A/S126, and A/F128; (ix) S/R99+A/F128 in combination with one or more amino acid substitution at a position selected from S/P/T39, T56, A/T114, A/S126 and N242; (x) S/P/T39+N242 in combination with one or more amino acid substitution at a position selected from Q/T/S/A37, A/V47, T56, I/V80, N/S85, D/E/Q87, S/R99, A/T114, A/S126, and A/F128; or (xi) S/P/T39+S/R99+A/F128 in combination with one or more amino acid substitution at a position selected from Q/T/S/A37, A/V47, T56, I/V80, N/S85, D/E/Q87, A/T114, A/S126, and N242; with the proviso that one or more of said two, three, or four or more substitutions is non-naturally occurring; and wherein the amino acid positions of the variant are numbered by correspondence with the amino acid sequence of SEQ ID NO:85. Yet another embodiment is directed to a subtilisin variant comprising an amino acid sequence comprising two, three, or four or more amino acid substitutions selected from: (i) 1A, 4I, 9S, 21V, 24F, 27K, 36A, 37T/N, 39E, 42T, 43V, 44S, 47V, 54S, 55M/G, 56N/Y, 74D, 80V, 85S, 87D, 99R, 102I, 114A/P/Q, 117I, 119V, 121S, 126T, 128A, 131T, 143A/T/Q, 144G, 158T, 159I, 160S, 169L, 182S, 188A, 190L, 197I, 198G, 212S/T/K, 224I/V, 231K, 232N, 237T, 242D/Q, 245L, 246S/K, 254T, 255N, 256L, and 257Y; (ii) 37T/N, 39E, 47V, 56N/Y, 80V, 85S, 87D, 99R, 114A/P/Q, 126T, 128A, and 242D/Q; (iii) 39E, 99R, 126T, and 128A; (iv) 39E in combination with one or more substitution at a position selected from 37T/N, 47V, 56N/Y, 80V, 85S, 87D, 99R, 114A/P/Q, 126T, 128A, and 242D/Q; (v) 56N/Y in combination with one or more substitution at a position selected from 37T/N, 39E, 47V, 80V, 85S, 87D, 99R, 114A/P/Q, 126T, 128A, and 242D/Q; (vi) 114A/P/Q in combination with one or more substitution at a position selected from 37T/N, 39E, 47V, 56N/Y, 80V, 85S, 87D, 99R, 126T, 128A, and 242D/Q; (vii) 126T in combination with one or more substitution at a position selected from 37T/N, 39E, 47V, 56N/Y, 80V, 85S, 87D, 99R, 114A/P/Q, 128A, and 242D/Q; (viii) 242D/Q in combination with one or more substitution at a position selected from 37T/N, 39E, 47V, 56N/Y, 80V, 85S, 87D, 99R, 114A/P/Q, 126T, and 128A; (ix) 99R+128A in combination with one or more substitution at a position selected from 39E, 56N/Y, 114A/P/Q, 126T, and 242D/Q; (x) 39E+242D/Q in combination with one or more substitution at a position selected from 37T/N, 47V, 56N/Y, 80V, 85S, 87D, 99R, 114A/P/Q, 126T, and 128A; or (xi) 39E+99R+128A in combination with one or more substitution at a position selected from 37T/N, 47V, 56N/Y, 80V, 85S, 87D, 114A/P/Q, 126T, and 242D/Q; with the proviso that one or more of said two, three, or four or more substitutions is non-naturally occurring; and wherein the amino acid positions of the variant are numbered by correspondence with the amino acid sequence of SEQ ID NO:85. Yet still another embodiment is directed to a subtilisin variant comprising an amino acid sequence comprising two, three, or four or more amino acid substitutions selected from: Q1A, V4I, T9S, I21V, S24F, R27K, S36A, Q/T/S/A37N/T, S/P/T39E, N42T, I43V, R44S, A47V, P54S, S/T55G/M, T56N/Y, N74D, I80V, N85S, E/Q87D, S99R, V102I, A/T114A/P/Q, M117I, I119V, N121S, A/S126T, F128A, I/S131T, Q/R143A/Q/T, D144G, N/S158T, V159I, G160S, M169L, N182S, S/T188A, I190L, V197I, N198G, N/P212K/S/T, A/V224I/V, R231K, Y232N, A/N237T, N242D/Q, K245L, N246K/S, S254T, S255N, Q256L, and F257Y; (ii) Q/T/S/A37N/T, S/P/T39E, A47V, T56N/Y, I80V, N85S, E/Q87D, S99R, A/T114A/P/Q, A/S126T, F128A, and N242D/Q; (iii) S/P/T39E, S99R, A/S126T, and F128A; (iv) S/P/T39E in combination with one or more substitution at a position selected from Q/T/S/A37N/T, A47V, T56N/Y, I80V, N85S, E/Q87D, S99R, A/T114A/P/Q, A/S126T, F128A, and N242D/Q; (v) T56N/Y in combination with one or more substitution at a position selected from Q/T/S/A37N/T, S/P/T39E, A47V, I80V, N85S, E/Q87D, S99R, A/T114A/P/Q, A/S126T, F128A, and N242D/Q; (vi) A/T114A/P/Q in combination with one or more substitution at a position selected from Q/T/S/A37N/T, S/P/T39E, A47V, T56N/Y, I80V, N85S, E/Q87D, S99R, A/S126T, F128A, and N242D/Q; (vii) A/S126T in combination with one or more substitution at a position selected from Q/T/S/A37N/T, S/P/T39E, A47V, T56N/Y, I80V, N85S, E/Q87D, S99R, A/T114A/P/Q, F128A, and N242D/Q; (viii) N242D/Q in combination with one or more substitution at a position selected from Q/T/S/A37N/T, S/P/T39E, A47V, T56N/Y, I80V, N85S, E/Q87D, S99R, A/T114A/P/Q, A/S126T, and F128A; (ix) S99R+F128A in combination with one or more substitution at a position selected from S/P/T39E, T56N/Y, A/T114A/P/Q, A/S126T and N242D/Q; (x) S/P/T39E+N242D/Q in combination with one or more substitution at a position selected from Q/T/S/A37T/N, A47V, T56N/Y, I80V, N85S, E/Q87D, S99R, A/T114A/P/Q, A/S126T, and F128A; or (xi) S/P/T39E+S99R+F128A in combination with one or more substitution at a position selected from Q/T/S/A37N/T, A47V, T56N/Y, I80V, N85S, E/Q87D, A/T114A/P/Q, A/S126T, and N242D/Q; with the proviso that one or more of said two, three, or four or more substitutions is non-naturally occurring; and wherein the amino acid positions of the variant are numbered by correspondence with the amino acid sequence of SEQ ID NO:85.

Another embodiment is directed to one or more subtilisin variant described herein, with the proviso: (i) that said two, three, or four or more variations versus SEQ ID NO:85 is not S39T+I21V+M122L+N177E; (ii) that said two, three, or four or more variations versus SEQ ID NO:85 is not S39E+N74D+D87E; (iii) that said two, three, or four or more variations versus SEQ ID NO:85 is not S39E+N74D+D87E+N253D; (iv) that said two, three, or four or more variations versus SEQ ID NO:85 is not I21V+S39E+N74D+D87E+M122L+N253D; (v) that said two, three, or four or more variations versus SEQ ID NO:85 is not Q37E+Q256E; and/or (vi) that said variation versus SEQ ID NO:85 at position 21 is not a valine when said variant comprises a variation versus SEQ ID NO:85 at one or more positions selected from: 4, 36, 42, 47, 56, 87, 99, 102, 114, 188, 224, 237, 242, and 255. An even further embodiment is directed to one or more subtilisin variant described herein, with the proviso: (i) that said two, three, or four or more substitutions is not S39T+I21V+M122L+N177E; (ii) that said two, three, or four or more substitutions is not S39E+N74D+D87E; (iii) that said two, three, or four or more substitutions is not S39E+N74D+D87E+N253D; (iv) that said two, three, or four or more substitutions is not I21V+S39E+N74D+D87E+M122L+N253D; (v) that said two, three, or four or more substitutions is not Q37E+Q256E; and/or (vi) that the substitution at position 21 is not a valine when said variant comprises a substitution at one or more positions selected from: 4, 36, 42, 47, 56, 87, 99, 102, 114, 188, 224, 237, 242, and 255.

A further embodiment is directed to a subtilisin variant comprising an amino acid sequence comprising: (i) one or more variation versus SEQ ID NO:85 at positions selected from 56, 114, and 126; (ii) one or more variation versus SEQ ID NO:85 at positions selected from 56N/Y, 114A/P/Q, and 126T; (iii) one or more amino acid substitution at positions selected from 56, 114, and 126; (iv) one or more amino acid substitution at positions selected from T56, A/T114, and A/S126; (v) one or more amino acids substitutions selected from: 56N/Y, 114A/P/Q, and 126T; or (vi) one or more amino acids substitutions selected from: T56N/Y, A/T114A/P/Q, and A/S126T; with the proviso that one or more of said two, three, or four or more variations or substitutions is non-naturally occurring; and wherein the amino acid positions of the variant are numbered by correspondence with the amino acid sequence of SEQ ID NO:85.

A still even further embodiment is directed to a subtilisin variant comprising an amino acid sequence comprising one or more amino acid substitutions selected from: N074D-I080V-N085S-E087D-S099R-V102I-S158T-V159I-N242D-F257Y; N074D-N085S-E087D-S099R-V102I-S126T-F128A-I190L-N242D-F257Y; R027K-S099R-V102I-T114Q-I119V-S126T-F128A-S158T-V159I-G160S-T188A-N242D; N074D-I080V-N085S-E087D-S099R-V102I-T114Q-S126T-F128A-N242D; R027K-S099R-V102I-S126T-F128A-R143A-S158T-V159I-G160S; N074D-I080V-N085S-E087D-S099R-V102I-I190L-N242D-F257Y; N074D-I080V-N085S-E087D-S099R-V102I-T114Q-R143A-I190L-N242D-F257Y; N074D-S099R-V102I-S126T-F128A-R143A-S158T-N212S; N074D-I080V-N085S-E087D-S099R-T114Q-S126T-F128A-R143A-N242D; N085S-E087D-S099R-T114Q-I119V-S126T-F128A-R143A-G160S-F257Y; N074D-I080V-S099R-V102I-T114Q-S126T-F128A-R143A-N212S-N242D; N074D-I080V-N085S-E087D-S099R-V102I-S126T-F128A-R143A-I190L-N242D-F257Y; N074D-I080V-S099R-V102I-T114Q-I119V-S126T-F128A-S158T-G160S-N212S-F257Y; I080V-N085S-E087D-S099R-V102I-T114Q-I119V-S126T-F128A-R143A-N242D; N085S-E087D-S099R-V102I-T114Q-S158T-V159I-G160S-N212S-N242D-F257Y; R027K-N074D-S099R-V102I-T114Q-S126T-F128A-T188A-I190L-N212S-N242D; R027K-N074D-S099R-V102I-I119V-S126T-F128A-R143A-G160S-N212S-N242D; N042T-I080V-N085S-E087D-T114A-F128A-R143Q-D144G-S158T-V159I-G160S-N198G; I021V-I080V-N085S-E087D-M117I-F128A-S131T-R143Q-D144G-A224V; N074D-I080V-N085S-E087D-S158T-N242D; Q001A-I080V-F128A-S131T-R143A-D144G-M169L-I190L-S254T-S255N-Q256L-F257Y; A037T-S039E-N042T-S099R-V102I-T114Q-S126T-F128A-N242D; A037T-S039E-S099R-V102I-T114Q-S126T-F128A-R143A-N212S-N242D; A037T-S039E-N074D-S099R-V102I-S126T-F128A-R143A-N212S-N242D; A037T-S039E-N042T-N074D-S099R-V102I-S126T-F128A-S158T-N242D; A037T-S039E-I043V-A047V-I080V-N085S-E087D-S099R-T114A-F128A; A037T-S039E-I043V-A047V-T055G-S099R-V102I-T114Q-S126T-F128A-N212S; A037T-S039E-I043V-A047V-I080V-N085S-E087D-S099R-T114Q-F128A-N242D; A037T-S039E-I043V-A047V-I080V-N085S-E087D-S099R-T114A-F128A-N242D; A037T-S039E-A047V-I080V-N085S-E087D-S099R-T114Q-F128A-R143A-N242D; S036A-S039E-I043V-A047V-T055M-I080V-N085S-E087D-S099R-T114A-F128A; A037T-S039E-I043V-A047V-I080V-N085S-E087D-S099R-V102I-T114Q-F128A; A037T-S039E-I043V-A047V-N074D-N085S-E087D-S099R-T114A-F128A-S158T-N212S; A037T-S039E-N074D-I080V-N085S-E087D-S099R-V102I-T114Q-S126T-F128A-R143A; A037T-S039E-I043V-A047V-T055G-N074D-I080V-S099R-V102I-T114Q-S126T-F128A; A037T-S039E-I043V-A047V-N074D-I080V-S099R-V102I-T114Q-S126T-F128A-N242D; R027K-A037T-S039E-A047V-T055G-T056Y-N074D-S099R-V102I-S126T-F128A-N242D; A037T-S039E-N042T-A047V-N074D-I080V-N085S-E087D-S099R-T114Q-F128A-N242D; A037T-S039E-I043V-A047V-I080V-N085S-E087D-S099R-T114A-S126T-F128A-N212S; A037T-S039E-I043V-A047V-I080V-N085S-E087D-S099R-T114A-S126T-F128A-N242D; A037T-S039E-I043V-A047V-I080V-N085S-E087D-S099R-T114Q-S126T-F128A-R143A; A037T-S039E-I043V-A047V-I080V-N085S-E087D-S099R-V102I-T114A-F128A-N212S; A037T-S039E-N042T-A047V-I080V-N085S-E087D-S099R-V102I-T114Q-F128A-N242D; A037T-S039E-I043V-A047V-T056Y-I080V-N085S-E087D-S099R-T114A-F128A-N242D; A037T-S039E-I043V-A047V-I080V-N085S-E087D-S099R-T114Q-F128A-N212S-N242D; A037T-S039E-A047V-I080V-N085S-E087D-S099R-V102I-T114Q-F128A-R143A-N242D; A037T-S039E-I043V-A047V-T055G-I080V-N085S-E087D-S099R-T 114Q-S126T-F128A; R027K-A037T-S039E-A047V-I080V-N085S-E087D-S099R-T114Q-F128A-N212S-N242D; T009S-A037T-S039E-I043V-A047V-I080V-N085S-E087D-S099R-T 114A-F128A-V197I; S039E-I043V-A047V-I080V-N085S-E087D-S099R-T114A-F128A-N 212S-K245L-N246S; A037T-S039E-I043V-A047V-I080V-N085S-E087D-S099R-T114A-F128A-I190L-A224V; S036A-S039E-I043V-A047V-I080V-N085S-E087D-S099R-T114A-N121S-F128A-R143Q; S036A-S039E-I043V-A047V-I080V-N085S-E087D-S099R-T114A-F128A-N212S-S255N; S039E-I043V-A047V-I080V-N085S-E087D-S099R-T114A-F128A-R143Q-N242D-N246K; A037T-S039E-I043V-A047V-N074D-N085S-E087D-S099R-T114A-S126T-F128A-N212S; A037T-S039E-I043V-A047V-N074D-I080V-N085S-E087D-S099R-T114A-F128A-N212S; A037T-S039E-N042T-A047V-I080V-N085S-E087D-S099R-T114A-F128A-S158T-N212S; V004I-A037T-S039E-I043V-A047V-I080V-N085S-E087D-S099R-T114A-F128A-N182S; A037T-S039E-I043V-A047V-N074D-N085S-E087D-S099R-T114A-S126T-F128A-S158T-N212S; A037T-S039E-I043V-A047V-N074D-I080V-N085S-E087D-S099R-T114A-F128A-S158T-N212S; A037T-S039E-I043V-A047V-N074D-N085S-E087D-S099R-T114A-F128A-N212S-N242Q-K245L; A037T-S039E-I043V-A047V-N074D-I080V-N085S-E087D-S099R-T114A-F128A-N212S-N242Q; A037T-S039E-I043V-A047V-N074D-I080V-N085S-E087D-S099R-T114A-F128A-N212S-K245L; A037T-S039E-A047V-S099R-V102I-T114A-I119V-S126T-F128A-R143A-S158T-G160S-N212S; A037T-S039E-N042T-A047V-T056Y-N074D-I080V-N085S-E087D-S099R-T114Q-S126T-F128A; S024F-A037T-S039E-A047V-N074D-I080V-N085S-E087D-S099R-T114Q-S126T-F128A-N212S; A037T-S039E-I043V-A047V-I080V-N085S-E087D-S099R-T114Q-S126T-F128A-R143A-N242D; A037T-S039E-I043V-A047V-I080V-N085S-E087D-S099R-T114A-S126T-F128A-A237T-N242D; A037T-S039E-I043V-A047V-I080V-N085S-E087D-S099R-V102I-T114Q-S126T-F128A-N242D; A037T-S039E-I043V-A047V-T055G-I080V-N085S-E087D-S099R-V102I-T114Q-F128A-R143A; A037T-S039E-N042T-A047V-T055G-I080V-N085S-E087D-S099R-T114Q-F128A-N212S-N242D; A037T-S039E-I043V-A047V-I080V-N085S-E087D-S099R-T114A-F128A; A037T-S039E-I043V-A047V-I080V-N085S-E087D-S099R-T114A-F128A-N242D; A037T-S039E-I043V-A047V-I080V-N085S-E087D-S099R-T114Q-F128A-N242D; A037T-S039E-I043V-A047V-I080V-N085S-E087D-S099R-T114A-S126T-F128A-N242D; A037T-S039E-I043V-A047V-T056Y-I080V-N085S-E087D-S099R-T114A-F128A-N242D; A037T-S039E-I043V-A047V-I080V-N085S-E087D-S099R-T114Q-S126T-F128A-R143A; A037T-S039E-I043V-A047V-T055G-I080V-N085S-E087D-S099R-T114Q-S126T-F128A; A037T-S039E-I043V-A047V-N074D-I080V-N085S-E087D-S099R-T114A-F128A-S158T-N212S; A037T-S039E-I043V-A047V-I080V-N085S-E087D-S099R-T114A-S126T-F128A-A237T-N242D; A037T-S039E-I043V-A047V-N074D-I080V-N085S-E087D-S099R-T114A-S126T-F128A-N242D; R027K-A037T-S039E-I043V-A047V-I080V-N085S-E087D-S099R-T114A-S126T-F128A-N242D; A037T-S039E-I043V-A047V-T056Y-I080V-N085S-E087D-S099R-T114A-S126T-F128A-R143A; A037T-S039E-I043V-A047V-I 080V-N085S-E087D-S099R-T114A-N121S-F128A-N246S-S255N; A037T-S039E-A047V-I080V-N085S-E087D-S099R-T114Q-F128A-R143A-N242D; A037T-S039E-I043V-A047V-N074D-N085S-E087D-S099R-T114A-F128A-S158T-N212S; A037T-S039E-I043V-A047V-I080V-N085S-E087D-S099R-V102I-T114Q-S126T-F128A-N242D; A037T-S039E-I043V-A047V-T056Y-I080V-N085S-E087D-S099R-T114Q-S126T-F128A-N242D; A037T-S039E-I043V-A047V-N074D-I080V-N085S-E087D-S099R-T114Q-I119V-S126T-F128A; A037T-S039E-I043V-A047V-T055G-I080V-N085S-E087D-S099R-V102I-T114A-S126T-F128A-S158T; A037T-S039E-I043V-A047V-I080V-N085S-E087D-S099R-T114A-S126T-F128A-R143A-S158T-N242D; A037T-S039E-N042T-I043V-R044S-A047V-N074D-I080V-N085S-E087D-S099R-T114A-S126T-F128A; A037T-S039E-N042T-I043V-R044S-A047V-I080V-N085S-E087D-S099R-T114A-S126T-F128A-N242D; A037T-S039E-I043V-A047V-I080V-N085S-E087D-S099R-V102I-T114A-I119V-S126T-F128A-F257Y; A037T-S039E-I043V-A047V-N074D-I080V-N085S-E087D-S099R-V102I-T114A-N121S-F128A-R143Q; A037T-S039E-I043V-A047V-N074D-N085S-E087D-S099R-T114A-S126T-F128A-S158T-N212S; S036A-S039E-I043V-A047V-N074D-I080V-N085S-E087D-S099R-T114A-F128A-A224V-S255N; A037T-S039E-I043V-A047V-T055G-N074D-I080V-N085S-E087D-S099R-T 114Q-S126T-F128A-N242D; A037T-S039E-I043V-A047V-T055G-I080V-N085S-E087D-S099R-V102I-T114Q-S126T-F128A-R143A; A037T-S039E-I043V-A047V-I080V-N085S-E087D-S099R-V102I-T114Q-S126T-F128A-R143A-N242D; A037T-S039E-I043V-A047V-T056Y-I080V-N085S-E087D-S099R-T114Q-S126T-F128A-S158T-N242D; A037T-S039E-I043V-A047V-T056Y-I080V-N085S-E087D-S099R-T114Q-I119V-S126T-F128A-F257Y; A037T-S039E-I043V-A047V-T056Y-I080V-N085S-E087D-S099R-T114Q-I119V-S126T-F128A-N242D; A037T-S039E-I043V-A047V-N074D-I080V-N085S-E087D-S099R-T114A-S126T-F128A-S158T-N212S-N242Q; A037T-S039E-I043V-A047V-N074D-I080V-N085S-E087D-S099R-T114A-S126T-F128A-T188A-I190L-F257Y; V004I-T009S-A037T-S039E-I043V-A047V-I080V-N085S-E087D-S099R-T114A-F128A-A224V-K245L-S255N; A037T-S039E-A047V-T056Y-I080V-N085S-E087D-S099R-T114Q-R143A-N242D; A037T-S039E-N042T-A047V-T056Y-N074D-I080V-N085S-E087D-S099R-T114Q-S126T-F128A; A037T-S039E-1043V-A047V-N074D-N085S-E087D-S099R-T114A-S126T-F128A-S158T-N212S-N242Q; A037T-S039E-I043V-A047V-N074D-N085S-E087D-S099R-T114A-S126T-F128A-S158T-N212S-K245L; A037T-S039E-I043V-A047V-I080V-N085S-E087D-S099R-T114P-S126T-F128A-R143T-S158T-N212K-N242D; A037T-S039E-I043V-A047V-T056Y-N074D-I080V-N085S-E087D-S099R-T114Q-S126T-F128A-R143A-N242D; A037T-S039E-I043V-A047V-T055G-T056Y-N074D-I080V-N085S-E087D-S099R-V102I-T114Q-S126T-F128A; S036A-A037N-S039E-I043V-A047V-I080V-N085S-E 087D-S099R-T114A-F128A-N242D-K245L-N246S-S255N; A037T-S039E-I043V-A047V-N074D-I080V-N085S-E087D-S099R-T114A-S126T-F128A-S158T-N212S-N242Q-K245L; A037T-S039E-I043V-A047V-I080V-N085S-E087D-S099R-T114A-I119V-S126T-F128A-S158T-G160S-N242D-F257Y; A037T-S039E-I043V-A047V-I080V-N085S-E087D-S099R-T114A-N121S-F128A-N182S-I190L-K245L-N246S-S255N; A037T-S039E-N074D-I080V-N085S-E087D-S099R-V102I-T114Q-S126T-F128A-R143A; A037T-S039E-I043V-A047V-T055G-N074D-I080V-S099R-V102I-T114Q-S126T-F128A; A037T-S039E-I043V-A047V-N074D-I080V-S099R-V102I-T114Q-S126T-F128A-N242D; A037T-S039E-A047V-N074D-I080V-N085S-E087D-S099R-V102I-T114Q-S126T-F128A-S158T-N242D; A037T-S039E-N042T-A047V-N074D-I080V-N085S-E087D-S099R-V102I-T114Q-S126T-F128A-N242D; A037T-S039E-I043V-A047V-N074D-N085S-E087D-S099R-T114A-S126T-F128A-S158T-N212S-N242Q-K245L; T009S-S036A-S039E-I043V-A047V-I080V-N085S-E087D-S099R-T114A-F128A-R143Q-N182S-T188A-I190L; A037T-S039E-I043V-A047V-T055G-N074D-I080V-N085S-E087D-S099R-V102I-T114Q-S126T-F128A-S158T-N242D; A037T-S039E-I043V-A047V-T055G-N074D-I080V-N085S-E087D-S099R-T114Q-S126T-F128A-R143A-S158T-N212S; A037T-S039E-I043V-A047V-I080V-N085S-E087D-S099R-T114A-F128A-R143Q-N212S-A224V-Y232N-K245L-N246S-S255N; V004I-T009S-A037T-S039E-I043V-A047V-I080V-N085S-E087D-S099R-T114A-N121S-F128A-R143Q-A224I-R231K-K245L; R027K-A037T-S039E-A047V-I080V-N085S-E087D-S099R-T114Q-I119V-S126T-F128A-G160S-N242D-F257Y; R027K-A037T-S039E-I043V-A047V-T056Y-I080V-N085S-E087D-S099R-T114Q-S126T-F128A-S158T-G160S-N242D-F257Y; R027K-A037T-S039E-I043V-A047V-T056Y-I080V-N085S-E087D-S099R-V102I-T114Q-S126T-F128A-T188A-I190L-F257Y; R027K-A037T-S039E-I043V-A047V-T056N-I080V-N085S-E087D-S099R-T114P-S126T-F128A-R143T-V159I-N212T-F257Y; A037T-S039E-I043V-A047V-T056Y-I080V-N085S-E087D-S099R-T114Q-I119V-S126T-F128A-S158T-V159I-G160S-F257Y; A037T-S039E-I043V-A047V-T055G-T056Y-N074D-I080V-N085S-E087D-S099R-V102I-T114Q-S126T-F128A-N242D; R027K-A037T-S039E-A047V-T056Y-N074D-I080V-N085S-E087D-S099R-T114Q-I119V-S126T-F128A-N242D-F257Y; A037T-S039E-N042T-N074D-S099R-V102I-S126T-F128A-S158T-N242D; R027K-A037T-S039E-A047V-T055G-T056Y-N074D-S099R-V102I-S126T-F128A-N242D; S039E-I043V-A047V-I080V-N085S-E087D-S099R-T114A-F128A-A224V-N242D-N246S-S255N; A037T-S039E-I043V-A047V-N074D-I080V-N085S-E087D-S099R-V102I-T114A-F128A-R143Q-S158T-A224V-S255N; A037T-S039E-I043V-A047V-P054S-TO56Y-A057Q-N074D-I080V-N085S-E087D-S099R-T114A-N121S-F128A-S158T-A224V-S255N; A037T-S039E-N042T-I043V-R044S-A047V-N074D-I080V-N085S-E087D-S099R-V102I-S126T-F128A-S 158T-G160S-N242D; S126T; T114Q; T056Y; S099R-F128A; S099R-S126T; S126T-F128A; T056Y-T114Q; S039E-E087D; S099R-F128A-N242D; S099R-S126T-N242D; S099R-S126T-F128A; T056Y-T114Q-N242D; S039E-S099R-S126T; S039E-S099R-F128A; S039E-E087D-N242D; T056Y-S099R-T114Q-F128A; T056Y-S099R-T114Q-S126T; S039E-T056Y-S099R-F128A; S039E-S099R-T114Q-F128A; S039E-E087D-S099R-S126T; S039E-N085S-S099R-F128A; S039E-S099R-T114A-S126T; S039E-E087D-S099R-F128A; S039E-S099R-S126T-F128A; S039E-T056Y-E087D-T114Q; S039E-E087D-S099R-S126T-F128A; S039E-T056Y-S099R-S126T-F128A; S039E-E087D-S099R-F128A-N242D; A037T-S039E-E087D-S099R-T114A-F128A; S039E-T056Y-E087D-S099R-S126T-F128A; S039E-S099R-T114Q-S126T-F128A-N242D; A037T-S039E-S099R-T114Q-S126T-F128A-N242D; A037T-S039E-T056Y-S099R-S126T-F128A-N242D; A037T-S039E-T056Y-N085S-E087D-S099R-T114Q-F128A; A037T-S039E-A047V-T056Y-S099R-T114Q-S126T-F128A; A037T-S039E-A047V-T056Y-S099R-T114A-S126T-F128A; A037T-S039E-T056Y-S099R-T114Q-S126T-F128A-N242D; S039E-A047V-E087D-S099R-T114Q-S126T-F128A-N242D; S039E-A047V-T056Y-N085S-E087D-S099R-T114Q-S126T-F128A; S039E-A047V-I080V-E087D-S099R-T114A-S126T-F128A-N242D; A037T-S039E-T056Y-N085S-S099R-T114Q-S126T-F128A-N242D; S039E-A047V-T056Y-E087D-S099R-T114A-S126T-F128A-N242D; S039E-A047V-T056Y-E087D-S099R-T114Q-S126T-F128A-N242D; A037T-S039E-A047V-1080V-N085S-E087D-S099R-T114A-S126T-F128A; A037T-S039E-1080V-N085S-E087D-S099R-T114Q-S126T-F128A-N242D; A037T-S039E-T056Y-N085S-E087D-S099R-T114A-S126T-F128A-N242D; A037T-S039E-A047V-T056Y-E087D-S099R-T114A-S126T-F128A-N242D; and combinations thereof, wherein the amino acid positions of the variant are numbered by correspondence with the amino acid sequence of SEQ ID NO:85.

One embodiment provides one or more subtilisin variant comprising an amino acid sequence comprising amino acid substitutions S39E+S99R+F128A, where the amino acid positions of the variant are numbered by correspondence with the amino acid sequence of SEQ ID NO:85. A still further embodiment provides one or more subtilisin variant comprising an amino acid sequence comprising amino acid substitutions S39E+S99R+F128A+N242D, where the amino acid positions of the variant are numbered by correspondence with the amino acid sequence of SEQ ID NO:85. Yet a further embodiment provides one or more subtilisin variant comprising an amino acid sequence comprising amino acid substitutions S39E+S99R+F128A+I43V, where the amino acid positions of the variant are numbered by correspondence with the amino acid sequence of SEQ ID NO:85. A still further embodiment provides one or more subtilisin variant comprising an amino acid sequence comprising amino acid substitutions S39E+S99R+F128A+A47V, where the amino acid positions of the variant are numbered by correspondence with the amino acid sequence of SEQ ID NO:85. Some embodiments provide one or more subtilisin variant comprising an amino acid sequence comprising amino acid substitutions S39E+S99R+F128A+I80V, where the amino acid positions of the variant are numbered by correspondence with the amino acid sequence of SEQ ID NO:85. A still further embodiment provides one or more subtilisin variant comprising an amino acid sequence comprising amino acid substitutions S39E+S99R+F128A and one or more amino acid substitution selected from: (i) Q1A, V4I, T9S, I21V, S24F, R27K, S36A, A37T/N, N42T, I43V, R44S, A47V, P54S, T55M/G, T56N/Y, N74D, I80V, N85S, E87D, V102I, T114A/P/Q, M117I, I119V, N121S, S126T, S131T, R143A/T/Q, D144G, S158T, V159I, G160S, M169L, N182S, T188A, I190L, V197I, N198G, N212S/T/K, A224I/V, R231K, Y232N, A237T, N242D/Q, K245L, N246S/K, A254T, S255N, Q256L, and F257Y; (ii) Q1A, I21V, R27K, N42T, N74D, I80V, N85S, E87D, V102I, T114A/Q, M117I, I119V, S126T, S131T, R143A/Q, D144G, S158T, V159I, G160S, M169L, T188A, I190L, N198G, N212S, A224V, N242D, S254T, S255N, Q256L, and F257Y; (iii) V4I, T9S, S24F, R27K, S36A, A37T/N, N42T, I43V, R44S, A47V, P54S, T55M/G, T56N/Y, A57Q, N74D, I80V, N85S, E87D, V102I, T114A/P/Q, I119V, N121S, S126T, R143A/T/Q, S158T, V159I, G160S, N182S, T188A, I190L, V197I, N212S/T/K, A224I/V, R231K, Y232N, A237T, N242D/Q, K245L, N246S/K, S255N, and F257Y; (iv) T9S, R27K, A37T/N, N42T, I43V, A47V, T55G, T56Y, N74D, I80V, N85S, E87D, V102I, T114A/Q, I119V, S126T, R143A/Q, S158T, G160S, N212S, N242D, K245L, N246S, S255N, and F257Y; (v) T9S, R27K, N42T, T55G, I119V, G160S, K245L, N246S, S255N, and F257Y; (vi) N74D, V102I, R143A/Q, S158T, and N212S; (vii) A37T/N, I43V, A47V, I80V, N85S, E87D, and T114A; (viii) N42T, T56Y, N74D, V102I, S126T, S158T, and N242D; (ix) T56Y, T114Q, S126T, and N242D; (x) I43V, A47V, I80V and N242D; (xi) I43V; (xii) A47V; (xiii) 180V; (xiv) N242D; or (xv) a combination of one or more of groups (i) to (xiv); where the amino acid positions of the variant are numbered by correspondence with the amino acid sequence of SEQ ID NO:85.

A further embodiment provides one or more subtilisin variant comprising an amino acid sequence comprising amino acid substitution N242D, where the amino acid positions of the variant are numbered by correspondence with the amino acid sequence of SEQ ID NO:85. A still further embodiment provides one or more subtilisin variant comprising an amino acid sequence comprising amino acid substitution N242D and one or more amino acid substitutions selected from: (i) Q1A, V4I, T9S, I21V, S24F, R27K, S36A, A37T/N, S39E, N42T, I43V, R44S, A47V, P54S, T55M/G, T56N/Y, N74D, I80V, N85S, E87D, S99R, V102I, T114A/P/Q, M117I, I119V, N121S, S126T, F128A, S131T, R143A/T/Q, D144G, S158T, V159I, G160S, M169L, N182S, T188A, I190L, V197I, N198G, N212S/T/K, A224I/V, R231K, Y232N, A237T, K245L, N246S/K, A254T, S255N, Q256L, and F257Y; (ii) Q1A, I21V, R27K, S39E, N42T, N74D, I80V, N85S, E87D, S99R, V102I, T114A/Q, M117I, I119V, S126T, F128A, S131T, R143A/Q, D144G, S158T, V159I, G160S, M169L, T188A, I190L, N198G, N212S, A224V, S254T, S255N, Q256L, and F257Y; (iii) V4I, T9S, S24F, R27K, S36A, A37T/N, S39E, N42T, I43V, R44S, A47V, P54S, T55M/G, T56N/Y, A57Q, N74D, I80V, N85S, E87D, S99R, V102I, T114A/P/Q, I119V, N121S, S126T, F128A, R143A/T/Q, S158T, V159I, G160S, N182S, T188A, I190L, V197I, N212S/T/K, A224I/V, R231K, Y232N, A237T, K245L, N246S/K, S255N, and F257Y; (iv) T9S, R27K, A37T/N, S39E, N42T, I43V, A47V, T55G, T56Y, N74D, I80V, N85S, E87D, S99R, V102I, T114A/Q, I119V, S126T, F128A, R143A/Q, S158T, G160S, N212S, K245L, N246S, S255N, and F257Y; (v) S39E, S99R, F128A, T56Y, T114Q, and S126T; (vi) S39E, N42T, T56Y, N74D, S99R, V102I, S126T, F128A, and S158T; (vii) I43V, A47V, and I80V; (viii) I43V; (ix) A47V; (x) 180V; and/or (xi) a combination of one or more of groups (i) to (x); where the amino acid positions of the variant are numbered by correspondence with the amino acid sequence of SEQ ID NO:85.

A further embodiment provides one or more subtilisin variant comprising an amino acid sequence comprising amino acid substitution 180V, where the amino acid positions of the variant are numbered by correspondence with the amino acid sequence of SEQ ID NO:85. A still further embodiment provides one or more subtilisin variant comprising an amino acid sequence comprising amino acid substitution I80V and one or more amino acid substitution selected from: (i) Q1A, V4I, T9S, I21V, S24F, R27K, S36A, A37T/N, S39E, N42T, I43V, R44S, A47V, P54S, T55M/G, T56N/Y, N74D, N85S, E87D, S99R, V102I, T114A/P/Q, M117I, I119V, N121S, S126T, F128A, S131T, R143A/T/Q, D144G, S158T, V159I, G160S, M169L, N182S, T188A, I190L, V197I, N198G, N212S/T/K, A224I/V, R231K, Y232N, A237T, N242D, K245L, N246S/K, A254T, S255N, Q256L, and F257Y; (ii) Q1A, I21V, R27K, S39E, N42T, N74D, N85S, E87D, S99R, V102I, T114A/Q, M117I, I119V, S126T, F128A, S131T, R143A/Q, D144G, S158T, V159I, G160S, M169L, T188A, I190L, N198G, N212S, A224V, N242D, S254T, S255N, Q256L, and F257Y; (iii) V4I, T9S, S24F, R27K, S36A, A37T/N, S39E, N42T, I43V, R44S, A47V, P54S, T55M/G, T56N/Y, A57Q, N74D, N85S, E87D, S99R, V102I, T114A/P/Q, I119V, N121S, S126T, F128A, R143A/T/Q, S158T, V159I, G160S, N182S, T188A, I190L, V197I, N212S/T/K, A224I/V, R231K, Y232N, A237T, N242D, K245L, N246S/K, S255N, and F257Y; (iv) T9S, R27K, A37T/N, S39E, N42T, I43V, A47V, T55G, T56Y, N74D, N85S, E87D, S99R, V102I, T114A/Q, I119V, S126T, F128A, R143A/Q, S158T, G160S, N212S, N242D, K245L, N246S, S255N, and F257Y; (v) S39E, S99R, F128A, T56Y, T114Q, S126T, and N242D; (vi) S39E, N42T, T56Y, N74D, S99R, V102I, S126T, F128A, S158T, and N242D; (vii) I43V, A47V, and N242D; (viii) I43V; (ix) A47V; (x) N242D; and/or (xi) a combination of one or more of groups (i) to (x); where the amino acid positions of the variant are numbered by correspondence with the amino acid sequence of SEQ ID NO:85.

A still further embodiment is directed to one or more subtilisin variant described herein, wherein said variant is a member of the Bacillus gibsonii-clade. Another embodiment is directed to one or more subtilisin variant described herein, wherein said variant further comprises a DXGIXXHSDLXXXGGASXXXXXPTTADLNXH GTH (SEQ ID NO:71) or DXGIXXHSDLXXXGGASXXXXXXTTADLXXHGTH (SEQ ID NO:72) motif, wherein the initial D is the active site Aspartic acid residue, the penultimate H is the active site Histidine, and X is any amino acid.

In another embodiment, one or more variant described herein is from a parent amino acid sequence, wherein said parent is a member of the Bacillus gibsonii-clade. In yet still another embodiment, one or more variant described herein is from a parent amino acid sequence comprising a DXGIXXHSDLXXXGGASXXXXXPTTADLNXHGTH (SEQ ID NO:71) or DXGIXXHSDLXXXGGASXXXXXXTTADLXXHGTH (SEQ ID NO:72) motif, wherein the initial D is the active site Aspartic acid residue, the penultimate H is the active site Histidine, and X is any amino acid. Yet in an even still further embodiment, one or more variant described herein is from a parent amino acid sequence, wherein said parent amino acid sequence has 70%, 75%, 80%, 85%, 86%, 87%, 88%, 89%, 90%, 91%, 92%, 93%, 94%, 95%, 96%, 97%, 98%, 99%, or 100% amino acid sequence identity to the amino acid sequence of SEQ ID NO:18 or 85.

An even still further embodiment provides one or more subtilisin variant comprising an amino acid sequence comprising one or more amino acid substitutions selected from N74D, I80V, V102I, T114Q, I119V, S126T, R143A, S158T, G160S, N212S, N242D, S255N, and F257Y and a DXGIXXHSDLXXXGGASXXXXXPTTADLNXHGTH (SEQ ID NO:71) or DXGIXXHSDLXXXGGASXXXXXXTTADLXXHGTH (SEQ ID NO:72) motif, where the initial D is the active site Aspartic acid residue, the penultimate H is the active site Histidine, and X is any amino acid, and where the amino acid positions of the variant are numbered by correspondence with the amino acid sequence of SEQ ID NO:85. Yet an even still further embodiment provides one or more subtilisin variant comprising an amino acid sequence comprising (i) one or more amino acid substitutions selected from N74D, I80V, V102I, T114Q, I119V, S126T, R143A, S158T, G160S, N212S, N242D, S255N, and F257Y; (ii) one or more amino acid substitutions selected from N42T, N85S, E87D, S99R, and F128A; and (iii) a DXGIXXHSDLXXXGGASXXXXXPTTADLNXHGTH (SEQ ID NO:71) or DXGIXXHSDLXXXGGASXXXXXXTTADLXXHGTH (SEQ ID NO:72) motif, where the initial D is the active site Aspartic acid residue, the penultimate H is the active site Histidine, and X is any amino acid, and where the amino acid positions of the variant are numbered by correspondence with the amino acid sequence of SEQ ID NO:85. Yet another embodiment, provides one or more subtilisin variant comprising an amino acid sequence comprising one or more amino acid substitutions selected from N74D, I80V, V102I, T114Q, I119V, S126T, R143A, S158T, G160S, N212S, N242D, S255N, and F257Y and a DXGIXXHSDLXXXGGASXXXXXPTTADLNXHGTH (SEQ ID NO:71) motif, where the initial D is the active site Aspartic acid residue, the penultimate H is the active site Histidine, and X is any amino acid, and where the amino acid positions of the variant are numbered by correspondence with the amino acid sequence of SEQ ID NO:85. A still yet further embodiment is directed to one or more subtilisin variant comprising an amino acid sequence comprising one or more amino acid substitutions selected from N74D, I80V, V102I, T114Q, I119V, S126T, R143A, S158T, G160S, N212S, N242D, S255N, and F257Y and a DXGIXXHSDLXXXGGASXXXXXXTTADLXXHGTH (SEQ ID NO:72) motif, where the initial D is the active site Aspartic acid residue, the penultimate H is the active site Histidine, and X is any amino acid, and where the amino acid positions of the variant are numbered by correspondence with the amino acid sequence of SEQ ID NO:85. A still further embodiment provides one or more subtilisin variant comprising an amino acid sequence comprising one or more amino acid substitutions selected from N74D, I80V, V102I, T114Q, I119V, S126T, R143A, S158T, G160S, N212S, N242D, S255N, and F257Y; one or more amino acid substitutions selected from N42T, N85S, E87D, S99R, and F128A; and a DXGIXXHSDLXXXGGASXXXXXPTTADLNXHGTH (SEQ ID NO:71) or DXGIXXHSDLXXXGGASXXXXXXTTADLXXHGTH (SEQ ID NO:72) motif, where the initial D is the active site Aspartic acid residue, the penultimate H is the active site Histidine, and X is any amino acid, and where the amino acid positions of the variant are numbered by correspondence with the amino acid sequence of SEQ ID NO:85.

In a further embodiment, one or more subtilisin variant described herein further comprises an amino acid sequence with 70%, 75%, 80%, 85%, 86%, 87%, 88%, 89%, 90%, 91%, 92%, 93%, 94%, 95%, 96%, 97%, 98%, 99% or less than 100% amino acid identity to the amino acid sequence of SEQ ID NO:18 or 85. In other embodiments, one or more subtilisin variant described herein further comprises an amino acid sequence with 70%, 75%, 80%, 85%, 90%, 95%, 96%, 97%, 98%, or 99% amino acid sequence identity to the amino acid sequence of SEQ ID NO:18 or 85. In some embodiments, one or more subtilisin variant described herein further comprises an amino acid sequence with 70%, 75%, 80%, 85%, 90%, 95%, 96%, 97%, 98%, or 99% amino acid sequence identity to the amino acid sequence of SEQ ID NO:85. In other embodiments, one or more subtilisin variant described herein further comprises an amino acid sequence with 80%, 85%, 90%, 95%, 96%, 97%, 98%, or 99% amino acid sequence identity to the amino acid sequence of SEQ ID NO:18 or 85. In still other embodiments, one or more subtilisin variant described herein further comprises an amino acid sequence with 80%, 85%, 90%, 95%, 96%, 97%, 98%, or 99% amino acid sequence identity to the amino acid sequence of SEQ ID NO:85. In further embodiments, one or more subtilisin variant described herein further comprises an amino acid sequence with 90%, 95%, 96%, 97%, 98%, or 99% amino acid sequence identity to the amino acid sequence of SEQ ID NO:18 or 85. In yet even still other embodiments, one or more subtilisin variant described herein further comprises an amino acid sequence with 90%, 95%, 96%, 97%, 98%, or 99% amino acid sequence identity to the amino acid sequence of SEQ ID NO:85.

As noted above, one or more subtilisin variant described herein has enzymatic activity (e.g., protease activity) and thus is useful in cleaning applications, including but not limited to, methods for cleaning dishware items, tableware items, fabrics, and items having hard surfaces (e.g., the hard surface of a table, table top, wall, furniture item, floor, ceiling, etc.). Some embodiments are directed to one or more cleaning composition comprising one or more subtilisin variant described herein. The enzymatic activity (e.g., protease enzyme activity) of one or more subtilisin variant described herein can be readily determined through procedures well known to those of ordinary skill in the art. The Examples presented infra describe methods for evaluating the enzymatic activity and cleaning performance. The performance of one or more subtilisin variant described herein in removing stains (e.g., a protein stain such as blood/milk/ink or egg yolk), cleaning hard surfaces, or cleaning laundry, dishware or tableware item(s) can be readily determined using procedures well known in the art and/or by using procedures, such as, for example, those set forth in the Examples. In some embodiments, one or more subtilisin variant described herein has protease activity in the presence of a surfactant. In other embodiments, the surfactant is selected from the group consisting of a non-ionic surfactant, an anionic surfactant, a cationic surfactant, a zwitterionic surfactant, an ampholytic surfactant, a semi-polar non-ionic surfactant, and a combination thereof. In some embodiments, the protease activity comprises casein hydrolysis activity. In some embodiments, the protease activity comprises dimethylcasein hydrolysis activity.

In other embodiments, one or more subtilisin variant described herein has an increase in proteolytic activity compared to the proteolytic activity of the protease having the sequence of SEQ ID NO:85 and/or an increase in cleaning activity compared to the cleaning activity of the protease having the sequence of SEQ ID NO:85. In still other embodiments, one or more subtilisin variant described herein has an increase in proteolytic activity compared to the proteolytic activity of the protease having the sequence of SEQ ID NO:18 and/or an increase in cleaning activity compared to the cleaning activity of the protease having the sequence of SEQ ID NO:18. In even further embodiments, one or more subtilisin variant described herein has one or more improved property when compared to a reference subtilisin; wherein the improved property is selected from improved protease activity, improved cleaning performance in detergent, and improved thermostability in detergent; and wherein said detergent is optionally a boron-free detergent. In yet an even further embodiment, one or more subtilisin variant described herein has one or more improved property when compared to a reference subtilisin; wherein the improved property is selected from improved protease activity, improved cleaning performance in detergent, and improved thermostability in detergent; and wherein said detergent is a boron-free detergent. In still other embodiments, one or more subtilisin variant described herein has one or more improved property when compared to a reference subtilisin; wherein the improved property is (i) improved protease activity, wherein said variant has a PI>1 on N-suc-AAPF-pNA or dimethyl casein substrate; (ii) improved cleaning performance in detergent, wherein said variant has a BMI and/or egg stain cleaning PI>1; and/or (iii) improved thermostability in detergent, wherein said variant has a stability PI>1; and wherein said detergent is optionally a boron-free detergent. In yet still an even further embodiment, one or more subtilisin variant described herein has one or more improved property when compared to a reference subtilisin; wherein the improved property is (i) improved protease activity, wherein said variant has a PI>1 on N-suc-AAPF-pNA or dimethyl casein substrate; (ii) improved cleaning performance in detergent, wherein said variant has a BMI and/or egg stain cleaning PI>1; and/or (iii) improved thermostability in detergent, wherein said variant has a stability PI>1; and wherein said detergent is a boron-free detergent. Another embodiment is directed to one or more subtilisin variant described herein, where protease activity is measured in accordance with the protease activity assay of Example 3; cleaning performance in detergent is measured in accordance with the cleaning performance in laundry and ADW detergents assay of Example 4; and/or thermostability in detergent is measured in accordance with the stability assay of Example 4. One or more subtilisin variant described herein can have protease activity over a broad range of pH conditions. In some embodiments, one or more subtilisin variant described herein has protease activity on azo-casein as a substrate. In some embodiments, one or more subtilisin variant described herein has protease activity at a pH of from about 4.0 to about 12.0. In some embodiments, one or more subtilisin variant described herein has protease activity at a pH of from about 8.0 to about 12.0. In some embodiments, one or more subtilisin variant described herein has at least 50%, 60%, 70%, 80% or 90% of maximal protease activity at a pH of from about 8.0 to about 12.0. In some embodiments, one or more subtilisin variant has protease activity at a pH above 8.0, 8.5, 9.0, 9.5, 10.0, 10.5, 11.0 or 11.5. In some embodiments, one or more subtilisin variant has protease activity at a pH below 12.0, 11.5, 11.0, 10.5, 10.0, 9.5, 9.0 or 8.5.

In some embodiments, one or more subtilisin variant described herein has protease activity at a temperature range from about 10° C. to about 90° C. In some embodiments, one or more subtilisin variant described herein has protease activity at a temperature range of from about 50° C. to about 75° C. In some embodiments, one or more subtilisin variant described herein has at least 50%, 60%, 70%, 80% or 90% of maximal protease activity at a temperature of from about 50° C. to about 75° C. In some embodiments, one or more subtilisin variant described herein has activity at a temperature above 50° C., 55° C., 60° C., 65° C., or 70° C. In some embodiments, one or more subtilisin variant described herein has activity at a temperature below 75° C., 70° C., 65° C., 60° C., or 55° C.

In some embodiments, one or more subtilisin variant described herein demonstrates cleaning performance in a cleaning composition. Cleaning compositions often include ingredients harmful to the stability and performance of enzymes, making cleaning compositions a harsh environment for enzymes, e.g. serine proteases, to retain function. Thus, it is not trivial for an enzyme to be put in a cleaning composition and expect enzymatic function (e.g. serine protease activity, such as demonstrated by cleaning performance). In some embodiments, one or more subtilisin variant described herein demonstrates cleaning performance in automatic dishwashing (ADW) detergent compositions. In some embodiments, the cleaning performance in automatic dishwashing (ADW) detergent compositions includes cleaning of egg yolk stains. In some embodiments, one or more subtilisin variant described herein demonstrates cleaning performance in laundry detergent compositions. In some embodiments, the cleaning performance in laundry detergent compositions includes cleaning of blood/milk/ink stains. In one or more cleaning composition described herein, one or more subtilisin variant described herein demonstrates cleaning performance with or without a bleach component.

One or more subtilisin variant described herein can be subject to various changes, such as one or more amino acid insertion, deletion, and/or substitution, either conservative or non-conservative, including where such changes do not substantially alter the enzymatic activity of the polypeptide. Similarly, one or more nucleic acid described herein can also be subject to various changes, such as one or more substitutions of one or more nucleotides in one or more codons such that a particular codon encodes the same or a different amino acid, resulting in either a silent variation (e.g., when the encoded amino acid is not altered by the nucleotide mutation) or non-silent variation, one or more deletions of one or more nucleic acids (or codons) in the sequence, one or more additions or insertions of one or more nucleic acids (or codons) in the sequence, and/or cleavage of or one or more truncations of one or more nucleic acids (or codons) in the sequence. Many such changes in the nucleic acid sequence may not substantially alter the enzymatic activity of the resulting encoded polypeptide enzyme compared to the polypeptide enzyme encoded by the original nucleic acid sequence. One or more nucleic acid sequence described herein can also be modified to include one or more codons that provide for optimum expression in an expression system (e.g., bacterial expression system), while, if desired, said one or more codons still encode the same amino acid(s).

Some embodiments are directed to one or more polypeptide having the desired enzymatic activity (e.g., protease enzyme activity or cleaning performance activity) which comprise sequences having the amino acid substitutions and/or variations described herein and also which comprise one or more additional amino acid substitution or variation, such as conservative and non-conservative substitutions or variations, wherein the polypeptide exhibits, maintains, or approximately maintains the desired enzymatic activity (e.g., proteolytic activity). In some embodiments, the proteolytic activity is reflected in the cleaning activity or performance of one or more subtilisin variant described herein. For example, an amino acid substitution may include, but is not limited to, one or more non-conservative substitution, and/or one or more conservative amino acid substitution. A conservative amino acid residue substitution typically involves exchanging a member within one functional class of amino acid residues for a residue that belongs to the same functional class (conservative amino acid residues are considered functionally homologous or conserved in calculating percent functional homology). For example, alanine, glycine, serine, and threonine are functionally similar and thus may serve as conservative amino acid substitutions for one another. Aspartic acid and glutamic acid may serve as conservative substitutions for one another. Asparagine and glutamine may serve as conservative substitutions for one another. Arginine, lysine, and histidine may serve as conservative substitutions for one another. Isoleucine, leucine, methionine, and valine may serve as conservative substitutions for one another. Phenylalanine, tyrosine, and tryptophan may serve as conservative substitutions for one another.

Other conservative amino acid substitution groups can be envisioned. For example, amino acids can be grouped by similar function or chemical structure or composition (e.g., acidic, basic, aliphatic, aromatic, sulfur-containing). For instance, an aliphatic grouping may comprise: Glycine (G), Alanine (A), Valine (V), Leucine (L), Isoleucine (I). Other groups containing amino acids that are considered conservative substitutions for one another include: aromatic: Phenylalanine (F), Tyrosine (Y), Tryptophan (W); sulfur-containing: Methionine (M), Cysteine (C); Basic: Arginine (R), Lysine (K), Histidine (H); Acidic: Aspartic acid (D), Glutamic acid (E); non-polar uncharged residues, Cysteine (C), Methionine (M), and Proline (P); hydrophilic uncharged residues: Serine (S), Threonine (T), Asparagine (N), and Glutamine (Q). Additional groupings of amino acids are well-known to those of skill in the art and described in various standard textbooks. Listing of a polypeptide sequence herein, in conjunction with the above substitution groups, provides an express listing of all conservatively substituted polypeptide sequences.

More conservative substitutions exist within the amino acid residue classes described above, which also or alternatively can be suitable. Conservation groups for substitutions that are more conservative include: valine-leucine-isoleucine, phenylalanine-tyrosine, lysine-arginine, alanine-valine, and asparagine-glutamine.

Conservative substitutions or variations of one or more subtilisin variant described herein includes substitutions or variations of a small percentage, sometimes less than 5%, 4%, 3%, 2%, or 1%, or less than 10, 9, 8, 7, 6, 5, 4, 3, 2, or 1 amino acid with a conservatively selected amino acid of the same conservative substitution group.

One or more nucleic acid described herein is useful in recombinant production (e.g., expression) of one or more subtilisin variant described herein, typically through expression of a plasmid expression vector comprising a sequence encoding one or more subtilisin variant described herein.

Some embodiments are directed to one or more nucleic acid sequence having at least 50, 60, 65, 70, 75, 80, 85, 90, 91, 92, 93, 94, 95, 96, 97, 98, 99 or 100% nucleic acid sequence identity to the nucleic acid sequence of SEQ ID NO: 15, 21, 23, 25, 27, 29, 31, 33, 35, 37, 39, 41, 43, 45, 49, 51, 53, 55, 57, 59, 61, 63, 65, 67, or 69. Other embodiments are directed to one or more polynucleotide comprising a complementary nucleic acid sequence to the nucleic acid sequence of SEQ ID NO: 15, 21, 23, 25, 27, 29, 31, 33, 35, 37, 39, 41, 43, 45, 49, 51, 53, 55, 57, 59, 61, 63, 65, 67, or 69. Still other embodiments are directed to one or more polynucleotide comprising a nucleic acid sequence that encodes one or more subtilisin variant described herein. In even further embodiments, one or more polynucleotide or nucleic acid described herein is an isolated, recombinant, substantially pure, and/or non-naturally occurring polynucleotide or nucleic acid.

Some embodiments are directed to a synthetically derived nucleic acid comprising a nucleotide sequence encoding one or more subtilisin variant described herein. In some embodiments, one or more subtilisin variant described herein is expressed recombinantly with a homologous pro-peptide sequence (e.g., Bgi02446 pro-peptide).

Nucleic acids of the invention can be generated by using any suitable synthesis, manipulation, and/or isolation techniques, or combinations thereof. For example, a polynucleotide of the invention may be produced using standard nucleic acid synthesis techniques, such as solid-phase synthesis techniques that are well-known to those skilled in the art. In such techniques, fragments of up to 50 or more nucleotide bases are typically synthesized, then joined (e.g., by enzymatic or chemical ligation methods) to form essentially any desired continuous nucleic acid sequence. The synthesis of the nucleic acids of the invention can be also facilitated by any suitable method known in the art, including but not limited to chemical synthesis using the classical phosphoramidite method (See e.g., Beaucage et al. Tetrahedron Letters 22:1859-69 [1981]); or the method described by Matthes et al. (See, Matthes et al., EMBO J. 3:801-805 [1984]), as is typically practiced in automated synthetic methods. Nucleic acids of the invention also can be produced by using an automatic DNA synthesizer. Customized nucleic acids can be ordered from a variety of commercial sources (e.g., The Midland Certified Reagent Company, the Great American Gene Company, Operon Technologies Inc., and DNA2.0). Other techniques for synthesizing nucleic acids and related principles are known in the art (See e.g., Itakura et al., Ann. Rev. Biochem. 53:323 [1984]; and Itakura et al., Science 198:1056 [1984]).

As indicated above, recombinant DNA techniques useful in modification of nucleic acids are well known in the art. For example, techniques such as restriction endonuclease digestion, ligation, reverse transcription and cDNA production, and polymerase chain reaction (e.g., PCR) are known and readily employed by those of skill in the art. One or more nucleotide described herein may also be obtained by screening cDNA libraries using one or more oligonucleotide probes that can hybridize to or PCR-amplify polynucleotides which encode one or more subtilisin variant described herein. Procedures for screening and isolating cDNA clones and PCR amplification procedures are well known to those of skill in the art and described in standard references known to those skilled in the art. Some nucleic acids described herein can be obtained by altering a naturally occurring polynucleotide backbone (e.g., that encodes an enzyme or parent protease) by, for example, a known mutagenesis procedure (e.g., site-directed mutagenesis, site saturation mutagenesis, and in vitro recombination). A variety of methods are known in the art that are suitable for generating modified polynucleotides that can encode one or more subtilisin variant described herein, including, but not limited to, for example, site-saturation mutagenesis, scanning mutagenesis, insertional mutagenesis, deletion mutagenesis, random mutagenesis, site-directed mutagenesis, and directed-evolution, as well as various other recombinatorial approaches.

Some embodiments provide one or more vector comprising one or more polynucleotide described herein; one or more expression vector or expression cassette comprising one or more nucleic acid or polynucleotide described herein; one or more isolated, substantially pure, or recombinant DNA construct comprising one or more nucleic acid or polynucleotide described herein; one or more isolated or recombinant cell comprising one or more polynucleotide described herein; and one or more composition comprising one or more such vector, nucleic acid, expression vector, expression cassette, DNA construct, cell, cell culture, or any combination or mixture thereof.

Some embodiments provide one or more recombinant cell comprising one or more vector (e.g., expression vector or DNA construct) described herein which comprise one or more nucleic acid or polynucleotide described herein. Some such recombinant cells are transformed or transfected with such one or more vector, although other methods are available and known in the art. Such cells are typically referred to as host cells. Some such cells comprise bacterial cells, including, but are not limited to Bacillus sp. cells, such as B. subtilis cells. The invention also provides recombinant cells (e.g., recombinant host cells) comprising one or more subtilisin variant described herein.

Other embodiments provide one or more vector comprising one or more nucleic acid or polynucleotide described herein. In some embodiments, the vector is an expression vector or expression cassette in which one or more polynucleotide sequence described herein is operably linked to one or more additional nucleic acid segment required for efficient gene expression (e.g., a promoter operably linked to a polynucleotide described herein). A vector may include a transcription terminator and/or a selection gene, such as an antibiotic resistance gene, that enables continuous cultural maintenance of plasmid-infected host cells by growth in antimicrobial-containing media.

An expression vector may be derived from plasmid or viral DNA, or in alternative embodiments, contains elements of both. Exemplary vectors include, but are not limited to pC194, pJH101, pE194, pHP13 (See, Harwood and Cutting [eds.], Chapter 3, Molecular Biological Methods for Bacillus, John Wiley & Sons [1990]; suitable replicating plasmids for B. subtilis include those listed on p. 92) See also, Perego, Integrational Vectors for Genetic Manipulations in B. subtilis, in Sonenshein et al., [eds.] B. subtilis and Other Gram-Positive Bacteria: Biochemistry, Physiology and Molecular Genetics, American Society for Microbiology, Washington, D.C. [1993], pp. 615-624), and p2JM103BBI.

For expression and production of a protein of interest (e.g., serine protease polypeptide) in a cell, at least one expression vector comprising at least one copy of a polynucleotide encoding the serine protease polypeptide, and in some instances comprising multiple copies, is transformed into the cell under conditions suitable for expression of the serine protease. In some embodiments, one or more polynucleotide sequence described herein (as well as other sequences included in the vector) is integrated into the genome of the host cell; while in other embodiments, a plasmid vector comprising one or more polynucleotide sequence described herein remains as autonomous extra-chromosomal element within the cell. The invention provides both extrachromosomal nucleic acid elements as well as incoming nucleotide sequences that are integrated into the host cell genome. One or more vector described herein is useful for producing one or more subtilisin variant described herein. In some embodiments, a polynucleotide construct encoding one or more subtilisin variant described herein is present on an integrating vector that enables the integration and optionally the amplification of the polynucleotide into the host chromosome. Examples of sites for integration are well known to those skilled in the art. In some embodiments, transcription of one or more polynucleotide described herein is effectuated by a promoter that is the wild-type promoter for the selected precursor protease. In other embodiments, the promoter is heterologous to the precursor protease, but is functional in the host cell. Examples of suitable promoters for use in bacterial host cells include, but are not limited to the amyE, amyQ, amyL, pstS, sacB, pSPAC, pAprE, pVeg, pHpaII promoters; the promoter of the B. stearothermophilus maltogenic amylase gene; the B. amyloliquefaciens (BAN) amylase gene; the B. subtilis alkaline protease gene; the B. clausii alkaline protease gene the B. pumilis xylosidase gene; the B. thuringiensis cryIIIA; and the B. lichenformis alpha-amylase gene. Additional promoters include, but are not limited to, the A4 promoter, as well as phage Lambda PR or PL promoters, and the E. coli lac, trp or tac promoters.

One or more subtilisin variant described herein can be produced in host cells of any suitable microorganism, including bacteria and fungi. In some embodiments, one or more subtilisin variant described herein can be produced in Gram-positive bacteria. In some embodiments, the host cells are Bacillus spp., Streptomyces spp., Escherichia spp., Aspergillus spp., Trichoderma spp., Pseudomonas spp., Corynebacterium spp., Saccharomyces spp., or Pichia spp. In some embodiments, one or more subtilisin variant described herein is produced by Bacillus sp. host cells. Examples of Bacillus sp. host cells include, but are not limited to B. lichenformis, B. lentus, B. subtilis, B. amyloliquefaciens, B. lentus, B. brevis, B. stearothermophilus, B. alkalophilus, B. coagulans, B. circulans, B. pumilis, B. thuringiensis, B. clausii, B. megaterium, Myceliopthera spp, and Yarrowia spp, as well as other organisms within the genus Bacillus. In some embodiments, B. subtilis host cells are used for production of serine protease polypeptides. USPNs 5,264,366 and 4,760,025 (RE 34,606) describe various Bacillus host strains that can be used for producing one or more subtilisin variant described herein, although other suitable strains can be used.

Several bacterial strains that can be used to produce one or more subtilisin variant described herein include non-recombinant (i.e., wild-type) Bacillus sp. strains, as well as variants of naturally-occurring strains and/or recombinant strains. In some embodiments, the host strain is a recombinant strain, wherein a polynucleotide encoding a polypeptide of interest has been introduced into the host. In some embodiments, the host strain is a B. subtilis host strain and particularly a recombinant B. subtilis host strain. Numerous B. subtilis strains are known, including, but not limited to for example, 1A6 (ATCC 39085), 168 (1A01), SB19, W23, Ts85, B637, PB1753 through PB1758, PB3360, JH642, 1A243 (ATCC 39,087), ATCC 21332, ATCC 6051, MI113, DE100 (ATCC 39,094), GX4931, PBT 110, and PEP 211strain (See e.g., Hoch et al., Genetics 73:215-228 [1973]; See also, USPNs 4,450,235 and 4,302,544, and EP 0134048). The use of B. subtilis as an expression host cell is well known in the art (See e.g., Palva et al., Gene 19:81-87 [1982]; Fahnestock and Fischer, J. Bacteriol., 165:796-804 [1986]; and Wang et al., Gene 69:39-47 [1988]).

In some embodiments, the Bacillus host cell is a Bacillus sp. that includes a mutation or deletion in at least one of the following genes, degU, degS, degR and degQ. In some embodiments, the mutation is in a degU gene, and in some embodiments the mutation is degU(Hy)32 (See e.g., Msadek et al., J. Bacteriol. 172:824-834 [1990]; and Olmos et al., Mol. Gen. Genet. 253:562-567 [1997]). In some embodiments, the Bacillus host comprises a mutation or deletion in scoC4 (See e.g., Caldwell et al., J. Bacteriol. 183:7329-7340 [2001]); spoIIE (See e.g., Arigoni et al., Mol. Microbiol. 31:1407-1415 [1999]); and/or oppA or other genes of the opp operon (See e.g., Perego et al., Mol. Microbiol. 5:173-185 [1991]). Indeed, it is contemplated that any mutation in the opp operon that causes the same phenotype as a mutation in the oppA gene will find use in some embodiments of the altered Bacillus strain of the invention. In some embodiments, these mutations occur alone, while in other embodiments, combinations of mutations are present. In some embodiments, an altered Bacillus host cell strain that can be used to produce one or more subtilisin variant described herein is a Bacillus host strain that already includes a mutation in one or more of the above-mentioned genes. In addition, Bacillus sp. host cells that comprise mutation(s) and/or deletions of endogenous protease genes find use. In some embodiments, the Bacillus host cell comprises a deletion of the aprE and the nprE genes. In other embodiments, the Bacillus sp. host cell comprises a deletion of 5 protease genes, while in other embodiments, the Bacillus sp. host cell comprises a deletion of 9 protease genes (See e.g., US 2005/0202535).

Host cells are transformed with one or more nucleic acid described herein using any suitable method known in the art. Methods for introducing a nucleic acid (e.g., DNA) into Bacillus cells or E. coli cells utilizing plasmid DNA constructs or vectors and transforming such plasmid DNA constructs or vectors into such cells are well known. In some embodiments, the plasmids are subsequently isolated from E. coli cells and transformed into Bacillus cells. However, it is not essential to use intervening microorganisms such as E. coli, and in some embodiments, a DNA construct or vector is directly introduced into a Bacillus host.

Those of skill in the art are well aware of suitable methods for introducing one or more nucleic acid sequence described herein into Bacillus cells (See e.g., Ferrari et al., “Genetics,” in Harwood et al. [eds.], Bacillus, Plenum Publishing Corp. [1989], pp. 57-72; Saunders et al., J. Bacteriol. 157:718-726 [1984]; Hoch et al., J. Bacteriol. 93:1925-1937 [1967]; Mann et al., Current Microbiol. 13:131-135 [1986]; Holubova, Folia Microbiol. 30:97 [1985]; Chang et al., Mol. Gen. Genet. 168:11-115 [1979]; Vorobjeva et al., FEMS Microbiol. Lett. 7:261-263 [1980]; Smith et al., Appl. Env. Microbiol. 51:634 [1986]; Fisher et al., Arch. Microbiol. 139:213-217 [1981]; and McDonald, J. Gen. Microbiol. 130:203 [1985]). Indeed, such methods as transformation, including protoplast transformation and transfection, transduction, and protoplast fusion are well known and suited for use herein. Methods known in the art to transform Bacillus cells include such methods as plasmid marker rescue transformation, which involves the uptake of a donor plasmid by competent cells carrying a partially homologous resident plasmid (See, Contente et al., Plasmid 2:555-571 [1979]; Haima et al., Mol. Gen. Genet. 223:185-191 [1990]; Weinrauch et al., J. Bacteriol. 154:1077-1087 [1983]; and Weinrauch et al., J. Bacteriol. 169:1205-1211 [1987]). In this method, the incoming donor plasmid recombines with the homologous region of the resident “helper” plasmid in a process that mimics chromosomal transformation.

In addition to commonly used methods, in some embodiments, host cells are directly transformed with a DNA construct or vector comprising one or more nucleic acid described herein (i.e., an intermediate cell is not used to amplify, or otherwise process, the DNA construct or vector prior to introduction into the host cell). Introduction of one or more DNA construct or vector described herein into the host cell includes those physical and chemical methods known in the art to introduce a nucleic acid sequence (e.g., DNA sequence) into a host cell without insertion into the host genome. Such methods include, but are not limited to calcium chloride precipitation, electroporation, naked DNA, liposomes and the like. In additional embodiments, DNA constructs or vector are co-transformed with a plasmid, without being inserted into the plasmid. In further embodiments, a selective marker is deleted from the altered Bacillus strain by methods known in the art (See, Stahl et al., J. Bacteriol. 158:411-418 [1984]; and Palmeros et al., Gene 247:255-264 [2000]).

In some embodiments, the transformed cells described herein are cultured in conventional nutrient media. The suitable specific culture conditions, such as temperature, pH and the like are known to those skilled in the art and are well described in the scientific literature. In some embodiments, the invention provides a culture (e.g., cell culture) comprising one or more subtilisin variant described herein or one or more nucleic acid described herein.

In some embodiments, host cells transformed with one or more polynucleotide sequence described herein are cultured in a suitable nutrient medium under conditions permitting the expression of one or more subtilisin variant described herein, after which the resulting variant is recovered from the culture. In some embodiments, the variant produced by the cells is recovered from the culture medium by conventional procedures, including, but not limited to for example, separating the host cells from the medium by centrifugation or filtration, precipitating the proteinaceous components of the supernatant or filtrate by means of a salt (e.g., ammonium sulfate), chromatographic purification (e.g., ion exchange, gel filtration, affinity, etc.).

In some embodiments, a serine protease polypeptide produced by a recombinant host cell is secreted into the culture medium. A nucleic acid sequence that encodes a purification facilitating domain may be used to facilitate purification of proteins. A vector or DNA construct comprising a polynucleotide sequence encoding a serine protease polypeptide may further comprise a nucleic acid sequence encoding a purification facilitating domain to facilitate purification of the serine protease polypeptide (See e.g., Kroll et al., DNA Cell Biol. 12:441-53 [1993]). Such purification facilitating domains include, but are not limited to, for example, metal chelating peptides such as histidine-tryptophan modules that allow purification on immobilized metals (See, Porath, Protein Expr. Purif. 3:263-281 [1992]), protein A domains that allow purification on immobilized immunoglobulin, and the domain utilized in the FLAGS extension/affinity purification system. The inclusion of a cleavable linker sequence such as Factor XA or enterokinase (e.g., sequences available from Invitrogen, San Diego, CA) between the purification domain and the heterologous protein also find use to facilitate purification.

Assays for detecting and measuring the enzymatic activity of an enzyme, such as one or more subtilisin variant described herein, are well known. Various assays for detecting and measuring activity of proteases (e.g., one or more subtilisin variant described herein), are also known to those of ordinary skill in the art. In particular, assays are available for measuring protease activity that are based on the release of acid-soluble peptides from casein or hemoglobin, measured as absorbance at 280 nm or colorimetrically using the Folin method. Other exemplary assays involve the solubilization of chromogenic substrates (See e.g., Ward, “Proteinases,” in Fogarty (ed.), Microbial Enzymes and Biotechnology, Applied Science, London, [1983], pp. 251-317). Other exemplary assays include, but are not limited to succinyl-Ala-Ala-Pro-Phe-para nitroanilide assay (suc-AAPF-pNA) and the 2,4,6-trinitrobenzene sulfonate sodium salt assay (TNBS assay). Numerous additional references known to those in the art provide suitable methods (See e.g., Wells et al., Nucleic Acids Res. 11:7911-7925 [1983]; Christianson et al., Anal. Biochem. 223:119-129 [1994]; and Hsia et al., Anal Biochem. 242:221-227 [1999]).

A variety of methods can be used to determine the level of production of a mature protease (e.g., one or more mature subtilisin variant described herein) in a host cell. Such methods include, but are not limited to, for example, methods that utilize either polyclonal or monoclonal antibodies specific for the protease. Exemplary methods include, but are not limited to enzyme-linked immunosorbent assays (ELISA), radioimmunoassays (RIA), fluorescent immunoassays (FIA), and fluorescent activated cell sorting (FACS). These and other assays are well known in the art (See e.g., Maddox et al., J. Exp. Med. 158:1211 [1983]).

Some other embodiments provide methods for making or producing one or more mature subtilisin variant described herein. A mature serine protease polypeptide does not include a signal peptide or a propeptide sequence. Some methods comprise making or producing one or more subtilisin variant described herein in a recombinant bacterial host cell, such as for example, a Bacillus sp. cell (e.g., a B. subtilis cell). Some embodiments provide a method of producing one or more subtilisin variant described herein comprising cultivating a recombinant host cell comprising a recombinant expression vector comprising a nucleic acid encoding one or more subtilisin variant described herein under conditions conducive to the production of the variant. Some such methods further comprise recovering the variant from the culture.

Some embodiments provide one or more method of producing one or more subtilisin variant described herein, comprising: (a) introducing a recombinant expression vector comprising a nucleic acid encoding one or more subtilisin variant described herein into a population of cells (e.g., bacterial cells, such as B. subtilis cells); and (b) culturing the cells in a culture medium under conditions conducive to produce the variant encoded by the expression vector. Some such methods further comprise: (c) isolating variant from the cells or the culture medium.

Unless otherwise noted, all component or composition levels provided herein are made in reference to the active level of that component or composition, and are exclusive of impurities, for example, residual solvents or by-products, which may be present in commercially available sources. Enzyme component weights are based on total active protein. All percentages and ratios are calculated by weight unless otherwise indicated. All percentages and ratios are calculated based on the total composition unless otherwise indicated. Compositions of the invention include cleaning compositions, such as detergent compositions. In the exemplified detergent compositions, the enzymes levels are expressed by pure enzyme by weight of the total composition and unless otherwise specified, the detergent ingredients are expressed by weight of the total compositions.

While not essential for the purposes of the present invention, the non-limiting list of adjuncts illustrated hereinafter are suitable for use in the instant cleaning compositions. In some embodiments, these adjuncts are incorporated for example, to assist or enhance cleaning performance, for treatment of the substrate to be cleaned, or to modify the aesthetics of the cleaning composition as is the case with perfumes, colorants, dyes or the like. One embodiment is directed to a composition comprising one or more adjunct material and one or more subtilisin variant described herein. The precise nature of the adjunct materials that re employed in any particular composition, and levels of incorporation thereof, will depend on the physical form of the composition and the nature of the cleaning operation for which it is to be used.

Suitable adjunct materials include, but are not limited to, bleach catalysts, an additional enzyme, enzyme stabilizers (including, for example, an enzyme stabilizing system), chelants, optical brighteners, soil release polymers, dye transfer agents, dispersants, suds suppressors, dyes, perfumes, colorants, filler salts, photoactivators, fluorescers, fabric conditioners, hydrolyzable surfactants, preservatives, anti-oxidants, anti-shrinkage agents, anti-wrinkle agents, germicides, fungicides, color speckles, silvercare, anti-tarnish and/or anti-corrosion agents, alkalinity sources, solubilizing agents, carriers, processing aids, pigments, and pH control agents, surfactants, builders, chelating agents, dye transfer inhibiting agents, deposition aids, dispersants, additional enzymes, and enzyme stabilizers, catalytic materials, bleach activators, bleach boosters, hydrogen peroxide, sources of hydrogen peroxide, preformed peracids, polymeric dispersing agents, clay soil removal/anti-redeposition agents, brighteners, suds suppressors, dyes, perfumes, structure elasticizing agents, fabric softeners, carriers, hydrotropes, processing aids and/or pigments. Suitable examples of other adjunct materials and levels of use can be found in U.S. Pat. Nos. 5,576,282; 6,306,812; 6,326,348; 6,610,642; 6,605,458; 5,705,464; 5,710,115; 5,698,504; 5,695,679; 5,686,014; and 5,646,101. In embodiments in which one or more adjunct material is not compatible with one or more subtilisin variant described herein suitable methods of keeping the adjunct material(s) and variant(s) separated (i.e., not in contact with each other) can be employed until combination of the two components is appropriate. Such separation methods include any suitable method known in the art (e.g., gelcaps, encapsulation, tablets, physical separation, etc.). The aforementioned adjunct materials may constitute the balance of the cleaning compositions described herein.

One or more cleaning composition described herein is advantageously employed for example, in laundry applications, hard surface cleaning applications, dishwashing applications, including automatic dishwashing and hand dishwashing, as well as cosmetic applications such as dentures, teeth, hair and skin cleaning and disinfecting applications, such as, for example, but not limited to, disinfecting an automatic dishwashing or laundry machine. The enzymes of the present invention are also suited for use in contact lens cleaning and wound debridement applications. In addition, due to the unique advantages of increased effectiveness in lower temperature solutions, the enzymes of the present invention are ideally suited for laundry applications. Furthermore, the enzymes of the present invention find use in granular and liquid compositions.

Another embodiment is directed to a composition comprising one or more subtilisin variant described herein. In some embodiments, the composition is a cleaning composition. In other embodiments, the composition is a detergent composition. In yet other embodiments, the composition is selected from a laundry detergent composition, an automatic dishwashing (ADW) composition, a hand (manual) dishwashing detergent composition, a hard surface cleaning composition, an eyeglass cleaning composition, a medical instrument cleaning composition, a disinfectant (e.g., malodor or microbial) composition, and a personal care cleaning composition. In still other embodiments, the composition is a laundry detergent composition, an ADW composition, or a hand (manual) dishwashing detergent composition. Even still further embodiments are directed to fabric cleaning compositions, while other embodiments are directed to non-fabric cleaning compositions. In some embodiments, the cleaning composition is boron-free. In other embodiments, the cleaning composition is phosphate-free. In still other embodiments, the composition comprises one or more subtilisin variant described herein and one or more of an excipient, adjunct material, and/or additional enzyme.

In yet still a further embodiment, the composition described herein contains phosphate, is phosphate-free, contains boron, is boron-free, or combinations thereof. In other embodiments, the composition is a boron-free composition. In some embodiments, a boron-free composition is a composition to which a borate stabilizer has not been added. In another embodiment, a boron-free composition is a composition that contains less than 5.5% boron. In a still further embodiment, a boron-free composition is a composition that contains less than 4.5% boron. In yet still another embodiment, a boron-free composition is a composition that contains less than 3.5% boron. In yet still a further embodiment, a boron-free composition is a composition that contains less than 2.5% boron. In even further embodiments, a boron-free composition is a composition that contains less than 1.5% boron. In another embodiment, a boron-free composition is a composition that contains less than 1.0% boron. In still further embodiments, a boron-free composition is a composition that contains less than 0.5% boron. In still further embodiments, a boron-free composition is a composition substantially-free of boron.

One or more subtilisin variant described herein also finds use in cleaning additive products. In some embodiments, low temperature solution cleaning applications find use. Some embodiments provide cleaning additive products comprising one or more subtilisin variant described herein, which additive is ideally suited for inclusion in a wash process when additional bleaching effectiveness is desired. Such instances include, but are not limited to low temperature solution cleaning applications. In some embodiments, the additive product is in its simplest form, one or more subtilisin variant described herein. In some embodiments, the additive is packaged in dosage form for addition to a cleaning process. In some embodiments, the additive is packaged in dosage form for addition to a cleaning process where a source of peroxygen is employed and increased bleaching effectiveness is desired.

Exemplary fillers or carriers for granular compositions include, but are not limited to, for example, various salts of sulfate, carbonate and silicate; talc; and clay. Exemplary fillers or carriers for liquid compositions include, but are not limited to, for example, water or low molecular weight primary and secondary alcohols including polyols and diols (e.g., methanol, ethanol, propanol and isopropanol). In some embodiments, the compositions contain from about 5% to about 90% of such filler or carrier. Acidic fillers may be included in such compositions to reduce the pH of the resulting solution in the cleaning method or application.

In another embodiment, one or more composition described herein is in a form selected from gel, tablet, powder, granular, solid, liquid, unit dose, and combinations thereof. In yet another embodiment, one or more composition described herein is in a form selected from a low water compact formula, low water HIDL or UD, or high water formula or HIDL. In some embodiments, the cleaning composition describe herein is in a unit dose form. In other embodiments, the unit does form is selected from pills, tablets, capsules, gelcaps, sachets, pouches, multi-compartment pouches, and pre-measured powders or liquids. In some embodiments, the unit dose format is designed to provide controlled release of the ingredients within a multi-compartment pouch (or other unit dose format). Suitable unit dose and controlled release formats are described, for example, in EP2100949; WO 02/102955; U.S. Pat. Nos. 4,765,916; 4,972,017; and WO 04/111178. In some embodiments, the unit dose form is a tablet or powder contained in a water-soluble film or pouch.

The present cleaning compositions or cleaning additives comprise an effective amount of one or more subtilisin variant described herein, alone or in combination with one or more additional enzyme. Typically the present cleaning compositions comprise at least about 0.0001 weight percent, from about 0.0001 to about 10, from about 0.001 to about 1, or from about 0.01 to about 0.1 weight percent of one or more subtilisin variant described herein. In another embodiment, one or more cleaning composition described herein comprises from about 0.01 to about 10 mg, about 0.01 to about 5 mg, about 0.01 to about 2 mg, about 0.01 to about 1 mg, about 0.05 to about 1 mg, about 0.5 to about 10 mg, about 0.5 to about 5 mg, about 0.5 to about 4 mg, about 0.5 to about 4 mg, about 0.5 to about 3 mg, about 0.5 to about 2 mg, about 0.5 to about 1 mg, about 0.1 to about 10 mg, about 0.1 to about 5 mg, about 0.1 to about 4 mg, about 0.1 to about 3 mg, about 0.1 to about 2 mg, about 0.1 to about 2 mg, about 0.1 to about 1 mg, or about 0.1 to about 0.5 mg of one or more subtilisin variant described herein per gram of composition.

In some embodiments, one or more subtilisin variant described herein cleans at low temperatures. In other embodiments, one or more composition described herein cleans at low temperatures. In other embodiments, one or more composition described herein comprises an effective amount of one or more subtilisin variant described herein as useful or effective for cleaning a surface in need of proteinaceous stain removal.

The cleaning compositions herein are typically formulated such that, during use in aqueous cleaning operations, the wash water will have a pH of from about 4.0 to about 11.5, or even from about 5.0 to about 11.5, or even from about 5.0 to about 8.0, or even from about 7.5 to about 10.5. Liquid product formulations are typically formulated to have a pH from about 3.0 to about 9.0 or even from about 3 to about 5. Granular laundry products are typically formulated to have a pH from about 9 to about 11. Some embodiments provide one or more cleaning composition formulated to have an alkaline pH under wash conditions, such as a pH of from about 8.0 to about 12.0, or from about 8.5 to about 11.0, or from about 9.0 to about 11.0. In some embodiments, one or more cleaning composition described herein is formulated to have a neutral pH under wash conditions, such as a pH of from about 5.0 to about 8.0, or from about 5.5 to about 8.0, or from about 6.0 to about 8.0, or from about 6.0 to about 7.5. In some embodiments, the neutral pH conditions can be measured when the cleaning composition is dissolved 1:100 (wt:wt) in de-ionised water at 20° C., measured using a conventional pH meter. Techniques for controlling pH at recommended usage levels include the use of buffers, alkalis, acids, etc., and are well known to those skilled in the art.

In some embodiments, when one or more subtilisin variant described herein is employed in a granular composition or liquid, it is desirable for the variant to be in the form of an encapsulated particle to protect the variant from other components of the granular composition during storage. In addition, encapsulation is also a means of controlling the availability of the variant during the cleaning process. In some embodiments, encapsulation enhances the performance of variant and/or additional enzymes. In this regard, one or more subtilisin variant described herein is encapsulated with any suitable encapsulating material known in the art. In some embodiments, the encapsulating material typically encapsulates at least part of the variant. Typically, the encapsulating material is water-soluble and/or water-dispersible. In some embodiments, the encapsulating material has a glass transition temperature (Tg) of 0° C. or higher. Tg is described in more detail in WO97/11151. The encapsulating material is typically selected from carbohydrates, natural or synthetic gums, chitin, chitosan, cellulose and cellulose derivatives, silicates, phosphates, borates, polyvinyl alcohol, polyethylene glycol, paraffin waxes, and combinations thereof. When the encapsulating material is a carbohydrate, it is typically selected from monosaccharides, oligosaccharides, polysaccharides, and combinations thereof. In some typical embodiments, the encapsulating material is a starch (See e.g., EP0922499; U.S. Pat. Nos. 4,977,252; 5,354,559, and 5,935,826). In some embodiments, the encapsulating material is a microsphere made from plastic such as thermoplastics, acrylonitrile, methacrylonitrile, polyacrylonitrile, polymethacrylonitrile and mixtures thereof; commercially available microspheres that find use include, but are not limited to those supplied by EXPANCEL® (Stockviksverken, Sweden), and PM6545, PM6550, PM7220, PM7228, EXTENDOSPHERES®, LUXSIL®, Q-CEL®, and SPHERICEL® (PQ Corp., Valley Forge, PA).

There are a variety of wash conditions including varying detergent formulations, wash water volumes, wash water temperatures, and lengths of wash time, to which proteases involved in washing are exposed. A low detergent concentration system includes detergents where less than about 800 ppm of the detergent components are present in the wash water. A medium detergent concentration includes detergents where between about 800 ppm and about 2000 ppm of the detergent components are present in the wash water. A high detergent concentration system includes detergents where greater than about 2000 ppm of the detergent components are present in the wash water. In some embodiments, the “cold water washing” of the present invention utilizes “cold water detergent” suitable for washing at temperatures from about 10° C. to about 40° C., or from about 20° C. to about 30° C., or from about 15° C. to about 25° C., as well as all other combinations within the range of about 15° C. to about 35° C., and all ranges within 10° C. to 40° C.

Different geographies typically have different water hardness. Water hardness is usually described in terms of the grains per gallon mixed Ca²⁺/Mg²⁺. Hardness is a measure of the amount of calcium (Ca²⁺) and magnesium (Mg²⁺) in the water. Most water in the United States is hard, but the degree of hardness varies. Moderately hard (60-120 ppm) to hard (121-181 ppm) water has 60 to 181 parts per million.

| Water | Grains per gallon | Parts per million |
|---|---|---|
| Soft | less than 1.0 | less than 17 |
| Slightly hard | 1.0 to 3.5 | 17 to 60 |
| Moderately hard | 3.5 to 7.0 | 60 to 120 |
| Hard | 7.0 to 10.5 | 120 to 180 |
| Very hard | greater than 10.5 | greater than 180 |

In some embodiments, one or more subtilisin variant described herein shows surprising wash performance in at least one set of wash conditions (e.g., water temperature, water hardness, and/or detergent concentration). In some embodiments, one or more subtilisin variant described herein is comparable in wash performance to other serine protease polypeptide proteases. In some embodiments, one or more subtilisin variant described herein exhibits enhanced oxidative stability, enhanced thermal stability, enhanced cleaning capabilities under various conditions, and/or enhanced chelator stability.

Other embodiments are directed to one or more cleaning composition comprising from about 0.00001% to about 10% by weight composition of one or more subtilisin variant described herein and from about 99.999% to about 90.0% by weight composition of one or more adjunct material. Other embodiments provide one or more cleaning composition comprises from about 0.0001% to about 10%, about 0.001% to about 5%, about 0.001% to about 2%, about 0.005% to about 0.5% by weight composition of one or more subtilisin variant described herein and from about 99.9999% to about 90.0%, about 99.999% to about 98%, about 99.995% to about 99.5% by weight composition of one or more adjunct material.

In other embodiments, the composition described herein comprises one or more subtilisin variant described herein and one or more additional enzyme. The one or more additional enzyme is selected from additional serine proteases, acyl transferases, alpha-amylases, beta-amylases, alpha-galactosidases, arabinosidases, aryl esterases, beta-galactosidases, carrageenases, catalases, cellobiohydrolases, cellulases, chondroitinases, cutinases, endo-beta-1,4-glucanases, endo-beta-mannanases, esterases, exo-mannanases, galactanases, glucoamylases, hemicellulases, hyaluronidases, keratinases, laccases, lactases, ligninases, lipases, lipoxygenases, mannanases, metalloproteases, non-serine proteases, oxidases, pectate lyases, pectin acetyl esterases, pectinases, pentosanases, perhydrolase, peroxidases, phenoloxidases, phosphatases, phospholipases, phytases, polygalacturonases, pullulanases, reductases, rhamnogalacturonases, beta-glucanases, tannases, transglutaminases, xylan acetyl-esterases, xylanases, xyloglucanases, and xylosidases, or any combinations or mixtures thereof. Some embodiments are directed to a combination of enzymes (i.e., a “cocktail”) comprising conventional enzymes like amylase, lipase, cutinase and/or cellulase in conjunction with one or more subtilisin variant described herein and/or one or more additional serine protease.

In another embodiment, one or more composition described herein comprises one or more subtilisin variant described herein and one or more additional protease. Suitable proteases include those of animal, vegetable or microbial origin. In some embodiments, microbial proteases are used. In some embodiments, chemically or genetically modified mutants are included. In some embodiments, the protease is a serine protease, preferably an alkaline microbial protease or a trypsin-like protease. Examples of alkaline proteases include subtilisins, especially those derived from Bacillus (e.g., subtilisin, lentus, amyloliquefaciens, subtilisin Carlsberg, subtilisin 309, subtilisin 147 and subtilisin 168). Exemplary additional proteases include but are not limited to those described in WO92/21760, WO95/23221, WO2008/010925, WO09/149200, WO09/149144, WO09/149145, WO 10/056640, WO10/056653, WO2010/0566356, WO11/072099, WO2011/13022, WO11/140364, WO 12/151534, WO2015/038792, WO2015/089447, WO2015/089441, US Publ. No. 2008/0090747, U.S. Pat. Nos. 5,801,039, 5,340,735, 5,500,364, 5,855,625, RE 34,606, 5,955,340, 5,700,676 6,312,936, 6,482,628, 8,530,219, U.S. Provisional Appl Nos. 62/180,673 and 62/161,077, and PCT Appl Nos. PCT/US2015/021813, PCT/US2015/055900, PCT/US2015/057497, PCT/US2015/057492, PCT/US2015/057512, PCT/US2015/057526, PCT/US2015/057520, PCT/US2015/057502, PCT/US2016/022282, and PCT/US16/32514, as well as metalloproteases described in WO1999014341, WO1999033960, WO1999014342, WO1999034003, WO2007044993, WO2009058303, WO 2009058661, WO2014071410, WO2014194032, WO2014194034, WO 2014194054, and WO 2014/194117. Additional protease examples include, but are not limited to trypsin (e.g., of porcine or bovine origin), and the Fusarium protease described in WO89/06270. Exemplary commercial proteases include, but are not limited to MAXATASE®, MAXACAL™, MAXAPEM™, OPTICLEAN®, OPTIMASE®, PROPERASE®, PURAFECT®, PURAFECT® OXP, PURAMAX™, EXCELLASE™, PREFERENZ™ proteases (e.g. P100, P110, P280), EFFECTENZ™ proteases (e.g. P1000, P1050, P2000), EXCELLENZ™ proteases (e.g. P1000), ULTIMASE®, and PURAFAST™ (DuPont); ALCALASE®, BLAZE®, BLAZE® EVITY®, BLAZE® EVITY® 16L, CORONASE®, SAVINASE®, SAVINASE® ULTRA, SAVINASE® EVITY®, SAVINASE® EVERIS®, PRIMASE®, DURAZYM™, POLARZYME®, OVOZYME®, KANNASE®, LIQUANASE®, LIQUANASE EVERIS®, NEUTRASE®, RELASE® and ESPERASE® (Novozymes); BLAP™ and BLAP™ variants (Henkel); and KAP (B. alkalophilus subtilisin (Kao). Exemplary metalloproteases include nprE, the recombinant form of neutral metalloprotease expressed in B. subtilis (See e.g., WO07/044993), and PMN, the purified neutral metalloprotease from B. amyloliquefaciens.

Another embodiment is directed to a composition comprising one or more subtilisin variant described herein and one or more lipase. In some embodiments, the composition comprises from about 0.00001% to about 10%, about 0.0001% to about 10%, about 0.001% to about 5%, about 0.001% to about 2%, or about 0.005% to about 0.5% lipase by weight composition. Exemplary lipases include, but are not limited to those of bacterial or fungal origin. An exemplary lipase can be a chemically or genetically modified mutant. Exemplary lipases include, but are not limited to H. lanuginosa lipase (See e.g., EP258068 and EP305216), Rhizomucor miehei lipase (See e.g., EP 238 023), Candida lipase, such as C. antarctica lipase (e.g., the C. antarctica lipase A or B; See e.g., EP214761), Pseudomonas lipases such as P. alcaligenes and P. pseudoalcaligenes lipase (See e.g., EP218 272), P. cepacia lipase (See e.g., EP 331 376), P. stutzeri lipase (See e.g., GB 1,372,034), P. fluorescens lipase, Bacillus lipase (e.g., B. subtilis lipase [Dartois et al., Biochem. Biophys. Acta 1131:253-260 [1993]); B. stearothermophilus lipase [See e.g., JP 64/744992]; and B. pumilus lipase (See e.g., WO91/16422). Exemplary cloned lipases include, but not limited to Penicillium camembertii lipase (See, Yamaguchi et al., Gene 103:61-67 [1991]), Geotricum candidum lipase (See, Schimada et al., J. Biochem., 106:383-388 [1989]), and various Rhizopus lipases such as R. delemar lipase (See, Hass et al., Gene 109:117-113 [1991]), a R. niveus lipase (Kugimiya et al., Biosci. Biotech. Biochem. 56:716-719 [1992]), and R. oryzae lipase. Other types of lipase polypeptide enzymes such as cutinases may also find use in some embodiments, including but not limited to the cutinase derived from Pseudomonas mendocina (See, WO88/09367), and the cutinase derived from Fusarium solanipisi (See, WO90/09446). Exemplary commercial lipases include, but are not limited to M1 LIPASE™ LUMA FAST™, and LIPOMAX™ (DuPont); LIPEX®, LIPOCLEAN®, LIPOLASE® and LIPOLASE® ULTRA (Novozymes); and LIPASE P™ (Amano Pharmaceutical Co. Ltd).

A still further embodiment is directed to a composition comprising one or more subtilisin variant described herein and one or more amylase. In one embodiment, the composition comprises from about 0.00001% to about 10%, about 0.0001% to about 10%, about 0.001% to about 5%, about 0.001% to about 2%, or about 0.005% to about 0.5% amylase by weight composition. Any amylase (e.g., alpha and/or beta) suitable for use in alkaline solutions may be useful to include in such composition. An exemplary amylase can be a chemically or genetically modified mutant. Exemplary amylases include, but are not limited to those of bacterial or fungal origin, such as, for example, amylases described in GB 1,296,839, WO9100353, WO9402597, WO94183314, WO9510603, WO9526397, WO9535382, WO9605295, WO9623873, WO9623874, WO 9630481, WO9710342, WO9741213, WO9743424, WO9813481, WO 9826078, WO9902702, WO 9909183, WO9919467, WO9923211, WO9929876, WO9942567, WO 9943793, WO9943794, WO 9946399, WO0029560, WO0060058, WO0060059, WO0060060, WO 0114532, WO0134784, WO 0164852, WO0166712, WO0188107, WO0196537, WO02092797, WO 0210355, WO0231124, WO 2004055178, WO2004113551, WO2005001064, WO2005003311, WO 2005018336, WO2005019443, WO2005066338, WO2006002643, WO2006012899, WO2006012902, WO2006031554, WO 2006063594, WO2006066594, WO2006066596, WO2006136161, WO 2008000825, WO2008088493, WO2008092919, WO2008101894, WO2008/112459, WO2009061380, WO2009061381, WO 2009100102, WO2009140504, WO2009149419, WO 2010/059413, WO 2010088447, WO2010091221, WO2010104675, WO2010115021, WO10115028, WO2010117511, WO 2011076123, WO2011076897, WO2011080352, WO2011080353, WO 2011080354, WO2011082425, WO2011082429, WO 2011087836, WO2011098531, WO2013063460, WO2013184577, WO 2014099523, WO2014164777, and WO2015077126. Exemplary commercial amylases include, but are not limited to AMPLIFY®, DURAMYL®, TERMAMYL®, FUNGAMYL®, STAINZYME®, STAINZYME PLUS®, STAINZYME PLUS®, STAINZYME ULTRA® EVITY®, and BAN™ (Novozymes); EFFECTENZ™ S 1000, POWERASE™, PREFERENZ™ S 100, PREFERENZ™ S 110, EXCELLENZ™ S 2000, RAPIDASE® and MAXAMYL® P (DuPont).

Yet a still further embodiment is directed to a composition comprising one or more subtilisin variant described herein and one or more cellulase. In one embodiment, the composition comprises from about 0.00001% to about 10%, 0.0001% to about 10%, about 0.001% to about 5%, about 0.001% to about 2%, or about 0.005% to about 0.5% cellulase by weight of composition. Any suitable cellulase may find used in a composition described herein. An exemplary cellulase can be a chemically or genetically modified mutant. Exemplary cellulases include but are not limited, to those of bacterial or fungal origin, such as, for example, is described in WO2005054475, WO2005056787, U.S. Pat. Nos. 7,449,318, 7,833,773, 4,435,307; EP 0495257; and U.S. Provisional Appl. No. 62/296,678. Exemplary commercial cellulases include, but are not limited to, CELLUCLEAN®, CELLUZYME®, CAREZYME®, ENDOLASE®, RENOZYME®, and CAREZYME® PREMIUM (Novozymes); REVITALENZ™ 100, REVITALENZ™ 200/220, and REVITALENZ® 2000 (DuPont); and KAC-500(B)™ (Kao Corporation). In some embodiments, cellulases are incorporated as portions or fragments of mature wild-type or variant cellulases, wherein a portion of the N-terminus is deleted (see, e.g., U.S. Pat. No. 5,874,276).

An even still further embodiment is directed to a composition comprising one or more subtilisin variant described herein and one or more mannanase. In one embodiment, the composition comprises from about 0.00001% to about 10%, about 0.0001% to about 10%, about 0.001% to about 5%, about 0.001% to about 2%, or about 0.005% to about 0.5% mannanase by weight composition. An exemplary mannanase can be a chemically or genetically modified mutant. Exemplary mannanases include, but are not limited to, those of bacterial or fungal origin, such as, for example, as is described in WO 2016/007929; U.S. Pat. Nos. 6,566,114; 6,602,842; and 6,440,991; and U.S. Provisional Appl. Nos. 62/251,516, 62/278,383, and 62/278,387. Exemplary commercial mannanases include, but are not limited to MANNAWAY® (Novozymes) and EFFECTENZ™ M 1000, PREFERENZ® M 100, MANNASTAR®, and PURABRITE™ (DuPont).

A yet even still further embodiment is directed to a composition comprising one or more subtilisin variant described herein and one or more peroxidase and/or oxidase enzyme. In one embodiment, the composition comprises from about 0.00001% to about 10%, about 0.0001% to about 10%, about 0.001% to about 5%, about 0.001% to about 2%, or about 0.005% to about 0.5% peroxidase or oxidase by weight composition. A peroxidase may be used in combination with hydrogen peroxide or a source thereof (e.g., a percarbonate, perborate or persulfate) and an oxidase may be used in combination with oxygen. Peroxidases and oxidases are used for “solution bleaching” (i.e., to prevent transfer of a textile dye from a dyed fabric to another fabric when the fabrics are washed together in a wash liquor), alone or in combination with an enhancing agent (see, e.g., WO94/12621 and WO95/01426). An exemplary peroxidase and/or oxidase can be a chemically or genetically modified mutant. Exemplary peroxidases/oxidases include, but are not limited to those of plant, bacterial, or fungal origin.

In some embodiments, additional enzymes find use, including but not limited to perhydrolases (See e.g., WO2005/056782, WO2007106293, WO2008063400, WO 2008106214, and WO2008106215).

In yet another embodiment, the one or more subtilisin variant described herein and one or more additional enzyme contained in one or more composition described herein may each independently range to about 10%, wherein the balance of the cleaning composition is one or more adjunct material.

In some embodiments, an effective amount of one or more subtilisin variant provided herein is included in compositions useful for cleaning a variety of surfaces in need of proteinaceous stain removal. Such cleaning compositions include cleaning compositions for such applications as cleaning hard surfaces, fabrics, and dishes. Indeed, some embodiments provide fabric cleaning compositions; while other embodiments provide non-fabric cleaning compositions. Some embodiment also provide cleaning compositions suitable for personal care, including oral care (including dentrifices, toothpastes, mouthwashes, etc., as well as denture cleaning compositions), skin, and hair cleaning compositions. It is intended that the present invention encompass detergent compositions in any form (i.e., liquid, granular, bar, semi-solid, gels, emulsions, tablets, capsules, etc.).

By way of example, several cleaning compositions wherein one or more subtilisin variant described herein finds use are described in greater detail below. In some embodiments, the cleaning composition is formulated for use in laundry machine washing method(s), wherein the composition contains at least one surfactant and at least one builder compound, as well as one or more adjunct material selected from organic polymeric compounds, bleaching agents, additional enzymes, suds suppressors, dispersants, lime-soap dispersants, soil suspension and anti-redeposition agents and corrosion inhibitors. In some embodiments, laundry compositions also contain softening agents (i.e., as an additional adjunct material).

The compositions described herein also find use in detergent additive products in solid or liquid form. Such additive products are intended to supplement and/or boost the performance of a conventional detergent composition and can be added at any stage of the cleaning process. In some embodiments, the density of the laundry detergent compositions herein ranges from about 400 to about 1200 g/liter, while in other embodiments it ranges from about 500 to about 950 g/liter of composition measured at 20° C.

In embodiments formulated as compositions for use in manual dishwashing methods, the compositions contain at least one surfactant and at least one additional adjunct material selected from organic polymeric compounds, suds enhancing agents, group II metal ions, solvents, hydrotropes and additional enzymes.

In some embodiments, various cleaning compositions such as those provided in U.S. Pat. No. 6,605,458, find use with one or more subtilisin variant described herein. Thus, in some embodiments, the compositions comprising one or more subtilisin variant described herein is a compact granular fabric cleaning composition. In other embodiments, the composition is a granular fabric cleaning composition useful in the laundering of colored fabrics. In further embodiments, the composition is a granular fabric cleaning composition which provides softening through the wash capacity. In additional embodiments, the composition is a heavy duty liquid fabric cleaning composition. In some embodiments, the compositions comprising one or more subtilisin variant described herein are fabric cleaning compositions such as those described in USPNs 6,610,642 and 6,376,450. In addition, one or more subtilisin variant described herein finds use in granular laundry detergent compositions of particular utility under European or Japanese washing conditions (See e.g., U.S. Pat. No. 6,610,642).

Yet further embodiments provide a dishwashing composition comprising one or more subtilisin variant described herein. Thus, in some embodiments, a composition comprising one or more subtilisin variant described herein is a hard surface cleaning composition such as those in USPNs 6,610,642 and 6,376,450. Some further embodiments provide an oral care composition comprising one or more subtilisin variant described herein such as those in USPNs 6,376,450, and 6,376,450.

One or more cleaning composition described herein can be formulated into any suitable form and prepared by any process chosen by the formulator (See e.g., U.S. Pat. Nos. 5,879,584; 5,691,297; 5,574,005; 5,569,645; 5,565,422; 5,516,448; 5,489,392; and 5,486,303. When a low pH cleaning composition is desired, the pH of such composition is adjusted via the addition of a material such as monoethanolamine or an acidic material such as HCl.

In some embodiments, one or more cleaning composition described herein comprises an acidifying particle or an amino carboxylic builder. Examples of an amino carboxylic builder include aminocarboxylic acids, salts and derivatives thereof. In some embodiment, the amino carboxylic builder is an aminopolycarboxylic builder, such as glycine-N,N-diacetic acid or derivative of general formula MOOC—CHR-N(CH₂COOM)₂ where R is C₁-12 alkyl and M is alkali metal. In some embodiments, the amino carboxylic builder can be methylglycine diacetic acid (MGDA), GLDA (glutamic-N,N-diacetic acid), iminodisuccinic acid (IDS), carboxymethyl inulin and salts and derivatives thereof, aspartic acid-N-monoacetic acid (ASMA), aspartic acid-N,N-diacetic acid (ASDA), aspartic acid-N-monopropionic acid (ASMP), iminodisuccinic acid (IDA), N-(2-sulfomethyl) aspartic acid (SMAS), N-(2-sulfoethyl)aspartic acid (SEAS), N-(2-sulfomethyl)glutamic acid (SMGL), N-(2-sulfoethyl) glutamic acid (SEGL), IDS (iminodiacetic acid) and salts and derivatives thereof such as N-methyliminodiacetic acid (MIDA), alpha-alanine-N,N-diacetic acid (alpha-ALDA), serine-N,N-diacetic acid (SEDA), isoserine-N,Ndiacetic acid (ISDA), phenylalanine-N,N-diacetic acid (PHDA), anthranilic acid-N,N-diacetic acid (ANDA), sulfanilic acid-N,N-diacetic acid (SLDA), taurine-N,N-diacetic acid (TUDA) and sulfomethyl-N,N-diacetic acid (SMDA) and alkali metal salts and derivative thereof. In some embodiments, the acidifying particle has a weight geometric mean particle size of from about 400μ to about 1200μ and a bulk density of at least 550 g/L. In some embodiments, the acidifying particle comprises at least about 5% of the builder.

In some embodiments, the acidifying particle can comprise any acid, including organic acids and mineral acids. Organic acids can have one or two carboxyls and in some instances up to 15 carbons, especially up to 10 carbons, such as formic, acetic, propionic, capric, oxalic, succinic, adipic, maleic, fumaric, sebacic, malic, lactic, glycolic, tartaric and glyoxylic acids. In some embodiments, the acid is citric acid. Mineral acids include hydrochloric and sulphuric acid. In some instances, the acidifying particle is a highly active particle comprising a high level of amino carboxylic builder. Sulphuric acid has been found to further contribute to the stability of the final particle.

In some embodiments, one or more cleaning composition described herein comprises at least one surfactant and/or a surfactant system wherein the surfactant is selected from nonionic surfactants, anionic surfactants, cationic surfactants, ampholytic surfactants, zwitterionic surfactants, semi-polar nonionic surfactants and mixtures thereof. In some embodiments, the surfactant is present at a level of from about 0.1% to about 60%, while in alternative embodiments the level is from about 1% to about 50%, while in still further embodiments the level is from about 5% to about 40%, by weight of the cleaning composition.

In some embodiments, one or more cleaning composition described herein comprises one or more detergent builders or builder systems. In some embodiments incorporating at least one builder, the cleaning compositions comprise at least about 1%, from about 3% to about 60% or even from about 5% to about 40% builder by weight of the cleaning composition. Builders include, but are not limited to, the alkali metal, ammonium and alkanolammonium salts of polyphosphates, alkali metal silicates, alkaline earth and alkali metal carbonates, aluminosilicates, polycarboxylate compounds, ether hydroxypolycarboxylates, copolymers of maleic anhydride with ethylene or vinyl methyl ether, 1,3,5-trihydroxy benzene-2,4,6-trisulphonic acid, and carboxymethyloxysuccinic acid, the various alkali metal, ammonium and substituted ammonium salts of polyacetic acids such as ethylenediamine tetraacetic acid and nitrilotriacetic acid, as well as polycarboxylates such as mellitic acid, succinic acid, citric acid, oxydisuccinic acid, polymaleic acid, benzene 1,3,5-tricarboxylic acid, carboxymethyloxysuccinic acid, and soluble salts thereof. Indeed, it is contemplated that any suitable builder will find use in one or more embodiment described herein.

In some embodiments, the builders form water-soluble hardness ion complexes (e.g., sequestering builders), such as citrates and polyphosphates (e.g., sodium tripolyphosphate and sodium tripolyphospate hexahydrate, potassium tripolyphosphate, and mixed sodium and potassium tripolyphosphate, etc.). It is contemplated that any suitable builder will find use in one or more composition described herein, including those known in the art (See e.g., EP 2 100 949).

In some embodiments, builders for use herein include phosphate builders and non-phosphate builders. In some embodiments, the builder is a phosphate builder. In some embodiments, the builder is a non-phosphate builder. If present, builders are used in a level of from 0.1% to 80%, or from 5 to 60%, or from 10 to 50% by weight of the composition. In some embodiments the product comprises a mixture of phosphate and non-phosphate builders. Suitable phosphate builders include mono-phosphates, di-phosphates, tri-polyphosphates or oligomeric-poylphosphates, including the alkali metal salts of these compounds, including the sodium salts. In some embodiments, a builder can be sodium tripolyphosphate (STPP). Additionally, the composition can comprise carbonate and/or citrate, preferably citrate that helps to achieve a neutral pH. Other suitable non-phosphate builders include homopolymers and copolymers of polycarboxylic acids and their partially or completely neutralized salts, monomeric polycarboxylic acids and hydroxycarboxylic acids and their salts. In some embodiments, salts of the above mentioned compounds include the ammonium and/or alkali metal salts, i.e. the lithium, sodium, and potassium salts, including sodium salts. Suitable polycarboxylic acids include acyclic, alicyclic, hetero-cyclic and aromatic carboxylic acids, wherein in some embodiments, they can contain at least two carboxyl groups which are in each case separated from one another by, in some instances, no more than two carbon atoms.

In some embodiments, one or more composition described herein contains at least one chelating agent. Suitable chelating agents include, but are not limited to copper, iron and/or manganese chelating agents and mixtures thereof. In embodiments in which at least one chelating agent is used, the cleaning compositions comprise from about 0.1% to about 15% or even from about 3.0% to about 10% chelating agent by weight composition.

In some still further embodiments, the cleaning compositions provided herein contain at least one deposition aid. Suitable deposition aids include, but are not limited to, polyethylene glycol, polypropylene glycol, polycarboxylate, soil release polymers such as polytelephthalic acid, clays such as kaolinite, montmorillonite, atapulgite, illite, bentonite, halloysite, and mixtures thereof.

In some embodiments, anti-redeposition agents find use in some embodiments described herein. In some embodiments, non-ionic surfactants find use. For example, in automatic dishwashing embodiments, non-ionic surfactants find use for surface modification purposes, in particular for sheeting, to avoid filming and spotting and to improve shine. These non-ionic surfactants also find use in preventing the re-deposition of soils. In some embodiments, the anti-redeposition agent is a non-ionic surfactant as known in the art (See e.g., EP 2 100 949). In some embodiments, the non-ionic surfactant can be ethoxylated nonionic surfactants, epoxy-capped poly(oxyalkylated) alcohols and amine oxides surfactants.

In some embodiments, one or more cleaning composition described herein includes one or more dye transfer inhibiting agents. Suitable polymeric dye transfer inhibiting agents include, but are not limited to, polyvinylpyrrolidone polymers, polyamine N-oxide polymers, copolymers of N-vinylpyrrolidone and N-vinylimidazole, polyvinyloxazolidones and polyvinylimidazoles or mixtures thereof. In embodiments in which at least one dye transfer inhibiting agent is used, the cleaning composition comprises from about 0.0001% to about 10%, from about 0.01% to about 5%, or even from about 0.1% to about 3% by weight composition.

In some embodiments, silicates are included in one or more composition described herein. In some such embodiments, sodium silicates (e.g., sodium disilicate, sodium metasilicate, and crystalline phyllosilicates) find use. In some embodiments, silicates are present at a level of from about 1% to about 20%. In some embodiments, silicates are present at a level of from about 5% to about 15% by weight of the composition.

In some still additional embodiments, one or more cleaning composition described herein contains a dispersant. Suitable water-soluble organic materials include, but are not limited to the homo- or co-polymeric acids or their salts, in which the polycarboxylic acid comprises at least two carboxyl radicals separated from each other by not more than two carbon atoms.

In some further embodiments, the enzymes used in one or more composition described herein are stabilized by any suitable technique. In some embodiments, the enzymes employed herein are stabilized by the presence of water-soluble sources of calcium and/or magnesium ions in the finished compositions that provide such ions to the enzymes. In some embodiments, the enzyme stabilizers include oligosaccharides, polysaccharides, and inorganic divalent metal salts, including alkaline earth metals, such as calcium salts, such as calcium formate. It is contemplated that various techniques for enzyme stabilization will find use in the present invention. For example, in some embodiments, the enzymes employed herein are stabilized by the presence of water-soluble sources of zinc (II), calcium (II) and/or magnesium (II) ions in the finished compositions that provide such ions to the enzymes, as well as other metal ions (e.g., barium (II), scandium (II), iron (II), manganese (II), aluminum (III), Tin (II), cobalt (II), copper (II), nickel (II), and oxovanadium (IV). Chlorides and sulfates also find use in some embodiments of the present invention. Examples of suitable oligosaccharides and polysaccharides (e.g., dextrins) are known in the art (See e.g., WO07/145964). In some embodiments, reversible protease inhibitors also find use, such as boron-containing compounds (e.g., borate, 4-formyl phenyl boronic acid, and phenyl-boronic acid derivatives (such for example, those described in WO96/41859) and/or a peptide aldehyde, such as, for example, is further described in WO2009/118375 and WO2013004636.

In some embodiments, bleaches, bleach activators and/or bleach catalysts are present in one or more composition described herein. In some embodiments, one or more composition described herein comprises inorganic and/or organic bleaching compound(s). Inorganic bleaches include, but are not limited to perhydrate salts (e.g., perborate, percarbonate, perphosphate, persulfate, and persilicate salts). In some embodiments, inorganic perhydrate salts are alkali metal salts. In some embodiments, inorganic perhydrate salts are included as the crystalline solid, without additional protection, although in some other embodiments, the salt is coated. Any suitable salt known in the art finds use herein (See e.g., EP 2 100 949).

In some embodiments, bleach activators are used in one or more composition described herein. Bleach activators are typically organic peracid precursors that enhance the bleaching action in the course of cleaning at temperatures of 60° C. and below. Bleach activators suitable for use herein include compounds which, under perhydrolysis conditions, give aliphatic peroxoycarboxylic acids having preferably from about 1 to about 10 carbon atoms, in particular from about 2 to about 4 carbon atoms, and/or optionally substituted perbenzoic acid. Additional bleach activators are known in the art and find use herein (See e.g., EP2100 949).

In addition, in some embodiments and as further described herein, one or more composition described herein further comprises at least one bleach catalyst. In some embodiments, the manganese triazacyclononane and related complexes find use, as well as cobalt, copper, manganese, and iron complexes. Additional bleach catalysts find use herein (See e.g., U.S. Pat. Nos. 4,246,612; 5,227,084; 4,810,410, WO 99/06521, and EP2100949).

In some embodiments, one or more composition described herein contains one or more catalytic metal complexes. In some embodiments, a metal-containing bleach catalyst finds use herein. In some embodiments, the metal bleach catalyst comprises a catalyst system comprising a transition metal cation of defined bleach catalytic activity (e.g., copper, iron, titanium, ruthenium, tungsten, molybdenum, or manganese cations), an auxiliary metal cation having little or no bleach catalytic activity (e.g., zinc or aluminum cations), and a sequestrate having defined stability constants for the catalytic and auxiliary metal cations, particularly ethylenediaminetetraacetic acid, ethylenediaminetetra (methylenephosphonic acid) and water-soluble salts thereof are used (See e.g., U.S. Pat. No. 4,430,243). In some embodiments, one or more composition described herein is catalyzed by means of a manganese compound. Such compounds and levels of use are well known in the art (See e.g., U.S. Pat. No. 5,576,282). In additional embodiments, cobalt bleach catalysts find use in one or more composition described herein. Various cobalt bleach catalysts are known in the art (See e.g., USPNs 5,597,936 and 5,595,967) and are readily prepared by known procedures.

In some additional embodiments, one or more composition described herein includes a transition metal complex of a macropolycyclic rigid ligand (MRL). As a practical matter, and not by way of limitation, in some embodiments, one or more composition and cleaning processes described herein are adjusted to provide on the order of at least one part per hundred million of the active MRL species in the aqueous washing medium, and in some embodiments, provides from about 0.005 ppm to about 25 ppm, from about 0.05 ppm to about 10 ppm, or from about 0.1 ppm to about 5 ppm of the MRL in the wash liquor. In some embodiments, transition-metals in the instant transition-metal bleach catalyst include, but are not limited to manganese, iron and chromium. MRLs also include, but are not limited to special ultra-rigid ligands that are cross-bridged (e.g., 5,12-diethyl-1,5,8,12-tetraazabicyclo[6.6.2]hexadecane). Suitable transition metal MRLs are readily prepared by known procedures (See e.g., WO2000/32601, and U.S. Pat. No. 6,225,464).

In some embodiments, one or more composition described herein comprises metal care agents. Metal care agents find use in preventing and/or reducing the tarnishing, corrosion, and/or oxidation of metals, including aluminum, stainless steel, and non-ferrous metals (e.g., silver and copper). Suitable metal care agents include those described in EP 2100949, WO 9426860 and WO94/26859). In some embodiments, the metal care agent is a zinc salt. In some further embodiments, one or more composition described herein comprises from about 0.1% to about 5% by weight of one or more metal care agent.

In some embodiments, the cleaning composition is a high density liquid (HDL) composition comprising one or more subtilisin variant described herein. The HDL liquid laundry detergent can comprise a detersive surfactant (10%-40% wt/wt), including an anionic detersive surfactant (selected from a group of linear or branched or random chain, substituted or unsubstituted alkyl sulphates, alkyl sulphonates, alkyl alkoxylated sulphate, alkyl phosphates, alkyl phosphonates, alkyl carboxylates, and/or mixtures thereof); and optionally non-ionic surfactant (selected from a group of linear or branched or random chain, substituted or unsubstituted alkyl alkoxylated alcohol, for example a C₈-C₁₈ alkyl ethoxylated alcohol and/or C₆-C₁₂ alkyl phenol alkoxylates), optionally wherein the weight ratio of anionic detersive surfactant (with a hydrophilic index (HIc) of from 6.0 to 9) to non-ionic detersive surfactant is greater than 1:1. Suitable detersive surfactants also include cationic detersive surfactants (selected from a group of alkyl pyridinium compounds, alkyl quarternary ammonium compounds, alkyl quarternary phosphonium compounds, alkyl ternary sulphonium compounds, and/or mixtures thereof); zwitterionic and/or amphoteric detersive surfactants (selected from a group of alkanolamine sulpho-betaines); ampholytic surfactants; semi-polar non-ionic surfactants and mixtures thereof.

The composition can comprise optionally, a surfactancy boosting polymer consisting of amphiphilic alkoxylated grease cleaning polymers (selected from a group of alkoxylated polymers having branched hydrophilic and hydrophobic properties, such as alkoxylated polyalkylenimines in the range of 0.05 wt %-10 wt %) and/or random graft polymers (typically comprising of hydrophilic backbone comprising monomers selected from the group consisting of: unsaturated C₁-C₆ carboxylic acids, ethers, alcohols, aldehydes, ketones, esters, sugar units, alkoxy units, maleic anhydride, saturated polyalcohols such as glycerol, and mixtures thereof; and hydrophobic side chain(s) selected from the group consisting of: C₄-C₂₅ alkyl group, polypropylene, polybutylene, vinyl ester of a saturated C₁-C₆ mono-carboxylic acid, C₁-C₆ alkyl ester of acrylic or methacrylic acid, and mixtures thereof.

The composition can comprise additional polymers such as soil release polymers (include anionically end-capped polyesters, for example SRP1, polymers comprising at least one monomer unit selected from saccharide, dicarboxylic acid, polyol and combinations thereof, in random or block configuration, ethylene terephthalate-based polymers and co-polymers thereof in random or block configuration, for example Repel-o-tex SF, SF-2 and SRP6, Texcare SRA100, SRA300, SRN100, SRN170, SRN240, SRN300 and SRN325, Marloquest SL), anti-redeposition polymers (0.1 wt % to 10 wt %, include carboxylate polymers, such as polymers comprising at least one monomer selected from acrylic acid, maleic acid (or maleic anhydride), fumaric acid, itaconic acid, aconitic acid, mesaconic acid, citraconic acid, methylenemalonic acid, and any mixture thereof, vinylpyrrolidone homopolymer, and/or polyethylene glycol, molecular weight in the range of from 500 to 100,000 Da); cellulosic polymer (including those selected from alkyl cellulose, alkyl alkoxyalkyl cellulose, carboxyalkyl cellulose, alkyl carboxyalkyl cellulose examples of which include carboxymethyl cellulose, methyl cellulose, methyl hydroxyethyl cellulose, methyl carboxymethyl cellulose, and mixures thereof) and polymeric carboxylate (such as maleate/acrylate random copolymer or polyacrylate homopolymer).

The composition can further comprise saturated or unsaturated fatty acid, preferably saturated or unsaturated C₁₂-C₂₄ fatty acid (0 wt % to 10 wt %); deposition aids (examples for which include polysaccharides, preferably cellulosic polymers, poly diallyl dimethyl ammonium halides (DADMAC), and co-polymers of DAD MAC with vinyl pyrrolidone, acrylamides, imidazoles, imidazolinium halides, and mixtures thereof, in random or block configuration, cationic guar gum, cationic cellulose such as cationic hydoxyethyl cellulose, cationic starch, cationic polyacylamides, and mixtures thereof.

The composition can further comprise dye transfer inhibiting agents examples of which include manganese phthalocyanine, peroxidases, polyvinylpyrrolidone polymers, polyamine N-oxide polymers, copolymers of N-vinylpyrrolidone and N-vinylimidazole, polyvinyloxazolidones and polyvinylimidazoles and/or mixtures thereof, chelating agents examples of which include ethylene-diamine-tetraacetic acid (EDTA); diethylene triamine penta methylene phosphonic acid (DTPMP); hydroxy-ethane diphosphonic acid (HEDP); ethylenediamine N,N′-disuccinic acid (EDDS); methyl glycine diacetic acid (MGDA); diethylene triamine penta acetic acid (DTPA); propylene diamine tetracetic acid (PDT A); 2-hydroxypyridine-N-oxide (HPNO); or methyl glycine diacetic acid (MGDA); glutamic acid N,N-diacetic acid (N,N-dicarboxymethyl glutamic acid tetrasodium salt (GLDA); nitrilotriacetic acid (NTA); 4,5-dihydroxy-m-benzenedisulfonic acid; citric acid and any salts thereof; N-hydroxyethylethylenediaminetri-acetic acid (HEDTA), triethylenetetraaminehexaacetic acid (TTHA), N-hydroxyethyliminodiacetic acid (HEIDA), dihydroxyethylglycine (DHEG), ethylenediaminetetrapropionic acid (EDTP) and derivatives thereof.

The composition may optionally include enzymes (generally about 0.01 wt % active enzyme to 0.5 wt % active enzyme) selected from proteases, amylases, lipases, cellulases, choline oxidases, peroxidases/oxidases, pectate lyases, mannanases, cutinases, laccases, phospholipases, lysophospholipases, acyltransferases, perhydrolases, arylesterases, and any mixture thereof. The composition may comprise an enzyme stabilizer (examples of which include polyols such as propylene glycol or glycerol, sugar or sugar alcohol, lactic acid, reversible protease inhibitor, boric acid, or a boric acid derivative, e.g., an aromatic borate ester, or a phenyl boronic acid derivative such as 4-formylphenyl boronic acid).

The composition can further comprise silicone or fatty-acid based suds suppressors; heuing dyes, calcium and magnesium cations, visual signaling ingredients, anti-foam (0.001 wt % to about 4.0 wt %), and/or structurant/thickener (0.01 wt % to 5 wt %, selected from the group consisting of diglycerides and triglycerides, ethylene glycol distearate, microcrystalline cellulose, cellulose based materials, microfiber cellulose, biopolymers, xanthan gum, gellan gum, and mixtures thereof).

The composition can be any liquid form, for example a liquid or gel form, or any combination thereof.

In some embodiments, one or more composition described herein is provided in unit dose form, including tablets, capsules, sachets, pouches, and multi-compartment pouches. In some embodiments, the unit dose format is designed to provide controlled release of the ingredients within a multi-compartment pouch (or other unit dose format). Suitable unit dose and controlled release formats are known in the art (See e.g., EP 2100949, WO 02/102955, U.S. Pat. Nos. 4,765,916, 4,972,017, and WO 04/111178 for materials suitable for use in unit dose and controlled release formats). In some embodiments, the unit dose form is provided by tablets wrapped with a water-soluble film or water-soluble pouches. Various unit dose formats are provided in EP2100947 and WO2013/165725 and are known in the art.

In some embodiments, the cleaning composition is a high density powder (HDD) composition having a variant serine protease polypeptide protease. The HDD powder laundry detergent can comprise a detersive surfactant including anionic detersive surfactants (e.g., linear or branched or random chain, substituted or unsubstituted alkyl sulphates, alkyl sulphonates, alkyl alkoxylated sulphate, alkyl phosphates, alkyl phosphonates, alkyl carboxylates and/or mixtures thereof), non-ionic detersive surfactant (e.g., linear or branched or random chain, substituted or unsubstituted C₈-C₁₈ alkyl ethoxylates, and/or C₆-C₁₂ alkyl phenol alkoxylates), cationic detersive surfactants (e.g., alkyl pyridinium compounds, alkyl quaternary ammonium compounds, alkyl quaternary phosphonium compounds, alkyl ternary sulphonium compounds, and mixtures thereof), zwitterionic and/or amphoteric detersive surfactants (e.g., alkanolamine sulpho-betaines); ampholytic surfactants; semi-polar non-ionic surfactants and mixtures thereof; builders (phosphate free builders (e.g., zeolite builders examples of which include zeolite A, zeolite X, zeolite P and zeolite MAP in the range of 0 wt % to less than 10 wt %]; phosphate builders [examples of which include sodium tri-polyphosphate in the range of 0 wt % to less than 10 wt %]; citric acid, citrate salts and nitrilotriacetic acid or salt thereof in the range of less than 15 wt %); silicate salt (e.g., sodium or potassium silicate or sodium meta-silicate in the range of 0 wt % to less than 10 wt %, or layered silicate (SKS-6)); carbonate salt (e.g., sodium carbonate and/or sodium bicarbonate in the range of 0 wt % to less than 10 wt %); and bleaching agents (including photobleaches, (e.g., sulfonated zinc phthalocyanines, sulfonated aluminum phthalocyanines, xanthenes dyes, and mixtures thereof); hydrophobic or hydrophilic bleach activators (e.g., dodecanoyl oxybenzene sulfonate, decanoyl oxybenzene sulfonate, decanoyl oxybenzoic acid or salts thereof, 3,5,5-trimethy hexanoyl oxybenzene sulfonate, tetraacetyl ethylene diamine-TAED, and nonanoyloxybenzene sulfonate-NOBS, nitrile quats, and mixtures thereof); sources of hydrogen peroxide (e.g., inorganic perhydrate salts examples of which include mono or tetra hydrate sodium salt of perborate, percarbonate, persulfate, perphosphate, or persilicate); preformed hydrophilic and/or hydrophobic peracids (e.g., percarboxylic acids and salts, percarbonic acids and salts, perimidic acids and salts, peroxymonosulfuric acids and salts), and mixtures thereof and/or bleach catalysts (e.g., imine bleach boosters (e.g., iminium cations and polyions); iminium zwitterions; modified amines; modified amine oxides; N-sulphonyl imines; N-phosphonyl imines; N-acyl imines; thiadiazole dioxides; perfluoroimines; cyclic sugar ketones and mixtures thereof, and metal-containing bleach catalyst (e.g., copper, iron, titanium, ruthenium, tungsten, molybdenum, or manganese cations along with an auxiliary metal cations such as zinc or aluminum and a sequestrate such as ethylenediaminetetraacetic acid, ethylenediaminetetra(methylenephosphonic acid) and water-soluble salts thereof).

The composition can further include enzymes, e.g., proteases, amylases, lipases, cellulases, choline oxidases, peroxidases/oxidases, pectate lyases, mannanases, cutinases, laccases, phospholipases, lysophospholipases, acyltransferase, perhydrolase, arylesterase, and any mixture thereof.

The composition can further comprise additional detergent ingredients including perfume microcapsules, starch encapsulated perfume accord, hueing agents, additional polymers including fabric integrity and cationic polymers, dye lock ingredients, fabric-softening agents, brighteners (for example C.I. Fluorescent brighteners), flocculating agents, chelating agents, alkoxylated polyamines, fabric deposition aids, and/or cyclodextrin.

In some embodiments, the cleaning composition is an automatic dishwashing (ADW) detergent composition having a serine protease of the present invention. The ADW detergent composition can comprise two or more non-ionic surfactants selected from a group of ethoxylated non-ionic surfactants, alcohol alkoxylated surfactants, epoxy-capped poly(oxyalkylated) alcohols, or amine oxide surfactants present in amounts from 0 to 10% by weight; builders in the range of 5-60% comprising either phosphate (mono-phosphates, di-phosphates, tri-polyphosphates or oligomeric-poylphosphates, preferred sodium tripolyphosphate-STPP or phosphate-free builders [amino acid based compounds, examples of which include MGDA (methyl-glycine-diacetic acid), and salts and derivatives thereof, GLDA (glutamic-N,Ndiacetic acid) and salts and derivatives thereof, IDS (iminodisuccinic acid) and salts and derivatives thereof, carboxy methyl inulin and salts and derivatives thereof and mixtures thereof, nitrilotriacetic acid (NTA), diethylene triamine penta acetic acid (DTPA), B-alaninediacetic acid (B-ADA) and their salts], homopolymers and copolymers of poly-carboxylic acids and their partially or completely neutralized salts, monomeric polycarboxylic acids and hydroxycarboxylic acids and their salts in the range of 0.5% to 50% by weight; sulfonated/carboxylated polymers (provide dimensional stability to the product) in the range of about 0.1% to about 50% by weight; drying aids in the range of about 0.1% to about 10% by weight (selected from polyesters, especially anionic polyesters optionally together with further monomers with 3 to 6 functionalities which are conducive to polycondensation, specifically acid, alcohol or ester functionalities, polycarbonate-, polyurethane- and/or polyurea-polyorganosiloxane compounds or precursor compounds thereof of the reactive cyclic carbonate and urea type); silicates in the range from about 1% to about 20% by weight (sodium or potassium silicates for example sodium disilicate, sodium meta-silicate and crystalline phyllosilicates); bleach-inorganic (for example perhydrate salts such as perborate, percarbonate, perphosphate, persulfate and persilicate salts) and organic (for example organic peroxyacids including diacyl and tetraacylperoxides, especially diperoxydodecanedioc acid, diperoxytetradecanedioc acid, and diperoxyhexadecanedioc acid); bleach activators-organic peracid precursors in the range from about 0.1% to about 10% by weight; bleach catalysts (selected from manganese triazacyclononane and related complexes, Co, Cu, Mn and Fe bispyridylamine and related complexes, and pentamine acetate cobalt(III) and related complexes); metal care agents in the range from about 0.1% to 5% by weight (selected from benzatriazoles, metal salts and complexes, and/or silicates); enzymes in the range from about 0.01 to 5.0 mg of active enzyme per gram of automatic dishwashing detergent composition (acyl transferases, alpha-amylases, beta-amylases, alpha-galactosidases, arabinosidases, aryl esterases, beta-galactosidases, carrageenases, catalases, cellobiohydrolases, cellulases, chondroitinases, cutinases, endo-beta-1,4-glucanases, endo-beta-mannanases, esterases, exo-mannanases, galactanases, glucoamylases, hemicellulases, hyaluronidases, keratinases, laccases, lactases, ligninases, lipases, lipoxygenases, mannanases, oxidases, pectate lyases, pectin acetyl esterases, pectinases, pentosanases, peroxidases, phenoloxidases, phosphatases, phospholipases, phytases, polygalacturonases, proteases, pullulanases, reductases, rhamnogalacturonases, beta-glucanases, tannases, transglutaminases, xylan acetyl-esterases, xylanases, xyloglucanases, and xylosidases, and any mixture thereof); and enzyme stabilizer components (selected from oligosaccharides, polysaccharides and inorganic divalent metal salts).

Representative detergent formulations that can include one or more subtilisin variant described herein can be found in WO2013063460, pages 78-152, and in particular the tables of pages 94 to 152. Serine proteases are typically incorporated into a detergent composition at a level of from 0.00001% to 10% of enzyme protein by weight of the composition. In some embodiments, the detergent composition comprises more than 0.0001%, 0.001%, 0.01%, or 0.1% of the serine protease by weight of the composition. In some embodiments, the detergent composition comprises less than 1%, 0.1%, 0.01%, or 0.001% of the serine protease by weight of the composition.

Also provided are compositions and methods of treating fabrics (e.g., to desize a textile) using one or more subtilisin variant described herein. Fabric-treating methods are well known in the art (see, e.g., U.S. Pat. No. 6,077,316). For example, the feel and appearance of a fabric can be improved by a method comprising contacting the fabric with a serine protease in a solution. The fabric can be treated with the solution under pressure.

One or more subtilisin variant described herein can be applied during or after the weaving of a textile, or during the desizing stage, or one or more additional fabric processing steps. During the weaving of textiles, the threads are exposed to considerable mechanical strain. Prior to weaving on mechanical looms, warp yarns are often coated with sizing starch or starch derivatives to increase their tensile strength and to prevent breaking. One or more subtilisin variant described herein can be applied during or after the weaving to remove these sizing starch or starch derivatives. After weaving, one or more subtilisin variant described herein can be used to remove the size coating before further processing the fabric to ensure a homogeneous and wash-proof result.

One or more subtilisin variant described herein can be used alone or with other desizing chemical reagents and/or desizing enzymes to desize fabrics, including cotton-containing fabrics, as detergent additives, e.g., in aqueous compositions. An amylase also can be used in compositions and methods for producing a stonewashed look on indigo-dyed denim fabric and garments. For the manufacture of clothes, the fabric can be cut and sewn into clothes or garments, which are afterwards finished. In particular, for the manufacture of denim jeans, different enzymatic finishing methods have been developed. The finishing of denim garment normally is initiated with an enzymatic desizing step, during which garments are subjected to the action of proteolytic enzymes to provide softness to the fabric and make the cotton more accessible to the subsequent enzymatic finishing steps. The serine protease can be used in methods of finishing denim garments (e.g., a “bio-stoning process”), enzymatic desizing and providing softness to fabrics, and/or finishing process.

One or more subtilisin variant described herein finds further use in the enzyme aided removal of proteins from animals and their subsequent degradation or disposal, such as feathers, skin, hair, hide, and the like. In some instances, immersion of the animal carcass in a solution comprising one or more subtilisin variant described herein can act to protect the skin from damage in comparison to the traditional immersion in scalding water or the defeathering process. In one embodiment, feathers can be sprayed with one or more subtilisin variant described herein under conditions suitable for digesting or initiating degradation of the plumage. In some embodiments, one or more subtilisin variant described herein can be used, as above, in combination with an oxidizing agent.

In some embodiments, removal of the oil or fat associated with raw feathers is assisted by using one or more subtilisin variant described herein. In some embodiments, the serine protease polypeptides are used in compositions for cleaning the feathers as well as to sanitize and partially dehydrate the fibers. In yet other embodiments, one or more subtilisin variant described herein find use in recovering protein from plumage. In some other embodiments, the serine protease polypeptides are applied in a wash solution in combination with 95% ethanol or other polar organic solvent with or without a surfactant at about 0.5% (v/v). In yet other embodiments, the disclosed protease polypeptides find use in recovering protein from plumage. The disclosed protease polypeptides may be used alone or in combination in suitable feather processing and proteolytic methods, such as those disclosed in PCT/EP2013/065362, PCT/EP2013/065363, and PCT/EP2013/065364, which are hereby incorporated by reference. In some embodiments, the recovered protein can be subsequently used in animal or fish feed.

In a further aspect of the invention, one or more subtilisin variant described herein can be used as a component of an animal feed composition, animal feed additive and/or pet food. Another embodiment relates to a method for preparing such an animal feed, animal feed additive and/or pet food composition comprising mixing one or more subtilisin variant described herein with one or more animal feed ingredient and/or animal feed additive ingredient and/or pet food ingredient. Furthermore, one or more subtilisin variant described herein may be used in the preparation of an animal feed and/or animal feed additive and/or pet food composition.

The term “animal” includes all non-ruminant and ruminant animals. In a particular embodiment, the animal is a non-ruminant animal, such as a horse and a mono-gastric animal. Examples of mono-gastric animals include, but are not limited to, pigs and swine, such as piglets, growing pigs, sows; poultry such as turkeys, ducks, chicken, broiler chicks, layers; fish such as salmon, trout, tilapia, catfish and carps; and crustaceans such as shrimps and prawns. In a further embodiment the animal is a ruminant animal including, but not limited to, cattle, young calves, goats, sheep, giraffes, bison, moose, elk, yaks, water buffalo, deer, camels, alpacas, llamas, antelope, pronghorn and nilgai.

In the present context, it is intended that the term “pet food” is understood to mean a food for a household animal such as, but not limited to, dogs, cats, gerbils, hamsters, chinchillas, fancy rats, guinea pigs; avian pets, such as canaries, parakeets, and parrots; reptile pets, such as turtles, lizards and snakes; and aquatic pets, such as tropical fish and frogs.

The terms “animal feed composition,” “feedstuff” and “fodder” are used interchangeably and can comprise one or more feed materials selected from the group comprising a) cereals, such as small grains (e.g., wheat, barley, rye, oats and combinations thereof) and/or large grains such as maize or sorghum; b) by products from cereals, such as corn gluten meal, Distillers Dried Grain Solubles (DDGS) (particularly corn based Distillers Dried Grain Solubles (cDDGS), wheat bran, wheat middlings, wheat shorts, rice bran, rice hulls, oat hulls, palm kernel, and citrus pulp; c) protein obtained from sources such as soya, sunflower, peanut, lupin, peas, fava beans, cotton, canola, fish meal, dried plasma protein, meat and bone meal, potato protein, whey, copra, sesame; d) oils and fats obtained from vegetable and animal sources; e) minerals and vitamins.

One or more subtilisin variant described herein finds further use in the enzyme aided bleaching of paper pulps such as chemical pulps, semi-chemical pulps, kraft pulps, mechanical pulps or pulps prepared by the sulfite method. In general terms, paper pulps are incubated with one or more subtilisin variant described herein under conditions suitable for bleaching the paper pulp. In some embodiments, the pulps are chlorine free pulps bleached with oxygen, ozone, peroxide or peroxyacids. In some embodiments, the subtilisin variants are used in enzyme aided bleaching of pulps produced by modified or continuous pulping methods that exhibit low lignin contents. In some other embodiments, the subtilisin variants are applied alone or preferably in combination with xylanase and/or endoglucanase and/or alpha-galactosidase and/or cellobiohydrolase enzymes.

One or more subtilisin variant described herein finds further use in the enzyme aided debridement of tissue. This involves the removal of dead or damaged tissue, for example, removal from wounds to aid in healing.

One or more subtilisin variant described herein finds further use in tissue culture. In particular, one or more subtilisin variant described herein can be used to suspend or resuspend cells adherent to a cell culture wall, such as during the process of harvesting cells. One or more subtilisin variant described herein can be used to cleave protein bonds between cultured cells and the dish, allowing cells to become suspended in solution.

One or more subtilisin variant described herein find further use as a food additive, a digestive aide, or a food processing aid.

### EXAMPLES

The following examples are provided to demonstrate and illustrate certain preferred embodiments and aspects of the present disclosure and should not be construed as limiting.

In the experimental disclosure which follows, the following abbreviations apply: ADW (automatic dish washing); BMI (blood, milk and ink), BSA (bovine serum albumin); CAPS (N-cyclohexyl-3-aminopropanesulfonic acid); CHES (N-cyclohexyl-2-aminoethanesulfonic acid); DMC (dimethyl casein); HDD (heavy duty dry/powder); HDL (heavy duty liquid); HEPES (4-(2-hydroxyethyl)-1-piperazineethanesulfonic acid); MTP (microtiter plate); ND (not done); OD (optical density); PAS (polyacryl swatches); PCR (polymerase chain reaction); ppm (parts per million); QS (quantity sufficient); rpm (revolutions per minute); AAPF (succinyl-Ala-Ala-Pro-Phe-p-nitroanilide); TNBSA (2,4,6-trinitrobenzene sulfonic acid); v/v (volume to volume); w/v (weight to volume).

### Example 1

### Heterologous Expression of B. gibsonii-Clade Subtilisins

DNA manipulations to generate B. gibsonii-clade subtilisins were carried out using conventional molecular biology techniques (see, e.g., Sambrook et al, Molecular Cloning: Cold Spring Harbor Laboratory Press). All subtilisins were expressed and recovered as described in the subsequent examples. A series of artificial DNA sequences were generated, coding for mature B. gibsonii-clade subtilisin sequences that introduce multiple amino acid modifications into the sequence of the wildtype B. gibsonii-clade Bgi02446 protease, which wildtype protease is more fully described in International Patent Application No. PCT/US2014/070107, and as AprBG in Deng et al. J. Microbiol. Biotechnol. (2014), 24(2), 197-198 (accession number AGS78407.1).

### Example 2

### Expression of B. gibsonii-Clade BSP-00801 of Mature Subtilisin Using Either the Native Bgi02446 Propeptide Sequence, or the Propeptide Sequence from B. lentus Subtilisin

The processed mature enzyme BSP-00801, expressed under the control of B. lentus pro-sequence (SEQ ID NO:5), was characterized and showed the amino acid sequence to be 269 amino acids with QQ at the N-terminus (SEQ ID NO:18 for the amino acid sequence and SEQ ID NO:15 for the nucleotide sequence). The processed mature enzyme of BSP-00801, expressed under the control of the Bgi02446 pro-sequence (SEQ ID NO:4), was characterized and showed the amino acid sequence to be 268 amino acids with a single Q at the N-terminus (SEQ ID NO:20). The amino acid sequence of the preproenzyme of BSP-00801 is SEQ ID NO:16. The amino acid sequence of the proenzyme is SEQ ID NO:17.

The processed mature enzyme Bgi02446, expressed under the control of B. lentus pro-sequence (SEQ ID NO:5), was characterized and showed the amino acid sequence to be 269 amino acids with QQ at the N-terminus (SEQ ID NO:85). The processed mature enzyme of Bgi02446, expressed under the control of the Bgi02446 pro-sequence (SEQ ID NO:4), was characterized and showed the amino acid sequence to be 268 amino acids with a single Q at the N-terminus (SEQ ID NO:19).

The amino acid sequences of the processed mature enzymes identified in Tables 3, 4, and 5 were expressed under the control of the Bgi02446 pro-sequence and, based on the knowledge of the pro-mature junction in homologous serine proteases such as BPN′ (Wells et al., 1983. Nucleic Acids Res, 11: 7911-25), and PB92 protease (van der Laan et al. 1991. Appl Environ Microbiol, 57:901-909), were predicted to be 269 amino acid with a QQTVP at the N-terminus, but instead QTVP was observed.

The processed mature enzyme BSP-00801 (SEQ ID NO:18) used in Example 5 was expressed under the control of B. lentus pro-sequence (SEQ ID NO:5) in accordance with the process that follows. A more detailed description of the process for expressing BSP-00801 mature enzyme via the Bgi02446 pro-sequence and the B. lentus pro-sequence is set forth in U.S. Provisional Patent Application No. 62/181,192, filed Jun. 17, 2015.

DNA cassettes comprising B. subtilis aprE promoter (SEQ ID NO:1), the B. subtilis aprE signal peptide sequence (SEQ ID NO:2 for nucleotide sequence and SEQ ID NO:3 for the amino acid sequence), the pro sequence from either B. gibsonii Bgi02446 (SEQ ID NO:4) or from B. lentus (SEQ ID NO:5), and the sequence corresponding to the gene for B. gibsoni-clade subtilisin BSP-00801 (SEQ ID NO:6) were synthesized by amplification using primers listed on Table 1. Using techniques known in the art, PCR fragments were assembled using Gibson Assembly (SGI DNA Cat #GA1100-10) to make the final expression cassettes. The cells were transformed and grown on skim milk plates under chloramphenicol selection.

TABLE 1
Primers used to construct expression cassettes encoding Bgi02446 pro-
peptide or B. lentas pro-peptide fusions to B. gibsoni-clade subtilisin
BSP-00801 mature gene.

| Primer | Sequence | SEQ ID NO: |
|---|---|---|
| EL1664 | GAGGATGCAGAAGTAACGACAATGCAACAAACAGTGCCATGG | 7 |
| EL1665 | CCAAGGCCGGTTTTTTATGTATCTAGATTAGCGTGTTGCCGCTTCTG | 8 |
| | CATTG | |
| EL1666 | GAAGAAGACATTGAACTGTCTATTCAACAAACAGTGCCATGG | 9 |
| EL1667 | CAATGCAGAAGCGGCAACACGCTAATCTAGATACATAAAAAACCG | 10 |
| | GCCTTGG | |
| EL1668 | CCATGGCACTGTTTGTTGCATTGTCGTTACTTCTGCATCCTC | 11 |
| EL1669 | CCATGGCACTGTTTGTTGAATAGACAGTTCAATGTCTTCTTC | 12 |

The B. gibsonii-clade subtilisins set forth in Tables 3, 4, and 5 were produced in B. subtilis using an expression cassette consisting of the B. subtilis aprE promoter, the B. subtilis aprE signal peptide sequence (SEQ ID NO:3), the Bgi02446 pro-sequence, the mature protease sequences for each of the artificial sequences, and a BPN′ terminator. Each expression cassette was cloned into the pHYT replicating shuttle vector and transformed into a suitable B. subtilis strain. The pHYT vector was derived from pHY300PLK (Takara) by adding a terminator (SEQ ID NO:13) after the tetracycline resistance gene using the BstEII and EcoRI sites. The HindIII site in pHY300PLK was also removed using a linker (SEQ ID NO:14) cloned into the BamHI and HindIII sites.

To produce the B. gibsonii-clade subtilisins set forth in Tables 3, 4, and 5, the B. subtilis host strains transformed with the various pHYT plasmids were cultivated in an enriched semi-defined media based on MOPs buffer, with urea as major nitrogen source, glucose as the main carbon source, and supplemented with 1% soytone for robust cell growth. After incubation, the secreted proteases were isolated from the growth medium by centrifugation and filtration. Clarified culture supernatants were used for assays and purification as described below.

### Example 3

### Protease Activity of B. gibsonii-Clade Subtilisins

The protease activity of Bgi02446 and B. gibsonii-clade subtilisins set forth in Tables 3, 4, and 5 were tested by measuring the hydrolysis of dimethyl casein (DMC) substrate. The reagent solutions used for the DMC assay were: 2.5% w/v DMC (Sigma C-9801) in 100 mM sodium carbonate buffer pH 9.5, 0.075% TNBSA (Thermo Scientific) in Reagent A. Reagent A: 45.4 g Na₂B₄O₇·10H₂O (Merck) in 15 mL 4 N NaOH to reach a final volume of 1000 mL in deionised water. Protease supernatants were diluted in dilution solution: 10 mM NaCl, 0.1 mM CaCl₂), 0.005% Tween-80 to the desired concentration to achieve a linear response during hydrolysis over 5 min. A 96-well microtiter plate (MTP) was filled with 95 μl DMC substrate followed by the addition of 5 μl diluted protease supernatant. 100 μL of TNBSA in Reagent A was then added with slow mixing. Activity was measured at 405 nm over 5 min using a SpectraMax plate reader in kinetic mode at RT. The absorbance of a blank containing no protease was subtracted from each sample reading. The protease activity was expressed as mOD/min.

The protease activity of Bgi02446 and B. gibsonii-clade subtilisins set forth in Table 7 was tested by measuring hydrolysis of N-suc-AAPF-pNA or dimethyl casein (DMC). The reagent solutions used for the AAPF hydrolysis assay were: 100 mM Tris/HCl pH 8.6, containing 0.005% TWEEN®-80 (Tris dilution buffer); 100 mM Tris buffer pH 8.6, containing 10 mM CaCl₂) and 0.005% TWEEN®-80 (Tris/Ca buffer); and 160 mM suc-AAPF-pNA in DMSO (suc-AAPF-pNA stock solution) (Sigma: S-7388). To prepare a substrate working solution, 1 ml suc-AAPF-pNA stock solution was added to 100 ml Tris/Ca buffer and mixed well. An enzyme sample was added to a MTP plate (Greiner 781101) containing 1 mg/suc-AAPF-pNA working solution and assayed for activity at 405 nm over 3 minutes using a SpectraMax plate reader in kinetic mode at RT. The absorbance of a blank containing no protease was subtracted from each sample reading. The protease activity was expressed as mOD min-.

### Example 4

### Measurement of Cleaning Performance and Stability of B. gibsonii-Clade Subtilisins

The concentration of the proteases in culture supernatant was determined by UHPLC using a Zorbax 300 SB-C₃ column. Culture supernatant was diluted appropriately in dilution buffer (Tris 25 mM, pH 7.4, 5 mM CaCl₂). The samples were eluted from the column with a gradient of Buffer A (0.1% Trifluoroacetic acid) and Buffer B (0.07% Acetonitrile). The protein concentration of the samples was calculated based on a standard curve of the purified parent enzyme.

The cleaning performance of each B. gibsonii-clade protease was measured in dish based applications (ADW) using GSM-B formula (see Table 2), pH 10.5 and egg yolk microswatches (PAS-38, Center for Testmaterials BV, Vlaardingen, Netherlands). The pre-punched PAS-38 swatches that were used in the ADW performance assays were either rinsed or unrinsed. To prepare rinsed PAS38 swatches, 180p 10 mM CAPS buffer of pH 11 was added to MTPs containing PAS38 microswatches. The plates were sealed and incubated in an iEMS incubator for 30 min at 60° C. and 1100 rpm shaking. After incubation the buffer was removed, and the swatches were rinsed with deionized water to remove any residual buffer. The plates were air dried prior to use in the performance assay. The microswatch plates were filled prior to enzyme addition with 3 g/l GSM-B solution in 374 ppm water hardness.

Laundry (HDL) cleaning performance of each B. gibsonii-clade protease was tested using BMI microswatches (blood/milk/ink on cotton) (EMPA-116, Center for Testmaterials BV, Vlaardingen, Netherlands). Pre-punched (to fit on MTP) and filled microswatch-containing plates were used. The microswatch plates were filled prior to enzyme addition with 2.7 g/l Persil Non-Bio (Unilever) liquid detergent in 250 ppm water hardness, which is a commercial liquid detergent that does not contain boron or enzymes and which was purchased for use in this test.

Following incubation (PAS-38 swatches incubated for 30 minutes at 40° C. and EMPA116 swatches incubated for 15 minutes at 25° C.), absorbance was read at 405 nm for EMPA-116 and PAS-38 swatches, using the SpectraMax plate reader. Absorbance results were obtained by subtracting the value for a blank control (no enzyme) from each sample value (hereinafter “blank subtracted absorbance”). For each condition and B. gibsonii-clade protease, a performance index (PI) was calculated by dividing the blank subtracted absorbance by that of the parent protease at the same concentration. The value for the parent protease was determined from a standard curve of the parent protease which was included in the test and which was fitted to a Langmuir fit or Hill Sigmoidal fit.

TABLE 2
GSM-B pH 10.5 Phosphate-Free ADW Detergent Ingredients

| Component | Weight % |
|---|---|
| Sodium citrate dehydrate | 30.0 |
| Maleic acid/acrylic acid copolymer sodium salt | 12.0 |
| (SOKALAN ® CP5; BASF) | |
| Sodium perborate monohydrate | 5.0 |
| TAED | 2.0 |
| Sodium disilicate: Protil A (Cognis) | 25.0 |
| Linear fatty alcohol ethoxylate | 2.0 |
| Sodium carbonate anhydrous | add to 100 |

To measure the stability, appropriate dilutions of B. gibsonii-clade proteases were made in stress buffer. The proteolytic activity of the proteases was subsequently measured before and after a heat incubation step using the DMC assay described in Example 3. The temperature and duration of the heat incubation step were chosen such that the reference protease showed ˜30% residual activity. Stability was measured in Tris-EDTA (50 mM Tris pH9; 1 mM EDTA; 0.005% Tween) buffered condition. % Residual activities were calculated by taking a ratio of the stressed to unstressed activity and multiplying by 100. Stability PIs were obtained by dividing the residual activity of the B. gibsonii-clade protease by that of the parent protease.

B. gibsoni-clade subtilisin BSP-00801, shown on Table 4, was utilized as a parent protease and subjected to further mutagenesis to provide the BSP-00801 variants set forth in Table 5. ND as utilized in the Tables set forth hereinbelow means not determined.

TABLE 3
Cleaning Performance And Stability of B. gibsonii-clade Subtilisins

| | | PI versus Bgi02446 | | | | |
|---|---|---|---|---|---|---|
| B. gibsonii- | | ADW pH 10.5 | ADW pH 10.5 | Stability | Boron-free | Amino |
| clade | | Rinsed | Unrinsed | in | HDL pH 8.2, | Acid |
| Subtilisin | Mutations With Respect To Bgi02446 | Egg stain | Egg Stain | EDTA | BMI Stain | SEQ ID NO: |
| BSP-02389 | N074D-I080V-N085S-E087D-S099R-V102I-S158T-V159I- | 1.3 | 5.3 | 1.5 | 0.9 | 98 |
| | N242D-F257Y | | | | | |
| BSP-02411 | N074D-N085S-E087D-S099R-V102I-S126T-F128A-I190L- | 1.5 | 5.0 | 1.4 | 1.2 | 103 |
| | N242D-F257Y | | | | | |
| BSP-02301 | R027K-S099R-V102I-T114Q-I119V-S126T-F128A-S158T- | 1.4 | 4.9 | 2.7 | 1.0 | 106 |
| | V159I-G160S-T188A-N242D | | | | | |
| BSP-03033 | N074D-I080V-N085S-E087D-S099R-V102I-T114Q-S126T- | 1.4 | 4.7 | 1.1 | 1.0 | 114 |
| | F128A-N242D | | | | | |
| BSP-03027 | R027K-S099R-V102I-S126T-F128A-R143A-S158T-V159I- | 1.2 | 4.6 | 2.2 | 0.7 | 107 |
| | G160S | | | | | |
| BSP-03007 | N074D-I080V-N085S-E087D-S099R-V102I-I190L-N242D- | 1.2 | 4.5 | 1.4 | 0.6 | 102 |
| | F257Y | | | | | |
| BSP-02380 | N074D-I080V-N085S-E087D-S099R-V102I-T114Q-R143A- | 1.3 | 4.2 | 1.0 | 0.9 | 101 |
| | I190L-N242D-F257Y | | | | | |
| BSP-02195 | N074D-S099R-V102I-S126T-F128A-R143A-S158T-N212S | 1.3 | 4.2 | 7.5 | 0.8 | 104 |
| BSP-03024 | N074D-I080V-N085S-E087D-S099R-T114Q-S126T-F128A- | 1.3 | 4.1 | 1.4 | 1.2 | 113 |
| | R143A-N242D | | | | | |
| BSP-02725 | N085S-E087D-S099R-T114Q-I119V-S126T-F128A-R143A- | 1.3 | 4.1 | 1.7 | 0.8 | 111 |
| | G160S-F257Y | | | | | |
| BSP-02227 | N074D-I080V-S099R-V102I-T114Q-S126T-F128A-R143A- | 1.4 | 4.0 | 2.4 | 1.1 | 110 |
| | N212S-N242D | | | | | |
| BSP-02354 | N074D-I080V-N085S-E087D-S099R-V102I-S126T-F128A- | 1.4 | 4.0 | 1.5 | 1.1 | 100 |
| | R143A-I190L-N242D-F257Y | | | | | |
| BSP-02400 | N074D-I080V-S099R-V102I-T114Q-I119V-S126T-F128A- | 1.3 | 3.8 | 3.0 | 0.9 | 105 |
| | S158T-G160S-N212S-F257Y | | | | | |
| BSP-03048 | I080V-N085S-E087D-S099R-V102I-T114Q-I119V-S126T- | 1.3 | 3.7 | 1.6 | 0.8 | 117 |
| | F128A-R143A-N242D | | | | | |
| BSP-02355 | N085S-E087D-S099R-V102I-T114Q-S158T-V159I-G160S- | 1.0 | 3.6 | 3.9 | 0.6 | 99 |
| | N212S-N242D-F257Y | | | | | |
| BSP-02391 | R027K-N074D-S099R-V102I-T114Q-S126T-F128A- | 1.1 | 3.5 | 3.2 | 0.9 | 109 |
| | T188A-I190L-N212S-N242D | | | | | |
| BSP-02412 | R027K-N074D-S099R-V102I-I119V-S126T-F128A-R143A- | 1.3 | 2.9 | 3.1 | 1.1 | 108 |
| | G160S-N212S-N242D | | | | | |
| BSP-01757 | N042T-I080V-N085S-E087D-T114A-F128A-R143Q- | 1.2 | 1.6 | 1.6 | ND | 95 |
| | D144G-S158T-V159I-G160S-N198G | | | | | |
| BSP-01537 | I021V-I080V-N085S-E087D-M117I-F128A-S131T-R143Q- | 1.3 | 1.4 | 1.3 | ND | 94 |
| | D144G-A224V | | | | | |
| BSP-02320 | N074D-I080V-N085S-E087D-S158T-N242D | 1.2 | 1.2 | 1.1 | 1.4 | 97 |
| BSP-01531 | Q001A-I080V-F128A-S131T-R143A-D144G-M169L- | 1.3 | 1.2 | 2.0 | ND | 96 |
| | I190L-S254T-S255N-Q256L-F257Y | | | | | |

TABLE 4
Cleaning Performance And Stability of B. gibsonii-clade Subtilisins

| | | PI versus Bgi02446 | | | | | |
|---|---|---|---|---|---|---|---|
| | | ADW | ADW | | Boron-free | Amino | |
| B. gibsonii- | | pH 10.5 | pH 10.5 | Stabil- | HDL pH | acid | Nucleotide |
| clade | | Rinsed Egg | Unrinsed | ity in | 8.2, BMI | SEQ | SEQ |
| Subtilisin | Mutations With Respect to Bgi02446 | stain | Egg stain | EDTA | Stain | ID NO: | ID NO: |
| BSP-02209 | A037T-S039E-N042T-S099R-V102I-T114Q-S126T- | 1.7 | 7.2 | 7.9 | 1.0 | 118 | — |
| | F128A-N242D | | | | | | |
| BSP-02107 | A037T-S039E-S099R-V102I-T114Q-S126T-F128A- | 1.5 | 4.7 | 3.9 | 1.1 | 116 | — |
| | R143A-N212S-N242D | | | | | | |
| BSP-02233 | A037T-S039E-N074D-S099R-V102I-S126T-F128A- | 1.2 | 2.8 | 3.8 | 1.2 | 117 | — |
| | R143A-N212S-N242D | | | | | | |
| BSP-02235 | A037T-S039E-N042T-N074D-S099R-V102I-S126T- | 1.6 | 5.8 | 3.6 | 1.1 | 26 | 25 |
| | F128A-S158T-N242D | | | | | | |
| BSP-00801 | A037T-S039E-I043V-A047V-I080V-N085S-E087D- | 1.3 | 4.5 | 2.4 | 0.9 | 18 | 15 |
| | S099R-T114A-F128A | | | | | | |
| BSP-02112 | A037T-S039E-I043V-A047V-T055G-S099R-V102I- | 1.3 | 4.1 | 3.5 | 0.8 | 120 | — |
| | T114Q-S126T-F128A-N212S | | | | | | |
| BSP-02508 | A037T-S039E-I043V-A047V-I080V-N085S-E087D- | 1.6 | 5.4 | 2.3 | 1.1 | 42 | 41 |
| | S099R-T114Q-F128A-N242D | | | | | | |
| BSP-02513 | A037T-S039E-I043V-A047V-I080V-N085S-E087D- | 1.4 | 4.7 | 1.7 | 1.2 | 138 | — |
| | S099R-T114A-F128A-N242D | | | | | | |
| BSP-02542 | A037T-S039E-A047V-I080V-N085S-E087D-S099R- | 1.4 | 4.8 | 2.6 | 1.5 | 128 | — |
| | T114Q-F128A-R143A-N242D | | | | | | |
| BSP-02781 | S036A-S039E-I043V-A047V-T055M-I080V-N085S- | 1.3 | 4.4 | 1.8 | 1.1 | 140 | — |
| | E087D-S099R-T114A-F128A | | | | | | |
| BSP-03079 | A037T-S039E-I043V-A047V-I080V-N085S-E087D- | 1.3 | 4.4 | 2.5 | 0.9 | 135 | — |
| | S099R-V102I-T114Q-F128A | | | | | | |
| BSP-02060 | A037T-S039E-I043V-A047V-N074D-N085S-E087D- | 1.4 | 4.6 | 3.8 | 1.3 | 146 | — |
| | S099R-T114A-F128A-S158T-N212S | | | | | | |
| BSP-02132 | A037T-S039E-N074D-I080V-N085S-E087D-S099R- | 1.7 | 5.6 | 4.1 | 1.6 | 121 | — |
| | V102I-T114Q-S126T-F128A-R143A | | | | | | |
| BSP-02203 | A037T-S039E-I043V-A047V-T055G-N074D-I080V- | 1.5 | 5.1 | 3.6 | 1.0 | 24 | 23 |
| | S099R-V102I-T114Q-S126T-F128A | | | | | | |
| BSP-02249 | A037T-S039E-I043V-A047V-N074D-I080V-S099R- | 1.6 | 5.8 | 3.8 | 1.0 | 28 | 27 |
| | V102I-T114Q-S126T-F128A-N242D | | | | | | |
| BSP-02310 | R027K-A037T-S039E-A047V-T055G-T056Y-N074D- | 1.6 | 5.8 | 3.5 | 1.2 | 30 | 29 |
| | S099R-V102I-S126T-F128A-N242D | | | | | | |
| BSP-02443 | A037T-S039E-N042T-A047V-N074D-I080V-N085S- | 1.4 | 4.2 | 4.0 | 1.2 | 124 | — |
| | E087D-S099R-T114Q-F128A-N242D | | | | | | |
| BSP-02471 | A037T-S039E-I043V-A047V-I080V-N085S-E087D- | 1.2 | 4.3 | 2.8 | 1.1 | 155 | — |
| | S099R-T114A-S126T-F128A-N212S | | | | | | |
| BSP-02480 | A037T-S039E-I043V-A047V-I080V-N085S-E087D- | 1.5 | 5.6 | 2.1 | 1.1 | 38 | 37 |
| | S099R-T114A-S126T-F128A-N242D | | | | | | |
| BSP-02507 | A037T-S039E-I043V-A047V-I080V-N085S-E087D- | 1.8 | 6.2 | 2.7 | 1.3 | 40 | 39 |
| | S099R-T114Q-S126T-F128A-R143A | | | | | | |
| BSP-02514 | A037T-S039E-I043V-A047V-I080V-N085S-E087D- | 1.2 | 3.3 | 2.3 | 1.0 | 154 | — |
| | S099R-V102I-T114A-F128A-N212S | | | | | | |
| BSP-02524 | A037T-S039E-N042T-A047V-I080V-N085S-E087D- | 1.5 | 4.3 | 2.3 | 1.2 | 125 | — |
| | S099R-V102I-T114Q-F128A-N242D | | | | | | |
| BSP-02525 | A037T-S039E-I043V-A047V-T056Y-I080V-N085S- | 1.5 | 4.9 | 2.3 | 1.1 | 139 | — |
| | E087D-S099R-T114A-F128A-N242D | | | | | | |
| BSP-02539 | A037T-S039E-I043V-A047V-I080V-N085S-E087D- | 1.3 | 3.8 | 3.1 | 1.2 | 136 | — |
| | S099R-T114Q-F128A-N212S-N242D | | | | | | |
| BSP-02565 | A037T-S039E-A047V-I080V-N085S-E087D-S099R- | 1.5 | 3.5 | 2.4 | 1.4 | 129 | — |
| | V102I-T114Q-F128A-R143A-N242D | | | | | | |
| BSP-02569 | A037T-S039E-I043V-A047V-T055G-I080V-N085S- | 1.4 | 5.6 | 2.5 | 0.9 | 52 | 51 |
| | E087D-S099R-T114Q-S126T-F128A | | | | | | |
| BSP-02677 | R027K-A037T-S039E-A047V-I080V-N085S-E087D- | 1.3 | 3.9 | 3.5 | 0.9 | 127 | — |
| | S099R-T114Q-F128A-N212S-N242D | | | | | | |
| BSP-02768 | T009S-A037T-S039E-I043V-A047V-I080V-N085S- | 1.3 | 3.6 | 2.9 | 0.8 | 156 | — |
| | E087D-S099R-T114A-F128A-V197I | | | | | | |
| BSP-02791 | S039E-I043V-A047V-I080V-N085S-E087D-S099R- | 1.1 | 2.9 | 2.0 | 0.8 | 143 | — |
| | T114A-F128A-N212S-K245L-N246S | | | | | | |
| BSP-02805 | A037T-S039E-I043V-A047V-I080V-N085S-E087D- | 1.4 | 4.3 | 2.3 | 1.0 | 157 | — |
| | S099R-T114A-F128A-I190L-A224V | | | | | | |
| BSP-02814 | S036A-S039E-I043V-A047V-I080V-N085S-E087D- | 1.5 | 4.3 | 1.8 | 1.0 | 141 | — |
| | S099R-T114A-N121S-F128A-R143Q | | | | | | |
| BSP-02828 | S036A-S039E-I043V-A047V-I080V-N085S-E087D- | 1.3 | 4.2 | 4.6 | 0.9 | 142 | — |
| | S099R-T114A-F128A-N212S-S255N | | | | | | |
| BSP-02829 | S039E-I043V-A047V-I080V-N085S-E087D-S099R- | 1.3 | 4.3 | 2.9 | 1.0 | 144 | — |
| | T114A-F128A-R143Q-N242D-N246K | | | | | | |
| BSP-02979 | A037T-S039E-I043V-A047V-N074D-N085S-E087D- | 1.3 | 4.3 | 4.7 | 0.8 | 147 | — |
| | S099R-T114A-S126T-F128A-N212S | | | | | | |
| BSP-03009 | A037T-S039E-I043V-A047V-N074D-I080V-N085S- | 1.2 | 3.5 | 4.3 | 0.9 | 149 | — |
| | E087D-S099R-T114A-F128A-N212S | | | | | | |
| BSP-03070 | A037T-S039E-N042T-A047V-I080V-N085S-E087D- | 1.1 | 3.2 | 2.7 | 0.8 | 153 | — |
| | S099R-T114A-F128A-S158T-N212S | | | | | | |
| BSP-03098 | V004I-A037T-S039E-I043V-A047V-I080V-N085S- | 1.4 | 4.5 | 2.2 | 1.1 | 158 | — |
| | E087D-S099R-T114A-F128A-N182S | | | | | | |
| BSP-02035 | A037T-S039E-I043V-A047V-N074D-N085S-E087D- | 1.4 | 4.8 | 3.6 | 1.1 | 145 | — |
| | S099R-T114A-S126T-F128A-S158T-N212S | | | | | | |
| BSP-02043 | A037T-S039E-I043V-A047V-N074D-I080V-N085S- | 1.2 | 3.9 | 4.0 | 1.2 | 148 | — |
| | E087D-S099R-T114A-F128A-S158T-N212S | | | | | | |
| BSP-02052 | A037T-S039E-I043V-A047V-N074D-N085S-E087D- | 1.2 | 3.0 | 4.2 | 1.5 | 150 | — |
| | S099R-T114A-F128A-N212S-N242Q-K245L | | | | | | |
| BSP-02073 | A037T-S039E-I043V-A047V-N074D-I080V-N085S- | 1.3 | 3.6 | 3.7 | 1.3 | 152 | — |
| | E087D-S099R-T114A-F128A-N212S-N242Q | | | | | | |
| BSP-02086 | A037T-S039E-I043V-A047V-N074D-I080V-N085S- | 1.4 | 3.8 | 2.9 | 1.3 | 151 | — |
| | E087D-S099R-T114A-F128A-N212S-K245L | | | | | | |
| BSP-02364 | A037T-S039E-A047V-S099R-V102I-T114A-I119V- | 1.3 | 2.7 | 4.0 | 1.1 | 119 | — |
| | S126T-F128A-R143A-S158T-G160S-N212S | | | | | | |
| BSP-02423 | A037T-S039E-N042T-A047V-T056Y-N074D-I080V- | 1.5 | 6.5 | 4.0 | 1.3 | 32 | 31 |
| | N085S-E087D-S099R-T114Q-S126T-F128A | | | | | | |
| BSP-02438 | S024F-A037T-S039E-A047V-N074D-I080V-N085S- | 1.3 | 4.8 | 5.0 | 1.0 | 122 | — |
| | E087D-S099R-T114Q-S126T-F128A-N212S | | | | | | |
| BSP-02500 | A037T-S039E-I043V-A047V-I080V-N085S-E087D- | 1.7 | 5.9 | 2.0 | 1.2 | 130 | — |
| | S099R-T114Q-S126T-F128A-R143A-N242D | | | | | | |
| BSP-02504 | A037T-S039E-I043V-A047V-I080V-N085S-E087D- | 1.7 | 5.7 | 2.9 | 1.1 | 137 | — |
| | S099R-T114A-S126T-F128A-A237T-N242D | | | | | | |
| BSP-02521 | A037T-S039E-I043V-A047V-I080V-N085S-E087D- | 1.5 | 4.8 | 2.1 | 1.2 | 133 | — |
| | S099R-V102I-T114Q-S126T-F128A-N242D | | | | | | |
| BSP-02537 | A037T-S039E-I043V-A047V-T055G-I080V-N085S- | 1.4 | 4.6 | 2.1 | 1.2 | 134 | — |
| | E087D-S099R-V102I-T114Q-F128A-R143A | | | | | | |
| BSP-02540 | A037T-S039E-N042T-A047V-T055G-I080V-N085S- | 1.2 | 3.0 | 3.2 | 1.1 | 126 | — |
| | E087D-S099R-T114Q-F128A-N212S-N242D | | | | | | |

TABLE 5
Cleaning Performance And Stability of B. gibsonii-clade Subtilisins

| | | | PI versus BSP-00801 | | | | | |
|---|---|---|---|---|---|---|---|---|
| | | | ADW pH | ADW pH | | Boron-free | Amino | |
| B.gibsonii- | | | 10.5- | 10.5- | Stabil- | HDL pH | acid | Nucleotide |
| clade | Mutations With | Mutations With | Rinsed | Unrinsed | ity in | 8.2, BMI | SEQ | SEQ |
| Subtilisin | Respect to BSP-00801 | Respect to Bgi02446 | Egg Stain | Egg stain | EDTA | stain | ID NO: | ID NO: |
| BSP-00801 | Parent | A037T-S039E-I043V-A047V- | 1.0 | 1.0 | 1.0 | 1.0 | 18 | 15 |
| | | I080V-N085S-E087D-S099R- | | | | | | |
| | | T114A-F128A | | | | | | |
| BSP-02470 | N242D | A037T-S039E-I043V-A047V- | 1.1 | 1.1 | 1.1 | 1.1 | 173 | — |
| | | I080V-N085S-E087D-S099R- | | | | | | |
| | | T114A-F128A-N242D | | | | | | |
| BSP-02508 | A114Q-N242D | A037T-S039E-I043V-A047V- | 1.2 | 1.2 | 1.1 | 1.1 | 42 | 41 |
| | | I080V-N085S-E087D-S099R- | | | | | | |
| | | T114Q-F128A-N242D | | | | | | |
| BSP-02480 | S126T-N242D | A037T-S039E-I043V-A047V- | 1.1 | 1.2 | 1.1 | 1.1 | 38 | 37 |
| | | I080V-N085S-E087D-S099R- | | | | | | |
| | | T114A-S126T-F128A-N242D | | | | | | |
| BSP-02525 | T056Y-N242D | A037T-S039E-I043V-A047V- | 1.1 | 1.1 | 1.1 | 1.1 | 139 | — |
| | | T056Y-I080V-N085S-E087D- | | | | | | |
| | | S099R-T114A-F128A-N242D | | | | | | |
| BSP-02507 | A114Q-S126T-R143A | A037T-S039E-I043V-A047V- | 1.3 | 1.3 | 1.3 | 1.3 | 40 | 39 |
| | | I080V-N085S-E087D-S099R- | | | | | | |
| | | T114Q-S126T-F128A-R143A | | | | | | |
| BSP-02569 | T055G-A114Q-S126T | A037T-S039E-I043V-A047V- | 1.1 | 1.2 | 1.2 | 0.9 | 52 | 51 |
| | | T055G-I080V-N085S-E087D- | | | | | | |
| | | S099R-T114Q-S126T-F128A | | | | | | |
| BSP-02096 | N074D-S158T-N212S | A037T-S039E-I043V-A047V- | 1.1 | 1.1 | 1.6 | 1.1 | 163 | — |
| | | N074D-I080V-N085S-E087D- | | | | | | |
| | | S099R-T114A-F128A-S158T- | | | | | | |
| | | N212S | | | | | | |
| BSP-02504 | S126T-A237T-N242D | A037T-S039E-I043V-A047V- | 1.3 | 1.2 | 1.4 | 1.1 | 137 | — |
| | | I080V-N085S-E087D-S099R- | | | | | | |
| | | T114A-S126T-F128A-A237T- | | | | | | |
| | | N242D | | | | | | |
| BSP-02635 | N074D-S126T-N242D | A037T-S039E-I043V-A047V- | 1.1 | 1.2 | 1.6 | 1.1 | 56 | 55 |
| | | N074D-I080V-N085S-E087D- | | | | | | |
| | | S099R-T114A-S126T-F128A- | | | | | | |
| | | N242D | | | | | | |
| | | R027K-A037T-S039E-I043V- | | | | | | |
| BSP-02676 | R027K-S126T-N242D | A047V-I080V-N085S-E087D- | 1.2 | 1.1 | 1.2 | 1.1 | 60 | 59 |
| | | S099R-T114A-S126T-F128A- | | | | | | |
| | | N242D | | | | | | |
| BSP-02684 | T056Y-S126T-R143A | A037T-S039E-I043V-A047V- | 1.1 | 1.1 | 1.2 | 1.2 | 62 | 61 |
| | | T056Y-I080V-N085S-E087D- | | | | | | |
| | | S099R-T114A-S126T-F128A- | | | | | | |
| | | R143A | | | | | | |
| BSP-02903 | N121S-N246S-S255N | A037T-S039E-I043V-A047V- | 1.1 | 1.1 | 1.2 | 1.0 | 196 | — |
| | | I080V-N085S-E087D-S099R- | | | | | | |
| | | T114A-N121S-F128A-N246S- | | | | | | |
| | | S255N | | | | | | |
| BSP-02542 | V043I-A114Q-R143A-N242D | A037T-S039E-A047V-I080V- | 1.1 | 1.0 | 1.2 | 1.4 | 128 | — |
| | | N085S-E087D-S099R-T114Q- | | | | | | |
| | | F128A-R143A-N242D | | | | | | |
| BSP-02060 | N074D-V080I-S158T-N212S | A037T-S039E-I043V-A047V- | 1.0 | 1.0 | 1.8 | 1.2 | 146 | — |
| | | N074D-N085S-E087D-S099R- | | | | | | |
| | | T114A-F128A-S158T-N212S | | | | | | |
| BSP-02521 | V102I-A114Q-S126T-N242D | A037T-S039E-I043V-A047V- | 1.1 | 1.0 | 1.1 | 1.2 | 133 | — |
| | | I080V-N085S-E087D-S099R- | | | | | | |
| | | V102I-T114Q-S126T-F128A- | | | | | | |
| | | N242D | | | | | | |
| BSP-02983 | T056Y-A114Q-S126T-N242D | A037T-S039E-I043V-A047V- | 1.1 | 1.3 | 1.2 | 1.0 | 66 | 65 |
| | | T056Y-I080V-N085S-E087D- | | | | | | |
| | | S099R-T114Q-S126T-F128A- | | | | | | |
| | | N242D | | | | | | |
| BSP-03096 | N074D-A114Q-I119V-S126T | A037T-S039E-I043V-A047V- | 1.1 | 1.0 | 1.6 | 1.1 | 197 | — |
| | | N074D-I080V-N085S-E087D- | | | | | | |
| | | S099R-T114Q-I119V-S126T- | | | | | | |
| | | F128A | | | | | | |
| BSP-02445 | T055G-V102I-S126T-S158T | A037T-S039E-I043V-A047V- | 1.0 | 1.0 | 1.2 | 0.8 | 36 | 35 |
| | | T055G-I080V-N085S-E087D- | | | | | | |
| | | S099R-V102I-T114A-S126T- | | | | | | |
| | | F128A-S158T | | | | | | |
| BSP-02472 | S126T-R143A-S158T-N242D | A037T-S039E-I043V-A047V- | 1.1 | 1.1 | 1.3 | 1.4 | 174 | — |
| | | I080V-N085S-E087D-S099R- | | | | | | |
| | | T114A-S126T-F128A-R143A- | | | | | | |
| | | S158T-N242D | | | | | | |
| BSP-02552 | N042T-R044S-N074D-S126T | A037T-S039E-N042T-I043V- | 1.2 | 1.2 | 1.8 | 1.2 | 46 | 45 |
| | | R044S-A047V-N074D-I080V- | | | | | | |
| | | N085S-E087D-S099R-T114A- | | | | | | |
| | | S126T-F128A | | | | | | |
| BSP-02567 | N042T-R044S-S126T-N242D | A037T-S039E-N042T-I043V- | 1.2 | 1.2 | 1.3 | 1.1 | 50 | 49 |
| | | R044S-A047V-I080V-N085S- | | | | | | |
| | | E087D-S099R-T114A-S126T- | | | | | | |
| | | F128A-N242D | | | | | | |
| BSP-02660 | V102I-I119V-S126T-F257Y | A037T-S039E-I043V-A047V- | 1.1 | 1.0 | 1.3 | 0.9 | 183 | — |
| | | I080V-N085S-E087D-S099R- | | | | | | |
| | | V102I-T114A-I119V-S126T- | | | | | | |
| | | F128A-F257Y | | | | | | |
| BSP-02890 | N074D-V102I-N121S-R143Q | A037T-S039E-I043V-A047V- | 1.1 | 1.1 | 1.3 | 1.0 | 195 | — |
| | | N074D-I080V-N085S-E087D- | | | | | | |
| | | S099R-V102I-T114A-N121S- | | | | | | |
| | | F128A-R143Q | | | | | | |
| BSP-02035 | N074D-V080I-S126T-S158T- | A037T-S039E-I043V-A047V- | 1.1 | 1.0 | 1.8 | 1.1 | | |
| | N212S | N074D-N085S-E087D-S099R- | | | | | | |
| | | T114A-S126T-F128A-S158T- | | | | | | |
| | | N212S | | | | | | |
| BSP-02802 | S036A-T037A-N074D-A224V- | S036A-S039E-I043V-A047V- | 1.0 | 1.0 | 2.6 | 1.3 | 188 | — |
| | S255N | N074D-I080V-N085S-E087D- | | | | | | |
| | | S099R-T114A-F128A-A224V- | | | | | | |
| | | S255N | | | | | | |
| BSP-02446 | T055G-N074D-A114Q-S126T- | A037T-S039E-I043V-A047V- | 1.0 | 1.2 | 1.6 | 1.1 | 170 | — |
| | N242D | T055G-N074D-I080V-N085S- | | | | | | |
| | | E087D-S099R-T114Q-S126T- | | | | | | |
| | | F128A-N242D | | | | | | |
| BSP-02456 | T055G-V102I-A114Q-S126T- | A037T-S039E-I043V-A047V- | 1.1 | 1.0 | 1.0 | 1.2 | 171 | — |
| | R143A | T055G-I080V-N085S-E087D- | | | | | | |
| | | S099R-V102I-T114Q-S126T- | | | | | | |
| | | F128A-R143A | | | | | | |
| BSP-02467 | V102I-A114Q-S126T-R143A- | A037T-S039E-I043V-A047V- | 1.1 | 1.1 | 1.4 | 1.1 | 172 | — |
| | N242D | I080V-N085S-E087D-S099R- | | | | | | |
| | | V102I-T114Q-S126T-F128A- | | | | | | |
| | | R143A-N242D | | | | | | |
| BSP-02550 | T056Y-A114Q-S126T-S158T- | A037T-S039E-I043V-A047V- | 1.2 | 1.2 | 1.1 | 1.1 | 44 | 43 |
| | N242D | T056Y-I080V-N085S-E087D- | | | | | | |
| | | S099R-T114Q-S126T-F128A- | | | | | | |
| | | S158T-N242D | | | | | | |
| BSP-02579 | T056Y-A114Q-I119V-S126T- | A037T-S039E-I043V-A047V- | 1.1 | 1.2 | 1.4 | 0.7 | 178 | — |
| | F257Y | T056Y-I080V-N085S-E087D- | | | | | | |
| | | S099R-T114Q-I119V-S126T- | | | | | | |
| | | F128A-F257Y | | | | | | |
| BSP-03095 | T056Y-A114Q-I119V-S126T- | A037T-S039E-I043V-A047V- | 1.2 | 1.1 | 1.1 | 1.1 | 68 | 67 |
| | N242D | T056Y-I080V-N085S-E087D- | | | | | | |
| | | S099R-T114Q-I119V-S126T- | | | | | | |
| | | F128A-N242D | | | | | | |
| BSP-02023 | N074D-S126T-S158T-N212S- | A037T-S039E-I043V-A047V- | 1.1 | 1.1 | 1.7 | 1.1 | 160 | — |
| | N242Q | N074D-I080V-N085S-E087D- | | | | | | |
| | | S099R-T114A-S126T-F128A- | | | | | | |
| | | S158T-N212S-N242Q | | | | | | |
| BSP-02733 | N074D-S126T-T188A-I190L- | A037T-S039E-I043V-A047V- | 1.1 | 1.1 | 2.3 | 1.1 | 186 | — |
| | F257Y | N074D-I080V-N085S-E087D- | | | | | | |
| | | S099R-T114A-S126T-F128A- | | | | | | |
| | | T188A-I190L-F257Y | | | | | | |
| BSP-02795 | V004I-T009S-A224V-K245L- | V004I-T009S-A037T-S039E- | 1.1 | 1.0 | 1.3 | 0.8 | 64 | 63 |
| | S255N | I043V-A047V-I080V-N085S- | | | | | | |
| | | E087D-S099R-T114A-F128A- | | | | | | |
| | | A224V-K245L-S255N | | | | | | |
| BSP-02138 | V043I-T056Y-A114Q-A128F- | A037T-S039E-A047V-T056Y- | 1.1 | 1.4 | 1.3 | 1.1 | 166 | — |
| | R143A-N242D | I080V-N085S-E087D-S099R- | | | | | | |
| | | T114Q-R143A-N242D | | | | | | |
| BSP-02423 | N042T-V043I-T056Y-N074D- | A037T-S039E-N042T-A047V- | 1.1 | 1.4 | 1.9 | 1.2 | 32 | 31 |
| | A114Q-S126T | T056Y -N074D-I080V-N085S- | | | | | | |
| | | E087D-S099R -T114Q-S126T- | | | | | | |
| | | F128A | | | | | | |
| BSP-02058 | N074D-V080I-S126T-S158T- | A037T-S039E-I043V-A047V- | 1.1 | 1.1 | 1.9 | 1.0 | 161 | — |
| | N212S-N242Q | N074D -N085S-E087D-S099R- | | | | | | |
| | | T114A-S126T -F128A-S158T- | | | | | | |
| | | N212S-N242Q | | | | | | |
| BSP-02098 | N074D-V080I-S126T-S158T- | A037T-S039E-I043V-A047V- | 1.1 | 1.2 | 1.7 | 1.0 | 165 | — |
| | N212S-K245L | N074D -N085S-E087D-S099R- | | | | | | |
| | | T114A-S126T-F128A-S158T- | | | | | | |
| | | N212S-K245L | | | | | | |
| BSP-02557 | A114P-S126T-R143T-S158T- | A037T-S039E-I043V-A047V- | 1.2 | 1.3 | 1.4 | 1.3 | 175 | — |
| | N212K-N242D | I080V-N085S-E087D-S099R- | | | | | | |
| | | T114P-S126T-F128A-R143T- | | | | | | |
| | | S158T-N212K-N242D | | | | | | |
| BSP-02563 | T056Y-N074D-A114Q-S126T- | A037T-S039E-I043V-A047V- | 1.0 | 1.1 | 1.8 | 1.4 | 177 | — |
| | R143A-N242D | T056Y-N074D-I080V-N085S- | | | | | | |
| | | E087D-S099R-T114Q-S126T- | | | | | | |
| | | F128A-R143A-N242D | | | | | | |
| BSP-02593 | T055G-T056Y-N074D-V102I- | A037T-S039E-I043V-A047V- | 1.0 | 1.1 | 1.5 | 1.1 | 179 | — |
| | A114Q-S126T | T055G-T056Y-N074D-I080V- | | | | | | |
| | | N085S-E087D-S099R-V102I- | | | | | | |
| | | T114Q-S126T-F128A | | | | | | |
| BSP-02748 | S036A-T037N-N242D-K245L- | S036A-A037N-S039E-I043V- | 1.2 | 1.0 | 1.2 | 0.9 | 187 | — |
| | N246S-S255N | A047V-I080V-N085S-E087D- | | | | | | |
| | | S099R-T114A-F128A-N242D- | | | | | | |
| | | K245L-N246S-S255N | | | | | | |
| BSP-02094 | N074D-S126T-S158T-N212S- | A037T-S039E-I043V-A047V- | 1.0 | 1.0 | 1.6 | 1.0 | 162 | — |
| | N242Q-K245L | N074D-I080V-N085S-E087D- | | | | | | |
| | | S099R-T114A-S126T-F128A- | | | | | | |
| | | S158T-N212S-N242Q-K245L | | | | | | |
| BSP-02605 | I119V-S126T-S158T-G160S- | A037T-S039E-I043V-A047V- | 1.1 | 1.1 | 1.2 | 1.1 | 180 | — |
| | N242D-F257Y | I080V-N085S-E087D-S099R- | | | | | | |
| | | T114A-I119V-S126T-F128A- | | | | | | |
| | | S158T-G160S-N242D-F257Y | | | | | | |
| BSP-02885 | N121S-N182S-I190L-K245L- | A037T-S039E-I043V-A047V- | 1.1 | 1.0 | 1.2 | 1.0 | 193 | — |
| | N246S-S255N | I080V-N085S-E087D-S099R- | | | | | | |
| | | T114A-N121S-F128A-N182S- | | | | | | |
| | | I190L-K245L-N246S-S255N | | | | | | |
| BSP-02132 | V043I-V047A-N074D-V102I- | A037T-S039E-N074D-I080V- | 1.3 | 1.2 | 2.0 | 1.6 | 121 | — |
| | A114Q-S126T-R143A | N085S-E087D-S099R-V102I- | | | | | | |
| | | T114Q-S126T-F128A-R143A | | | | | | |
| BSP-02203 | T055G-N074D-S085N-D087E- | A037T-S039E-I043V-A047V- | 1.1 | 1.1 | 1.7 | 0.9 | 24 | 23 |
| | V102I-A114Q-S126T | T055G-N074D-I080V-S099R- | | | | | | |
| | | V102I-T114Q-S126T-F128A | | | | | | |
| BSP-02249 | N074D-S085N-D087E-V102I- | A037T-S039E-I043V-A047V- | 1.2 | 1.3 | 1.9 | 1.0 | 28 | 27 |
| | A114Q-S126T-N242D | N074D-I080V-S099R-V102I- | | | | | | |
| | | T114Q-S126T-F128A-N242D | | | | | | |
| BSP-02106 | V043I-N074D-V102I-A114Q- | A037T-S039E-A047V-N074D- | 1.2 | 1.2 | 1.8 | 1.0 | 22 | 21 |
| | S126T-S158T-N242D | I080V-N085S-E087D-S099R- | | | | | | |
| | | V102I-T114Q-S126T-F128A- | | | | | | |
| | | S158T-N242D | | | | | | |
| BSP-02559 | N042T-V043I-N074D-V102I- | A037T-S039E-N042T-A047V- | 1.1 | 1.1 | 1.8 | 1.1 | 176 | — |
| | A114Q-S126T-N242D | N074D-I080V-N085S-E087D- | | | | | | |
| | | S099R-V102I-T114Q-S126T- | | | | | | |
| | | F128A-N242D | | | | | | |
| BSP-02097 | N074D-V080I-S126T-S158T- | A037T-S039E-I043V-A047V- | 1.0 | 1.1 | 1.7 | 1.1 | 1.6 | — |
| | N212S-N242Q-K245L | N074D-N085S-E087D-S099R- | | | | | | |
| | | T114A-S126T-F128A-S158T- | | | | | | |
| | | N212S-N242Q-K245L | | | | | | |
| BSP-02848 | T009S-S036A-T037A-R143Q- | T009S-S036A-S039E-I043V- | 1.1 | 1.1 | 1.5 | 1.1 | 189 | — |
| | N182S-T188A-I190L | A047V-I080V-N085S-E087D- | | | | | | |
| | | S099R-T114A-F128A-R143Q- | | | | | | |
| | | N182S-T188A-I190L | | | | | | |
| BSP-02422 | T055G-N074D-V102I-A114Q- | A037T-S039E-I043V-A047V- | | | | | | |
| | S126T-S158T-N242D | T055G-N074D-I080V-N085S- | 1.1 | 1.1 | 1.6 | 1.1 | 168 | — |
| | | E087D-S099R-V102I-T114Q- | | | | | | |
| | | S126T-F128A-S158T-N242D | | | | | | |
| BSP-02427 | T055G-N074D-A114Q-S126T- | A037T-S039E-I043V-A047V- | 1.1 | 1.1 | 2.1 | 1.0 | 169 | — |
| | R143A-S158T-N212S | T055G-N074D-I080V-N085S- | | | | | | |
| | | E087D-S099R-T114Q-S126T- | | | | | | |
| | | F128A-R143A-S158T-N212S | | | | | | |
| BSP-02862 | R143Q-N212S-A224V-Y232N- | A037T-S039E-I043V-A047V- | 1.1 | 1.0 | 1.0 | 1.2 | 191 | — |
| | K245L-N246S-S255N | I080V-N085S-E087D-S099R- | | | | | | |
| | | T114A-F128A-R143Q-N212S- | | | | | | |
| | | A224V-Y232N-K245L-N246S- | | | | | | |
| | | S255N | | | | | | |
| BSP-02887 | V004I-T009S-N121S-R143Q- | V004I-T009S-A037T-S039E- | 1.2 | 1.1 | 1.3 | 1.0 | 194 | — |
| | A224I-R231K-K245L | I043V-A047V-I080V-N085S- | | | | | | |
| | | E087D-S099R-T114A-N121S- | | | | | | |
| | | F128A-R143Q-A224I-R231K- | | | | | | |
| | | K245L | | | | | | |
| BSP-02689 | R027K-V043I-A114Q-I119V- | R027K-A037T-S039E-A047V- | 1.2 | 1.0 | 1.4 | 0.9 | 185 | — |
| | S126T-G160S-N242D-F257Y | I080V-N085S-E087D-S099R- | | | | | | |
| | | T114Q-I119V-S126T-F128A- | | | | | | |
| | | G160S-N242D-F257Y | | | | | | |
| BSP-02611 | R027K-T056Y-A114Q-S126T- | R027K-A037T-S039E-I043V- | 1.1 | 1.1 | 1.3 | 1.2 | 54 | 53 |
| | S158T-G160S-N242D-F257Y | A047V-T056Y-I080V-N085S- | | | | | | |
| | | E087D-S099R-T114Q-S126T- | | | | | | |
| | | F128A-S158T-G160S-N242D- | | | | | | |
| | | F257Y | | | | | | |
| BSP-02620 | R027K-T056Y-V102I-A114Q- | R027K-A037T-S039E-I043V- | 1.1 | 1.0 | 1.4 | 0.8 | 181 | — |
| | S126T-T188A-I190L-F257Y | A047V-T056Y-I080V-N085S- | | | | | | |
| | | E087D-S099R-V102I-T114Q- | | | | | | |
| | | S126T-F128A-T188A-I190L- | | | | | | |
| | | F257Y | | | | | | |
| BSP-02622 | R027K-T056N-A114P-S126T- | R027K-A037T-S039E-I043V- | 1.1 | 1.2 | 1.7 | 1.2 | 182 | — |
| | R143T-V159I-N212T-F257Y | A047V-T056N-I080V-N085S- | | | | | | |
| | | E087D-S099R-T114P-S126T- | | | | | | |
| | | F128A-R143T-V159I-N212T- | | | | | | |
| | | F257Y | | | | | | |
| BSP-02681 | T056Y-A114Q-I119V-S126T- | A037T-S039E-I043V-A047V- | 1.2 | 1.0 | 1.5 | 1.0 | 184 | — |
| | S158T-V159I-G160S-F257Y | T056Y-I080V-N085S-E087D- | | | | | | |
| | | S099R-T114Q-I119V-S126T- | | | | | | |
| | | F128A-S158T-V159I-G160S- | | | | | | |
| | | F257Y | | | | | | |
| BSP-02429 | T055G-T056Y-N074D-V102I- | A037T-S039E-I043V-A047V- | 1.0 | 1.1 | 2.0 | 1.0 | 34 | 33 |
| | A114Q-S126T-N242D | T055G-T056Y-N074D-I080V- | | | | | | |
| | | N085S-E087D-S099R-V102I- | | | | | | |
| | | T114Q-S126T-F128A-N242D | | | | | | |
| BSP-02658 | R027K-V043I-T056Y-N074D- | R027K-A037T-S039E-A047V- | 1.1 | 1.3 | 1.8 | 1.0 | 58 | 57 |
| | A114Q-I119V-S126T-N242D- | T056Y-N074D-I080V-N085S- | | | | | | |
| | F257Y | E087D-S099R-T114Q-I119V- | | | | | | |
| | | S126T-F128A-N242D-F257Y | | | | | | |
| BSP-02235 | N042T-V043I-V047A-N074D- | A037T-S039E-N042T-N074D- | 1.2 | 1.3 | 1.8 | 1.1 | 26 | 25 |
| | V080I-S085N-D087E-V102I- | S099R-V102I-S126T-F128A- | | | | | | |
| | A114T-S126T-S158T-N242D | S158T-N242D | | | | | | |
| BSP-02310 | R027K-V043I-V047A-T055G- | R027K-A037T-S039E-A047V- | 1.2 | 1.3 | 1.7 | 1.1 | 30 | 29 |
| | T056Y-N074D-V080I-S085N- | T055G-T056Y-N074D-S099R- | | | | | | |
| | D087E-V102I-A114T-S126T- | V102I-S126T-F128A-N242D | | | | | | |
| | N242D | | | | | | | |
| BSP-02872 | T037A-A224V-N242D-N246S- | S039E-I043V-A047V-I080V- | 0.7 | 0.7 | 1.2 | 0.9 | 192 | — |
| | S255N | N085S-E087D-S099R-T114A- | | | | | | |
| | | F128A-A224V-N242D-N246S- | | | | | | |
| | | S255N | | | | | | |
| BSP-03159 | N074D-V102I-R143Q-S158T- | A037T-S039E-I043V-A047V- | 1.1 | 1.0 | 1.9 | 1.1 | 70 | 69 |
| | A224V-S255N | N074D-I080V-N085S-E087D- | | | | | | |
| | | S099R-V102I-T114A-F128A- | | | | | | |
| | | R143Q-S158T-A224V-S255N | | | | | | |
| BSP-02861 | P054S-T056Y-A057Q-N074D- | A037T-S039E-I043V-A047V- | 1.1 | 1.0 | 1.6 | 1.2 | 190 | — |
| | N121S-S158T-A224V-S255N | P054S-T056Y-A057Q-N074D- | | | | | | |
| | | I080V-N085S-E087D-S099R- | | | | | | |
| | | T114A-N121S-F128A-S158T- | | | | | | |
| | | A224V-S255N | | | | | | |
| BSP-02345 | N042T-R044S-N074D-V102I- | A037T-S039E-N042T-I043V- | 1.1 | 1.2 | 2.2 | 1.1 | 167 | — |
| | A114T-S126T-S158T-G160S- | R044S-A047V-N074D-I080V- | | | | | | |
| | N242D | N085S-E087D-S099R-V102I- | | | | | | |
| | | S126T-F128A-S158T-G160S- | | | | | | |
| | | N242D | | | | | | |

### Example 5

### Crystallographic Structure of B. gibsoni-Clade Subtilisin BSP-00801

B. gibsoni-clade subtilisin BSP-00801 was produced in accordance with the process set forth in Examples 1 and 2, under the control of B. lentus pro-sequence.

For the purification of B. gibsoni-clade subtilisin BSP-00801, culture supernatant was dialyzed against 25 mM MES, pH 5.4 with 1 mM calcium chloride. The sample was then filtered and applied to an anion exchange chromatography column (Source 15S, GE Healthcare, US) equilibrated with 25 mM MES pH 5.4 and 1 mM calcium chloride. Protein was eluted with a linear sodium chloride gradient. Protein of interest was isolated and propylene glycol was added to 40% final concentration.

The three-dimensional structure of B. gibsoni-clade subtilisin BSP-00801 (SEQ ID NO:18) was determined using X-ray crystallography. The structure of BSP-00801 subtilisin was determined in the space group P6₁22 having 1 molecule in the asymmetric unit with unit cell dimension a=75.19, b=75.19, c=202.3 Å and γ=120⁰ to a resolution of 2.5 Å. The crystals were obtained by the hanging drop method starting with a 22.2 mg/mL protein in 25 mM MES pH 5.4+1 mM Calcium chloride+40% Propylene Glycol solution. The reservoir solution contained 2.0 M Ammonium sulfate+5% iso-Propanol. Equal amounts of protein solution and reservoir were mixed and allowed to equilibrate. Data was collected on a Bruker X8 Proteum system (Bruker Axis Inc., Madison, WI, USA). The structure was determined using molecular replacement with the coordinates of B. lentus subtilisin pdb entry 1JEA as a starting model. The coordinates for BSP-00801 were fitted in the resulting electron density using the program COOT (Emsley, P et al. Acta Cryst. D66 486-501, 2010). After fitting and refitting adjustments, the coordinates were refined using the REFMAC program with standard defaults in the CCP4 software suite. The final model had good stereochemistry and an R-work of 0.19 and R-free of 0.25 for all data to 2.5 A.

The structure of the B. gibsonii-clade subtilisin BSP-00801 is compared with the main chain fold of another commercial subtilisin: subtilisin BPN′ and B. lentus subtilisin in FIG. 1. The structures are homologous having a common catalytic triad which in the case of BSP-00801 corresponds to residues Asp 32, His 62 and Ser 215.

The substitutions present in the BSP-00801 variant with respect to the parent B. gibsonii-clade Bgi02446 (A37T/N; S39E; I43V; A47V; 180V; N85S; E87D; S99R; T114A; F128A) are shown in FIG. 2. These substitutions are found to be distributed in two regions of the structure. Like most subtilisins, BSP-00801 has two calcium binding sites. The first site is characterized as having very high affinity and has been associated with the stability of the molecule in detergents. Eight of the ten substitutions found in BSP-00801 when compared to Bgi02446 are situated either on the loop that forms the tight binding site, or along a surface extending from this calcium site, as shown in FIG. 2. The surface just mentioned consists of a series of external loops which include the following residues: 25-27, 37-43, 47-48, 55-57, 72-74, 85-87 and 114-117. Three loops for the substrate binding region corresponding to sub-sites S4-S1, they correspond to the following residues in BSP-00801: 97-102, 124-130 and 154-161. The other two substitutions, S99R and F128A, are found on two of these loops, as shown in FIG. 3.

Additional sites have been identified that further enhance the performance of the B. gibsonii-clade subtilisin BSP-00801 variants and six of these sites are found to be equally distributed along the two regions previously identified. FIG. 4 is in the same orientation as FIG. 2 to facilitate comparison. In FIG. 4, the ten original substitutions are shown in light gray and the side chains of the residues at the six additional sites are shown as black sticks. The sites N42T, S56Y and N74D are situated along the same surface where eight of the BSP-00801 substitutions are found. FIG. 5 shows in a similar view as FIG. 3 that allows viewing of the N74D site for comparison with three additional sites V102I, S126T and S158T, which are found on the loops forming the sub-sites S4-S1.

One additional substitution, not found in the regions mentioned above, is shown in FIG. 13. In this figure, the location of the N242D substitution can be seen relative to some of the other sites which are visible in this orientation. Surprisingly, this site is situated far removed from the substrate binding surface and the surface near the first calcium binding site. It is found closer to the second calcium binding site.

### Example 6

### Identification of Homologous Proteases

The amino acid sequence (269 residues) of the predicted mature form of BSP- 00801 (SEQ ID NO: 18) was subjected to a BLAST search (Altschul et al., Nucleic Acids Res, 25:3389-402, 1997) against the NCBI non-redundant protein database. A similar search was run against the Genome Quest Patent database with search parameters set to default values using SEQ TD NO:18 as the query sequence. Subsets of the search results are shown in Tables 6-1 and 6-2. Percent identity (PID) for both search sets was defined as the number of identical residues divided by the number of aligned residues in the pairwise alignment. The column labeled “Sequence Length” refers to the length (in amino acids) of the protein sequences associated with the listed Accession Nos., while the column labeled “Aligned Length” refers to the length (in amino acids) of the aligned protein sequences, which was used for the PID calculation.

TABLE 6-1
PID Shared by BSP-00801 with Entries in NCBI non-Redundant Protein Database

| | | | Sequence | Alignment |
|---|---|---|---|---|
| Accession No. | PID | Organism | Length | Length |
| AGS78407 | 96.3 | Bacillus gibsonii | 375 | 269 |
| WP_054704207 | 80.6 | Bacillus sp. JCM 19041 | 375 | 268 |
| P41362 | 79.5 | Bacillus clausii | 380 | 268 |
| P27693 | 79.1 | Bacillus alcalophilus | 380 | 268 |
| P29600 | 78.7 | Bacillus lentus | 269 | 268 |
| AFR78140 | 78.7 | Synthetic construct | 269 | 268 |
| BAA06157 | 78.0 | Bacillus sp. Sendai | 382 | 268 |
| WP_054794820 | 77.6 | Bacillus sp. JCM 19035 | 376 | 268 |
| GAF11832 | 77.2 | Bacillus sp. JCM 19045 | 403 | 268 |
| AFK08970 | 77.2 | Bacillus lehensis | 378 | 268 |
| BAA25184 | 77.2 | Bacillus sp. AprN | 379 | 269 |
| GAF21819 | 76.9 | Bacillus sp. JCM 19047 | 379 | 268 |
| ADK62564 | 65.8 | Bacillus sp. B001 | 375 | 269 |
| BAA02443 | 63.8 | Bacillus halodurans | 361 | 268 |
| BAA05540 | 63.8 | Bacillus sp. AprM | 361 | 269 |
| ADD64465 | 63.1 | Bacillus sp. JB99 | 361 | 268 |
| ABI26631 | 62.7 | Bacillus clausii | 361 | 268 |
| GAE36608 | 61.3 | Bacillus akibai JCM 9157 | 373 | 266 |
| BAA06158 | 60.7 | Bacillus sp. ALP I | 374 | 272 |
| WP_012957236.1 | 60.7 | Bacillus pseudofirmus OF4 | 374 | 272 |
| AAC43580 | 59.9 | Bacillus sp. SprC | 378 | 272 |
| WP_003327717.1 | 58.4 | Bacillus atrophaeus 1942 | 382 | 274 |
| (previously known as | | (subtilisin E) | | |
| YP_003972439) | | | | |
| BAD11988 | 57.78 | Bacillus sp. KSM-LD1 | 376 | 272 |
| CAA24990 | 56.9 | Bacillus amyloliquefaciens | 376 | 274 |
| AGL34969 | 56.5 | Bacillus subtilis | 361 | 253 |
| BAD21128 | 56.4 | Bacillus sp. KSM-LD1 SB | 377 | 273 |
| BAD02409 | 56.3 | Bacillus sp. KSM-LD1 | 404 | 272 |
| BAN09118 | 56.2 | Bacillus subtilis | 381 | 274 |
| AAX53176 | 56.2 | Bacillus subtilis AP01 | 381 | 274 |
| AAC63365 | 55.9 | Bacillus subtilis | 382 | 272 |
| ACI32816 | 55.8 | Bacillus subtilis | 274 | 274 |
| CAA74536 | 55.8 | Bacillus subtilis str. 168 | 381 | 274 |
| WP_010333625 | 55.5 | Bacillus mojavensis | 381 | 274 |
| WP_010329279 | 55.1 | Bacillus vallismortis | 381 | 274 |
| ABY25856 | 55.1 | Geobacillus stearothermophilus | 382 | 274 |
| AFP23380 | 54.6 | Bacillus lehensis | 276 | 273 |
| CAJ70731 | 54.6 | Bacillus licheniformis | 379 | 273 |
| WP_007497196 | 54.6 | Bacillus stratosphericus | 383 | 273 |
| AAC43581 | 54..2 | Bacillus sp. SprD | 379 | 273 |
| WP_006636716 | 53.5 | Bacillus sonorensis | 378 | 273 |
| ADN04910 | 54.2 | Bacillus circulans | 275 | 273 |
| ADK11996 | 54.2 | Bacillus pumilus | 383 | 273 |

TABLE 6-2
Percent Identity (PID) Shared by BSP-00801 with Entries in Genome Quest Database

| Patent - SEQ ID NO/Accession No. | | | Sequence | Alignment |
|---|---|---|---|---|
| listed in Genome Quest | PID | Organism | Length | Length |
| US20090275493-0004 | 95.9 | B. gibsonii | 269 | 269 |
| WO03054184-0001 | 95.9 | B. gibsonii | 383 | 269 |
| WO2007131657-0001 | 94.8 | B. gibsonii | 383 | 269 |
| US20090275493-0003 | 94.8 | B. gibsonii | 269 | 269 |
| WO2008086916-0001 | 93.7 | B. gibsonii | 383 | 269 |
| WO2012119955-0001 | 89.6 | Bacillus sp. | 269 | 269 |
| WO03054185-0001 | 89.6 | B. gibsonii | 383 | 269 |
| DE102007044415_AWJ12891 | 89.6 | B. gibsonii | 269 | 269 |
| WO2011110625-0002 | 89.2 | Bacillus sp. | 269 | 269 |
| WO2012119955-0005 | 88.5 | Bacillus sp. | 269 | 269 |
| U.S. Pat. No. 7,507,569-0002 | 88.4 | Artificial Sequence | 269 | 268 |
| WO2012119955-0004 | 88.1 | Bacillus sp. | 269 | 269 |
| WO2012119955-0006 | 88.1 | Bacillus sp. | 269 | 269 |
| U.S. Pat. No. 7,642,080-0002 | 87.4 | Bacillus sp. strain Zi344 | 381 | 269 |
| U.S. Pat. No. 7,507,569-0004 | 86.6 | Artificial Sequence | 269 | 268 |
| U.S. Pat. No. 7,507,569-0006 | 86.2 | Artificial Sequence | 269 | 268 |
| U.S. Pat. No. 7,642,080-0006 | 85.5 | Bacillus sp. strain p203 | 383 | 269 |
| U.S. Pat. No. 7,642,080-0004 | 85.5 | Bacillus sp. strain EP655 | 383 | 269 |

### Example 7

### Sequence Analysis of B. gibsonii-Clade Subtilisins

An alignment of the amino acid sequences of the predicted mature forms of the B. gibsonii-clade subtilisins of Table 3; B. gibsonii-clade subtilisins DSM 9728, DSM 9729, DSM 9730, DSM 9731 and Bgi02446 disclosed in International Patent Application No. PCT/US2014/070107; and the amino acid sequences of multiple proteases listed in Tables 6-1 and 6-2 is shown in FIG. 6A-F. An alignment of the amino acid sequences of the predicted mature forms of the B. gibsonii-clade subtilisins of Table 4; B. gibsonii-clade subtilisin Bgi02446; and the amino acid sequences of multiple proteases listed in Table 6-1 is shown in FIG. 7A-F. An alignment of the amino acid sequences of the predicted mature forms of multiple B. gibsonii-clade BSP-00801 variant subtilisins of Table 5; B. gibsonii-clade subtilisin Bgi02446; and the amino acid sequences of multiple proteases listed in Table 6-1 is shown in FIG. 8A-F. The sequences were aligned using CLUSTALW software (Thompson et al., Nucleic Acids Research, 22:4673-4680, 1994) with default parameters.

The phylogenetic tree set forth in FIG. 9 was built using the amino acid sequences of the predicted mature forms of the B. gibsonii-clade subtilisins of Table 3; the B. gibsonii-clade subtilisins DSM 9728, DSM 9729, DSM 9730, DSM 9731 and Bgi02446 disclosed in International Patent Application No. PCT/US2014/070107; and the amino acid sequences of multiple proteases listed in Tables 6-1 and 6-2. As seen in FIG. 9, the Table 3 subtilisins cluster in the same region as the DSM 9728, DSM 9729, DSM 9730, DSM 9731 and Bgi02446 subtilisins disclosed in WO2015/089447 that form the B. gibsonii-clade, and therefore the Table 3 subtilisins are part of the B. gibsonii-clade.

The phylogenetic tree set forth in FIG. 10 was built using the amino acid sequences of the predicted mature forms of the B. gibsonii-clade subtilisins of Table 4; B. gibsonii-clade subtilisin Bgi02446; and the amino acid sequences of multiple proteases listed in Table 7-1. As seen in FIGS. 9 and 10, the Table 4 subtilisins cluster in the same region as the DSM 9728, DSM 9729, DSM 9730, DSM 9731 and Bgi02446 subtilisins, and therefore the Table 4 subtilisins are part of the B. gibsonii-clade.

The phylogenetic tree set forth in FIG. 11 was built using the amino acid sequences of the predicted mature forms of multiple B. gibsonii-clade BSP-00801 variant subtilisins of Table 5; B. gibsonii-clade subtilisin Bgi02446; and the amino acid sequences of multiple proteases listed in Table 7-1. As seen in FIGS. 9 and 11, the Table 5 subtilisins cluster in the same region as the DSM 9728, DSM 9729, DSM 9730, DSM 9731 and Bgi02446 subtilisins, and therefore the Table 5 subtilisins are part of the B. gibsonii-clade. When a phylogenetic tree of all of the B. gibsonii-clade BSP-00801 variant subtilisins of Table 5 was generated they all fell in the B. gibsonii-clade (data not shown).

The sequences were entered in the Vector NTI Advance suite and a Guide Tree was created using the Neighbor Joining (NJ) method (Saitou and Nei, Mol Biol Evol, 4:406-425, 1987). The NJ method works on a matrix of distances between all pairs of sequences to be analyzed. These distances are related to the degree of divergence between the sequences. The Guide Tree is calculated after the sequences are aligned. The tree construction was calculated using the following parameters: Kimura's correction for sequence distance and ignoring positions with gaps. The MEGA 6 program was used to display the phylogenetic trees shown in FIGS. 9-11.

### Example 8

### Unique Features of B. gibsonii-Clade Proteases Listed in Table 3

The amino acid sequences of the predicted mature forms of the B. gibsonii-clade subtilisins of Table 3 and Bgi02446 were aligned using CLUSTALW software (Thompson et al., Nucleic Acids Research, 22:4673-4680, 1994) with default parameters. The alignment showed that the Table 3 subtilisins share a motif extending between Asp(D)32 and His(H)65, which alignment is set forth in FIG. 12A-C. In all these enzymes, the catalytic triad is formed by Asp (D)32, His (H)62 and Ser (S)215. The motif DXGIXXHSDLXXXGGASXXXXXPTTADLNXHGTH (SEQ ID NO:71) or DXGIXXHSDLXXXGGASXXXXXXTTADLXXHGTH (SEQ ID NO:72) contains the sequence TTADL that is unique to the B. gibsonii-clade subtilisins of Table 3 as well as the previously identified B. gibsonii subtilisins disclosed in WO2015/089447. All of the B. gibsonii-clade subtilisins aligned in FIG. 6A-F share the motif DXGIXXHSDLXXXGG ASXXXXXPTTADLNXHGTH (SEQ ID NO:71) or DXGIXXHSDLXXXGGASXXXXXXTTADLXXHGTH (SEQ ID NO:72), which motifs are more fully described in WO2015/089447

### Example 9

### Measuring Cleaning Performance and Stability of Additional B. gibsonii-Clade Subtilisins

The additional B. gibsonii-clade subtilisins shown below in Table 7 were tested in the microswatch scale cleaning performance and stability tests described on Example 3. These proteins were generated as described in Example 2 using the B. lentus propeptide sequence for expression.

TABLE 7
Cleaning Performance and Stability of B. gibsonii-clade Subtilisins

| | | PI versus Bgi02446 | | | | |
|---|---|---|---|---|---|---|
| | | ADW | ADW | | Boron-free | Amino |
| B. gibsonii- | | pH 10.5 | pH 10.5 | Stabil- | HDL pH | acid |
| clade | | Rinsed | Unrinsed | ity in | 8.2, BMI | SEQ |
| Subtilisin | Mutations With Respect To Bgi02446 | Egg stain | Egg stain | EDTA | Stain | ID NO: |
| BSP-03386 | S099R | 1.0 | 3.4 | 0.8 | 0.6 | 205 |
| BSP-03486 | I080V | 1.1 | 3.4 | 0.8 | 0.6 | 212 |
| BSP-03385 | E087D | 1.0 | 1.1 | 0.8 | 1.0 | 204 |
| BSP-03390 | F128A | 1.0 | 1.1 | 0.9 | 1.0 | 208 |
| BSP-03392 | N242D | 1.0 | 1.6 | 0.9 | 1.3 | 209 |
| BSP-03388 | T114A | 0.9 | 1.1 | 1.0 | 1.2 | 206 |
| BSP-03467 | N085S | 1.0 | 1.2 | 1.1 | 1.0 | 211 |
| BSP-03389 | S126T | 0.9 | 1.0 | 1.1 | 1.0 | 207 |
| BSP-03380 | A037T | 1.0 | 1.2 | 1.1 | 1.0 | 200 |
| BSP-03407 | T114Q | 1.0 | 1.2 | 1.2 | 1.0 | 210 |
| BSP-03383 | A047V | 1.0 | 1.0 | 1.2 | 1.0 | 202 |
| BSP-03384 | T056Y | 1.0 | 1.0 | 1.2 | 1.1 | 203 |
| BSP-03381 | S039E | 1.8 | 1.0 | 1.6 | 1.0 | 201 |
| BSP-03344 | S099R-F128A | 1.2 | 1.1 | 3.4 | 1.3 | 223 |
| BSP-03331 | S099R-S126T | 1.2 | 7.0 | 0.8 | 0.9 | 219 |
| BSP-03396 | S126T-F128A | 1.1 | 5.3 | 1.1 | 0.6 | 229 |
| BSP-03346 | T056Y-T114Q | 1.1 | 1.1 | 1.3 | 1.3 | 224 |
| BSP-03304 | S039E-E087D | 1.2 | 1.0 | 1.7 | 1.0 | 213 |
| BSP-03372 | S099R-F128A-N242D | 1.2 | 1.1 | 3.3 | 1.4 | 226 |
| BSP-03333 | S099R-S126T-N242D | 1.3 | 4.1 | 0.7 | 0.9 | 220 |
| BSP-03435 | S099R-S126T-F128A | 1.1 | 3.0 | 1.1 | 0.7 | 232 |
| BSP-03343 | T056Y-T114Q-N242D | 1.2 | 4.3 | 1.2 | 0.8 | 222 |
| BSP-03397 | S039E-S099R-S126T | 1.4 | 1.2 | 1.6 | 1.4 | 230 |
| BSP-03398 | S039E-S099R-F128A | 1.0 | 3.9 | 2.9 | 0.8 | 231 |
| BSP-03309 | S039E-E087D-N242D | 1.2 | 4.5 | 2.9 | 1.1 | 214 |
| BSP-03321 | T056Y-S099R-T114Q-F128A | 1.2 | 1.1 | 3.6 | 1.6 | 217 |
| BSP-03351 | T056Y-S099R-T114Q-S126T | 1.6 | 2.3 | 1.3 | 0.8 | 225 |
| BSP-03662 | S039E-T056Y-S099R-F128A | 1.6 | 5.4 | 1.7 | 0.6 | 252 |
| BSP-03575 | S039E-S099R-T114Q-F128A | 1.6 | 5.8 | 2.8 | 1.3 | 244 |
| BSP-03324 | S039E-E087D-S099R-S126T | 1.6 | 3.9 | 2.8 | 1.3 | 218 |
| BSP-03563 | S039E-N085S-S099R-F128A | 1.4 | 5.9 | 2.8 | 0.8 | 243 |
| BSP-03678 | S039E-S099R-T114A-S126T | 2.0 | 4.3 | 2.9 | 1.4 | 255 |
| BSP-03311 | S039E-E087D-S099R-F128A | 1.5 | 4.4 | 2.9 | 0.7 | 215 |
| BSP-03393 | S039E-S099R-S126T-F128A | 1.1 | 8.2 | 3.1 | 1.3 | 227 |
| BSP-03334 | S039E-T056Y-E087D-T114Q | 1.6 | 4.0 | 3.4 | 1.1 | 221 |
| BSP-03476 | S039E-E087D-S099R-S126T-F128A | 1.5 | 1.1 | 3.4 | 1.5 | 235 |
| BSP-03394 | S039E-T056Y-S099R-S126T-F128A | 1.7 | 4.0 | 3.1 | 1.3 | 228 |
| BSP-03318 | S039E-E087D-S099R-F128A-N242D | 1.6 | 3.9 | 3.4 | 1.0 | 216 |
| BSP-03512 | A037T-S039E-E087D-S099R- | 1.5 | 4.9 | 3.4 | 1.4 | 240 |
| | T114A-F128A | | | | | |
| BSP-03477 | S039E-T056Y-E087D-S099R- | 1.5 | 4.7 | 2.9 | 1.2 | 236 |
| | S126T-F128A | | | | | |
| BSP-03688 | S039E-S099R-T114Q-S126T- | 1.5 | 3.6 | 3.2 | 1.1 | 256 |
| | F128A-N242D | | | | | |
| BSP-03473 | A037T-S039E-S099R-T114Q- | 1.5 | 2.9 | 3.6 | 1.1 | 234 |
| | S126T-F128A-N242D | | | | | |
| BSP-03493 | A037T-S039E-T056Y-S099R- | 1.7 | 2.7 | 3.7 | 1.5 | 238 |
| | S126T-F128A-N242D | | | | | |
| BSP-03653 | A037T-S039E-T056Y-N085S- | 1.4 | 2.5 | 3.8 | 1.4 | 251 |
| | E087D-S099R-T114Q-F128A | | | | | |
| BSP-03559 | A037T-S039E-A047V-T056Y- | 1.2 | 5.6 | 2.9 | 1.2 | 241 |
| | S099R-T114Q-S126T-F128A | | | | | |
| BSP-03623 | A037T-S039E-A047V-T056Y- | 1.5 | 3.8 | 3.2 | 1.4 | 246 |
| | S099R-T114A-S126T-F128A | | | | | |
| BSP-03604 | A037T-S039E-T056Y-S099R- | 1.6 | 4.9 | 3.4 | 1.0 | 245 |
| | T114Q-S126T-F128A-N242D | | | | | |
| BSP-03562 | S039E-A047V-E087D-S099R- | 1.7 | 2.2 | 3.7 | 1.2 | 242 |
| | T114Q-S126T-F128A-N242D | | | | | |
| BSP-03652 | S039E-A047V-T056Y-N085S- | 1.5 | 2.8 | 3.7 | 1.2 | 250 |
| | E087D-S099R-T114Q-S126T-F128A | | | | | |
| BSP-03663 | S039E-A047V-I080V-E087D- | 1.4 | 6.1 | 3.2 | 1.2 | 253 |
| | S099R-T114A-S126T-F128A-N242D | | | | | |
| BSP-03639 | A037T-S039E-T056Y-N085S- | 1.4 | 3.8 | 3.2 | 1.2 | 248 |
| | S099R-T114Q-S126T-F128A-N242D | | | | | |
| BSP-03631 | S039E-A047V-T056Y-E087D- | 1.4 | 3.6 | 3.3 | 1.2 | 247 |
| | S099R-T114A-S126T-F128A-N242D | | | | | |
| BSP-03463 | S039E-A047V-T056Y-E087D- | 1.6 | 4.2 | 3.6 | 1.5 | 233 |
| | S099R-T114Q-S126T-F128A-N242D | | | | | |
| BSP-03503 | A037T-S039E-A047V-I080V- | 2.1 | 2.9 | 3.8 | 1.2 | 239 |
| | N085S-E087D-S099R-T114A-S126T-F128A | | | | | |
| BSP-03482 | A037T-S039E-I080V-N085S- | 1.6 | 4.1 | 3.1 | 1.0 | 237 |
| | E087D-S099R-T114Q-S126T-F128A-N242D | | | | | |
| BSP-03675 | A037T-S039E-T056Y-N085S- | 1.6 | 6.7 | 3.3 | 1.5 | 254 |
| | E087D-S099R-T114A-S126T-F128A-N242D | | | | | |
| BSP-03647 | A037T-S039E-A047V-T056Y- | 1.6 | 3.7 | 3.8 | 1.5 | 249 |
| | E087D-S099R-T114A-S126T-F128A-N242D | | | | | |

### Example 10

### Sequence Analysis of B. gibsonii-Clade Subtilisins

An alignment of the amino acid sequences of Bgi02446, B. gibsonii-clade BSP-00801 variant subtilisin and mature forms of multiple B. gibsonii-clade variant subtilisins of Table 7 is shown in FIG. 14A-F. The sequences were aligned using CLUSTALW software (Thompson et al., Nucleic Acids Research, 22:4673-4680, 1994) with default parameters. The phylogenetic tree set forth in FIG. 15 was built using the amino acid sequences of the mature forms of the B. gibsonii-clade subtilisins of Table 7, the B. gibsonii-clade subtilisins DSM 9728, DSM 9729, DSM 9730, DSM 9731 and Bgi02446 disclosed in WO2015/089447; the amino acid sequences of multiple proteases listed in Tables 6-1 and 6-2, and the amino acid sequences of BgAP variants NH-12, NH-14, MT 1, MT2, MIFi (described in Martinez et al, Biotechnology and Bioengineering, 2012).

We claim:

1. An isolated subtilisin variant comprising an amino acid sequence comprising two, three, or four or more variations versus SEQ ID NO:85 at positions selected from:
(i) 1, 4, 9, 21, 24, 27, 36, 37, 39, 42, 43, 44, 47, 54, 55, 56, 74, 80, 85, 87, 99, 102, 114, 117, 119, 121, 126, 128, 131, 143, 144, 158, 159, 160, 169, 182, 188, 190, 197, 198,212, 224, 231, 232, 237, 242, 245, 246, 254, 255, 256, and 257;
(ii) 37, 39, 47, 56, 80, 85, 87, 99, 114, 126, 128, and 242;
(iii) 39, 99, 126, and 128;
(iv) 39 in combination with one or more variation at a position selected from 37, 47, 56, 80, 85, 87, 99, 114, 126, 128, and 242;
(v) 56 in combination with one or more variation at a position selected from 37, 39, 47, 80, 85, 87, 99, 114, 126, 128, and 242;
(vi) 114 in combination with one or more variation at a position selected from 37, 39, 47, 56, 80, 85, 87, 99, 126, 128, and 242;
(vii) 126 in combination with one or more variation at a position selected from 37, 39, 47, 56, 80, 85, 87, 99, 114, 128, and 242;
(viii) 242 in combination with one or more variation at a position selected from 37, 39, 47, 56, 80, 85, 87, 99, 114, 126, and 128;
(ix) 99+128 in combination with one or more variation at a position selected from 39, 56, 114, 126 and 242;
(x) 39+242 in combination with one or more variation at a position selected from 37, 47, 56, 80, 85, 87, 99, 114, 126, and 128; or
(xi) 39+99+128 in combination with one or more variation at a position selected from 37, 47, 56, 80, 85, 87, 114, 126, and 242;
with the proviso that one or more of said two, three, or four or more variations is non-naturally occurring;
wherein the amino acid positions of the variant are numbered by correspondence with the amino acid sequence of SEQ ID NO:85.

2. The isolated subtilisin variant of claim 1, wherein said variant comprises an amino acid sequence comprising two, three, or four or more amino acid variations versus SEQ ID NO:85 selected from:
(i) 1A, 4I, 9S, 21V, 24F, 27K, 36A, 37T/N, 39E, 42T, 43V, 44S, 47V, 54S, 55M/G, 56N/Y, 74D, 80V, 85S, 87D, 99R, 102I, 114A/P/Q, 117I, 119V, 121S, 126T, 128A, 131T, 143A/T/Q, 144G, 158T, 159I, 160S, 169L, 182S, 188A, 190L, 197I, 198G, 212S/T/K, 224I/V, 231K, 232N, 237T, 242D/Q, 245L, 246S/K, 254T, 255N, 256L, and 257Y;
(ii) 37T/N, 39E, 47V, 56N/Y, 80V, 85S, 87D, 99R, 114A/P/Q, 126T, 128A, and 242D/Q;
or
(iii) 39E, 99R, 126T, and 128A;
(iv) 39E in combination with one or more variation at a position selected from 37T/N, 47V, 56N/Y, 80V, 85S, 87D, 99R, 114A/P/Q, 126T, 128A, and 242D/Q;
(v) 56N/Y in combination with one or more variation at a position selected from 37T/N, 39E, 47V, 80V, 85S, 87D, 99R, 114A/P/Q, 126T, 128A, and 242D/Q;
(vi) 114A/P/Q in combination with one or more variation at a position selected from 37T/N, 39E, 47V, 56N/Y, 80V, 85S, 87D, 99R, 126T, 128A, and 242D/Q;
(vii) 126T in combination with one or more variation at a position selected from 37T/N, 39E, 47V, 56N/Y, 80V, 85S, 87D, 99R, 114A/P/Q, 128A, and 242D/Q;
(viii) 242D/Q in combination with one or more variation at a position selected from 37T/N, 39E, 47V, 56N/Y, 80V, 85S, 87D, 99R, 114A/P/Q, 126T, and 128A;
(ix) 99R+128A in combination with one or more variation at a position selected from 39E, 56N/Y, 114A/P/Q, 126T, and 242D/Q;
(x) 39E+242D/Q in combination with one or more variation at a position selected from 37T/N, 47V, 56N/Y, 80V, 85S, 87D, 99R, 114A/P/Q, 126T, and 128A; or
(xi) 39E+99R+128A in combination with one or more variation at a position selected from 37T/N, 47V, 56N/Y, 80V, 85S, 87D, 114A/P/Q, 126T, and 242D/Q.

3. An isolated subtilisin variant comprising an amino acid sequence comprising two, three, or four or more amino acid substitutions at positions selected from:
(i) 1, 4, 9, 21, 24, 27, 36, 37, 39, 42, 43, 44, 47, 54, 55, 56, 74, 80, 85, 87, 99, 102, 114, 117, 119, 121, 126, 128, 131, 143, 144, 158, 159, 160, 169, 182, 188, 190, 197, 198,212, 224, 231, 232, 237, 242, 245, 246, 254, 255, 256, and 257;
(ii) 37, 39, 47, 56, 80, 85, 87, 99, 114, 126, 128, and 242;
(iii) 39, 99, 126, and 128;
(iv) 39 in combination with one or more amino acid substitution at a position selected from 37, 47, 56, 80, 85, 87, 99, 114, 126, 128, and 242;
(v) 56 in combination with one or more amino acid substitution at a position selected from 37, 39, 47, 80, 85, 87, 99, 114, 126, 128, and 242;
(vi) 114 in combination with one or more amino acid substitution at a position selected from 37, 39, 47, 56, 80, 85, 87, 99, 126, 128, and 242;
(vii) A/S126 in combination with one or more amino acid substitution at a position selected from 37, 39, 47, 56, 80, 85, 87, 99, 114, 128, and 242;
(viii) N242 in combination with one or more amino acid substitution at a position selected from 37, 39, 47, 56, 80, 85, 87, 99, 114, 126, and 128;
(ix) 99+128 in combination with one or more amino acid substitution at a position selected from 39, 56, 114, 126 and 242;
(x) 39+242 in combination with one or more amino acid substitution at a position selected from 37, 47, 56, 80, 85, 87, 99, 114, 126, and 128; or
(xi) 39+99+128 in combination with one or more amino acid substitution at a position selected from 37, 47, 56, 80, 85, 87, 114, 126, and 242;
with the proviso that one or more of said two, three, or four or more substitutions is non-naturally occurring;
wherein the amino acid positions of the variant are numbered by correspondence with the amino acid sequence of SEQ ID NO:85.

4. The isolated subtilisin variant of claim 3, wherein said variant comprises an amino acid sequence comprising two, three, or four or more amino acid substitutions selected from:
(i) Q1, V4, S/T9, I/V21, S24, K/R27, S36, Q/T/S/A37, S/P/T39, N/T42, I43, R/S44, A/V47, P/S54, S/T55, T56, N74, I/V80, N/585, D/E/Q87, S/R99, I/V102, A/T114, M117, I119, N121, A/S126, A/F128, I/S/T131, Q/R143, D/G144, N/S158, I/V159, G160, M169, N/5182, S/T188, 1190, V197, G/N198, N/P212, A/V224, K/R231, N/Y232, A/N237, N242, K245, N246, S254, S255, Q256, and F257;
(ii) Q/T/S/A37, S/P/T39, A/V47, T56, I/V80, N/S85, D/E/Q87, S/R99, A/T114, A/S126, A/F128, and N242;
(iii) S/P/T39, S/R99, A/S126, and A/F128;
(iv) S/P/T39 in combination with one or more amino acid substitution at a position selected from Q/T/S/A37, A/V47, T56, I/V80, N/S85, D/E/Q87, S/R99, A/T114, A/S126, A/F128, and N242;
(v) T56 in combination with one or more amino acid substitution at a position selected from Q/T/S/A37, S/P/T39, A/V47, I/V80, N/S85, D/E/Q87, S/R99, A/T114, A/S126, A/F128, and N242;
(vi) A/T114 in combination with one or more amino acid substitution at a position selected from Q/T/S/A37, S/P/T39, A/V47, T56, I/V80, N/S85, D/E/Q87, S/R99, A/S126, A/F128, and N242;
(vii) A/S126 in combination with one or more amino acid substitution at a position selected from Q/T/S/A37, S/P/T39, A/V47, T56, I/V80, N/S85, D/E/Q87, S/R99, A/T114, A/F128, and N242;
(viii) N242 in combination with one or more amino acid substitution at a position selected from Q/T/S/A37, S/P/T39, A/V47, T56, I/V80, N/S85, D/E/Q87, S/R99, A/T114, A/S126, and A/F128;
(ix) S/R99+A/F128 in combination with one or more amino acid substitution at a position selected from S/P/T39, T56, A/T114, A/S126 and N242;
(x) S/P/T39+N242 in combination with one or more amino acid substitution at a position selected from Q/T/S/A37, A/V47, T56, I/V80, N/S85, D/E/Q87, S/R99, A/T114, A/S126, and A/F128; or
(xi) S/P/T39+S/R99+A/F128 in combination with one or more amino acid substitution at a position selected from Q/T/S/A37, A/V47, T56, I/V80, N/S85, D/E/Q87, A/T114, A/S126, and N242.

5. The isolated subtilisin variant of claim 3, wherein said variant comprises an amino acid sequence comprising two, three, or four or more amino acid substitutions selected from:
(i) 1A, 4I, 9S, 21V, 24F, 27K, 36A, 37T/N, 39E, 42T, 43V, 44S, 47V, 54S, 55M/G, 56N/Y, 74D, 80V, 85S, 87D, 99R, 102I, 114A/P/Q, 117I, 119V, 121S, 126T, 128A, 131T, 143A/T/Q, 144G, 158T, 159I, 160S, 169L, 182S, 188A, 190L, 197I, 198G, 212S/T/K, 224I/V, 231K, 232N, 237T, 242D/Q, 245L, 246S/K, 254T, 255N, 256L, and 257Y;
(ii) 37T/N, 39E, 47V, 56N/Y, 80V, 85S, 87D, 99R, 114A/P/Q, 126T, 128A, and 242D/Q;
(iii) 39E, 99R, 126T, and 128A;
(iv) 39E in combination with one or more substitution at a position selected from 37T/N, 47V, 56N/Y, 80V, 85S, 87D, 99R, 114A/P/Q, 126T, 128A, and 242D/Q;
(v) 56N/Y in combination with one or more substitution at a position selected from 37T/N, 39E, 47V, 80V, 85S, 87D, 99R, 114A/P/Q, 126T, 128A, and 242D/Q;
(vi) 114A/P/Q in combination with one or more substitution at a position selected from 37T/N, 39E, 47V, 56N/Y, 80V, 85S, 87D, 99R, 126T, 128A, and 242D/Q;
(vii) 126T in combination with one or more substitution at a position selected from 37T/N, 39E, 47V, 56N/Y, 80V, 85S, 87D, 99R, 114A/P/Q, 128A, and 242D/Q;
(viii) 242D/Q in combination with one or more substitution at a position selected from 37T/N, 39E, 47V, 56N/Y, 80V, 85S, 87D, 99R, 114A/P/Q, 126T, and 128A;
(ix) 99R+128A in combination with one or more substitution at a position selected from 39E, 56N/Y, 114A/P/Q, 126T, and 242D/Q;
(x) 39E+242D/Q in combination with one or more substitution at a position selected from 37T/N, 47V, 56N/Y, 80V, 85S, 87D, 99R, 114A/P/Q, 126T, and 128A; or
(xi) 39E+99R+128A in combination with one or more substitution at a position selected from 37T/N, 47V, 56N/Y, 80V, 85S, 87D, 114A/P/Q, 126T, and 242D/Q.

6. The isolated subtilisin of any one of claims 3-5, wherein said variant comprises an amino acid sequence comprising two, three, or four or more amino acid substitutions selected from:
(i) Q1A, V4I, T9S, I21V, S24F, R27K, S36A, Q/T/S/A37N/T, S/P/T39E, N42T, I43V, R44S, A47V, P54S, S/T55G/M, T56N/Y, N74D, I80V, N85S, E/Q87D, S99R, V102I, A/T114A/P/Q, M117I, I119V, N121S, A/S126T, F128A, I/S131T, Q/R143A/Q/T, D144G, N/S158T, V159I, G160S, M169L, N182S, S/T188A, I190L, V197I, N198G, N/P212K/S/T, A/V224I/V, R231K, Y232N, A/N237T, N242D/Q, K245L, N246K/S, S254T, S255N, Q256L, and F257Y;
(ii) Q/T/S/A37N/T, S/P/T39E, A47V, T56N/Y, I80V, N85S, E/Q87D, S99R, A/T114A/P/Q, A/S126T, F128A, and N242D/Q;
(iii) S/P/T39E, S99R, A/S126T, and F128A;
(iv) S/P/T39E in combination with one or more substitution at a position selected from Q/T/S/A37N/T, A47V, T56N/Y, I80V, N85S, E/Q87D, S99R, A/T114A/P/Q, A/S126T, F128A, and N242D/Q;
(v) T56N/Y in combination with one or more substitution at a position selected from Q/T/S/A37N/T, S/P/T39E, A47V, I80V, N85S, E/Q87D, S99R, A/T114A/P/Q, A/S126T, F128A, and N242D/Q;
(vi) A/T114A/P/Q in combination with one or more substitution at a position selected from Q/T/S/A37N/T, S/P/T39E, A47V, T56N/Y, I80V, N85S, E/Q87D, S99R, A/S126T, F128A, and N242D/Q;
(vii) A/S126T in combination with one or more substitution at a position selected from Q/T/S/A37N/T, S/P/T39E, A47V, T56N/Y, I80V, N85S, E/Q87D, S99R, A/T114A/P/Q, F128A, and N242D/Q;
(viii) N242D/Q in combination with one or more substitution at a position selected from Q/T/S/A37N/T, S/P/T39E, A47V, T56N/Y, I80V, N85S, E/Q87D, S99R, A/T114A/P/Q, A/S126T, and F128A;
(ix) S99R+F128A in combination with one or more substitution at a position selected from S/P/T39E, T56N/Y, A/T114A/P/Q, A/S126T and N242D/Q;
(x) S/P/T39E+N242D/Q in combination with one or more substitution at a position selected from Q/T/S/A37T/N, A47V, T56N/Y, I80V, N85S, E/Q87D, S99R, A/T114A/P/Q, A/S126T, and F128A; or
(xi) S/P/T39E+S99R+F128A in combination with one or more substitution at a position selected from Q/T/S/A37N/T, A47V, T56N/Y, I80V, N85S, E/Q87D, A/T114A/P/Q, A/S126T, and N242D/Q.

7. The isolated subtilisin of claim 1 or 2, with the proviso:
(i) that said two, three, or four or more variations versus SEQ ID NO:85 is not S39T+I21V+M122L+N177E;
(ii) that said two, three, or four or more variations versus SEQ ID NO:85 is not S39E+N74D+D87E;
(iii) that said two, three, or four or more variations versus SEQ ID NO:85 is not S39E+N74D+D87E+N253D;
(iv) that said two, three, or four or more variations versus SEQ ID NO:85 is not I21V+S39E+N74D+D87E+M122L+N253D;
(v) that said two, three, or four or more variations versus SEQ ID NO:85 is not Q37E+Q256E; and/or
(vi) that said variation versus SEQ ID NO:85 at position 21 is not a valine when said variant comprises a variation versus SEQ ID NO:85 at one or more positions selected from: 4, 36, 42, 47, 56, 87, 99, 102, 114, 188, 224, 237, 242, and 255.

8. The isolated subtilisin of any one of claims 3-6, with the proviso:
(i) that said two, three, or four or more substitutions is not S39T+I21V+M122L+N177E;
(ii) that said two, three, or four or more substitutions is not S39E+N74D+D87E;
(iii) that said two, three, or four or more substitutions is not S39E+N74D+D87E+N253D;
(iv) that said two, three, or four or more substitutions is not I21V+S39E+N74D+D87E+M122L+N253D;
(v) that said two, three, or four or more substitutions is not Q37E+Q256E; and/or
(vi) that the substitution at position 21 is not a valine when said variant comprises a substitution at one or more positions selected from: 4, 36, 42, 47, 56, 87, 99, 102, 114, 188, 224, 237, 242, and 255.

9. An isolated subtilisin variant comprising an amino acid sequence comprising:
(i) one or more variation versus SEQ ID NO:85 at positions selected from 56, 114, and 126;
(ii) one or more variation versus SEQ ID NO:85 at positions selected from 56N/Y, 114A/P/Q, and 126T;
(iii) one or more amino acid substitution at positions selected from 56, 114, and 126;
(iv) one or more amino acid substitution at positions selected from T56, A/T114, and A/S126;
(v) one or more amino acids substitutions selected from: 56N/Y, 114A/P/Q, and 126T;
or
(vi) one or more amino acids substitutions selected from: T56N/Y, A/T114A/P/Q, and A/S126T;
with the proviso that one or more of said two, three, or four or more substitutions or variations is non-naturally occurring;
wherein the amino acid positions of the variant are numbered by correspondence with the amino acid sequence of SEQ ID NO:85.

10. The isolated subtilisin variant of any preceding claim, wherein said variant comprises an amino acid sequence comprising one or more amino acid substitutions selected from: N074D-I080V-N085S-E087D-S099R-V102I-S158T-V159I-N242D-F257Y; N074D-N085S-E087D-S099R-V102I-S126T-F128A-I190L-N242D-F257Y; R027K-S099R-V102I-T114Q-I119V-S126T-F128A-S158T-V159I-G160S-T188A-N242D; N074D-I080V-N085S-E087D-S099R-V102I-T114Q-S126T-F128A-N242D; R027K-S099R-V102I-S126T-F128A-R143A-S158T-V159I-G160S; N074D-I080V-N085S-E087D-S099R-V102I-I190L-N242D-F257Y; N074D-I080V-N085S-E087D-S099R-V102I-T114Q-R143A-I190L-N242D-F257Y; N074D-S099R-V102I-S126T-F128A-R143A-S158T-N212S; N074D-I080V-N085S-E087D-S099R-T114Q-S126T-F128A-R143A-N242D; N085S-E087D-S099R-T114Q-I119V-S126T-F128A-R143A-G160S-F257Y; N074D-I080V-S099R-V102I-T114Q-S126T-F128A-R143A-N212S-N242D; N074D-I080V-N085S-E087D-S099R-V102I-S126T-F128A-R143A-I190L-N242D-F257Y; N074D-I080V-S099R-V102I-T114Q-I119V-S126T-F128A-S158T-G160S-N212S-F257Y; I080V-N085S-E087D-S099R-V102I-T114Q-I119V-S126T-F128A-R143A-N242D; N085S-E087D-S099R-V102I-T114Q-S158T-V159I-G160S-N212S-N242D-F257Y; R027K-N074D-S099R-V102I-T114Q-S126T-F128A-T 188A-I190L-N212S-N242D; R027K-N074D-S099R-V102I-I119V-S126T-F128A-R143A-G160S-N212S-N242D; N042T-I080V-N085S-E087D-T114A-F128A-R143Q-D144G-S158T-V159I-G160S-N198G; I021V-I080V-N085S-E087D-M117I-F128A-S131T-R143Q-D144G-A224V; N074D-I080V-N085S-E087D-S158T-N242D; Q001A-I080V-F128A-S131T-R143A-D144G-M169L-I190L-S254T-S255N-Q256L-F257Y; A037T-S039E-N042T-S099R-V102I-T114Q-S126T-F128A-N242D; A037T-S039E-S099R-V102I-T114Q-S126T-F128A-R143A-N212S-N242D; A037T-S039E-N074D-S099R-V102I-S126T-F128A-R143A-N212S-N242D; A037T-S039E-N042T-N074D-S099R-V102I-S126T-F128A-S158T-N242D; A037T-S039E-I043V-A047V-I080V-N085S-E087D-S099R-T114A-F128A; A037T-S039E-I043V-A047V-T055G-S099R-V102I-T114Q-S126T-F128A-N212S; A037T-S039E-I043V-A047V-I080V-N085S-E087D-S099R-T114Q-F128A-N242D; A037T-S039E-I043V-A047V-I080V-N085S-E087D-S099R-T114A-F128A-N242D; A037T-S039E-A047V-I080V-N085S-E087D-S099R-T114Q-F128A-R143A-N242D; S036A-S039E-I043V-A047V-T055M-I080V-N085S-E087D-S099R-T114A-F128A; A037T-S039E-I043V-A047V-I080V-N085S-E087D-S099R-V102I-T114Q-F128A; A037T-S039E-I043V-A047V-N074D-N085S-E087D-S099R-T114A-F128A-S158T-N212S; A037T-S039E-N074D-I080V-N085S-E087D-S099R-V102I-T114Q-S126T-F128A-R143A; A037T-S039E-I043V-A047V-T055G-N074D-I080V-S099R-V102I-T114Q-S126T-F128A; A037T-S039E-I043V-A047V-N074D-I080V-S099R-V102I-T114Q-S126T-F128A-N242D; R027K-A037T-S039E-A047V-T055G-T056Y-N074D-S099R-V102I-S126T-F128A-N242D; A037T-S039E-N042T-A047V-N074D-I080V-N085S-E087D-S099R-T114Q-F128A-N242D; A037T-S039E-I043V-A047V-I080V-N085S-E087D-S099R-T114A-S126T-F128A-N212S; A037T-S039E-I043V-A047V-I080V-N085S-E087D-S099R-T114A-S126T-F128A-N242D; A037T-S039E-I043V-A047V-I080V-N085S-E087D-S099R-T114Q-S126T-F128A-R143A; A037T-S039E-I043V-A047V-I080V-N085S-E087D-S099R-V102I-T114A-F128A-N212S; A037T-S039E-N042T-A047V-I080V-N085S-E087D-S099R-V102I-T114Q-F128A-N242D; A037T-S039E-I043V-A047V-T056Y-I080V-N085S-E087D-S099R-T114A-F128A-N242D; A037T-S039E-I043V-A047V-I080V-N085S-E087D-S099R-T114Q-F128A-N212S-N242D; A037T-S039E-A047V-I080V-N085S-E087D-S099R-V102I-T114Q-F128A-R143A-N242D; A037T-S039E-I043V-A047V-T055G-I080V-N085S-E087D-S099R-T114Q-S126T-F128A; R027K-A037T-S039E-A047V-I080V-N085S-E087D-S099R-T114Q-F128A-N212S-N242D; T009S-A037T-S039E-I043V-A047V-I080V-N085S-E087D-S099R-T 114A-F128A-V197I; S039E-I043V-A047V-I080V-N085S-E087D-S099R-T114A-F128A-N212S-K245L-N246S; A037T-S039E-I043V-A047V-I080V-N085S-E087D-S099R-T114A-F128A-I190L-A224V; S036A-S039E-I043V-A047V-I080V-N085S-E087D-S099R-T114A-N121S-F128A-R143Q; S036A-S039E-I043V-A047V-I080V-N085S-E087D-S099R-T114A-F128A-N212S-S255N; S039E-I043V-A047V-I080V-N085S-E087D-S099R-T114A-F128A-R143Q-N242D-N246K; A037T-S039E-I043V-A047V-N074D-N085S-E087D-S099R-T114A-S126T-F128A-N212S; A037T-S039E-I043V-A047V-N074D-I080V-N085S-E087D-S099R-T114A-F128A-N212S; A037T-S039E-N042T-A047V-I080V-N085S-E087D-S099R-T114A-F128A-S158T-N212S; V004I-A037T-S039E-I043V-A047V-I080V-N085S-E087D-S099R-T114A-F128A-N182S; A037T-S039E-I043V-A047V-N074D-N085S-E087D-S099R-T114A-S126T-F128A-S158T-N212S; A037T-S039E-I043V-A047V-N074D-I080V-N085S-E087D-S099R-T114A-F128A-S158T-N212S; A037T-S039E-I043V-A047V-N074D-N085S-E087D-S099R-T114A-F128A-N212S-N242Q-K245L; A037T-S039E-I043V-A047V-N074D-I080V-N085S-E087D-S099R-T114A-F128A-N212S-N242Q; A037T-S039E-I043V-A047V-N074D-I080V-N085S-E087D-S099R-T114A-F128A-N212S-K245L; A037T-S039E-A047V-S099R-V102I-T114A-I119V-S126T-F128A-R143A-S158T-G160S-N212S; A037T-S039E-N042T-A047V-T056Y-N074D-I080V-N085S-E087D-S099R-T114Q-S126T-F128A; S024F-A037T-S039E-A047V-N074D-I080V-N085S-E087D-S099R-T114Q-S126T-F128A-N212S; A037T-S039E-I043V-A047V-I080V-N085S-E087D-S099R-T114Q-S126T-F128A-R143A-N242D; A037T-S039E-I043V-A047V-I080V-N085S-E087D-S099R-T114A-S126T-F128A-A237T-N242D; A037T-S039E-I043V-A047V-I080V-N085S-E087D-S099R-V102I-T114Q-S126T-F128A-N242D; A037T-S039E-I043V-A047V-T055G-I080V-N085S-E087D-S099R-V102I-T114Q-F128A-R143A; A037T-S039E-N042T-A047V-T055G-I080V-N085S-E087D-S099R-T114Q-F128A-N212S-N242D; A037T-S039E-I043V-A047V-I080V-N085S-E087D-S099R-T114A-F128A; A037T-S039E-I043V-A047V-I080V-N085S-E087D-S099R-T114A-F128A-N242D; A037T-S039E-I043V-A047V-I080V-N085S-E087D-S099R-T114Q-F128A-N242D; A037T-S039E-I043V-A047V-I080V-N085S-E087D-S099R-T114A-S126T-F128A-N242D; A037T-S039E-I043V-A047V-T056Y-I080V-N085S-E087D-S099R-T114A-F128A-N242D; A037T-S039E-I043V-A047V-I080V-N085S-E087D-S099R-T114Q-S126T-F128A-R143A; A037T-S039E-I043V-A047V-T055G-I080V-N085S-E087D-S099R-T114Q-S126T-F128A; A037T-S039E-I043V-A047V-N074D-I080V-N085S-E087D-S099R-T114A-F128A-S158T-N212S; A037T-S039E-I043V-A047V-I080V-N085S-E087D-S099R-T114A-S126T-F128A-A237T-N242D; A037T-S039E-I043V-A047V-N074D-I080V-N085S-E087D-S099R-T114A-S126T-F128A-N242D; R027K-A037T-S039E-I043V-A047V-I080V-N085S-E087D-S099R-T114A-S126T-F128A-N242D; A037T-S039E-I043V-A047V-T056Y-I080V-N085S-E087D-S099R-T114A-S126T-F128A-R143A; A037T-S039E-I043V-A047V-I080V-N085S-E087D-S099R-T114A-N121S-F128A-N246S-S255N; A037T-S039E-A047V-I080V-N085S-E087D-S099R-T114Q-F128A-R143A-N242D; A037T-S039E-I043V-A047V-N074D-N085S-E087D-S099R-T114A-F128A-S158T-N212S; A037T-S039E-I043V-A047V-I080V-N085S-E087D-S099R-V102I-T114Q-S126T-F128A-N242D; A037T-S039E-I043V-A047V-T056Y-I080V-N085S-E087D-S099R-T114Q-S126T-F128A-N242D; A037T-S039E-I043V-A047V-N074D-I080V-N085S-E087D-S099R-T114Q-I119V-S126T-F128A; A037T-S039E-I043V-A047V-T055G-I080V-N085S-E087D-S099R-V102I-T114A-S126T-F128A-S158T; A037T-S039E-I043V-A047V-I080V-N085S-E087D-S099R-T114A-S126T-F128A-R143A-S158T-N242D; A037T-S039E-N042T-I043V-R044S-A047V-N074D-I080V-N085S-E087D-S099R-T114A-S126T-F128A; A037T-S039E-N042T-I043V-R044S-A047V-I080V-N085S-E087D-S099R-T114A-S126T-F128A-N242D; A037T-S039E-I043V-A047V-I080V-N085S-E087D-S099R-V102I-T114A-I119V-S126T-F128A-F257Y; A037T-S039E-I043V-A047V-N074D-I080V-N085S-E087D-S099R-V102I-T114A-N121S-F128A-R143Q; A037T-S039E-I043V-A047V-N074D-N085S-E087D-S099R-T114A-S126T-F128A-S158T-N212S; S036A-S039E-I043V-A047V-N074D-I080V-N085S-E087D-S099R-T114A-F128A-A224V-S255N; A037T-S039E-I043V-A047V-T055G-N074D-I080V-N085S-E087D-S099R-T 114Q-S126T-F128A-N242D; A037T-S039E-I043V-A047V-T055G-I080V-N085S-E087D-S099R-V102I-T114Q-S126T-F128A-R143A; A037T-S039E-I043V-A047V-I080V-N085S-E087D-S099R-V102I-T114Q-S126T-F128A-R143A-N242D; A037T-S039E-I043V-A047V-T056Y-I080V-N085S-E087D-S099R-T114Q-S126T-F128A-S158T-N242D; A037T-S039E-I043V-A047V-T056Y-I080V-N085S-E087D-S099R-T114Q-I119V-S126T-F128A-F257Y; A037T-S039E-I043V-A047V-T056Y-I080V-N085S-E087D-S099R-T114Q-I119V-S126T-F128A-N242D; A037T-S039E-I043V-A047V-N074D-I080V-N085S-E087D-S099R-T114A-S126T-F128A-S158T-N212S-N242Q; A037T-S039E-I043V-A047V-N074D-I080V-N085S-E087D-S099R-T114A-S126T-F128A-T188A-I190L-F257Y; V004I-T009S-A037T-S039E-I043V-A047V-I080V-N085S-E087D-S099R-T114A-F128A-A224V-K245L-S255N; A037T-S039E-A047V-T056Y-I080V-N085S-E087D-S099R-T114Q-R143A-N242D; A037T-S039E-N042T-A047V-T056Y-N074D-I080V-N085S-E087D-S099R-T114Q-S126T-F128A; A037T-S039E-I043V-A047V-N074D-N085S-E087D-S099R-T114A-S126T-F128A-S158T-N212S-N242Q; A037T-S039E-I043V-A047V-N074D-N085S-E087D-S099R-T114A-S126T-F128A-S158T-N212S-K245L; A037T-S039E-I043V-A047V-I080V-N085S-E087D-S099R-T114P-S126T-F128A-R143T-S158T-N212K-N242D; A037T-S039E-I043V-A047V-T056Y-N074D-I080V-N085S-E087D-S099R-T114Q-S126T-F128A-R143A-N242D; A037T-S039E-I043V-A047V-T055G-T056Y-N074D-I080V-N085S-E087D-S099R-V102I-T114Q-S126T-F128A; S036A-A037N-S039E-I043V-A047V-I080V-N085S-E087D-S099R-T114A-F128A-N242D-K245L-N246S-S255N; A037T-S039E-I043V-A047V-N074D-I080V-N085S-E087D-S099R-T114A-S126T-F128A-S158T-N212S-N242Q-K245L; A037T-S039E-I043V-A047V-I080V-N085S-E087D-S099R-T114A-I119V-S126T-F128A-S158T-G160S-N242D-F257Y; A037T-S039E-I043V-A047V-I080V-N085S-E087D-S099R-T114A-N121S-F128A-N182S-I190L-K245L-N246S-S255N; A037T-S039E-N074D-I080V-N085S-E087D-S099R-V102I-T114Q-S126T-F128A-R143A; A037T-S039E-I043V-A047V-T055G-N074D-I080V-S099R-V102I-T114Q-S126T-F128A; A037T-S039E-I043V-A047V-N074D-I080V-S099R-V102I-T114Q-S126T-F128A-N242D; A037T-S039E-A047V-N074D-I080V-N085S-E087D-S099R-V102I-T114Q-S126T-F128A-S158T-N242D; A037T-S039E-N042T-A047V-N074D-I080V-N085S-E087D-S099R-V102I-T114Q-S126T-F128A-N242D; A037T-S039E-I043V-A047V-N074D-N085S-E087D-S099R-T114A-S126T-F128A-S158T-N212S-N242Q-K245L; T009S-S036A-S039E-I043V-A047V-I080V-N085S-E087D-S099R-T114A-F128A-R143Q-N182S-T188A-I190L; A037T-S039E-I043V-A047V-T055G-N074D-I080V-N085S-E087D-S099R-V102I-T114Q-S126T-F128A-S158T-N242D; A037T-S039E-I043V-A047V-T055G-N074D-I080V-N085S-E087D-S099R-T114Q-S126T-F128A-R143A-S158T-N212S; A037T-S039E-I043V-A047V-I080V-N085S-E087D-S099R-T114A-F128A-R143Q-N212S-A224V-Y232N-K245L-N246S-S255N; V004I-T009S-A037T-S039E-I043V-A047V-I080V-N085S-E087D-S099R-T114A-N121S-F128A-R143Q-A224I-R231K-K245L; R027K-A037T-S039E-A047V-I080V-N085S-E087D-S099R-T114Q-I119V-S126T-F128A-G160S-N242D-F257Y; R027K-A037T-S039E-I043V-A047V-T056Y-I080V-N085S-E087D-S099R-T114Q-S126T-F128A-S158T-G160S-N242D-F257Y; R027K-A037T-S039E-I043V-A047V-T056Y-I080V-N085S-E087D-S099R-V102I-T114Q-S126T-F128A-T188A-I190L-F257Y; R027K-A037T-S039E-I043V-A047V-T056N-I080V-N085S-E087D-S099R-T114P-S126T-F128A-R143T-V159I-N212T-F257Y; A037T-S039E-I043V-A047V-T056Y-I080V-N085S-E087D-S099R-T114Q-I119V-S126T-F128A-S158T-V159I-G160S-F257Y; A037T-S039E-I043V-A047V-T055G-T056Y-N074D-I080V-N085S-E087D-S099R-V102I-T114Q-S126T-F128A-N242D; R027K-A037T-S039E-A047V-T056Y-N074D-I080V-N085S-E087D-S099R-T114Q-I119V-S126T-F128A-N242D-F257Y; A037T-S039E-N042T-N074D-S099R-V102I-S126T-F128A-S158T-N242D; R027K-A037T-S039E-A047V-T055G-T056Y-N074D-S099R-V102I-S126T-F128A-N242D; S039E-I043V-A047V-I080V-N085S-E087D-S099R-T114A-F128A-A224V-N242D-N246S-S255N; A037T-S039E-I043V-A047V-N074D-I080V-N085S-E087D-S099R-V102I-T114A-F128A-R143Q-S158T-A224V-S255N; A037T-S039E-I043V-A047V-P054S-T056Y-A057Q-N074D-I080V-N085S-E087D-S099R-T114A-N121S-F128A-S158T-A224V-S255N; A037T-S039E-N042T-I043V-R044S-A047V-N074D-I080V-N085S-E087D-S099R-V102I-S126T-F128A-S158T-G160S-N242D; S126T; T114Q; T056Y; S099R-F128A; S099R-S126T; S126T-F128A; T056Y-T114Q; S039E-E087D; S099R-F128A-N242D; S099R-S126T-N242D; S099R-S126T-F128A; T056Y-T114Q-N242D; S039E-S099R-S126T; S039E-S099R-F128A; S039E-E087D-N242D; T056Y-S099R-T114Q-F128A; T056Y-S099R-T114Q-S126T; S039E-T056Y-S099R-F128A; S039E-S099R-T114Q-F128A; S039E-E087D-S099R-S126T; S039E-N085S-S099R-F128A; S039E-S099R-T114A-S126T; S039E-E087D-S099R-F128A; S039E-S099R-S126T-F128A; S039E-T056Y-E087D-T114Q; S039E-E087D-S099R-S126T-F128A; S039E-T056Y-S099R-S126T-F128A; S039E-E087D-S099R-F128A-N242D; A037T-S039E-E087D-S099R-T114A-F128A; S039E-T056Y-E087D-S099R-S126T-F128A; S039E-S099R-T114Q-S126T-F128A-N242D; A037T-S039E-S099R-T114Q-S126T-F128A-N242D; A037T-S039E-T056Y-S099R-S126T-F128A-N242D; A037T-S039E-T056Y-N085S-E087D-S099R-T114Q-F128A; A037T-S039E-A047V-T056Y-S099R-T114Q-S126T-F128A; A037T-S039E-A047V-T056Y-S099R-T114A-S126T-F128A; A037T-S039E-T056Y-S099R-T114Q-S126T-F128A-N242D; S039E-A047V-E087D-S099R-T114Q-S126T-F128A-N242D; S039E-A047V-T056Y-N085S-E087D-S099R-T114Q-S126T-F128A; S039E-A047V-I080V-E087D-S099R-T114A-S126T-F128A-N242D; A037T-S039E-T056Y-N085S-S099R-T114Q-S126T-F128A-N242D; S039E-A047V-T056Y-E087D-S099R-T114A-S126T-F128A-N242D; S039E-A047V-T056Y-E087D-S099R-T114Q-S126T-F128A-N242D; A037T-S039E-A047V-I080V-N085S-E087D-S099R-T114A-S126T-F128A; A037T-S039E-I080V-N085S-E087D-S099R-T114Q-S126T-F128A-N242D; A037T-S039E-T056Y-N085S-E087D-S099R-T114A-S126T-F128A-N242D; A037T-S039E-A047V-T056Y-E087D-S099R-T114A-S126T-F128A-N242D; and combinations thereof.

11. The isolated subtilisin variant of any preceding claim, wherein said variant further comprises a DXGIXXHSDLXXXGGASXXXXXPTTADLNXHGTH (SEQ ID NO:71) or DXGIXXHSDLXXXGGASXXXXXXTTADLXXHGTH (SEQ ID NO:72) motif, wherein the initial D is the active site Aspartic acid residue, the penultimate H is the active site Histidine, and X is any amino acid.
